(19) United States
(12) Patent Application Publication
(10) Pub. No.: US 2024/0059688 A1
(43) Pub. Date: Feb. 22, 2024

(54) CONOLIDINE ANALOGUES AS SELECTIVE ACKR3 MODULATORS FOR THE TREATMENT OF CANCER

(71) Applicants: Luxembourg Institute of Health (LIH), Strassen (LU); Research Triangle Institute (RTI International), Durham, NC (US)

(72) Inventors: Andy CHEVIGNÉ, Saint Léger (BE); Martyna SZPAKOWSKA, Luxembourg (LU); Ojas NAMJOSHI, Campbell, CA (US); Bruce Edward BLOUGH, Raleigh, NC (US); Ann Marie DECKER, Durham, NC (US); Max Marc Roger MEYRATH, Leudelange (LU)

(73) Assignees: Luxembourg Institute of Health (LIH), Strassen (LU); Research Triangle Institute (RTI International), Durham, NC (US)

(21) Appl. No.: 18/258,873

(22) PCT Filed: Dec. 22, 2021
(86) PCT No.: PCT/EP2021/087174

§ 371 Date: Jun. 22, 2023

(30) Foreign Application Priority Data
Dec. 22, 2020 (EP) 20216606.2

(51) Int. Cl. C07D 471/18; C07D 401/06; C07D 487/04; C07D 403/06; C07D 209/14; C07D 403/12; G01N 33/68; G01N 33/58; A61P 25/04

(52) U.S. Cl. CPC C07D 471/18; C07D 401/06; C07D 487/04; C07D 403/06; C07D 209/14; C07D 403/12; G01N 33/6893; G01N 33/58; A61P 25/04; G01N 2800/52

(57) ABSTRACT

The present application discloses compounds of e.g. formulae (2), (1A), (1B) or (1C) as selective atypical chemokine receptor 3 (ACKR3) modulators for the treatment of e.g. cancer, atherosclerotic vascular disease, cardiovascular diseases, fibrosis (e.g. cardiac fibrosis), inflammatory or autoimmune diseases and conditions, conditions of excessive or abnormal vascularization (e.g. wound healing), stem cell differentiation and mobilization disorders, brain and neuronal dysfunctions (e.g. Alzheimer's disease, multiple sclerosis and demyelinating diseases), kidney dysfunction, renal dysfunction, preeclampsia, human immunodeficiency virus (HIV) infection and obesity. Further provided are said compounds for use in methods for in vitro or ex vivo diagnosis, prediction, prognosis and/or monitoring of a disease or condition characterized by an aberrant level of ACKR3 polypeptide, as well as for use in in vitro methods for identifying an agent useful as a therapeutic. An exemplary compound is e.g. WW-1.

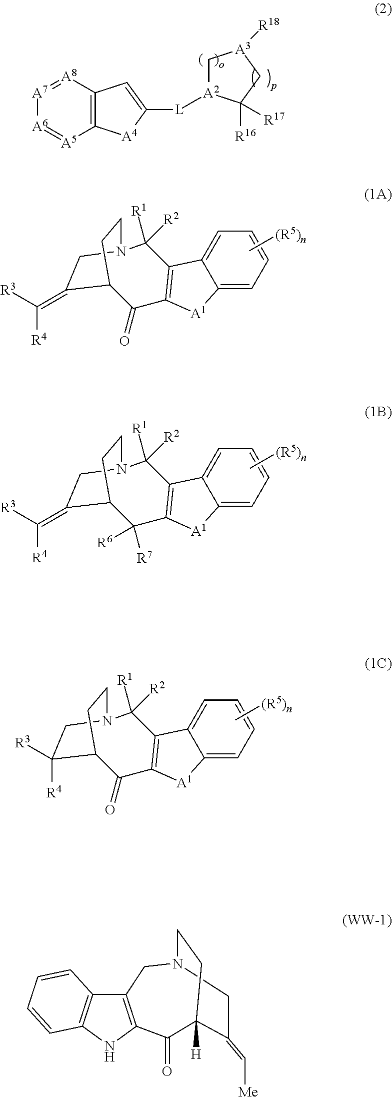

### FIELD

The invention is broadly in the medical field, and provides novel atypical chemokine receptor 3 (ACKR3) modulating molecules useful in different fields including diagnosis and therapy, both as such and fused with other agents, and further provides methods and uses of said ACKR3 modulating molecules.

### BACKGROUND

Opioid receptors are G protein-coupled receptors (GPCRs) expressed by the central nervous system and immune cells that play a central role in modulating analgesia, reward processing, as well as stress, anxiety or depression. The family of opioid receptors consists of three classical receptors: mu (μ or MOR), delta (δ or DOR), kappa (κ or KOR); and the non-classical nociceptin receptor (NOP, or orphanin FQ receptor).

All endogenous opioid peptides derive from proteolytic cleavage of large protein precursors and are mainly produced in the central nervous system (CNS), but also in the adrenal and pituitary gland and by several types of immune cells. With some exceptions, these ligands trigger downstream signalling responses via G proteins, which is followed by β-arrestin recruitment, leading to receptor desensitization and internalization. Opioid receptors can also be modulated by non-peptide opioids such as morphine, fentanyl or naloxone. Opioid receptors represent attractive targets for pharmaceuticals and opioid receptor modulators remain the most widely used analgesics in the clinic. However, the use of these medicaments is often associated with tolerance, dependence and various adverse effects (e.g. respiratory depression) or misuse.

Opioid receptor expression, signalling and desensitization are furthermore influenced by their interactions with other GPCRs, notably chemokine receptors. Chemokine receptors bind to chemokines, which are small (8-14 kDa) secreted chemo-attractant cytokines, chemokines, regulating cellular processes like migration, adhesion and growth and thereby playing a crucial role in inflammatory and developmental processes. To date, nearly 50 chemokines and 20 classical receptors have been identified in humans. Similar to opioid receptor-ligand network, many chemokine receptors recognize multiple chemokines, and, vice versa, many chemokines activate more than one receptor. Recently, a new family, called atypical chemokine receptors (ACKRs), has emerged as small subgroup of chemokine receptors. ACKRs bind chemokines without triggering G protein signalling but instead participate in chemotactic events by transporting or capturing the chemokines or internalizing and degrading the ligands in order to resolve inflammatory processes or to shape appropriate chemokine gradients.

ACKR3, formerly CXCR7, is expressed in various cells such as B and T lymphocytes, neurons and endothelial cells and plays a crucial role in many processes including cardiovascular and neuronal development as well as in migration and homing of hematopoietic stem/progenitor cells. An increasing number of studies point to the involvement of ACKR3 in cardiovascular diseases and in many cancers. ACKR3 is expressed in various cancer cell types as well as on tumour-associated vasculature and accumulating evidence demonstrates its involvement in metastasis development. ACKR3 was also shown to be upregulated upon infection by several cancer-inducing viruses including HHV-8, EBV, HTLV-1 and to play an important role in cell transformation and proliferation. Due to its unusual biology, it has recently been classified as an atypical chemokine receptor. Indeed, ACKR3 binds two endogenous chemokines, C-X-C motif chemokine 12 (CXCL12) and C-X-C motif chemokine 11 (CXCL11), which are also recognized by C-X-C motif chemokine receptor 4 (CXCR4) and C-X-C motif chemokine receptor 3 (CXCR3), respectively but unlike conventional chemokine receptors, ACKR3 does not activate the canonical G protein pathways and is proposed to trigger β-arrestin-dependent signalling. In addition, through its continuous cycling between the plasma membrane and endosomal compartments and its capacity to efficiently internalise and degrade chemokines, ACKR3 functions as a scavenger receptor regulating the availability of CXCL12 and CXCL11 for CXCR4 and CXCR3. Moreover, ACKR3 was proposed to modulate the activity of CXCR4 by forming heterodimers or competing for intracellular effector proteins involved in signal transduction.

In view of the above, there is an urgent need to explore new ways to modulate disorders involving ACKR3.

### SUMMARY

Present inventors have identified and generated chemical compounds capable of binding the chemokine receptor ACKR3.

It was previously demonstrated that modulation of ACKR3 can alter levels of endogenous opioid peptides in the treatment of disorders linked with endogenous opioid peptide dysregulation, like distress dysfunction diseases or conditions such as depression or chronic pain, with a potentially improved safety profile.

To this end, present inventors have identified and developed selective ACKR3 modulators. On the one hand it was found that conolidine and conolidine analogues are capable of activating ACKR3, which provides additional proof of the correlation between ACKR3 and pain modulation. Thus, in addition to their previously described effect as analgesics conolidine and its analogues can be applied for other diseases and disorders modulated through ACKR3, such as treatment of distress dysfunction diseases or conditions, cancers, atherosclerotic vascular disease, cardiovascular diseases, fibrosis (e.g. cardiac fibrosis), inflammatory or autoimmune diseases and conditions, conditions of excessive or abnormal vascularization (e.g. wound healing), stem cell differentiation and mobilization disorders, brain and neuronal dysfunctions (e.g. Alzheimer's disease, multiple sclerosis and demyelinating diseases), kidney dysfunction, renal dysfunction, preeclampsia and obesity. The present inventors have further developed novel conolidine analogues, which can also be applied for this purpose.

Additionally, the present inventors have generated chemical compounds which are structurally different from conolidine, but also potent activators of ACKR3. While peptide ACKR3 modulators have been developed previously (such as in WO2020225070), peptide drugs often have undesirable physicochemical properties, such as variable solubility, low bioavailability and limited stability making systemic delivery difficult. Moreover, small molecules have a much lower risk of immunogenic side effects and have an important cost advantage. These ACKR3 modulators are useful in the treatment of pain and ACKR3 mediated diseases and disorders including distress dysfunction diseases or conditions, cancers, atherosclerotic vascular disease, cardiovascular diseases, fibrosis (e.g. cardiac fibrosis), inflammatory or autoimmune diseases and conditions, conditions of excessive or abnormal vascularization (e.g. wound healing), stem cell differentiation and mobilization disorders, brain and neuronal dysfunctions (e.g. Alzheimer's disease, multiple sclerosis and demyelinating diseases), kidney dysfunction, renal dysfunction, preeclampsia and obesity.

Accordingly, the invention provides novel selective ACKR3 modulators. More particularly, the invention provides compounds of formula (2); or a stereoisomer, enantiomer, racemic, thereof

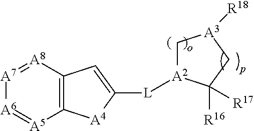

wherein,
o is an integer selected from 0, 1, 2 or 3;
p is an integer selected from 0, 1, 2, 3 or 4;
A² is selected from N or CR¹⁹;
A³ is selected from N or CR²¹;
A⁴ is selected from NR¹¹, O, S or CR²⁴ R²⁵;
A⁵ is selected from N or CR¹²;
A⁶ is selected from N or CR¹³;
A⁷ is selected from N or CR¹⁴;
A⁸ is selected from N or CR¹;
wherein at least one of A² or A³ is N;
wherein at most one of A⁵ to A⁸ is N;
L is selected from —C═O, —C(O)—NH—, and CHR²¹;
R¹¹ is selected from the group consisting of hydrogen, deuterium, alkyl, —S(O)₂R²², aryl, —S(O)R²², —SO₂NR²²R²³; and wherein said alkyl or aryl can be unsubstituted or substituted with one or more Z¹;
R¹² is selected from the group consisting of hydrogen, deuterium, halogen, —OR²³, cyano, —C(O)R²³, alkyl, trifluoromethyl, trifluoromethoxy, cycloalkyl, aryl, heterocyclyl, heteroaryl, arylalkyl, and nitro, wherein said alkyl, cycloalkyl, aryl, heterocyclyl, heteroaryl, or arylalkyl is substituted by one or more Z^{1a};
R¹³ is selected from the group consisting of hydrogen, deuterium, halogen, —OR²³, cyano, —C(O)R²³, alkyl, trifluoromethyl, trifluoromethoxy, cycloalkyl, aryl, heterocyclyl, heteroaryl, arylalkyl, and nitro, wherein said alkyl, cycloalkyl, aryl, heterocyclyl, heteroaryl, or arylalkyl is substituted by one or more Z^{1b};
R¹⁴ is selected from the group consisting of hydrogen, deuterium, halogen, —OR²³, cyano, —C(O)R², alkyl, trifluoromethyl, trifluoromethoxy, cycloalkyl, aryl, heterocyclyl, heteroaryl, arylalkyl, and nitro, wherein said alkyl, cycloalkyl, aryl, heterocyclyl, heteroaryl, or arylalkyl is substituted by one or more Z^{1c};
R¹⁵ is selected from the group consisting of hydrogen, deuterium, halogen, —OR²³, cyano, —C(O)R², alkyl, trifluoromethyl, trifluoromethoxy, cycloalkyl, aryl, heterocyclyl, heteroaryl, arylalkyl, and nitro, wherein said alkyl, cycloalkyl, aryl, heterocyclyl, heteroaryl, or arylalkyl is substituted by one or more Z^{1d};
R¹⁶ is selected from the group consisting of hydrogen, deuterium, alkyl, halogen, and —OR²³;
R¹⁷ is selected from the group consisting of hydrogen, deuterium, alkyl, halogen, and —OR²³;
or R¹⁶ and R¹⁷ together with the carbon atom to which they are attached from a group selected from —C═CH-alkyl, —C═N—OH, —C═N—O—Si(CH₃)₂C(CH₃)₃, or a saturated or unsaturated 3-, 4-, 5-, 6- or 7-membered ring;
R¹⁸ is selected from the group consisting of halogen, —NH₂, —NHR²², alkyl, deuterium, arylalkyl, —S(O)₂R²², —C(O)OR²³, —S(O)R²², —(CH₂)₂—O—(CH₂)₂—NH₂, heteroaryl, cycloalkyl, aryl, and heterocyclyl; and wherein said alkyl, arylalkyl, heteroaryl, cycloalkyl, aryl, heterocyclyl, or arylalkyl can be unsubstituted or substituted with one or more Z².
R¹⁹ is selected from the group consisting of hydrogen, alkyl, halogen, and —OR²³;
R²⁰ is selected from the group consisting of hydrogen, alkyl, halogen, and —OR²³;
R²¹ is selected from the group consisting of —OH, —COOR²³, —C(O)NH₂, hydrogen, and —OR²³;
each R²² is independently selected from the group consisting of alkyl, aryl, tolyl, cycloalkyl, arylalkyl, heterocyclyl, and heteroaryl;
each R²³ is independently selected from the group consisting of hydrogen, alkyl, aryl, cycloalkyl, arylalkyl, heterocyclyl, and heteroaryl;
R²⁴ is selected from the group consisting of hydrogen, deuterium, halogen, —OR²³ cyano, —C(O)R²³, alkyl, trifluoromethyl, trifluoromethoxy, cycloalkyl, aryl, heterocyclyl, heteroaryl, arylalkyl, and nitro;
R²⁵ is selected from the group consisting of hydrogen, deuterium, halogen, —OR²³, cyano, —C(O)R²³, alkyl, trifluoromethyl, trifluoromethoxy, cycloalkyl, aryl, heterocyclyl, heteroaryl, arylalkyl, and nitro;
each Z¹ is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z^{1a} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z^{1b} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z^{1c} is independently selected from the group consisting of —OR²³, halogen, alkyl, —NH₂, —NR²², —COOR²³, cycloalkyl, trifluoromethyl, trifluoromethoxy, aryl, arylalkyl, heterocyclyl, heteroaryl, —OH, cyano and nitro;
each Z^{1d} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z² is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
or ethyl {(3Z)-3-({[tert-butyl(dimethyl)silyl]oxy}imino-1-[(4-methylphenyl)sulfonyl]piperidin-4-yl}(3-formyl-1H-indol-2-yl)acetate;
or ethyl (3-formyl-1H-indol-2-yl){(3Z)-3-(hydroxyamino)-1-[(4-methylphenyl)sulfonyl]piperidin-4-yl}acetate;
or a solvate, hydrate, pharmaceutically acceptable salt or prodrug thereof,
and with the proviso that when A³ is N, R¹⁸ is not methyl, p-methoxy-benzyl or phenyl sulfone;
and with the proviso that the said compound is not
(5-chloro-1H-indol-2-yl)-(4-methylpiperazin-1-yl)methanone;
(7-amino-5-chloro-1H-indol-2-yl)-(4-methylpiperazin-1-yl)methanone; or
(5-chloro-7-methyl-1H-indol-2-yl)-(3-pyrrolidin-1-ylazetidin-1-yl)methanone.

The invention also provides compounds of formula (2) as described hereinabove; or a stereoisomer, enantiomer, racemic, thereof
wherein,
o is an integer selected from 0, 1, 2 or 3;
p is an integer selected from 0, 1, 2, 3 or 4;
A² is selected from N or CR¹⁹;
A³ is selected from N or CR²⁰;
A⁴ is selected from NR¹¹, O, S or CR²⁴ R²⁵;
A⁵ is selected from N or CR¹²;
A⁶ is selected from N or CR¹³;
A⁷ is selected from N or CR¹⁴;
A⁸ is selected from N or CR¹⁵;
wherein at least one of A² or A³ is N;
wherein at most one of A⁵ to A⁸ is N;
L is selected from —C═O, —C(O)—NH—, and CHR²¹;
R¹¹ is selected from the group consisting of hydrogen, deuterium, alkyl, —S(O)₂R²², aryl, —S(O)R²², —SO₂NR²²R²³; and wherein said alkyl or aryl can be unsubstituted or substituted with one or more Z¹;
R¹² is selected from the group consisting of hydrogen, deuterium, halogen, —OR²³, cyano, —C(O)R²³, alkyl, trifluoromethyl, trifluoromethoxy, cycloalkyl, aryl, heterocyclyl, heteroaryl, arylalkyl, and nitro, wherein said alkyl, cycloalkyl, aryl, heterocyclyl, heteroaryl, or arylalkyl is substituted by one or more Z^{1a};
R¹³ is selected from the group consisting of hydrogen, deuterium, halogen, —OR²³, cyano, —C(O)R²³, alkyl, trifluoromethyl, trifluoromethoxy, cycloalkyl, aryl, heterocyclyl, heteroaryl, arylalkyl, and nitro, wherein said alkyl, cycloalkyl, aryl, heterocyclyl, heteroaryl, or arylalkyl is substituted by one or more Z^{1b};
R¹⁴ is selected from the group consisting of hydrogen, deuterium, halogen, —OR²³, cyano, —C(O)R²³ alkyl, trifluoromethyl, trifluoromethoxy, cycloalkyl, aryl, heterocyclyl, heteroaryl, arylalkyl, and nitro, wherein said alkyl, cycloalkyl, aryl, heterocyclyl, heteroaryl, or arylalkyl is substituted by one or more Z^{1c};
R¹⁵ is selected from the group consisting of hydrogen, deuterium, halogen, —OR²³, cyano, —C(O)R²³ alkyl, trifluoromethyl, trifluoromethoxy, cycloalkyl, aryl, heterocyclyl, heteroaryl, arylalkyl, and nitro, wherein said alkyl, cycloalkyl, aryl, heterocyclyl, heteroaryl, or arylalkyl is substituted by one or more Z^{1d}
R¹⁶ is selected from the group consisting of hydrogen, deuterium, alkyl, halogen, and —OR²³;
R¹⁷ is selected from the group consisting of hydrogen, deuterium, alkyl, halogen, and —OR²³;
or R¹⁶ and R¹⁷ together with the carbon atom to which they are attached from a group selected from —C═CH-alkyl, —C═N—OH, —C═N—O—Si(CH₃)₂C(CH₃)₃, or a saturated or unsaturated 3-, 4-, 5-, 6- or 7-membered ring;
R¹⁸ is selected from the group consisting of halogen, —NH₂, —NHR²², alkyl, deuterium, arylalkyl, —S(O)₂R²², —C(O)OR²³, —S(O)R²², —(CH₂)₂—O—(CH₂)₂—NH₂, heteroaryl, cycloalkyl, aryl, and heterocyclyl; and wherein said alkyl, arylalkyl, heteroaryl, cycloalkyl, aryl, heterocyclyl, or arylalkyl can be unsubstituted or substituted with one or more Z²;
R¹⁹ is selected from the group consisting of hydrogen, alkyl, halogen, and —OR²³;
R²⁰ is selected from the group consisting of hydrogen, alkyl, halogen, and —OR²³;
R²¹ is selected from the group consisting of —OH, —COOR²³, —C(O)NH₂, hydrogen, and —OR²³;
each R²² is independently selected from the group consisting of alkyl, aryl, tolyl, cycloalkyl, arylalkyl, heterocyclyl, and heteroaryl;
each R²³ is independently selected from the group consisting of hydrogen, alkyl, aryl, cycloalkyl, arylalkyl, heterocyclyl, and heteroaryl;
R²⁴ is selected from the group consisting of hydrogen, deuterium, halogen, —OR²³, cyano, —C(O)R²³, alkyl, trifluoromethyl, trifluoromethoxy, cycloalkyl, aryl, heterocyclyl, heteroaryl, arylalkyl, and nitro;
R²⁵ is selected from the group consisting of hydrogen, deuterium, halogen, —OR²³, cyano, —C(O)R²³, alkyl, trifluoromethyl, trifluoromethoxy, cycloalkyl, aryl, heterocyclyl, heteroaryl, arylalkyl, and nitro;
each Z¹ is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z^{1a} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z^{1b} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z^{1c} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z^{1d} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z² is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
or a solvate, hydrate, pharmaceutically acceptable salt or prodrug thereof, with the proviso that when A³ is C, then R¹⁸ is not alkyl or benzyl;
with the proviso that when A² is N, then R¹⁴ is not chloro, methyl or trifluoromethyl;
with the proviso that when L is —C(O)—NH—, then R¹⁵ is not bromo, —OR²³, phenyl or pyridyl; and with the proviso that when A³ is N, then R¹⁸ is not methyl, p-methoxy-benzyl, phenyl sulfone or diphenylmethyl;
and with the proviso that the said compound is not
(5-chloro-1H-indol-2-yl)-(4-methylpiperazin-1-yl)methanone;
(7-amino-5-chloro-1H-indol-2-yl)-(4-methylpiperazin-1-yl)methanone;
(5-chloro-7-methyl-1H-indol-2-yl)-(3-pyrrolidin-1-ylazetidin-1-yl)methanone;
tert-butyl 4-(1H-indole-2-carbonyl)piperazine-1-carboxylate;
tert-butyl 4-(1-methylindole-2-carbonyl)piperazine-1-carboxylate;
1H-indol-2-yl-[4-(1-phenylethyl)piperazin-1-yl]methanone;
(1-methylindol-2-yl)-[4-(1-phenylethyl)piperazin-1-yl]methanone;
[4-(1,3-benzodioxol-5-ylmethyl)piperazin-1-yl]-(1H-indol-2-yl)methanone;
[4-(2-hydroxy-2-methyl-propyl)piperazin-1-yl]-(5-methoxy-1H-indol-2-yl)methanone;
4-benzo[1,2,5]oxadiazol-5-yl-1H-indole-2-carboxylic acid [1-(2-azepan-1-yl-ethyl)-piperidin-4-yl]-amide;
4-benzo[1,3]dioxol-5-yl-1H-indole-2-carboxylic acid [1-(2-azepan-1-yl-ethyl)-piperidin-4-yl]-amide;
4-hydroxy-1H-indole-2-carboxylic acid [1-(2-azepan-1-yl-ethyl)-piperidin-4-yl]-amide;
Etyl 1-(1H-indole-2-carbonyl)piperidine-4-carboxylate; or
1-(1H-indole-2-carbonyl)piperidine-4-carboxylic acid.

The invention also provides a compound selected from the group consisting of ethyl 1-(1H-indol-3-ylmethyl)piperidine-4-carboxylate; and ethyl 3-[(4-methoxycarbonyl-1-piperidyl)methyl]-1H-indole-2-carboxylate.

A further and related aspect of the invention relates to methods of treatment which involve administrating the newly identified compounds of formula (2) as described herein or a compound selected from ethyl 1-(1H-indol-3-ylmethyl)piperidine-4-carboxylate and ethyl 3-[(4-methoxycarbonyl-1-piperidyl)methyl]-1H-indole-2-carboxylate. The compounds as described herein can be used in a method of treatment of pain and treatment of distress dysfunction diseases or conditions, cancers, atherosclerotic vascular disease, cardiovascular diseases, fibrosis (e.g. cardiac fibrosis), inflammatory or autoimmune diseases and conditions, conditions of excessive or abnormal vascularization (e.g. wound healing), stem cell differentiation and mobilization disorders, brain and neuronal dysfunctions (e.g. Alzheimer's disease, multiple sclerosis and demyelinating diseases), kidney dysfunction, renal dysfunction, preeclampsia and obesity in a subject and involve administering said compound to a subject in need thereof.

More particularly, the invention provides the compounds of formula (2) as detailed herein or a compound selected from ethyl 1-(1H-indol-3-ylmethyl)piperidine-4-carboxylate and ethyl 3-[(4-methoxycarbonyl-1-piperidyl)methyl]-1H-indole-2-carboxylate; for use as a medicament. In particular embodiments, the invention provides a compound of formula (2) as described herein, or a compound selected from ethyl 1-(1H-indol-3-ylmethyl)piperidine-4-carboxylate and ethyl 3-[(4-methoxycarbonyl-1-piperidyl)methyl]-1H-indole-2-carboxylate for use in the treatment of pain and treatment of distress dysfunction diseases or conditions, cancers, atherosclerotic vascular disease, cardiovascular diseases, fibrosis (e.g. cardiac fibrosis), inflammatory or autoimmune diseases and conditions, conditions of excessive or abnormal vascularization (e.g. wound healing), stem cell differentiation and mobilization disorders, brain and neuronal dysfunctions (e.g. Alzheimer's disease, multiple sclerosis and denyelinating diseases), kidney dysfunction, renal dysfunction, preeclampsia and obesity in a subject.

A further and related aspect of the invention provides pharmaceutical compositions comprising one or more of the compounds of formula (2) as described herein, or a compound selected from ethyl 1-(1H-indol-3-ylmethyl)piperidine-4-carboxylate and ethyl 3-[(4-methoxycarbonyl-1-piperidyl)methyl]-1H-indole-2-carboxylate and a pharmaceutically acceptable carrier.

A further aspect provides compounds of formula (2) or a stereoisomer, enantiomer, racemic, thereof

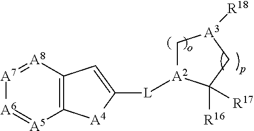

wherein,
o is an integer selected from 0, 1, 2 or 3;
p is an integer selected from 0, 1, 2, 3 or 4;
A² is selected from N or CR¹⁹;
A³ is selected from N or CR²⁰;
A⁴ is selected from NR¹¹, O, S or CR²⁴ R²⁵;
A⁵ is selected from N or CR¹²;
A⁶ is selected from N or CR¹³;
A⁷ is selected from N or CR¹⁴;
A⁸ is selected from N or CR¹⁵;
wherein at least one of A² or A³ is N;
wherein at most one of A⁵ to A⁸ is N;
L is selected from —C═O, —C(O)—NH—, and CHR²¹;
R¹¹ is selected from the group consisting of hydrogen, deuterium, alkyl, —S(O)₂R²², aryl, —S(O)R²², —SO₂NR²²R²³; and wherein said alkyl or aryl can be unsubstituted or substituted with one or more Z¹;
R¹² is selected from the group consisting of hydrogen, deuterium, halogen, —OR²³, cyano, —C(O)R²³ alkyl, trifluoromethyl, trifluoromethoxy, cycloalkyl, aryl, heterocyclyl, heteroaryl, arylalkyl, and nitro, wherein said alkyl, cycloalkyl, aryl, heterocyclyl, heteroaryl, or arylalkyl is substituted by one or more Z^{1a};
R¹³ is selected from the group consisting of hydrogen, deuterium, halogen, —OR²³, cyano, —C(O)R³, alkyl, trifluoromethyl, trifluoromethoxy, cycloalkyl, aryl, heterocyclyl, heteroaryl, arylalkyl, and nitro, wherein said alkyl, cycloalkyl, aryl, heterocyclyl, heteroaryl, or arylalkyl is substituted by one or more Z^{1b};
R¹⁴ is selected from the group consisting of hydrogen, deuterium, halogen, —OR²³, cyano, —C(O)R², alkyl, trifluoromethyl, trifluoromethoxy, cycloalkyl, aryl, heterocyclyl, heteroaryl, arylalkyl, and nitro, wherein said alkyl, cycloalkyl, aryl, heterocyclyl, heteroaryl, or arylalkyl is substituted by one or more Z^{1c};
R¹⁵ is selected from the group consisting of hydrogen, deuterium, halogen, —OR²³, cyano, —C(O)R², alkyl, trifluoromethyl, trifluoromethoxy, cycloalkyl, aryl, heterocyclyl, heteroaryl, arylalkyl, and nitro, wherein said alkyl, cycloalkyl, aryl, heterocyclyl, heteroaryl, or arylalkyl is substituted by one or more Z^{1d}
R¹⁶ is selected from the group consisting of hydrogen, deuterium, alkyl, halogen, and —OR²³;
R¹⁷ is selected from the group consisting of hydrogen, deuterium, alkyl, halogen, and —OR²³;
or R¹⁶ and R¹⁷ together with the carbon atom to which they are attached from a group selected from —C═CH-alkyl, —C═N—OH, —C═N—O—Si(CH₃)₂C(CH₃)₃, or a saturated or unsaturated 3-, 4-, 5-, 6- or 7-membered ring;
R¹⁸ is selected from the group consisting of hydrogen, deuterium, halogen, —NH₂, —NHR²², alkyl, arylalkyl, —S(O)₂R²², —C(O)OR²³, —S(O)R²², —(CH₂)₂—O—(CH₂)₂—NH₂, heteroaryl, cycloalkyl, aryl, and heterocyclyl; and wherein said alkyl, arylalkyl, heteroaryl, cycloalkyl, aryl, heterocyclyl, or arylalkyl can be unsubstituted or substituted with one or more Z;
R¹⁹ is selected from the group consisting of hydrogen, alkyl, halogen, and —OR²³;
R²⁰ is selected from the group consisting of hydrogen, alkyl, halogen, and —OR²³;
R²¹ is selected from the group consisting of —OH, —COOR²³, —C(O)NH, hydrogen, and —OR²³;
each R²² is independently selected from the group consisting of alkyl, aryl, tolyl, cycloalkyl, arylalkyl, heterocyclyl, and heteroaryl;
each R²³ is independently selected from the group consisting of hydrogen, alkyl, aryl, cycloalkyl, arylalkyl, heterocyclyl, and heteroaryl;
R²⁴ is selected from the group consisting of hydrogen, deuterium, halogen, —OR²³ cyano, —C(O)R³, alkyl, trifluoromethyl, trifluoromethoxy, cycloalkyl, aryl, heterocyclyl, heteroaryl, arylalkyl, and nitro;
R²⁵ is selected from the group consisting of hydrogen, deuterium, halogen, —OR²³, cyano, —C(O)R²³, alkyl, trifluoromethyl, trifluoromethoxy, cycloalkyl, aryl, heterocyclyl, heteroaryl, arylalkyl, and nitro;
each Z¹ is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z^{1a} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z^{1b} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z^{1c} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z^{1d} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z² is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
or ethyl {(3Z)-3-({[tert-butyl(dimethyl)silyl]oxy}imino-1-[(4-methylphenyl)sulfonyl]piperidin-4-yl}(3-formyl-1H-indol-2-yl)acetate;
or ethyl (3-formyl-1H-indol-2-yl){(3Z)-3-(hydroxyamino)-1-[(4-methylphenyl)sulfonyl]piperidin-4-yl}acetate;
or a solvate, hydrate, pharmaceutically acceptable salt or prodrug thereof;
for use in the treatment of pain and treatment of distress dysfunction diseases or conditions, cancers, atherosclerotic vascular disease, cardiovascular diseases, fibrosis (e.g. cardiac fibrosis), inflammatory or autoimmune diseases and conditions, conditions of excessive or abnormal vascularization (e.g. wound healing), stem cell differentiation and mobilization disorders, brain and neuronal dysfunctions (e.g. Alzheimer's disease, multiple sclerosis and demyelinating diseases), kidney dysfunction, renal dysfunction, preeclampsia, human immunodeficiency virus (HIV) infection and obesity in a subject. Yet another aspect provides compounds of formula (2) as described hereinabove or a stereoisomer, enantiomer, racemic, thereof
wherein,
o is an integer selected from 0, 1, 2 or 3;
p is an integer selected from 0, 1, 2, 3 or 4;
A² is selected from N or CR¹⁹;
A³ is selected from N or CR²⁰;
A⁴ is selected from NR¹¹, O, S or CR²⁴ R²⁵;
A⁵ is selected from N or CR¹²;
A⁶ is selected from N or CR¹³
A⁷ is selected from N or CR¹⁴;
A⁸ is selected from N or CR¹⁵;
wherein at least one of A² or A³ is N;
wherein at most one of A⁵ to A⁸ is N;
L is selected from —C═O, —C(O)—NH—, and CHR²¹;
R¹¹ is selected from the group consisting of hydrogen, deuterium, alkyl, —S(O)₂R²², aryl, —S(O)R²², —SO₂NR²²R²³; and wherein said alkyl or aryl can be unsubstituted or substituted with one or more Z¹;
R¹² is selected from the group consisting of hydrogen, deuterium, halogen, —OR²³, cyano, —C(O)R²³, alkyl, trifluoromethyl, trifluoromethoxy, cycloalkyl, aryl, heterocyclyl, heteroaryl, arylalkyl, and nitro, wherein said alkyl, trifluoromethyl, trifluoromethoxy, cycloalkyl, aryl, heterocyclyl, heteroaryl, or arylalkyl is substituted by one or more Z¹;
R¹³ is selected from the group consisting of hydrogen, deuterium, halogen, —OR², cyano, —C(O)R², alkyl, trifluoromethyl, trifluoromethoxy, cycloalkyl, aryl, heterocyclyl, heteroaryl, arylalkyl, and nitro, wherein said alkyl, cycloalkyl, aryl, heterocyclyl, heteroaryl, or arylalkyl is substituted by one or more Z^{1b};
R¹⁴ is selected from the group consisting of hydrogen, deuterium, halogen, —OR²³, cyano, —C(O)R²³, alkyl, trifluoromethyl, trifluoromethoxy, cycloalkyl, aryl, heterocyclyl, heteroaryl, arylalkyl, and nitro, wherein said alkyl, cycloalkyl, aryl, heterocyclyl, heteroaryl, or arylalkyl is substituted by one or more Z^{1c};
R¹⁵ is selected from the group consisting of hydrogen, deuterium, halogen, —OR²³, cyano, —C(O)R²³, alkyl, trifluoromethyl, trifluoromethoxy, cycloalkyl, aryl, heterocyclyl, heteroaryl, arylalkyl, and nitro, wherein said alkyl, cycloalkyl, aryl, heterocyclyl, heteroaryl, or arylalkyl is substituted by one or more Z^{1d};
R¹⁶ is selected from the group consisting of hydrogen, deuterium, alkyl, halogen, and —OR²³;
R¹⁷ is selected from the group consisting of hydrogen, deuterium, alkyl, halogen, and —OR²³;
or R¹⁶ and R¹⁷ together with the carbon atom to which they are attached from a group selected from —C═CH-alkyl, —C═N—OH, —C═N—O—Si(CH₃)₂C(CH₃)₃, or a saturated or unsaturated 3-, 4-, 5-, 6- or 7-membered ring;
R¹⁸ is selected from the group consisting of hydrogen, deuterium, halogen, —NH₂, —NHR²² alkyl, arylalkyl, —S(O)₂R²², —C(O)OR²³, —S(O)R²², —(CH₂)₂—O—(CH₂)₂—NH₂, heteroaryl, cycloalkyl, aryl, and heterocyclyl; and wherein said alkyl, arylalkyl, heteroaryl, cycloalkyl, aryl, heterocyclyl, or arylalkyl can be unsubstituted or substituted with oner or more Z;
R¹⁹ is selected from the group consisting of hydrogen, alkyl, halogen, and —OR²³;
R²⁰ is selected from the group consisting of hydrogen, alkyl, halogen, and —OR²³;
R²¹ is selected from the group consisting of —OH, —COOR²³, —C(O)NH, hydrogen, and —OR²³;
each R²² is independently selected from the group consisting of alkyl, aryl, tolyl, cycloalkyl, arylalkyl, heterocyclyl, and heteroaryl;
each R²³ is independently selected from the group consisting of hydrogen, alkyl, aryl, cycloalkyl, arylalkyl, heterocyclyl, and heteroaryl;
R²⁴ is selected from the group consisting of hydrogen, deuterium, halogen, —OR²³, cyano, —C(O)R²³, alkyl, trifluoromethyl, trifluoromethoxy, cycloalkyl, aryl, heterocyclyl, heteroaryl, arylalkyl, and nitro;
R²⁵ is selected from the group consisting of hydrogen, deuterium, halogen, —OR²³, cyano, —C(O)R²³, alkyl, trifluoromethyl, trifluoromethoxy, cycloalkyl, aryl, heterocyclyl, heteroaryl, arylalkyl, and nitro;
each Z^{1a} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z^{1b} is independently selected from the group consisting of —OR²³, halogen, alkyl, —NH₂, —NR²², —COOR²³, cycloalkyl, trifluoromethyl, trifluoromethoxy, aryl, arylalkyl, heterocyclyl, heteroaryl, —OH, cyano and nitro;
each Z^{1c} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z^{1d} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z¹ is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z² is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
or a solvate, hydrate, pharmaceutically acceptable salt or prodrug thereof;
with the proviso that when A³ is N, then R¹⁸ is not diphenylmethyl;
with the proviso that when A³ is C, then R¹⁸ is not alkyl or benzyl;
with the proviso that when A² is N, then R¹⁴ is not chloro, methyl or trifluoromethyl;
with the proviso that when L is —C(O)—NH—, then R¹⁵ is not bromo, —OR²³, phenyl, pyridyl,
and with the proviso that the said compound is not
tert-butyl 4-(1H-indole-2-carbonyl)piperazine-1-carboxylate;
tert-butyl 4-(1-methylindole-2-carbonyl)piperazine-1-carboxylate;
1H-indol-2-yl-[4-(1-phenylethyl)piperazin-1-yl]methanone;
(1-methylindol-2-yl)-[4-(1-phenylethyl)piperazin-1-yl]methanone;
[4-(1,3-benzodioxol-5-ylmethyl)piperazin-1-yl]-(1H-indol-2-yl)methanone;
[4-(2-hydroxy-2-methyl-propyl)piperazin-1-yl]-(5-methoxy-1H-indol-2-yl)methanone;
4-benzo[1,2,5]oxadiazol-5-yl-1H-indole-2-carboxylic acid [1-(2-azepan-1-yl-ethyl)-piperidin-4-yl]-amide;
4-benzo[1,3]dioxol-5-yl-1H-indole-2-carboxylic acid [1-(2-azepan-1-yl-ethyl)-piperidin-4-yl]-amide; or
4-hydroxy-1H-indole-2-carboxylic acid [1-(2-azepan-1-yl-ethyl)-piperidin-4-yl]-amide;
   for use in the treatment of pain and treatment of distress dysfunction diseases or conditions, cancers, atherosclerotic vascular disease, cardiovascular diseases, fibrosis (e.g. cardiac fibrosis), inflammatory or autoimmune diseases and conditions, conditions of excessive or abnormal vascularization (e.g. wound healing), stem cell differentiation and mobilization disorders, brain and neuronal dysfunctions (e.g. Alzheimer's disease, multiple sclerosis and demyelinating diseases), kidney dysfunction, renal dysfunction, preeclampsia, human immunodeficiency virus (HIV) infection and obesity in a subject.

In a further and related aspect the present invention thus provides Conolidine and Conolidine analogues for use in the treatment of distress dysfunction diseases or conditions, cancers, atherosclerotic vascular disease, cardiovascular diseases, fibrosis (e.g. cardiac fibrosis), inflammatory or autoimmune diseases and conditions, conditions of excessive or abnormal vascularization (e.g. wound healing), stem cell differentiation and mobilization disorders, brain and neuronal dysfunctions (e.g. Alzheimer's disease, multiple sclerosis and demyelinating diseases), kidney dysfunction, renal dysfunction, preeclampsia and obesity in a subject.

More particularly, the invention provides compounds of formula (1A) (1B) or (1C); or a stereoisomer, enantiomer, racemic, thereof,

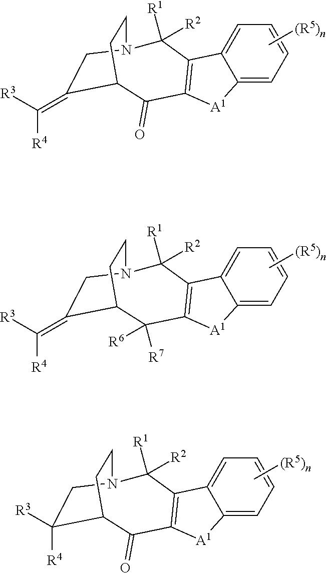

wherein,
n is an integer selected from 0, 1, 2 or 3;
A¹ is selected from the group consisting of a substituted nitrogen or carbon atom, substituents selected from the group consisting of hydrogen, deuterium, alkyl, heteroatom substituted alkyl, alkenyl, aryl, heteroaryl, cycloalkyl, heteroatom substituted cycloalkyl, S, SO, SO₂, OR⁹, NR⁹;
R¹ is selected from the group consisting of hydrogen, deuterium, alkyl, heteroatom substituted alkyl, alkenyl, aryl, heteroaryl, cycloalkyl and heteroatom substituted cycloalkyl;
R² is selected from the group consisting of hydrogen, deuterium, alkyl, heteroatom substituted alkyl, alkenyl, aryl, heteroaryl, cycloalkyl and heteroatom substituted cycloalkyl;
R³ is selected from the group consisting of hydrogen, deuterium, alkyl, heteroatom substituted alkyl, alkenyl, aryl, heteroaryl, halogen, cycloalkyl and heteroatom substituted cycloalkyl;
R⁴ is selected from the group consisting of hydrogen, deuterium, alkyl, heteroatom substituted alkyl, alkenyl, aryl, heteroaryl, halogen, and cycloalkyl and heteroatom substituted cycloalkyl;
R³ and R⁴ together with the atom to which they are attached can form a saturated or unsaturated 5-, 6-, or 7-membered ring;
R⁵ is selected from the group consisting of deuterium, halogen, alkyl, heteroatom substituted alkyl, alkenyl, aryl, heteroaryl, cycloalkyl and heteroatom substituted cycloalkyl;
R⁶ is selected from the group consisting of hydrogen, deuterium, NH₂, NR⁸R⁹, OR⁹, and R¹;
R⁷ is selected from the group consisting of hydrogen, deuterium, NH₂, NR⁸R⁹, OR⁹, and R¹;
or R⁶ and R⁷ together with the carbon atom to which they are attached from a group selected from the group consisting of —CH═CH₂, —CH═CH-alkyl, and —CH═N—OH;
R⁸ is selected from the group consisting of deuterium, alkyl, heteroatom substituted alkyl, alkenyl, aryl, heteroaryl, cycloalkyl and heteroatom substituted cycloalkyl;
R⁹ is selected from the group consisting of hydrogen, deuterium, alkyl, heteroatom substituted alkyl, alkenyl, aryl, heteroaryl, cycloalkyl and heteroatom substituted cycloalkyl;
or a solvate, hydrate, pharmaceutically acceptable salt or prodrug thereof,
for use in the treatment of distress dysfunction diseases or conditions, cancers, atherosclerotic vascular disease, cardiovascular diseases, fibrosis (e.g. cardiac fibrosis), inflammatory or autoimmune diseases and conditions, conditions of excessive or abnormal vascularization (e.g. wound healing), stem cell differentiation and mobilization disorders, brain and neuronal dysfunctions (e.g. Alzheimer's disease, multiple sclerosis and demyelinating diseases), kidney dysfunction, renal dysfunction, preeclampsia, human immunodeficiency virus (HIV) infection and obesity in a subject.

In a related aspect, the invention provides methods for the treatment of distress dysfunction diseases or conditions, cancers, atherosclerotic vascular disease, cardiovascular diseases, fibrosis (e.g. cardiac fibrosis), inflammatory or autoimmune diseases and conditions, conditions of excessive or abnormal vascularization (e.g. wound healing), stem cell differentiation and mobilization disorders, brain and neuronal dysfunctions (e.g. Alzheimer's disease, multiple sclerosis and demyelinating diseases), kidney dysfunction, renal dysfunction, preeclampsia, human immunodeficiency virus (HIV) infection and obesity in a subject, which involve, administering to said subject the compounds of formula (1A) (1B) or (1C); or a stereoisomer, enantiomer, racemic, thereof.

A further aspect provides a method for in vitro or ex vivo diagnosis, prediction, prognosis and/or monitoring of a disease or condition in a subject characterized by an aberrant level of ACKR3 polypeptide, comprising detecting said aberrant level of ACKR3 polypeptide using the compounds of the present invention. More particularly, the methods of the present invention comprise contacting a sample of said subject with a compound according to the present invention having a label and detecting the level of ACKR3 polypeptide in said sample, wherein an aberrant level of ACKR3 polypeptide in said sample is indicative of said disease or condition. More particularly, the methods of the invention comprise the steps of
obtaining a biological sample obtained from a subject,
contacting said biological sample with the compound of formula (1A), (1B), (1C), (2), or subgroups thereof, or a compound selected from ethyl 1-(1H-indol-3-ylmethyl)piperidine-4-carboxylate and ethyl 3-[(4-methoxycarbonyl-1-piperidyl)methyl]-1H-indole-2-carboxylate, wherein said compound is fused or covalently linked to a detectable label,
determining the level of ACKR3 polypeptide in said biological sample by detecting said compound, and
diagnosing, predicting, prognosing and/or monitoring the disease or condition based on the level of ACKR3 polypeptide.

The invention further provides a compound comprising the formula (1A), (1B), (1C), (2), or subgroups thereof, or a compound selected from ethyl 1-(1H-indol-3-ylmethyl)piperidine-4-carboxylate and ethyl 3-[(4-methoxycarbonyl-1-piperidyl)methyl]-1H-indole-2-carboxylate, which further comprises a label.

The invention further provides kits for diagnosing, predicting, prognosing and/or monitoring a disease or condition characterized by an aberrant level of ACKR3 polypeptide in a subject, the kit comprising:
(a) the compound according to formula (1A), (1B), (1C), (2), or subgroups thereof, or a compound selected from ethyl 1-(1H-indol-3-ylmethyl)piperidine-4-carboxylate and ethyl 3-[(4-methoxycarbonyl-1-piperidyl)methyl]-1H-indole-2-carboxylate; and
(b) a reference value of the level of ACKR3 polypeptide, wherein said reference value represents a known diagnosis, prediction and/or prognosis of the disease or condition characterized by an aberrant level of ACKR3 polypeptide.

### BRIEF DESCRIPTION OF DRAWINGS

The following detailed description, given by way of example, but not intended to limit the invention solely to the specific embodiments described, may best be understood in conjunction with the accompanying drawings.

FIG. 1 represents in Section (A) a graph plotting the logarithm of the concentration of compounds of the invention WW-1 (conolidine) and WW-12, as a function of the β-arrestin2 recruitment % max CXCL-12 in human ACKR3 barr2 receptor in U87 cells. Section (B) is a graph plotting the logarithm of the concentration of compounds of the invention WW-1 (conolidine) and WW-12 as a function of the β-arrestin1 recruitment % max CXCL-12 in human ACKR3 barr1 receptor in U87 cells. Section (C) is a graph plotting the logarithm of the concentration of compounds of the invention WW-1 (conolidine) and WW-12 as a function of the β-arrestin2 recruitment % max CXCL-12 in mouse ACKR3 barr2 receptor in U87 cells. Section (D) is a graph plotting the logarithm of the concentration of compounds of the invention WW-1 (conolidine) and WW-12, in function of the percentage of displacement of CXCL-12-AF647.

FIG. 2 represents in Section (A) a graph plotting the logarithm of the concentration of compounds of the invention WW-1 (conolidine), and WW-12, as a function of the β-arrestin2 recruitment % of Met-enkephalin in opioid receptor DOR. Section (B) is a graph plotting the logarithm of the concentration of compounds of the invention WW-1 (conolidine) and WW-12 as a function of the β-arrestin2 recruitment % of BAM-22 in opioid receptor MOR. Section (C) is a graph plotting the logarithm of the concentration of compounds of the invention WW-1 (conolidine), WW-12, as a function of the β-arrestin1 recruitment % of Dynorphin A in opioid receptor KOR. Section (D) is a graph plotting the logarithm of the concentration of compounds of the invention WW-1 (conolidine) and WW-12 as a function of the β-arrestin2 recruitment % of Nociceptin in opioid receptor NOP.

FIG. 3 represents in Section (A) a graph plotting activation of the 21 classical and 4 atypical chemokine receptors, and the 4 opioid receptors in the presence of compounds of the invention WW-1 (conolidine) and WW-12 at a concentration of 1 μM. Section (B) is a graph plotting activation of the 21 classical and 4 atypical chemokine receptors, and the 4 opioid receptors in the presence of compounds of the invention WW-1 (conolidine) and WW-12 at a concentration of 3 μM. Section (C) is a graph plotting activation of the 21 classical and 4 atypical chemokine receptors, and the 4 opioid receptors in the presence of compounds of the invention WW-1 (conolidine) and WW-12 at a concentration of 10 μM.

FIG. 4 represents in Section (A) a graph plotting the logarithm of the concentration of compounds of the invention WW-1 (conolidine) and WW-12, as a function of the β-arrestin1 recruitment % CCL-19 in ACKR4 barr1 receptors. Section (B) is a graph plotting the logarithm of the concentration of compounds of the invention WW-1 (conolidine) and WW-12 as a function of the β-arrestin1 recruitment % of CX3CL1 in CX3CR1 barr1 receptors. Section (C) is a graph plotting the logarithm of the concentration of compounds of the invention WW-1 (conolidine) and WW-12 as a function of the 3-arrestin1 recruitment % of CCL13 in CCR3 barr1 receptor.

FIG. 5 represents a graph comparing the ability of compounds of the invention WW-1 (conolidine), and WW-12 and to activate ACKR3 and the classical opioid receptors with reference synthetic molecules and approved pain medications suing NanoBiT β-arrestin1 recruitment assay.

FIG. 6 represents in Section (A) a graph plotting the internalization of ACKR3 in response to WW-1 (conolidine) and WW-12 in comparison to BAM22, and control peptide (1 μM) used as positive and negative controls monitored by flow cytometry using anti-ACKR3 mAb (clone 11G8). Section (B) is a graph plotting ACKR3 delivery to the early endosomes in response to WW-1 (conolidine) and WW12, CXCL12 (1 μM) or peptides and BAM22 (1 μM) monitored by NanoBRET-based assay in U87 cells using ACKR3-Nanoluciferase as donor and FYVE-mNeongreen as acceptor. Chemokine CXCL10 was used as negative control. *p<0.05, **p<0.01, ***p<0.001 by one-way ANOVA with Bonferroni's post hoc test.

FIG. 7 represents in Section (A) visualization of ACKR3-mediated uptake of Cy5-labeled BAM22 (250 nM) in competition with WW-1 (conolidine) and WW-12 (50 μM) visualized in U87.ACKR3 cells by imaging flow cytometry. Three representative cells per condition are shown out of 5000 single, in focus, living cells recorded. Scale bar: 10 μm. In section (B) a graph plotting the percentage of cells with a given number of distinguishable vesicle-like structures (spots) representative of three independent experiments presented in section (A). In section (C) a graph plotting ACKR3-mediated uptake of Cy5-labeled BAM22 (50 nM-1 μM) in competition with WW-1 (conolidine) (50 μM) or WW-12 (10 μM) visualized in U87.ACKR3 cells by imaging flow cytometry. In section (D) a graph plotting the uptake competition between Cy5-labeled BAM22 (250 nM) and varying concentrations of WW-1 (conolidine) and WW-12 (50, 10, 5, 1 μM) or CXCL12, BAM22 (1 μM) used as positive controls in U87.ACKR3 cells. Data are presented as mean S.E.M. of three independent experiments (n=3). *p<0.05, **p<0.01, ***p<0.001 by repeated measures one-way ANOVA with Dunnet's post hoc test (C) or one-way ANOVA with Bonferroni's post hoc test (D).

### DESCRIPTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

Where groups can be substituted, such groups may be substituted with one or more, and preferably one, two or three substituents. Preferred substituents may be selected from but not limited to, for example, the group comprising halo, hydroxyl, C₁₋₆alkyl, C₁₋₆alkoxy, trifluoromethyl, trifluoromethoxy, C₃₋₁₂cycloalkyl, C₆₋₁₂aryl, C₆₋₁₂arylC₁₋₆alkyl, heterocyclyl, heteroaryl, cyano, amino, nitro, carboxyl, and mono- or diC₁₋₆alkylamino.

The term “halo” or “halogen” as a group or part of a group is generic for fluoro, chloro, bromo, iodo.

The term “amino” refers to the group —NH₂.

The term “hydroxyl” or “hydroxy” as used herein refers to the group —OH.

The term “oxo” as used herein refers to the group ═O.

The term “nitro” as used herein refers to the group —NO₂.

The term “cyano” as used herein refers to the group —CN.

The term “carboxy” or “carboxyl” or “hydroxycarbonyl” as used herein refers to the group —CO₂H.

The term “aminocarbonyl” as used herein refers to the group —CO—NH₂.

The term “alkyl” by itself or as part of another substituent refers to a hydrocarbyl group of formula CₙH₂ₙ₊₁ wherein n is a number greater than or equal to 1. Alkyl groups may be linear or branched and may be substituted as indicated herein. Generally, alkyl groups of this invention comprise from 1 to 6 carbon atoms, preferably from 1 to 5 carbon atoms, preferably from 1 to 4 carbon atoms, more preferably from 1 to 3 carbon atoms, still more preferably 1 to 2 carbon atoms. When a subscript is used herein following a carbon atom, the subscript refers to the number of carbon atoms that the named group may contain. For example, the term “C₁₋₆alkyl”, as a group or part of a group, refers to a hydrocarbyl group of formula —CₙH₂ₙ₊₁ wherein n is a number ranging from 1 to 6. Thus, for example, “C₁₋₆alkyl” includes all linear or branched alkyl groups with between 1 and 6 carbon atoms, and thus includes methyl, ethyl, n-propyl, i-propyl, butyl and its isomers (e.g. n-butyl, i-butyl and t-butyl); pentyl and its isomers, hexyl and its isomers. For example, “C₁₋₅alkyl” includes all includes all linear or branched alkyl groups with between 1 and 5 carbon atoms, and thus includes methyl, ethyl, n-propyl, i-propyl, butyl and its isomers (e.g. n-butyl, i-butyl and t-butyl); pentyl and its isomers. For example, “C₁₋₄alkyl” includes all linear or branched alkyl groups with between 1 and 4 carbon atoms, and thus includes methyl, ethyl, n-propyl, i-propyl, butyl and its isomers (e.g. n-butyl, i-butyl and t-butyl). For example “C₁₋₃alkyl” includes all linear or branched alkyl groups with between 1 and 3 carbon atoms, and thus includes methyl, ethyl, n-propyl, i-propyl. A “substituted C₁₋₆alkyl” refers to a C₁₋₆alkyl group substituted with one or more substituent(s) (for example 1 to 3 substituent(s), for example 1, 2, or 3 substituent(s)) at any available point of attachment.

When the suffix “ene” is used in conjunction with an alkyl group, i.e. “alkylene”, this is intended to mean the alkyl group as defined herein having two single bonds as points of attachment to other groups. As used herein, the term “lkylene”, by itself or as part of another substituent, refers to C₁₋₆alkyl groups that are divalent, i.e., with two single bonds for attachment to two other groups. Alkylene groups may be linear or branched and may be substituted as indicated herein. Non-limiting examples of alkylene groups include methylene (—CH₂—), ethylene (—CH₂—CH₂—), methylmethylene (—CH(CH₃)—), 1-methyl-ethylene (—CH(CH₃)—CH₂—), n-propylene (—CH₂—CH₂—CH₂—), 2-methylpropylene (—CH₂—CH(CH₃)—CH₂—), 3-methylpropylene (—CH₂—CH₂—CH(CH₃)—), n-butylene (—CH₂—CH₂—CH₂—CH₂—), 2-methylbutylene (—CH₂—CH(CH₃)—CH₂—CH₂—), 4-methylbutylene (—CH₂—CH₂—CH₂—CH(CH₃)—), pentylene and its chain isomers, hexylene and its chain isomers.

When the term “alkyl” is used as a suffix following another term, as in “hydroxyalkyl,” this is intended to refer to an alkyl group, as defined above, being substituted with one or two (preferably one) substituent(s) selected from the other, specifically-named group, also as defined herein. The term “hydroxyC₁₋₆alkyl” therefore refers to a —R^{a}—OH group wherein R^{a} is C₁₋₆alkylene as defined herein.

The term “haloC₁₋₆alkyl” as a group or part of a group, refers to a C₁₋₆alkyl group having the meaning as defined above wherein one, two, or three hydrogen atoms are each replaced with a halogen as defined herein. Non-limiting examples of such haloC₁₋₆alkyl groups include chloromethyl, 1-bromoethyl, fluoromethyl, difluoromethyl, trifluoromethyl, 1,1,1-trifluoroethyl, trichloromethyl, tribromomethyl, and the like.

The term “alkoxy” or “alkyloxy”, as a group or part of a group, refers to a group having the formula —OR^{b} wherein R^{b} is C₁₋₆alkyl as defined herein above. Non-limiting examples of suitable C₁₋₆alkoxy include methoxy, ethoxy, propoxy, isopropoxy, butoxy, isobutoxy, sec-butoxy, tert-butoxy, pentyloxy and hexyloxy.

The term “cycloalkyl”, as a group or part of a group, refers to a cyclic alkyl group, that is a monovalent, saturated, hydrocarbyl group having 1 or more cyclic structure, and comprising from 3 to 12 carbon atoms, more preferably from 3 to 9 carbon atoms, more preferably from 3 to 7 carbon atoms; more preferably from 3 to 6 carbon atoms. Cycloalkyl includes all saturated hydrocarbon groups containing 1 or more rings, including monocyclic or bicyclic groups. The further rings of multi-ring cycloalkyls may be either fused, bridged and/or joined through one or more spiro atoms. When a subscript is used herein following a carbon atom, the subscript refers to the number of carbon atoms that the named group may contain. For example, the term “C₃₋₈cycloalkyl”, a cyclic alkyl group comprising from 3 to 8 carbon atoms. For example, the term “C₃₋₆cycloalkyl”, a cyclic alkyl group comprising from 3 to 6 carbon atoms. Examples of C₃₋₁₂cycloalkyl groups include but are not limited to cyclopropyl, cyclobutyl, cyclopentyl, cyclohexyl, cycloheptyl, cyclooctyl, bicycle[2.2.1]heptan-2yl, (1S,4R)-norbornan-2-yl, (1R,4R)-norbornan-2-yl, (1S,4S)-norbornan-2-yl, (1R,4S)-norbornan-2-yl.

When the suffix “ene” is used in conjunction with a cycloalkyl group, i.e. cycloalkylene, this is intended to mean the cycloalkyl group as defined herein having two single bonds as points of attachment to other groups. Non-limiting examples of “C₃₋₈cycloalkylene” include 1,2-cyclopropylene, 1,1-cyclopropylene, 1,1-cyclobutylene, 1,2-cyclobutylene, 1,3-cyclopentylene, 1,1-cyclopentylene, and 1,4-cyclohexylene.

Where an alkylene or cycloalkylene group is present, connectivity to the molecular structure of which it forms part may be through a common carbon atom or different carbon atom. To illustrate this applying the asterisk nomenclature of this invention, a C₃alkylene group may be for example *—CH₂CH₂CH₂—*, *—CH(—CH₂CH₃)—* or *—CH₂CH(—CH₃)—*. Likewise a C₃cycloalkylene group may be

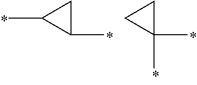

The term “cycloalkyloxy”, as a group or part of a group, refers to a group having the formula —OR wherein R is cycloalkyl as defined herein above.

As used herein, the term “alkenyl” as a group or a part of a group refers to straight or branched hydrocarbon chain containing at least one double bond. For example, the term “C₂₋₆alkenyl” means a straight or branched alkenyl containing at least two and at most 10 carbon atoms and containing at least one double bond. Multiple double bonds may be adjacent (═C═), conjugated (═C—C═), ore are non-adjacent and non-conjugated. Examples of “alkenyl” as used herein include, but are not limited to etheyl, 2-propenyl, 3-butenyl, 2-butenyl, 2-pentenyl, 3-pentenyl, 3-methyl-2-butenyl, 3-methylbut-2-enyl, 3-hexenyl, and 1,1-dimethylbut-2-enyl.

The term “aryl”, as a group or part of a group, refers to a polyunsaturated, aromatic hydrocarbyl group having a single ring (i.e. phenyl) or multiple aromatic rings fused together (e.g. naphthyl), or linked covalently, typically containing 6 to 12 atoms; preferably 6 to 10, wherein at least one ring is aromatic. The aromatic ring may optionally include one to two additional rings (either cycloalkyl, heterocyclyl or heteroaryl) fused thereto. Examples of suitable aryl include C₆₋₁₀aryl, more preferably C₆₋₁₂aryl. Non-limiting examples of C₆₋₁₂aryl comprise phenyl, biphenylyl, biphenylenyl, or 1- or 2-naphthanelyl; 1-, 2-, 3-, 4-, 5- or 6-tetralinyl (also known as “1,2,3,4-tetrahydronaphtalene); 1-, 2-, 3-, 4-, 5-, 6-, 7- or 8-azulenyl, 4-, 5-, 6 or 7-indenyl; 4- or 5-indanyl; 5-, 6-, 7- or 8-tetrahydronaphthyl; 1,2,3,4-tetrahydronaphthyl; and 1,4-dihydronaphthyl; 1-, 2-, 3-, 4- or 5-pyrenyl. A “substituted C₆₋₁₂aryl” refers to a C₆₋₁₂aryl group having one or more substituent(s) (for example 1, 2 or 3 substituent(s), or 1 to 2 substituent(s)), at any available point of attachment.

The term “heteroatom substituted alkyl” as used herein refers to an acyclic alkyl wherein one or more carbon atoms are replaced by an oxygen, nitrogen or sulphur atom, with the proviso that said chain may not contain two adjacent O atoms or two adjacent S atoms. This means that one or more —CH₃ of said acyclic alkyl can be replaced by —NH₂ and/or that one or more —CH₂— of said acyclic alkyl can be replaced by —NH—, —O— or —S—. Exemplary heteroatom substituted alkyl groups include, but are not limited to, alcohols, alkyl ethers, primary, secondary, and tertiary alkyl amines, amides, ketones, esters, and alkyl sulfides.

When the suffix “ene” is used in conjunction with an aryl group; i.e. arylene, this is intended to mean the aryl group as defined herein having two single bonds as points of attachment to other groups. Suitable “C₆₋₁₂arylene” groups include 1,4-phenylene, 1,2-phenylene, 1,3-phenylene, biphenylylene, naphthylene, indenylene, 1-, 2-, 5- or 6-tetralinylene, and the like. Where a carbon atom in an aryl group is replaced with a heteroatom, the resultant ring is referred to herein as a heteroaryl ring.

The term “aryloxy”, as a group or part of a group, refers to a group having the formula —OR^{b} wherein R^{g} is aryl as defined herein above.

The term “arylalkyl”, as a group or part of a group, means a C₁₋₆alkyl as defined herein, wherein at least one hydrogen atom is replaced by at least one C₆₋₁₂aryl as defined herein. Non-limiting examples of C₆₋₁₂arylC₁₋₆alkyl group include benzyl, phenethyl, dibenzylmethyl, methylphenylmethyl, 3-(2-naphthyl)-butyl, and the like.

The terms “heterocyclyl” or “heterocycloakyl” or “heterocyclo”, as a group or part of a group, refer to non-aromatic, fully saturated or partially unsaturated cyclic groups (for example, 3 to 7 member monocyclic, 7 to 11 member bicyclic, or comprising a total of 3 to 10 ring atoms) which have at least one heteroatom in at least one carbon atom-containing ring; wherein said ring may be fused to an aryl, cycloalkyl, heteroaryl or heterocyclyl ring. Each ring of the heterocyclyl group containing a heteroatom may have 1, 2, 3 or 4 heteroatoms selected from N, O and/or S, where the N and S heteroatoms may optionally be oxidized and the N heteroatoms may optionally be quaternized, and wherein at least one carbon atom of heterocyclyl can be oxidized to form at least one C═O. The heterocyclic group may be attached at any heteroatom or carbon atom of the ring or ring system, where valence allows. The rings of multi-ring heterocycles may be fused, bridged and/or joined through one or more spiro atoms.

Non limiting exemplary heterocyclic groups include aziridinyl, oxiranyl, thiiranyl, piperidinyl, azetidinyl, oxetanyl, pyrrolidinyl, thietanyl, 2-imidazolinyl, pyrazolidinyl imidazolidinyl, isoxazolinyl, oxazolidinyl, isoxazolidinyl, thiazolidinyl, isothiazolidinyl, piperidinyl, succinimidyl, 3H-indolyl, indolinyl, chromanyl (also known as 3,4-dihydrobenzo[b]pyranyl), isoindolinyl, 2H-pyrrolyl, 1-pyrrolinyl, 2-pyrrolinyl, 3-pyrrolinyl, 4H-quinolizinyl, 2-oxopiperazinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, 2,5-dioximidazolidinyl, 2-oxopiperidinyl, 2-oxopyrrolodinyl, indolinyl, tetrahydropyranyl, tetrahydrofuranyl, tetrahydrothiophenyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, thiomorpholin-4-ylsulfoxide, thiomorpholin-4-ylsulfone, 1,3-dioxolanyl, 1,4-oxathianyl, 1,4-dithianyl, 1,3,5-trioxanyl, 1H-pyrrolizinyl, tetrahydro-1,1-dioxothiophenyl, N-formylpiperazinyl, and morpholin-4-yl. The term “aziridinyl” as used herein includes aziridin-1-yl and aziridin-2-yl. The term “oxyranyl” as used herein includes oxyranyl-2-yl. The term “thiiranyl” as used herein includes thiiran-2-yl. The term “azetidinyl” as used herein includes azetidin-1-yl, azetidin-2-yl and azetidin-3-yl. The term “oxetanyl” as used herein includes oxetan-2-yl and oxetan-3-yl. The term “thietanyl” as used herein includes thietan-2-yl and thietan-3-yl. The term “pyrrolidinyl” as used herein includes pyrrolidin-1-yl, pyrrolidin-2-yl and pyrrolidin-3-yl. The term “tetrahydrofuranyl” as used herein includes tetrahydrofuran-2-yl and tetrahydrofuran-3-yl. The term “tetrahydrothiophenyl” as used herein includes tetrahydrothiophen-2-yl and tetrahydrothiophen-3-yl. The term “succinimidyl” as used herein includes succinimid-1-yl and succininmid-3-yl. The term “dihydropyrrolyl” as used herein includes 2,3-dihydropyrrol-1-yl, 2,3-dihydro-1H-pyrrol-2-yl, 2,3-dihydro-1H-pyrrol-3-yl, 2,5-dihydropyrrol-1-yl, 2,5-dihydro-1H-pyrrol-3-yl and 2,5-dihydropyrrol-5-yl. The term “2H-pyrrolyl” as used herein includes 2H-pyrrol-2-yl, 2H-pyrrol-3-yl, 2H-pyrrol-4-yl and 2H-pyrrol-5-yl. The term “3H-pyrrolyl” as used herein includes 3H-pyrrol-2-yl, 3H-pyrrol-3-yl, 3H-pyrrol-4-yl and 3H-pyrrol-5-yl. The term “dihydrofuranyl” as used herein includes 2,3-dihydrofuran-2-yl, 2,3-dihydrofuran-3-yl, 2,3-dihydrofuran-4-yl, 2,3-dihydrofuran-5-yl, 2,5-dihydrofuran-2-yl, 2,5-dihydrofuran-3-yl, 2,5-dihydrofuran-4-yl and 2,5-dihydrofuran-5-yl. The term “dihydrothiophenyl” as used herein includes 2,3-dihydrothiophen-2-yl, 2,3-dihydrothiophen-3-yl, 2,3-dihydrothiophen-4-yl, 2,3-dihydrothiophen-5-yl, 2,5-dihydrothiophen-2-yl, 2,5-dihydrothiophen-3-yl, 2,5-dihydrothiophen-4-yl and 2,5-dihydrothiophen-5-yl. The term “imidazolidinyl” as used herein includes imidazolidin-1-yl, imidazolidin-2-yl and imidazolidin-4-yl. The term “pyrazolidinyl” as used herein includes pyrazolidin-1-yl, pyrazolidin-3-yl and pyrazolidin-4-yl. The term “imidazolinyl” as used herein includes imidazolin-1-yl, imidazolin-2-yl, imidazolin-4-yl and imidazolin-5-yl. The term “pyrazolinyl” as used herein includes 1-pyrazolin-3-yl, 1-pyrazolin-4-yl, 2-pyrazolin-1-yl, 2-pyrazolin-3-yl, 2-pyrazolin-4-yl, 2-pyrazolin-5-yl, 3-pyrazolin-1-yl, 3-pyrazolin-2-yl, 3-pyrazolin-3-yl, 3-pyrazolin-4-yl and 3-pyrazolin-5-yl. The term “dioxolanyl” also known as “1,3-dioxolanyl” as used herein includes dioxolan-2-yl, dioxolan-4-yl and dioxolan-5-yl. The term “dioxolyl” also known as “1,3-dioxolyl” as used herein includes dioxol-2-yl, dioxol-4-yl and dioxol-5-yl. The term “oxazolidinyl” as used herein includes oxazolidin-2-yl, oxazolidin-3-yl, oxazolidin-4-yl and oxazolidin-5-yl. The term “isoxazolidinyl” as used herein includes isoxazolidin-2-yl, isoxazolidin-3-yl, isoxazolidin-4-yl and isoxazolidin-5-yl. The term “oxazolinyl” as used herein includes 2-oxazolinyl-2-yl, 2-oxazolinyl-4-yl, 2-oxazolinyl-5-yl, 3-oxazolinyl-2-yl, 3-oxazolinyl-4-yl, 3-oxazolinyl-5-yl, 4-oxazolinyl-2-yl, 4-oxazolinyl-3-yl, 4-oxazolinyl-4-yl and 4-oxazolinyl-5-yl. The term “isoxazolinyl” as used herein includes 2-isoxazolinyl-3-yl, 2-isoxazolinyl-4-yl, 2-isoxazolinyl-5-yl, 3-isoxazolinyl-3-yl, 3-isoxazolinyl-4-yl, 3-isoxazolinyl-5-yl, 4-isoxazolinyl-2-yl, 4-isoxazolinyl-3-yl, 4-isoxazolinyl-4-yl and 4-isoxazolinyl-5-yl. The term “thiazolidinyl” as used herein includes thiazolidin-2-yl, thiazolidin-3-yl, thiazolidin-4-yl and thiazolidin-5-yl. The term “isothiazolidinyl” as used herein includes isothiazolidin-2-yl, isothiazolidin-3-yl, isothiazolidin-4-yl and isothiazolidin-5-yl. The term “chromanyl” as used herein includes chroman-2-yl, chroman-3-yl, chroman-4-yl, chroman-5-yl, chroman-6-yl, chroman-7-yl and chroman-8-yl. The term “thiazolinyl” as used herein includes 2-thiazolinyl-2-yl, 2-thiazolinyl-4-yl, 2-thiazolinyl-5-yl, 3-thiazolinyl-2-yl, 3-thiazolinyl-4-yl, 3-thiazolinyl-5-yl, 4-thiazolinyl-2-yl, 4-thiazolinyl-3-yl, 4-thiazolinyl-4-yl and 4-thiazolinyl-5-yl. The term “isothiazolinyl” as used herein includes 2-isothiazolinyl-3-yl, 2-isothiazolinyl-4-yl, 2-isothiazolinyl-5-yl, 3-isothiazolinyl-3-yl, 3-isothiazolinyl-4-yl, 3-isothiazolinyl-5-yl, 4-isothiazolinyl-2-yl, 4-isothiazolinyl-3-yl, 4-isothiazolinyl-4-yl and 4-isothiazolinyl-5-yl. The term “piperidyl” also known as “piperidinyl” as used herein includes piperid-1-yl, piperid-2-yl, piperid-3-yl and piperid-4-yl. The term “dihydropyridinyl” as used herein includes 1,2-dihydropyridin-1-yl, 1,2-dihydropyridin-2-yl, 1,2-dihydropyridin-3-yl, 1,2-dihydropyridin-4-yl, 1,2-dihydropyridin-5-yl, 1,2-dihydropyridin-6-yl, 1,4-dihydropyridin-1-yl, 1,4-dihydropyridin-2-yl, 1,4-dihydropyridin-3-yl, 1,4-dihydropyridin-4-yl, 2,3-dihydropyridin-2-yl, 2,3-dihydropyridin-3-yl, 2,3-dihydropyridin-4-yl, 2,3-dihydropyridin-5-yl, 2,3-dihydropyridin-6-yl, 2,5-dihydropyridin-2-yl, 2,5-dihydropyridin-3-yl, 2,5-dihydropyridin-4-yl, 2,5-dihydropyridin-5-yl, 2,5-dihydropyridin-6-yl, 3,4-dihydropyridin-2-yl, 3,4-dihydropyridin-3-yl, 3,4-dihydropyridin-4-yl, 3,4-dihydropyridin-5-yl and 3,4-dihydropyridin-6-yl. The term “tetrahydropyridinyl” as used herein includes 1,2,3,4-tetrahydropyridin-1-yl, 1,2,3,4-tetrahydropyridin-2-yl, 1,2,3,4-tetrahydropyridin-3-yl, 1,2,3,4-tetrahydropyridin-4-yl, 1,2,3,4-tetrahydropyridin-5-yl, 1,2,3,4-tetrahydropyridin-6-yl, 1,2,3,6-tetrahydropyridin-1-yl, 1,2,3,6-tetrahydropyridin-2-yl, 1,2,3,6-tetrahydropyridin-3-yl, 1,2,3,6-tetrahydropyridin-4-yl, 1,2,3,6-tetrahydropyridin-5-yl, 1,2,3,6-tetrahydropyridin-6-yl, 2,3,4,5-tetrahydropyridin-2-yl, 2,3,4,5-tetrahydropyridin-3-yl, 2,3,4,5-tetrahydropyridin-3-yl, 2,3,4,5-tetrahydropyridin-4-yl, 2,3,4,5-tetrahydropyridin-5-yl and 2,3,4,5-tetrahydropyridin-6-yl. The term “tetrahydropyranyl” also known as “oxanyl” or “tetrahydro-2H-pyranyl”, as used herein includes tetrahydropyran-2-yl, tetrahydropyran-3-yl and tetrahydropyran-4-yl. The term “2H-pyranyl” as used herein includes 2H-pyran-2-yl, 2H-pyran-3-yl, 2H-pyran-4-yl, 2H-pyran-5-yl and 2H-pyran-6-yl. The term “4H-pyranyl” as used herein includes 4H-pyran-2-yl, 4H-pyran-3-yl and 4H-pyran-4-yl. The term “3,4-dihydro-2H-pyranyl” as used herein includes 3,4-dihydro-2H-pyran-2-yl, 3,4-dihydro-2H-pyran-3-yl, 3,4-dihydro-2H-pyran-4-yl, 3,4-dihydro-2H-pyran-5-yl and 3,4-dihydro-2H-pyran-6-yl. The term “3,6-dihydro-2H-pyranyl” as used herein includes 3,6-dihydro-2H-pyran-2-yl, 3,6-dihydro-2H-pyran-3-yl, 3,6-dihydro-2H-pyran-4-yl, 3,6-dihydro-2H-pyran-5-yl and 3,6-dihydro-2H-pyran-6-yl. The term “tetrahydrothiophenyl”, as used herein includes tetrahydrothiophen-2-yl, tetrahydrothiophenyl-3-yl and tetrahydrothiophenyl-4-yl. The term “2H-thiopyranyl” as used herein includes 2H-thiopyran-2-yl, 2H-thiopyran-3-yl, 2H-thiopyran-4-yl, 2H-thiopyran-5-yl and 2H-thiopyran-6-yl. The term “4H-thiopyranyl” as used herein includes 4H-thiopyran-2-yl, 4H-thiopyran-3-yl and 4H-thiopyran-4-yl. The term “3,4-dihydro-2H-thiopyranyl” as used herein includes 3,4-dihydro-2H-thiopyran-2-yl, 3,4-dihydro-2H-thiopyran-3-yl, 3,4-dihydro-2H-thiopyran-4-yl, 3,4-dihydro-2H-thiopyran-5-yl and 3,4-dihydro-2H-thiopyran-6-yl. The term “3,6-dihydro-2H-thiopyranyl” as used herein includes 3,6-dihydro-2H-thiopyran-2-yl, 3,6-dihydro-2H-thiopyran-3-yl, 3,6-dihydro-2H-thiopyran-4-yl, 3,6-dihydro-2H-thiopyran-5-yl and 3,6-dihydro-2H-thiopyran-6-yl. The term “piperazinyl” also known as “piperazidinyl” as used herein includes piperazin-1-yl and piperazin-2-yl. The term “morpholinyl” as used herein includes morpholin-2-yl, morpholin-3-yl and morpholin-4-yl. The term “thiomorpholinyl” as used herein includes thiomorpholin-2-yl, thiomorpholin-3-yl and thiomorpholin-4-yl. The term “dioxanyl” as used herein includes 1,2-dioxan-3-yl, 1,2-dioxan-4-yl, 1,3-dioxan-2-yl, 1,3-dioxan-4-yl, 1,3-dioxan-5-yl and 1,4-dioxan-2-yl. The term “dithianyl” as used herein includes 1,2-dithian-3-yl, 1,2-dithian-4-yl, 1,3-dithian-2-yl, 1,3-dithian-4-yl, 1,3-dithian-5-yl and 1,4-dithian-2-yl. The term “oxathianyl” as used herein includes oxathian-2-yl and oxathian-3-yl. The term “trioxanyl” as used herein includes 1,2,3-trioxan-4-yl, 1,2,3-trioxay-5-yl, 1,2,4-trioxay-3-yl, 1,2,4-trioxay-5-yl, 1,2,4-trioxay-6-yl and 1,3,4-trioxay-2-yl. The term “azepanyl” as used herein includes azepan-1-yl, azepan-2-yl, azepan-1-yl, azepan-3-yl and azepan-4-yl. The term “homopiperazinyl” as used herein includes homopiperazin-1-yl, homopiperazin-2-yl, homopiperazin-3-yl and homopiperazin-4-yl. The term “indolinyl” as used herein includes indolin-1-yl, indolin-2-yl, indolin-3-yl, indolin-4-yl, indolin-5-yl, indolin-6-yl, and indolin-7-yl. The term “quinolizinyl” as used herein includes quinolizidin-1-yl, quinolizidin-2-yl, quinolizidin-3-yl and quinolizidin-4-yl. The term “isoindolinyl” as used herein includes isoindolin-1-yl, isoindolin-2-yl, isoindolin-3-yl, isoindolin-4-yl, isoindolin-5-yl, isoindolin-6-yl, and isoindolin-7-yl. The term “3H-indolyl” as used herein includes 3H-indol-2-yl, 3H-indol-3-yl, 3H-indol-4-yl, 3H-indol-5-yl, 3H-indol-6-yl, and 3H-indol-7-yl. The term “quinolizinyl” as used herein includes quinolizidin-1-yl, quinolizidin-2-yl, quinolizidin-3-yl and quinolizidin-4-yl. The term “quinolizinyl” as used herein includes quinolizidin-1-yl, quinolizidin-2-yl, quinolizidin-3-yl and quinolizidin-4-yl. The term “tetrahydroquinolinyl” as used herein includes tetrahydroquinolin-1-yl, tetrahydroquinolin-2-yl, tetrahydroquinolin-3-yl, tetrahydroquinolin-4-yl, tetrahydroquinolin-5-yl, tetrahydroquinolin-6-yl, tetrahydroquinolin-7-yl and tetrahydroquinolin-8-yl. The term “tetrahydroisoquinolinyl” as used herein includes tetrahydroisoquinolin-1-yl, tetrahydroisoquinolin-2-yl, tetrahydroisoquinolin-3-yl, tetrahydroisoquinolin-4-yl, tetrahydroisoquinolin-5-yl, tetrahydroisoquinolin-6-yl, tetrahydroisoquinolin-7-yl and tetrahydroisoquinolin-8-yl. The term “1H-pyrrolizine” as used herein includes 1H-pyrrolizin-1-yl, 1H-pyrrolizin-2-yl, 1H-pyrrolizin-3-yl, 1H-pyrrolizin-5-yl, 1H-pyrrolizin-6-yl and 1H-pyrrolizin-7-yl. The term “3H-pyrrolizine” as used herein includes 3H-pyrrolizin-1-yl, 3H-pyrrolizin-2-yl, 3H-pyrrolizin-3-yl, 3H-pyrrolizin-5-yl, 3H-pyrrolizin-6-yl and 3H-pyrrolizin-7-yl.

The term “heteroatom substituted cycloalkyl” as used herein refers to a fully saturated heterocyclyl as defined herein.

When the suffix “ene” is used in conjunction with a heterocyclyl group, i.e. “heterocyclylene”, this is intended to mean the heterocyclyl group as defined herein having two single bonds as points of attachment to other groups.

The term “heterocyclyloxy”, as a group or part of a group, refers to a group having the formula —O—Rⁱ wherein Rⁱ is heterocyclyl as defined herein above.

The term “heteroaryl” as a group or part of a group, refers but is not limited to 5 to 12 carbon-atom aromatic rings or ring systems containing 1 or 2 rings which can be fused together or linked covalently, typically containing 5 to 6 atoms; at least one of which is aromatic in which one or more carbon atoms in one or more of these rings can be replaced by N, O and/or S atoms where the N and S heteroatoms may optionally be oxidized and the N heteroatoms may optionally be quaternized, and wherein at least one carbon atom of said heteroaryl can be oxidized to form at least one C═O. Such rings may be fused to an aryl, cycloalkyl, heteroaryl or heterocyclyl ring. Non-limiting examples of such heteroaryl, include: pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, benzo[d]oxazol-2(3H)-one, 2,3-dihydro-benzofuranyl, thienopyridinyl, purinyl, imidazo[1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl; preferably said heteroaryl group is selected from the group consisting of pyridyl, 1,3-benzodioxolyl, benzo[d]oxazol-2(3H)-one, 2,3-dihydro-benzofuranyl, pyrazinyl, pyrazolyl, pyrrolyl, isoxazolyl, thiophenyl, imidazolyl, benzimidazolyl, pyrimidinyl, triazolyl and thiazolyl.

The term “pyrrolyl” (also called azolyl) as used herein includes pyrrol-1-yl, pyrrol-2-yl and pyrrol-3-yl. The term “furanyl” (also called “furyl”) as used herein includes furan-2-yl and furan-3-yl (also called furan-2-yl and furan-3-yl). The term “thiophenyl” (also called “thienyl”) as used herein includes thiophen-2-yl and thiophen-3-yl (also called thien-2-yl and thien-3-yl). The term “pyrazolyl” (also called 1H-pyrazolyl and 1,2-diazolyl) as used herein includes pyrazol-1-yl, pyrazol-3-yl, pyrazol-4-yl and pyrazol-5-yl. The term “imidazolyl” as used herein includes imidazol-1-yl, imidazol-2-yl, imidazol-4-yl and imidazol-5-yl. The term “oxazolyl” (also called 1,3-oxazolyl) as used herein includes oxazol-2-yl, oxazol-4-yl and oxazol-5-yl. The term “isoxazolyl” (also called 1,2-oxazolyl), as used herein includes isoxazol-3-yl, isoxazol-4-yl, and isoxazol-5-yl. The term “thiazolyl” (also called 1,3-thiazolyl), as used herein includes thiazol-2-yl, thiazol-4-yl and thiazol-5-yl (also called 2-thiazolyl, 4-thiazolyl and 5-thiazolyl). The term “isothiazolyl” (also called 1,2-thiazolyl) as used herein includes isothiazol-3-yl, isothiazol-4-yl, and isothiazol-5-yl. The term “triazolyl” as used herein includes 1H-triazolyl and 4H-1,2,4-triazolyl, “1H-triazolyl” includes 1H-1,2,3-triazol-1-yl, 1H-1,2,3-triazol-4-yl, 1H-1,2,3-triazol-5-yl, 1H-1,2,4-triazol-1-yl, 1H-1,2,4-triazol-3-yl and 1H-1,2,4-triazol-5-yl. “4H-1,2,4-triazolyl” includes 4H-1,2,4-triazol-4-yl, and 4H-1,2,4-triazol-3-yl. The term “oxadiazolyl” as used herein includes 1,2,3-oxadiazol-4-yl, 1,2,3-oxadiazol-5-yl, 1,2,4-oxadiazol-3-yl, 1,2,4-oxadiazol-5-yl, 1,2,5-oxadiazol-3-yl and 1,3,4-oxadiazol-2-yl. The term “thiadiazolyl” as used herein includes 1,2,3-thiadiazol-4-yl, 1,2,3-thiadiazol-5-yl, 1,2,4-thiadiazol-3-yl, 1,2,4-thiadiazol-5-yl, 1,2,5-thiadiazol-3-yl (also called furazan-3-yl) and 1,3,4-thiadiazol-2-yl. The term “tetrazolyl” as used herein includes 1H-tetrazol-1-yl, 1H-tetrazol-5-yl, 2H-tetrazol-2-yl, and 2H-tetrazol-5-yl. The term “oxatriazolyl” as used herein includes 1,2,3,4-oxatriazol-5-yl and 1,2,3,5-oxatriazol-4-yl. The term “thiatriazolyl” as used herein includes 1,2,3,4-thiatriazol-5-yl and 1,2,3,5-thiatriazol-4-yl. The term “pyridinyl” (also called “pyridyl”) as used herein includes pyridin-2-yl, pyridin-3-yl and pyridin-4-yl (also called 2-pyridyl, 3-pyridyl and 4-pyridyl). The term “pyrimidyl” as used herein includes pyrimid-2-yl, pyrimid-4-yl, pyrimid-5-yl and pyrimid-6-yl. The term “pyrazinyl” as used herein includes pyrazin-2-yl and pyrazin-3-yl. The term “pyridazinyl as used herein includes pyridazin-3-yl and pyridazin-4-yl. The term “oxazinyl” (also called “1,4-oxazinyl”) as used herein includes 1,4-oxazin-4-yl and 1,4-oxazin-5-yl. The term “dioxinyl” (also called “1,4-dioxinyl”) as used herein includes 1,4-dioxin-2-yl and 1,4-dioxin-3-yl. The term “thiazinyl” (also called “1,4-thiazinyl”) as used herein includes 1,4-thiazin-2-yl, 1,4-thiazin-3-yl, 1,4-thiazin-4-yl, 1,4-thiazin-5-yl and 1,4-thiazin-6-yl. The term “triazinyl” as used herein includes 1,3,5-triazin-2-yl, 1,2,4-triazin-3-yl, 1,2,4-triazin-5-yl, 1,2,4-triazin-6-yl, 1,2,3-triazin-4-yl and 1,2,3-triazin-5-yl. The term “imidazo[2,1-b][1,3]thiazolyl” as used herein includes imidazo[2,1-b][1,3]thiazol-2-yl, imidazo[2,1-b][1,3]thiazol-3-yl, imidazo[2,1-b][1,3]thiazol-5-yl and imidazo[2,1-b][1,3]thiazol-6-yl.

The term “thieno[3,2-b]furanyl” as used herein includes thieno[3,2-b]furan-2-yl, thieno[3,2-b]furan-3-yl, thieno[3,2-b]furan-4-yl, and thieno[3,2-b]furan-5-yl. The term “thieno[3,2-b]thiophenyl” as used herein includes thieno[3,2-b]thien-2-yl, thieno[3,2-b]thien-3-yl, thieno[3,2-b]thien-5-yl and thieno[3,2-b]thien-6-yl. The term “thieno[2,3-d][1,3]thiazolyl” as used herein includes thieno[2,3-d][1,3]thiazol-2-yl, thieno[2,3-d][1,3]thiazol-5-yl and thieno[2,3-d][1,3]thiazol-6-yl. The term “thieno[2,3-d]imidazolyl” as used herein includes thieno[2,3-d]imidazol-2-yl, thieno[2,3-d]imidazol-4-yl and thieno[2,3-d]imidazol-5-yl. The term “tetrazolo[1,5-a]pyridinyl” as used herein includes tetrazolo[1,5-a]pyridine-5-yl, tetrazolo[1,5-a]pyridine-6-yl, tetrazolo[1,5-a]pyridine-7-yl, and tetrazolo[1,5-a]pyridine-8-yl. The term “indolyl” as used herein includes indol-1-yl, indol-2-yl, indol-3-yl,-indol-4-yl, indol-5-yl, indol-6-yl and indol-7-yl. The term “indolizinyl” as used herein includes indolizin-1-yl, indolizin-2-yl, indolizin-3-yl, indolizin-5-yl, indolizin-6-yl, indolizin-7-yl, and indolizin-8-yl. The term “isoindolyl” as used herein includes isoindol-1-yl, isoindol-2-yl, isoindol-3-yl, isoindol-4-yl, isoindol-5-yl, isoindol-6-yl and isoindol-7-yl. The term “benzofuranyl” (also called benzo[b]furanyl) as used herein includes benzofuran-2-yl, benzofuran-3-yl, benzofuran-4-yl, benzofuran-5-yl, benzofuran-6-yl and benzofuran-7-yl. The term “isobenzofuranyl” (also called benzo[c]furanyl) as used herein includes isobenzofuran-1-yl, isobenzofuran-3-yl, isobenzofuran-4-yl, isobenzofuran-5-yl, isobenzofuran-6-yl and isobenzofuran-7-yl. The term “benzothiophenyl” (also called benzo[b]thienyl) as used herein includes 2-benzo[b]thiophenyl, 3-benzo[b]thiophenyl, 4-benzo[b]thiophenyl, 5-benzo[b]thiophenyl, 6-benzo[b]thiophenyl and -7-benzo[b]thiophenyl (also called benzothien-2-yl, benzothien-3-yl, benzothien-4-yl, benzothien-5-yl, benzothien-6-yl and benzothien-7-yl). The term “isobenzothiophenyl” (also called benzo[c]thienyl) as used herein includes isobenzothien-1-yl, isobenzothien-3-yl, isobenzothien-4-yl, isobenzothien-5-yl, isobenzothien-6-yl and isobenzothien-7-yl. The term “indazolyl” (also called 1H-indazolyl or 2-azaindolyl) as used herein includes 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, and 2H-indazol-7-yl. The term “benzimidazolyl” as used herein includes benzimidazol-1-yl, benzimidazol-2-yl, benzimidazol-4-yl, benzimidazol-5-yl, benzimidazol-6-yl and benzimidazol-7-yl. The term “1,3-benzoxazolyl” as used herein includes 1,3-benzoxazol-2-yl, 1,3-benzoxazol-4-yl, 1,3-benzoxazol-5-yl, 1,3-benzoxazol-6-yl and 1,3-benzoxazol-7-yl. The term “1,2-benzisoxazolyl” as used herein includes 1,2-benzisoxazol-3-yl, 1,2-benzisoxazol-4-yl, 1,2-benzisoxazol-5-yl, 1,2-benzisoxazol-6-yl and 1,2-benzisoxazol-7-yl. The term “2,1-benzisoxazolyl” as used herein includes 2,1-benzisoxazol-3-yl, 2,1-benzisoxazol-4-yl, 2,1-benzisoxazol-5-yl, 2,1-benzisoxazol-6-yl and 2,1-benzisoxazol-7-yl. The term “1,3-benzothiazolyl” as used herein includes 1,3-benzothiazol-2-yl, 1,3-benzothiazol-4-yl, 1,3-benzothiazol-5-yl, 1,3-benzothiazol-6-yl and 1,3-benzothiazol-7-yl. The term “1,2-benzoisothiazolyl” as used herein includes 1,2-benzisothiazol-3-yl, 1,2-benzisothiazol-4-yl, 1,2-benzisothiazol-5-yl, 1,2-benzisothiazol-6-yl and 1,2-benzisothiazol-7-yl. The term “2,1-benzoisothiazolyl” as used herein includes 2,1-benzisothiazol-3-yl, 2,1-benzisothiazol-4-yl, 2,1-benzisothiazol-5-yl, 2,1-benzisothiazol-6-yl and 2,1-benzisothiazol-7-yl. The term “benzotriazolyl” as used herein includes benzotriazol-1-yl, benzotriazol-4-yl, benzotriazol-5-yl, benzotriazol-6-yl and benzotriazol-7-yl. The term “1,2,3-benzoxadiazolyl” as used herein includes 1,2,3-benzoxadiazol-4-yl, 1,2,3-benzoxadiazol-5-yl, 1,2,3-benzoxadiazol-6-yl and 1,2,3-benzoxadiazol-7-yl. The term “2,1,3-benzoxadiazolyl” as used herein includes 2,1,3-benzoxadiazol-4-yl, 2,1,3-benzoxadiazol-5-yl, 2,1,3-benzoxadiazol-6-yl and 2,1,3-benzoxadiazol-7-yl. The term “1,2,3-benzothiadiazolyl” as used herein includes 1,2,3-benzothiadiazol-4-yl, 1,2,3-benzothiadiazol-5-yl, 1,2,3-benzothiadiazol-6-yl and 1,2,3-benzothiadiazol-7-yl. The term “2,1,3-benzothiadiazolyl” as used herein includes 2,1,3-benzothiadiazol-4-yl, 2,1,3-benzothiadiazol-5-yl, 2,1,3-benzothiadiazol-6-yl and 2,1,3-benzothiadiazol-7-yl. The term “thienopyridinyl” as used herein includes thieno[2,3-b]pyridinyl, thieno[2,3-c]pyridinyl, thieno[3,2-c]pyridinyl and thieno[3,2-b]pyridinyl. The term “purinyl” as used herein includes purin-2-yl, purin-6-yl, purin-7-yl and purin-8-yl. The term “imidazo[1,2-a]pyridinyl”, as used herein includes imidazo[1,2-a]pyridin-2-yl, imidazo[1,2-a]pyridin-3-yl, imidazo[1,2-a]pyridin-4-yl, imidazo[1,2-a]pyridin-5-yl, imidazo[1,2-a]pyridin-6-yl and imidazo[1,2-a]pyridin-7-yl. The term “1,3-benzodioxolyl”, as used herein includes 1,3-benzodioxol-4-yl, 1,3-benzodioxol-5-yl, 1,3-benzodioxol-6-yl, and 1,3-benzodioxol-7-yl. The term “quinolinyl” as used herein includes quinolin-2-yl, quinolin-3-yl, quinolin-4-yl, quinolin-5-yl, quinolin-6-yl, quinolin-7-yl and quinolin-8-yl. The term “isoquinolinyl” as used herein includes isoquinolin-1-yl, isoquinolin-3-yl, isoquinolin-4-yl, isoquinolin-5-yl, isoquinolin-6-yl, isoquinolin-7-yl and isoquinolin-8-yl. The term “cinnolinyl” as used herein includes cinnolin-3-yl, cinnolin-4-yl, cinnolin-5-yl, cinnolin-6-yl, cinnolin-7-yl and cinnolin-8-yl. The term “quinazolinyl” as used herein includes quinazolin-2-yl, quinazolin-4-yl, quinazolin-5-yl, quinazolin-6-yl, quinazolin-7-yl and quinazolin-8-yl. The term “quinoxalinyl” as used herein includes quinoxalin-2-yl, quinoxalin-5-yl, and quinoxalin-6-yl.

When the suffix “ene” is used in conjunction with a heteroaryl group, i.e. “heteroarylene”, this is intended to mean the heteroaryl group as defined herein having two single bonds as points of attachment to other groups.

The term “heteroaryloxy”, as a group or part of a group, refers to a group having the formula —O—R^{k} wherein R^{k} is heteroaryl as defined herein above.

The term “mono- or di-alkylamino”, as a group or part of a group, refers to a group of formula —N(R^{o})(R^{p}) wherein R^{o} and R^{p} are each independently selected from hydrogen, or alkyl, wherein at least one of R^{o} or R^{p} is alkyl. Thus, alkylamino include mono-alkyl amino group (e.g. mono-alkylamino group such as methylamino and ethylamino), and di-alkylamino group (e.g. di-alkylamino group such as dimethylamino and diethylamino). Non-limiting examples of suitable mono- or di-alkylamino groups include n-propylamino, isopropylamino, n-butylamino, i-butylamino, sec-butylamino, t-butylamino, pentylamino, n-hexylamino, di-n-propylamino, di-i-propylamino, ethylmethylamino, methyl-n-propylamino, methyl-1-propylamino, n-butylmethylamino, i-butylmethylamino, t-butylmethylamino, ethyl-n-propylamino, ethyl-1-propylamino, n-butylethylamino, i-butylethylamino, t-butylethylamino, di-n-butylamino, di-i-butylamino, methylpentylamino, methylhexylamino, ethylpentylamino, ethylhexylamino, propylpentylamino, propylhexylamino, and the like.

The term “mono- or di-arylamino”, as a group or part of a group, refers to a group of formula —N(R^{q})(R^{r}) wherein R^{q} and R^{r} are each independently selected from hydrogen, aryl, or alkyl, wherein at least one of R^{q} or R^{r} is aryl.

The term “mono- or di-cycloalkylamino”, as a group or part of a group, refers to a group of formula —N(R^{s})(R^{t}) wherein R^{s} and R^{t} are each independently selected from hydrogen, cycloalkyl, or alkyl, wherein at least one of R^{s} or R^{t} is cycloalkyl.

The term “mono- or di-heteroarylamino”, as a group or part of a group, refers to a group of formula —N(R^{u})(R^{v}) wherein R^{u} and R^{v} are each independently selected from hydrogen, heteroaryl, or alkyl, wherein at least one of R^{u} or R^{v} is heteroaryl as defined herein.

The term “mono- or di-heterocyclylamino”, as a group or part of a group, refers to a group of formula —N(R^{w})(R^{x}) wherein R^{w} and R^{x} are each independently selected from hydrogen, heterocyclyl, or alkyl, wherein at least one of R^{w} or R^{x} is heterocyclyl as defined herein.

The term “alkyloxycarbonyl”, as a group or part of a group, refers to a group of formula —COO—R^{b}, wherein R^{b} is alkyl as defined herein.

The term “cycloalkyloxycarbonyl”, as a group or part of a group, refers to a group of formula —COO—R^{b}, wherein R^{b} is cycloalkyl as defined herein.

The term “aryloxycarbonyl”, as a group or part of a group, refers to a group of formula —COO—R^{b}, wherein R^{b} is aryl as defined herein.

The term “alkylcarbonyl”, as a group or part of a group, refers to a group of formula —CO—R^{b}, wherein R^{b} is alkyl as defined herein.

The term “cycloalkylcarbonyl”, as a group or part of a group, refers to a group of formula —CO—R^{b}, wherein R^{b} is cycloalkyl as defined herein.

The term “arylcarbonyl”, as a group or part of a group, refers to a group of formula —CO—R^{b}, wherein R^{b} is aryl as defined herein.

The term “alkylsulfonyl”, as a group or part of a group, refers to a group of formula —S(O)₂—R^{b}, wherein R^{b} is alkyl as defined herein.

The term “cycloalkylsulfonyl”, as a group or part of a group, refers to a group of formula —S(O)₂—R^{b}, wherein R^{b} is cycloalkyl as defined herein.

The term “arylsulfonyl”, as a group or part of a group, refers to a group of formula —S(O)₂—R^{b}, wherein R^{b} is aryl as defined herein.

The term “mono or di-C₁₋₆alkylaminocarbonylC₁₋₆alkyl”, as a group or part of a group, refers to a group of formula —R^{a}—CONR^{o}R^{p} wherein R^{o}R^{p} are each independently selected from hydrogen, or C₁₋₆alkyl, wherein at least one of R^{o} or R^{p} is C₁₋₆alkyl, and R^{a} is C₁₋₆alkylene as defined herein.

The term “a saturated or unsaturated 3-, 4-, 5-, 6- or 7-membered ring” as used herein encompasses saturated or unsaturated carbon only membered rings, as well as saturated or unsaturated heteroatoms containing rings. The term “a saturated 3-, 4-, 5-, 6-or 7-carbon membered ring” as used herein refers to saturated carbon only membered ring such as C₃₋₇cycloalkyl and C₃₋₇cycloalkylene.

Whenever used in the present invention the term “compounds of the invention” or a similar term is meant to include the compounds of general formula (1A) (1B), (1C) or (2) and any subgroup thereof.

This term also refers to the compounds as depicted in Table 1 and their derivatives pharmaceutically acceptable salts, solvates, hydrates, stereoisomeric forms, racemic mixtures, optical isomers, analogues, and prodrugs.

The term “prodrug” as used herein means the pharmacologically acceptable derivatives such as esters, amides and phosphates, such that the resulting in vivo biotransformation product of the derivative is the active drug. The reference by Goodman and Gilman (The Pharmacological Basis of Therapeutics, 8th Ed, McGraw-Hill, Int. Ed. 1992, “Biotransformation of Drugs”, p 13-15) describing pro-drugs generally is hereby incorporated. Pro-drugs of the compounds of the invention can be prepared by modifying functional groups present in said component in such a way that the modifications are cleaved, either in routine manipulation or in vivo, to the parent component. Typical examples of pro-drugs are described for instance in WO 99/33795, WO 99/33815, WO 99/33793 and WO 99/33792 all incorporated herein by reference. Pro-drugs are characterized by increased bio-availability and are readily metabolized into the active inhibitors in vivo. The term “prodrug”, as used herein, means any compound that will be modified to form a drug species, wherein the modification may take place either inside or outside of the body, and either before or after the pre-drug reaches the area of the body where administration of the drug is indicated.

Where a compound of formula (I) or (II) or any subgroup thereof contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of formula (I) containing, for example, an imino, keto, or oxime group, or so-called valence tautomerism in compounds which contain an aromatic moiety. It follows that a single compound may exhibit more than one type of isomerism.

Included within the scope of the present invention are all stereoisomers, diastereomers, geometric isomers and tautomeric forms of the compounds of formula (I) or (II) or any subgroups thereof, including compounds exhibiting more than one type of isomerism, and mixtures of one or more thereof. Also included are acid addition or base salts wherein the counterion is optically active, for example, d-lactate or /-lysine, or racemic, for example, dl-tartrate or dl-arginine.

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high performance liquid chromatography (HPLC).

The present invention includes all possible stereoisomers compounds of formula (I) or (II) and any subgroup thereof and includes not only racemic compounds but the individual enantiomers as well. When a compound is desired as a single enantiomer, such may be obtained by stereospecific synthesis, by resolution of the final product or any convenient intermediate, or by chiral chromatographic methods as each are known in the art. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994), incorporated by reference with regard to stereochemistry.

Present inventors have developed compounds which bind to the atypical chemokine receptor ACKR3 (also known as CXCR7) which are useful in the modulation of ACKR3 activity.

Modulation of ACKR3 can be used to affect and/or restore normal levels of endogenous opioid peptides in the treatment of disorders linked with endogenous opioid peptide dysregulation, like distress dysfunction diseases or conditions such as depression or chronic pain, with a potentially improved safety profile. To this end, present inventors developed compounds capable of specifically binding with ACKR3 and competing with the natural ligands of ACKR3 such that they can inhibit the scavenging function of ACKR3. In particular embodiments, the present invention provides compounds of formula (2); or a stereoisomer, enantiomer, racemic, thereof

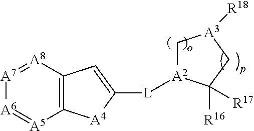

wherein,
o is an integer selected from 0, 1, 2 or 3;
p is an integer selected from 0, 1, 2, 3 or 4;
A² is selected from N or CR¹⁹;
A³ is selected from N or CR²¹;
A⁴ is selected from NR¹¹, O, S or CR²⁴ R²⁵;
A⁵ is selected from N or CR¹²;
A⁶ is selected from N or CR¹³;
A⁷ is selected from N or CR¹⁴;
A⁸ is selected from N or CR¹⁵;
wherein at least one of A² or A³ is N;
wherein at most one of A⁵ to A⁸ is N;
L is selected from —C═O, —C(O)—NH—, and CHR²¹;
R¹¹ is selected from the group consisting of hydrogen, deuterium, alkyl, —S(O)₂R²², aryl, —S(O)R²², —SO₂NR²²R²³; and wherein said alkyl or aryl can be unsubstituted or substituted with one or more Z¹;
R¹² is selected from the group consisting of hydrogen, deuterium, halogen, —OR²³, cyano, —C(O)R²³ alkyl, trifluoromethyl, trifluoromethoxy, cycloalkyl, aryl, heterocyclyl, heteroaryl, arylalkyl, and nitro, wherein said alkyl, cycloalkyl, aryl, heterocyclyl, heteroaryl, or arylalkyl is substituted by one or more Z^{1a};
R¹³ is selected from the group consisting of hydrogen, deuterium, halogen, —OR²³, cyano, —C(O)R²³ alkyl, trifluoromethyl, trifluoromethoxy, cycloalkyl, aryl, heterocyclyl, heteroaryl, arylalkyl, and nitro, wherein said alkyl, cycloalkyl, aryl, heterocyclyl, heteroaryl, or arylalkyl is substituted by one or more Z^{1b};
R¹⁴ is selected from the group consisting of hydrogen, deuterium, halogen, —OR²³, cyano, —C(O)R²³ alkyl, trifluoromethyl, trifluoromethoxy, cycloalkyl, aryl, heterocyclyl, heteroaryl, arylalkyl, and nitro, wherein said alkyl, cycloalkyl, aryl, heterocyclyl, heteroaryl, or arylalkyl is substituted by one or more Z^{1c};
R¹⁵ is selected from the group consisting of hydrogen, deuterium, halogen, —OR²³, cyano, —C(O)R²³, alkyl, trifluoromethyl, trifluoromethoxy, cycloalkyl, aryl, heterocyclyl, heteroaryl, arylalkyl, and nitro, wherein said alkyl, cycloalkyl, aryl, heterocyclyl, heteroaryl, or arylalkyl is substituted by one or more Z^{1d};
R¹⁶ is selected from the group consisting of hydrogen, deuterium, alkyl, halogen, and —OR²³;
R¹⁷ is selected from the group consisting of hydrogen, deuterium, alkyl, halogen, and —OR²³;
or R¹⁶ and R¹⁷ together with the carbon atom to which they are attached from a group selected from —C═CH-alkyl, —C═N—OH, —C═N—O—Si(CH₃)₂C(CH₃)₃, or a saturated or unsaturated 3-, 4-, 5-, 6- or 7-membered ring;
R¹⁸ is selected from the group consisting of halogen, —NH₂, —NHR²², alkyl, deuterium, arylalkyl, —S(O)₂R²², —C(O)OR²³, —S(O)R²², —(CH₂)₂—O—(CH₂)₂—NH₂, heteroaryl, cycloalkyl, aryl, and heterocyclyl; and wherein said alkyl, arylalkyl, heteroaryl, cycloalkyl, aryl, heterocyclyl, or arylalkyl can be unsubstituted or substituted with one or more Z²;
R¹⁹ is selected from the group consisting of hydrogen, alkyl, halogen, and —OR²³;
R²⁰ is selected from the group consisting of hydrogen, alkyl, halogen, and —OR²³;
R²¹ is selected from the group consisting of —OH, —COOR²³, —C(O)NH₂, hydrogen, and —OR²³;
each R²² is independently selected from the group consisting of alkyl, aryl, tolyl, cycloalkyl, arylalkyl, heterocyclyl, and heteroaryl;
each R²³ is independently selected from the group consisting of hydrogen, alkyl, aryl, cycloalkyl, arylalkyl, heterocyclyl, and heteroaryl;
R²⁴ is selected from the group consisting of hydrogen, deuterium, halogen, —OR²³, cyano, —C(O)R²³, alkyl, trifluoromethyl, trifluoromethoxy, cycloalkyl, aryl, heterocyclyl, heteroaryl, arylalkyl, and nitro;
R²⁵ is selected from the group consisting of hydrogen, deuterium, halogen, —OR²³, cyano, —C(O)R²³, alkyl, trifluoromethyl, trifluoromethoxy, cycloalkyl, aryl, heterocyclyl, heteroaryl, arylalkyl, and nitro;
each Z¹ is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z^{1a} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z^{1b} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z^{1c} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z^{1d} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z² is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
or ethyl {(3Z)-3-({[tert-butyl(dimethyl)silyl]oxy}imino-1-[(4-methylphenyl)sulfonyl]piperidin-4-yl}(3-formyl-1H-indol-2-yl)acetate;
or ethyl (3-formyl-1H-indol-2-yl){(3Z)-3-(hydroxyamino)-1-[(4-methylphenyl)sulfonyl]piperidin-4-yl}acetate;
or a solvate, hydrate, pharmaceutically acceptable salt or prodrug thereof,
and with the proviso that when A³ is N, R¹⁸ is not methyl, p-methoxy-benzyl or phenyl sulfone; and with the proviso that the said compound is not
(5-chloro-1H-indol-2-yl)-(4-methylpiperazin-1-yl)methanone;
(7-amino-5-chloro-1H-indol-2-yl)-(4-methylpiperazin-1-yl)methanone; or
(5-chloro-7-methyl-1H-indol-2-yl)-(3-pyrrolidin-1-ylazetidin-1-yl)methanone.

In some embodiments, for the compounds according to the present invention, when A³ is N, then R¹⁸ is not diphenylmethyl.

In some embodiments, for the compounds according to the present invention, when A³ is C, then R¹⁸ is not alkyl or benzyl.

In some embodiments, for the compounds according to the present invention, when A² is N, then R¹⁴ is not chloro, methyl or trifluoromethyl.

In some embodiments for the compounds according to the present invention, when L is —C(O)—NH—, then R¹⁵ is not bromo, —OR²³ phenyl, pyridyl.

In some embodiments the compound according to the present invention is not:
tert-butyl 4-(1H-indole-2-carbonyl)piperazine-1-carboxylate;
tert-butyl 4-(1-methylindole-2-carbonyl)piperazine-1-carboxylate;
1H-indol-2-yl-[4-(1-phenylethyl)piperazin-1-yl]methanone;
(1-methylindol-2-yl)-[4-(1-phenylethyl)piperazin-1-yl]methanone;
[4-(1,3-benzodioxol-5-ylmethyl)piperazin-1-yl]-(1H-indol-2-yl)methanone;
[4-(2-hydroxy-2-methyl-propyl)piperazin-1-yl]-(5-methoxy-1H-indol-2-yl)methanone;
4-benzo[1,2,5]oxadiazol-5-yl-1H-indole-2-carboxylic acid [1-(2-azepan-1-yl-ethyl)-piperidin-4-yl]-amide;
4-benzo[1,3]dioxol-5-yl-1H-indole-2-carboxylic acid [1-(2-azepan-1-yl-ethyl)-piperidin-4-yl]-amide; or
4-hydroxy-1H-indole-2-carboxylic acid [1-(2-azepan-1-yl-ethyl)-piperidin-4-yl]-amide.

In some embodiments the compounds of the present invention have structural formula (2A)

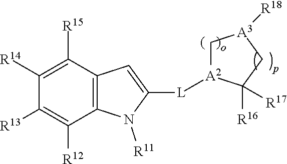

wherein o, p, A², A³, L, R¹¹, R¹², R¹³, R¹⁴, R¹⁵, R¹⁶, R¹⁷, and R¹⁸ have the same meaning as that defined herein.

In some embodiments the compounds of the present invention have structural formulae (2B), (2C), (2D), (2E), or (2F),

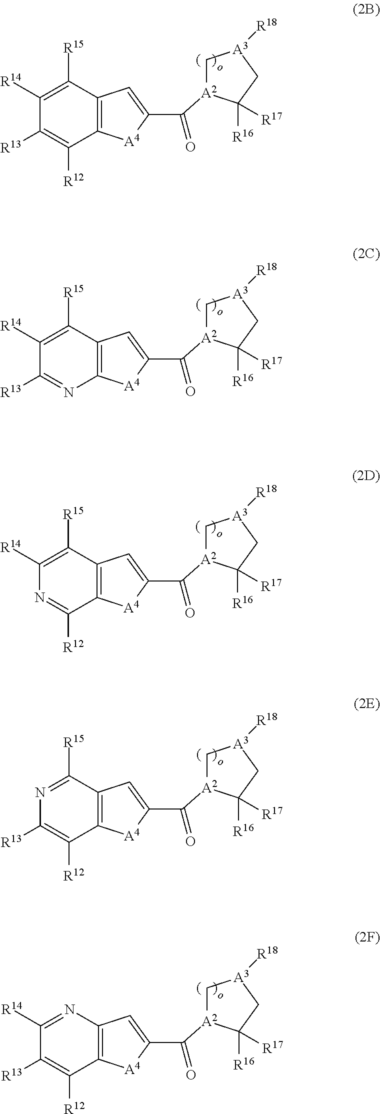

wherein o, A², A³, A⁴, R¹², R¹³, R¹⁴, R¹⁵, R¹⁶, R¹⁷, and R¹⁸ have the same meaning as that defined herein.

In some embodiments the compounds of the present invention have structural formulae (2G), or (2H),

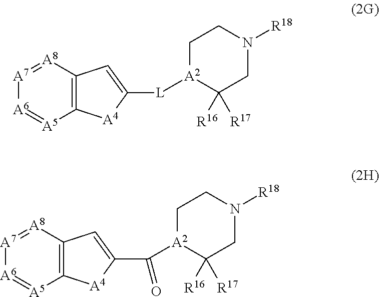

wherein A³, A⁴, A⁵, A⁶, A⁷, A⁸, L, R¹⁶, R¹⁷, and R¹⁸ have the same meaning as that defined herein.

In some embodiments the compounds of the present invention have structural formulae (2I), (2J), (2K), or (2L),

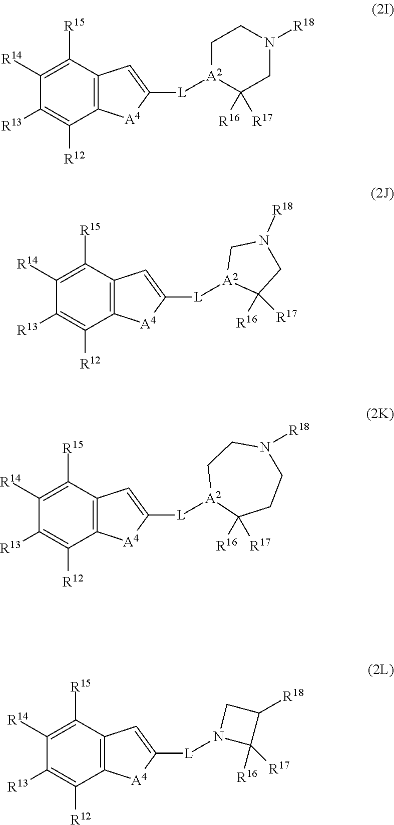

wherein A², A³, A⁴, L, R¹², R¹³, R¹⁴, R¹⁵, R¹⁶, R¹⁷, and R¹⁸ have the same meaning as that defined herein.

In some embodiments the compounds of the present invention have structural formula (2B), (2M), or (2N),

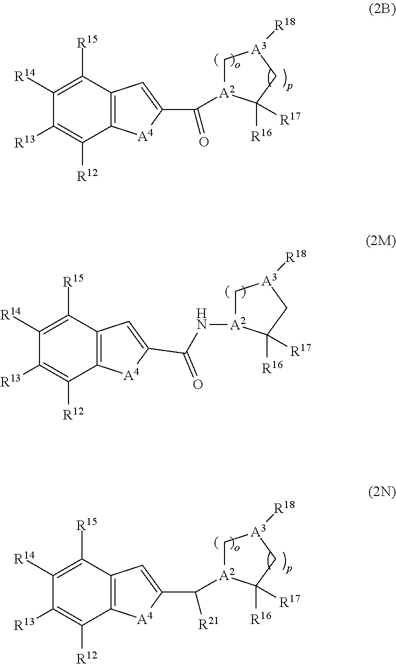

wherein o, A², A³, A⁴, R¹², R¹³, R¹⁴, R¹⁵, R¹⁶, R¹⁷, R¹⁸, and R²¹ have the same meaning as that defined herein.

In some embodiments the compounds of the present invention have structural formula (2P),

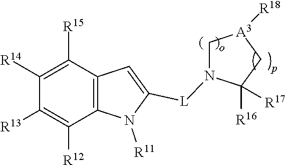

wherein o, L, A³, R¹¹, R¹², R¹³, R¹⁴, R¹⁵, R¹⁶, R¹⁷ and R¹⁸ have the same meaning as that defined herein.

According to particular embodiments, the present invention provides compounds of formula (2), and any subgroup thereof such as (2A), (2B), (2C), (2D), (2E), (2F), (2G), (2H), (2I), (2J), (2K), (2L), (2M), (2N), (2P)
wherein,
o is an integer selected from 1, 2;
p is 1;
A² is CR¹⁹; preferably is CH;
A³ is N; and
L is selected from —C═O, —C(O)—NH—, —CH₂—, —CH(OH)— and CH—COOR².

According to particular embodiments, the present invention provides compounds of formula (2), and any subgroup thereof such as (2A), (2B), (2C), (2D), (2E), (2F), (2G), (2H), (2I), (2J), (2K), (2L), (2M), (2N), (2P) wherein,
o is an integer selected from 0, 1, 2 or 3; preferably is 0, 1, or 2; preferably is 1;
p is an integer selected from 0, 1, 2, 3 or 4; preferably is 0, 1, 2, or 3; preferably is 0, 1, or 2; preferably is 1;
A² is selected from N or CR¹⁹;
A³ is selected from N or CR²⁰;
A⁴ is selected from NR¹¹, O, S or CR²⁴R²⁵; preferably is N, O, S or CHR²⁵; preferably is N, O, or S;
A⁵ is selected from N or CR¹²
A⁶ is selected from N or CR¹³;
A⁷ is selected from N or CR¹⁴;
A⁸ is selected from N or CR¹⁵;
wherein at least one of A² or A³ is N; preferably A² and A³ are N;
wherein at most one of A⁵ to A⁸ is N; preferably A⁵ to A⁸ are C; preferably A⁵ is N; preferably A⁶ is N; preferably A⁷ is N; preferably A⁸ is N;
L is selected from —C═O, —C(O)—NH—, and CHR²¹; preferably is —C═O, —C(O)—NH—, —CH₂—, —CH(OH)— and CH—COOR²³: preferably is —C═O;
R¹¹ is selected from the group consisting of hydrogen, deuterium, alkyl, —S(O)₂R²², aryl, —S(O)R²², and —SO₂NR²²R²³; and wherein said alkyl or aryl can be unsubstituted or substituted with one or more Z¹; preferably R¹¹ is hydrogen, deuterium, alkyl, —S(O)₂alkyl, —S(O)₂aryl, —S(O)₂cycloalkyl, aryl, —S(O)R²², —SO₂NHalkyl, —SO₂NHaryl, and —SO₂NHcycloalkyl; preferably is hydrogen, deuterium, alkyl, —S(O)₂alkyl, —S(O)₂aryl, —S(O)₂cycloalkyl, aryl, and —SO₂NHalkyl; preferably is hydrogen, deuterium, alkyl, —S(O)₂alkyl, —S(O)₂aryl, and —S(O)₂cycloalkyl; preferably is hydrogen, deuterium, alkyl, and —S(O)₂alkyl;
   preferably said alkyl or aryl can be unsubstituted or substituted with one, two or three Z¹;
R¹² is selected from the group consisting of hydrogen, deuterium, halogen, —OR²³, cyano, —C(O)R², alkyl, trifluoromethyl, trifluoromethoxy, cycloalkyl, aryl, heterocyclyl, heteroaryl, arylalkyl, and nitro, wherein said alkyl, cycloalkyl, aryl, heterocyclyl, heteroaryl, or arylalkyl is substituted by one or more Z^{1a}; preferably R¹² is hydrogen, deuterium, halogen, alkoxy, cycloalkyloxy, aryloxy, heterocycyloxy, heteroaryloxy, cyano, alkylcarbonyl cycloalkylcarbonyl, arylcarbonyl, alkyl, trifluoromethyl, trifluoromethoxy, cycloalkyl, aryl, heterocyclyl, heteroaryl, arylalkyl and nitro; preferably is hydrogen, deuterium, F, Br, Cl, I, alkoxy, aryloxy, cyano, alkylcarbonyl alkyl, trifluoromethyl, trifluoromethoxy, cycloalkyl, aryl, heterocyclyl, heteroaryl and arylalkyl; preferably is hydrogen, deuterium, F, Br, Cl, I, alkoxy, cyano, alkylcarbonyl alkyl, trifluoromethyl, trifluoromethoxy, and arylalkyl;
   preferably said alkyl, cycloalkyl, aryl, heterocyclyl, heteroaryl, or arylalkyl can be unsubstituted or substituted with one, two or three Z^{1a};
R¹³ is selected from the group consisting of hydrogen, deuterium, halogen, —OR²³, cyano, —C(O)R²³, alkyl, trifluoromethyl, trifluoromethoxy, cycloalkyl, aryl, heterocyclyl, heteroaryl, arylalkyl, and nitro, wherein said alkyl, cycloalkyl, aryl, heterocyclyl, heteroaryl, or arylalkyl is substituted by one or more Z^{1b}; preferably R¹³ is hydrogen, deuterium, halogen, alkoxy, cycloalkyloxy, aryloxy, heterocycyloxy, heteroaryloxy, cyano, alkylcarbonyl cycloalkylcarbonyl, arylcarbonyl, alkyl, trifluoromethyl, trifluoromethoxy, cycloalkyl, aryl, heterocyclyl, heteroaryl, arylalkyl and nitro; preferably is hydrogen, deuterium, F, Br, Cl, I, alkoxy, aryloxy, cyano, alkylcarbonyl alkyl, trifluoromethyl, trifluoromethoxy, cycloalkyl, aryl, heterocyclyl, heteroaryl and arylalkyl; preferably is hydrogen, deuterium, F, Br, Cl, I, alkoxy, cyano, alkylcarbonyl alkyl, trifluoromethyl, trifluoromethoxy, and arylalkyl;
   preferably said alkyl, cycloalkyl, aryl, heterocyclyl, heteroaryl, or arylalkyl can be unsubstituted or substituted with one, two or three Z^{1b};
R¹⁴ is selected from the group consisting of hydrogen, deuterium, halogen, —OR²³, cyano, —C(O)R²³, alkyl, trifluoromethyl, trifluoromethoxy, cycloalkyl, aryl, heterocyclyl, heteroaryl, arylalkyl, and nitro, wherein said alkyl, cycloalkyl, aryl, heterocyclyl, heteroaryl, or arylalkyl is substituted by one or more Z^{1c}; preferably R¹⁴ is hydrogen, deuterium, halogen, alkoxy, cycloalkyloxy, aryloxy, heterocycyloxy, heteroaryloxy, cyano, alkylcarbonyl cycloalkylcarbonyl, arylcarbonyl, alkyl, trifluoromethyl, trifluoromethoxy, cycloalkyl, aryl, heterocyclyl, heteroaryl, arylalkyl and nitro; preferably is hydrogen, deuterium, F, Br, Cl, I, alkoxy, aryloxy, cyano, alkylcarbonyl alkyl, trifluoromethyl, trifluoromethoxy, cycloalkyl, aryl, heterocyclyl, heteroaryl and arylalkyl; preferably is hydrogen, deuterium, F, Br, Cl, I, alkoxy, cyano, alkylcarbonyl alkyl, trifluoromethyl, trifluoromethoxy, and arylalkyl;
   preferably said alkyl, cycloalkyl, aryl, heterocyclyl, heteroaryl, or arylalkyl can be unsubstituted or substituted with one, two or three Z^{1c};
R¹⁵ is selected from the group consisting of hydrogen, deuterium, halogen, —OR²³, cyano, —C(O)R²³ alkyl, trifluoromethyl, trifluoromethoxy, cycloalkyl, aryl, heterocyclyl, heteroaryl, arylalkyl, and nitro, wherein said alkyl, cycloalkyl, aryl, heterocyclyl, heteroaryl, or arylalkyl is substituted by one or more Z^{1d}; preferably R¹⁵ is hydrogen, deuterium, halogen, alkoxy, cycloalkyloxy, aryloxy, heterocycyloxy, heteroaryloxy, cyano, alkylcarbonyl cycloalkylcarbonyl, arylcarbonyl, alkyl, trifluoromethyl, trifluoromethoxy, cycloalkyl, aryl, heterocyclyl, heteroaryl, arylalkyl and nitro; preferably is hydrogen, deuterium, F, Br, Cl, I, alkoxy, aryloxy, cyano, alkylcarbonyl alkyl, trifluoromethyl, trifluoromethoxy, cycloalkyl, aryl, heterocyclyl, heteroaryl and arylalkyl; preferably is hydrogen, deuterium, F, Br, Cl, I, alkoxy, cyano, alkylcarbonyl alkyl, trifluoromethyl, trifluoromethoxy, and arylalkyl;
   preferably said alkyl, cycloalkyl, aryl, heterocyclyl, heteroaryl, or arylalkyl can be unsubstituted or substituted with one, two or three Z^{1d};
R¹⁶ is selected from the group consisting of hydrogen, deuterium, alkyl, halogen, and —OR²³; preferably is hydrogen, deuterium, alkyl, halogen, alkoxy, cycloalkyloxy, aryloxy, heterocycyloxy, and heteroaryloxy; preferably is hydrogen, deuterium, alkyl, F. Br, Cl, I, alkoxy, cycloalkyloxy, aryloxy, heterocycyloxy, and heteroaryloxy; preferably is hydrogen, deuterium, alkyl, F, Br, Cl, I, and alkoxy;
R¹⁷ is selected from the group consisting of hydrogen, deuterium, alkyl, halogen, and —OR²³; preferably is hydrogen, deuterium, alkyl, halogen, alkoxy, cycloalkyloxy, aryloxy, heterocycyloxy, and heteroaryloxy; preferably is hydrogen, deuterium, alkyl, F, Br, Cl, I, alkoxy, cycloalkyloxy, aryloxy, heterocycyloxy, and heteroaryloxy; preferably is hydrogen, deuterium, alkyl, F, Br, Cl, I, and alkoxy;
or R¹⁶ and R¹⁷ together with the carbon atom to which they are attached from a group selected from —C═CH-alkyl, —C═N—OH, —C═N—O—Si(CH₃)₂C(CH₃)₃, or a saturated or unsaturated 3-, 4-, 5-, 6- or 7-membered ring; preferably —C═CH-alkyl, —C═N—OH, —C═N—O—Si(CH₃)₂C(CH₃)₃, or a saturated or unsaturated 3-, 4-, 5-, or 6-membered ring; preferably —C═CH—CH₃, —C═CH—CH₂CH₃, —C═N—OH, —C═N—O—Si(CH₃)₂C(CH₃)₃, or a saturated 3-, 4-, 5-, or 6-membered ring;
R¹⁸ is selected from the group consisting of halogen, —NH₂, —NHR²², alkyl, deuterium, arylalkyl, —S(O)₂R²², —C(O)OR²³, —S(O)R²², —(CH₂)₂—O—(CH₂)₂—NH₂, heteroaryl, cycloalkyl, aryl, heterocyclyl; and wherein said alkyl, arylalkyl, heteroaryl, cycloalkyl, aryl, heterocyclyl, or arylalkyl can be unsubstituted or substituted with one or more Z²; preferably halogen, —NH₂, mono-alkylamino, mono-arylamino, mono-cycloalkylamino, mono-heteroarylamino, mono-heterocyclcylamino, alkyl, deuterium, arylalkyl, alkyloxycarbonyl, alkylsulfonyl, cycloalkylsulfonyl, arylsulfonyl, —S(O)R²², —(CH₂)₂—O—(CH₂)₂—NH₂, heteroaryl, cycloalkyl, aryl, and heterocyclyl; preferably halogen, —NH₂, mono-alkylamino, mono-arylamino, mono-cycloalkylamino, alkyl, deuterium, arylalkyl, alkyloxycarbonyl, alkylsulfonyl, cycloalkylsulfonyl, heteroaryl, cycloalkyl, aryl, and heterocyclyl; preferably F, Br, Cl, I, —NH₂, mono-alkylamino, alkyl, deuterium, arylalkyl, alkyloxycarbonyl, alkylsulfonyl, —(CH₂)₂—O—(CH₂)₂—NH₂, heteroaryl, cycloalkyl, aryl, and heterocyclyl; preferably F, Br, Cl, I, —NH₂, mono-alkylamino, alkyl, deuterium, alkyloxycarbonyl, alkylsulfonyl, —(CH₂)₂—O—(CH₂)₂—NH₂, heteroaryl, and aryl;
   preferably said alkyl, arylalkyl, heteroaryl, cycloalkyl, aryl, heterocyclyl, or arylalkyl can be unsubstituted or substituted with one, two or three Z²;
R¹⁹ is selected from the group consisting of hydrogen, alkyl, halogen, and —OR²³; preferably is hydrogen, deuterium, alkyl, halogen, alkoxy, cycloalkyloxy, aryloxy, heterocycyloxy, and heteroaryloxy; preferably is hydrogen, deuterium, alkyl, F, Br, Cl, I, alkoxy, cycloalkyloxy, aryloxy, heterocycyloxy, and heteroaryloxy; preferably is hydrogen, deuterium, alkyl, F, Br, Cl, I, and alkoxy;
R²⁰ is selected from the group consisting of hydrogen, alkyl, halogen, and —OR²³; preferably is hydrogen, deuterium, alkyl, halogen, alkoxy, cycloalkyloxy, aryloxy, heterocycyloxy, and heteroaryloxy; preferably is hydrogen, deuterium, alkyl, F, Br, Cl, I, alkoxy, cycloalkyloxy, aryloxy, heterocycyloxy, and heteroaryloxy; preferably is hydrogen, deuterium, alkyl, F, Br, Cl, I, and alkoxy;
R²¹ is selected from the group consisting of —OH, COOR²³, —C(O)NH₂, hydrogen, and —OR²³; preferably —OH, alkyloxycarbonyl, cycloalkyloxycarbonyl, aryloxycarbonyl, —C(O)NH₂, hydrogen, alkoxy, cycloalkyloxy, aryloxy, heterocycyloxy, and heteroaryloxy; preferably —OH, alkyloxycarbonyl, —C(O)NH₂, hydrogen, alkoxy, cycloalkyloxy, and aryloxy; preferably —OH, alkyloxycarbonyl, hydrogen, and alkoxy;
each R²² is independently selected from the group consisting of alkyl, aryl, tolyl, cycloalkyl, arylalkyl, heterocyclyl, and heteroaryl; preferably alkyl, aryl, tolyl,cycloalkyl, and arylalkyl; preferably alkyl, aryl, tolyl and cycloalkyl;
each R²³ is independently selected from the group consisting of hydrogen, alkyl, aryl, cycloalkyl, arylalkyl, heterocyclyl, and heteroaryl; preferably alkyl, aryl, cycloalkyl, and arylalkyl; preferably alkyl, aryl, and cycloalkyl;
R²⁴ is selected from the group consisting of hydrogen, deuterium, halogen, —OR²³, cyano, —C(O)R²³, alkyl, trifluoromethyl, trifluoromethoxy, cycloalkyl, aryl, heterocyclyl, heteroaryl, arylalkyl, and nitro; preferably hydrogen, deuterium, halogen, alkoxy, cycloalkyloxy, aryloxy, heterocycyloxy, heteroaryloxy, cyano, alkylcarbonyl, cycloalkylcarbonyl, arylcarbonyl, alkyl, trifluoromethyl, trifluoromethoxy, cycloalkyl, aryl, heterocyclyl, heteroaryl, arylalkyl, and nitro; preferably hydrogen, deuterium, F, Br, Cl, I, alkoxy, cycloalkyloxy, aryloxy, cyano, alkylcarbonyl, alkyl, trifluoromethyl, trifluoromethoxy, cycloalkyl, aryl, heterocyclyl, heteroaryl, and arylalkyl; preferably hydrogen, deuterium, F, Br, Cl, I, alkoxy, cyano, alkylcarbonyl, alkyl, trifluoromethyl, trifluoromethoxy, cycloalkyl, and aryl;
R²⁵ is selected from the group consisting of hydrogen, deuterium, halogen, —OR²³, cyano, —C(O)R²³, alkyl, trifluoromethyl, trifluoromethoxy, cycloalkyl, aryl, heterocyclyl, heteroaryl, arylalkyl, and nitro; preferably hydrogen, deuterium, halogen, alkoxy, cycloalkyloxy, aryloxy, heterocycyloxy, heteroaryloxy, cyano, alkylcarbonyl, cycloalkylcarbonyl, arylcarbonyl, alkyl, trifluoromethyl, trifluoromethoxy, cycloalkyl, aryl, heterocyclyl, heteroaryl, arylalkyl, and nitro; preferably hydrogen, deuterium, F, Br, Cl, I, alkoxy, cycloalkyloxy, aryloxy, cyano, alkylcarbonyl, alkyl, trifluoromethyl, trifluoromethoxy, cycloalkyl, aryl, heterocyclyl, heteroaryl, and arylalkyl; preferably hydrogen, deuterium, F, Br, Cl, I, alkoxy, cyano, alkylcarbonyl, alkyl, trifluoromethyl, trifluoromethoxy, cycloalkyl, and aryl;
each Z¹ is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro; preferably alkoxy, cycloalkyloxy, aryloxy, heterocycyloxy, heteroaryloxy, halogen, alkyl, —NH₂, mono-alkylamino, mono-arylamino, mono-cycloalkylamino, mono-heteroarylamino, mono-heterocyclcylamino, alkyloxycarbonyl, cycloalkyloxycarbonyl, aryloxycarbonyl, cycloalkyl, trifluoromethyl, trifluoromethoxy, aryl, arylalkyl, heterocyclyl, heteroaryl, —OH, cyano and nitro; preferably alkoxy, cycloalkyloxy, aryloxy, halogen, alkyl, —NH₂, mono-alkylamino, mono-cycloalkylamino, alkyloxycarbonyl, cycloalkyl, trifluoromethyl, trifluoromethoxy, aryl, arylalkyl, heterocyclyl, heteroaryl, —OH, cyano and nitro; preferably alkoxy, cycloalkyloxy, aryloxy, F, Br, Cl, I, alkyl, —NH₂, mono-alkylamino, alkyloxycarbonyl, cycloalkyl, trifluoromethyl, trifluoromethoxy, aryl, arylalkyl, heterocyclyl, heteroaryl, and —OH; preferably alkoxy, F, Br, Cl, I, alkyl, —NH₂, mono-alkylamino, alkyloxycarbonyl, cycloalkyl, trifluoromethyl, trifluoromethoxy, aryl, and —OH;
each Z^{1a} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro; preferably alkoxy, cycloalkyloxy, aryloxy, heterocycyloxy, heteroaryloxy, halogen, alkyl, —NH₂, mono-alkylamino, mono-arylamino, mono-cycloalkylamino, mono-heteroarylamino, mono-heterocyclcylamino, alkyloxycarbonyl, cycloalkyloxycarbonyl, aryloxycarbonyl, cycloalkyl, trifluoromethyl, trifluoromethoxy, aryl, arylalkyl, heterocyclyl, heteroaryl, —OH, cyano and nitro; preferably alkoxy, cycloalkyloxy, aryloxy, halogen, alkyl, —NH₂, mono-alkylamino, mono-cycloalkylamino, alkyloxycarbonyl, cycloalkyl, trifluoromethyl, trifluoromethoxy, aryl, arylalkyl, heterocyclyl, heteroaryl, —OH, cyano and nitro; preferably alkoxy, cycloalkyloxy, aryloxy, F, Br, Cl, I, alkyl, —NH₂, mono-alkylamino, alkyloxycarbonyl, cycloalkyl, trifluoromethyl, trifluoromethoxy, aryl, arylalkyl, heterocyclyl, heteroaryl, and —OH; preferably alkoxy, F, Br, Cl, I, alkyl, —NH₂, mono-alkylamino, alkyloxycarbonyl, cycloalkyl, trifluoromethyl, trifluoromethoxy, aryl, and —OH;
each Z^{1b} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro; preferably alkoxy, cycloalkyloxy, aryloxy, heterocycyloxy, heteroaryloxy, halogen, alkyl, —NH₂, mono-alkylamino, mono-arylamino, mono-cycloalkylamino, mono-heteroarylamino, mono-heterocyclcylamino, alkyloxycarbonyl, cycloalkyloxycarbonyl, aryloxycarbonyl, cycloalkyl, trifluoromethyl, trifluoromethoxy, aryl, arylalkyl, heterocyclyl, heteroaryl, —OH, cyano and nitro; preferably alkoxy, cycloalkyloxy, aryloxy, halogen, alkyl, —NH₂, mono-alkylamino, mono-cycloalkylamino, alkyloxycarbonyl, cycloalkyl, trifluoromethyl, trifluoromethoxy, aryl, arylalkyl, heterocyclyl, heteroaryl, —OH, cyano and nitro; preferably alkoxy, cycloalkyloxy, aryloxy, F, Br, Cl, I, alkyl, —NH₂, mono-alkylamino, alkyloxycarbonyl, cycloalkyl, trifluoromethyl, trifluoromethoxy, aryl, arylalkyl, heterocyclyl, heteroaryl, and —OH; preferably alkoxy, F, Br, Cl, I, alkyl, —NH₂, mono-alkylamino, alkyloxycarbonyl, cycloalkyl, trifluoromethyl, trifluoromethoxy, aryl, and —OH;
each Z^{1c} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro; preferably alkoxy, cycloalkyloxy, aryloxy, heterocycyloxy, heteroaryloxy, halogen, alkyl, —NH₂, mono-alkylamino, mono-arylamino, mono-cycloalkylamino, mono-heteroarylamino, mono-heterocyclcylamino, alkyloxycarbonyl, cycloalkyloxycarbonyl, aryloxycarbonyl, cycloalkyl, trifluoromethyl, trifluoromethoxy, aryl, arylalkyl, heterocyclyl, heteroaryl, —OH, cyano and nitro; preferably alkoxy, cycloalkyloxy, aryloxy, halogen, alkyl, —NH₂, mono-alkylamino, mono-cycloalkylamino, alkyloxycarbonyl, cycloalkyl, trifluoromethyl, trifluoromethoxy, aryl, arylalkyl, heterocyclyl, heteroaryl, —OH, cyano and nitro; preferably alkoxy, cycloalkyloxy, aryloxy, F, Br, Cl, I, alkyl, —NH₂, mono-alkylamino, alkyloxycarbonyl, cycloalkyl, trifluoromethyl, trifluoromethoxy, aryl, arylalkyl, heterocyclyl, heteroaryl, and —OH; preferably alkoxy, F, Br, Cl, I, alkyl, —NH₂, mono-alkylamino, alkyloxycarbonyl, cycloalkyl, trifluoromethyl, trifluoromethoxy, aryl, and —OH;
each Z^{1d} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro; preferably alkoxy, cycloalkyloxy, aryloxy, heterocycyloxy, heteroaryloxy, halogen, alkyl, —NH₂, mono-alkylamino, mono-arylamino, mono-cycloalkylamino, mono-heteroarylamino, mono-heterocyclcylamino, alkyloxycarbonyl, cycloalkyloxycarbonyl, aryloxycarbonyl, cycloalkyl, trifluoromethyl, trifluoromethoxy, aryl, arylalkyl, heterocyclyl, heteroaryl, —OH, cyano and nitro; preferably alkoxy, cycloalkyloxy, aryloxy, halogen, alkyl, —NH₂, mono-alkylamino, mono-cycloalkylamino, alkyloxycarbonyl, cycloalkyl, trifluoromethyl, trifluoromethoxy, aryl, arylalkyl, heterocyclyl, heteroaryl, —OH, cyano and nitro; preferably alkoxy, cycloalkyloxy, aryloxy, F, Br, Cl, I, alkyl, —NH₂, mono-alkylamino, alkyloxycarbonyl, cycloalkyl, trifluoromethyl, trifluoromethoxy, aryl, arylalkyl, heterocyclyl, heteroaryl, and —OH; preferably alkoxy, F, Br, Cl, I, alkyl, —NH₂, mono-alkylamino, alkyloxycarbonyl, cycloalkyl, trifluoromethyl, trifluoromethoxy, aryl, and —OH;
each Z² is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro; preferably alkoxy, cycloalkyloxy, aryloxy, heterocycyloxy, heteroaryloxy, halogen, alkyl, —NH₂, mono-alkylamino, mono-arylamino, mono-cycloalkylamino, mono-heteroarylamino, mono-heterocyclcylamino, alkyloxycarbonyl, cycloalkyloxycarbonyl, aryloxycarbonyl, cycloalkyl, trifluoromethyl, trifluoromethoxy, aryl, arylalkyl, heterocyclyl, heteroaryl, —OH, cyano and nitro; preferably alkoxy, cycloalkyloxy, aryloxy, halogen, alkyl, —NH₂, mono-alkylamino, mono-cycloalkylamino, alkyloxycarbonyl, cycloalkyl, trifluoromethyl, trifluoromethoxy, aryl, arylalkyl, heterocyclyl, heteroaryl, —OH, cyano and nitro; preferably alkoxy, cycloalkyloxy, aryloxy, F, Br, Cl, I, alkyl, —NH₂, mono-alkylamino, alkyloxycarbonyl, cycloalkyl, trifluoromethyl, trifluoromethoxy, aryl, arylalkyl, heterocyclyl, heteroaryl, and —OH; preferably alkoxy, F, Br, Cl, I, alkyl, —NH₂, mono-alkylamino, alkyloxycarbonyl, cycloalkyl, trifluoromethyl, trifluoromethoxy, aryl, and —OH.

According to particular embodiments, the present invention provides compounds of formula (2), and any subgroup thereof such as (2A), (2B), (2C), (2D), (2E), (2F), (2G), (2H), (2I), (2J), (2K), (2L), (2M), (2N), (2P) wherein,
o is an integer selected from 0, 1, 2 or 3; preferably is 0, 1, or 2; preferably is 1;
p is an integer selected from 0, 1, 2, 3 or 4; preferably is 0, 1, 2, or 3; preferably is 0, 1, or 2; preferably is 1;
A² is selected from N or CR¹⁹;
A³ is selected from N or CR²⁰;
A⁴ is selected from NR¹¹, O, S or CR²⁴R²⁵; preferably is N, O, S or CHR²⁵; preferably is N, O, or S;
A⁵ is selected from N or CR¹²;
A⁶ is selected from N or CR¹³;
A⁷ is selected from N or CR¹⁴;
A⁸ is selected from N or CR¹⁵;
wherein at least one of A² or A³ is N; preferably A² and A³ are N;
wherein at most one of A⁵ to A⁸ is N; preferably A⁵ to A⁸ are C; preferably A⁵ is N; preferably A⁶ is N; preferably A⁷ is N: preferably A⁸ is N;
L is selected from —C═O, —C(O)—NH—, and CHR²¹; preferably is —C═O, —C(O)—NH—, —CH₂—, —CH(OH)— and CH—COOR²³; preferably is —C═O;
R¹¹ is selected from the group consisting of hydrogen, deuterium, C₁₋₆alkyl, —S(O)₂R²², C₆₋₁₂aryl, —S(O)R²², and —SO₂NR²²R²³; and wherein said C₁₋₆alkyl or C₆₋₁₂aryl can be unsubstituted or substituted with one or more Z¹; preferably R¹¹ is hydrogen, deuterium, C₁₋₆alkyl, —S(O)₂C₁₋₆alkyl, —S(O)₂C₆₋₁₂aryl, —S(O)₂C₃₋₆cycloalkyl, C₆₋₁₂aryl, —S(O)R²², —SO₂NHC₁₋₆alkyl, —SO₂NHC₆₋₁₂aryl, and —SO₂NHC₃₋₈cycloalkyl; preferably is hydrogen, deuterium, C₁₋₆alkyl, —S(O)₂C₁₋₆alkyl, —S(O)₂C₆₋₁₂aryl, —S(O)₂C₃₋₈cycloalkyl, C₆₋₁₂aryl, and —SO₂NH C₁₋₆alkyl; preferably is hydrogen, deuterium, C₁₋₄alkyl, —S(O)₂C₁₋₄alkyl, —S(O)₂C₆₋₁₀ aryl, —S(O)₂C₃₋₆cycloalkyl, C₆₋₁₀aryl, and —SO₂NH C₁₋₄alkyl; preferably is hydrogen, deuterium, C₁₋₆alkyl, —S(O)₂C₁₋₆alkyl, —S(O)₂C₆₋₁₂aryl, and —S(O)₂C₃₋₈cycloalkyl; preferably is hydrogen, deuterium, C₁₋₄alkyl, —S(O)₂C₁₋₄alkyl, —S(O)₂C₆₋₁₀aryl, and —S(O)₂C₃₋₆cycloalkyl; preferably is hydrogen, deuterium, C₁₋₆alkyl, and —S(O)₂C₁₋₆alkyl; preferably is hydrogen, deuterium, C₁₋₄alkyl, and —S(O)₂C₁₋₄alkyl;
   preferably said C₁₋₆alkyl or aryl can be unsubstituted or substituted with one, two or three Z¹;
R¹² is selected from the group consisting of hydrogen, deuterium, halogen, —OR²³, cyano, —C(O)R²³, C₁₋₆alkyl, trifluoromethyl, trifluoromethoxy, C₃₋₈cycloalkyl, C₆₋₁₂aryl, heterocyclyl, heteroaryl, C₆₋₁₂arylC₁₋₆alkyl, and nitro, wherein said C₁₋₆alkyl, C₃₋₈cycloalkyl, C₆₋₁₂aryl, heterocyclyl, heteroaryl, or C₆₋₁₂arylC₁₋₆alkyl is substituted by one or more Z^{1a}; preferably R¹² is hydrogen, deuterium, halogen, C₁₋₆alkoxy, C₃₋₈cycloalkyloxy, C₆₋₁₂aryloxy, heterocycyloxy, heteroaryloxy, cyano. C₁₋₆alkylcarbonyl, C₃₋₈cycloalkylcarbonyl, C₆₋₁₂arylcarbonyl, C₁₋₆alkyl, trifluoromethyl, trifluoromethoxy, C₃₋₈cycloalkyl, C₆₋₁₂aryl, heterocyclyl, heteroaryl, C₆₋₁₂arylC₁₋₆alkyl and nitro; preferably is hydrogen, deuterium, F, Br, Cl, I, C₁₋₆alkoxy, C₆₋₁₂aryloxy, cyano, C₁₋₆alkylcarbonyl, C₁₋₆alkyl, trifluoromethyl, trifluoromethoxy, C₃₋₈cycloalkyl, C₆₋₁₂aryl, heterocyclyl, heteroaryl and C₆₋₁₂arylC₁₋₆alkyl; preferably is hydrogen, deuterium, F, Br, Cl, I, C₁₋₄alkoxy, C₆₋₁₀ aryloxy, cyano, C₁₋₄alkylcarbonyl, C₁₋₄alkyl, trifluoromethyl, trifluoromethoxy, C₃₋₆cycloalkyl, C₆₋₁₀aryl, heterocyclyl, heteroaryl and C₆₋₁₀arylC₁₋₄alkyl; preferably is hydrogen, deuterium, F, Br, Cl, I, alkoxy, cyano, C₁₋₆alkylcarbonyl, C₁₋₆alkyl, trifluoromethyl, trifluoromethoxy, and C₆₋₁₂arylC₁₋₆alkyl; preferably is hydrogen, deuterium. F, Br, Cl, I, alkoxy, cyano, C₁₋₄alkylcarbonyl, C₁₋₄alkyl, trifluoromethyl, trifluoromethoxy, and C₆₋₁₀arylC₁₋₄alkyl;
   preferably said C₁₋₆alkyl, C₃₋₈cycloalkyl, C₆₋₁₂aryl, heterocyclyl, heteroaryl, or C₆₋₁₂arylC₁₋₆alkyl, can be unsubstituted or substituted with one, two or three Z^{1a};
R¹³ is selected from the group consisting of hydrogen, deuterium, halogen, —OR²³, cyano, —C(O)R²³, C₁₋₆alkyl, trifluoromethyl, trifluoromethoxy, C₃₋₈cycloalkyl, C₆₋₁₂aryl, heterocyclyl, heteroaryl, C₆₋₁₂arylC₁₋₆alkyl, and nitro, wherein said C₁₋₆alkyl, C₃₋₈cycloalkyl, C₆₋₁₂aryl, heterocyclyl, heteroaryl, or C₆₋₁₂arylC₁₋₆alkyl is substituted by one or more Z^{1b}; preferably R¹³ is hydrogen, deuterium, halogen, C₁₋₆alkoxy, C₃₋₆cycloalkyloxy, C₆₋₁₂aryloxy, heterocycyloxy, heteroaryloxy, cyano, C₁₋₆ alkylcarbonyl, C₃₋₆cycloalkylcarbonyl, C₆₋₁₂arylcarbonyl, C₁₋₆alkyl, trifluoromethyl, trifluoromethoxy, C₃₋₈cycloalkyl, C₆₋₁₂aryl, heterocyclyl, heteroaryl, C₆₋₁₂arylC₁₋₆alkyl and nitro; preferably is hydrogen, deuterium, F, Br, Cl, I, C₁₋₆alkoxy, C₆₋₁₂aryloxy, cyano, C₁₋₆alkylcarbonyl, C₁₋₆alkyl, trifluoromethyl, trifluoromethoxy, C₃₋₈cycloalkyl, C₆₋₁₂aryl, heterocyclyl, heteroaryl and C₆₋₁₂arylC₁₋₆alkyl; preferably is hydrogen, deuterium, F, Br, Cl, I, C₁₋₄alkoxy, C₆₋₁₀aryloxy, cyano, C₁₋₄alkylcarbonyl, C₁₋₄alkyl, trifluoromethyl, trifluoromethoxy, C₃₋₆cycloalkyl, C₆₋₁₀aryl, heterocyclyl, heteroaryl and C₆₋₁₀arylC₁₋₄alkyl; preferably is hydrogen, deuterium, F, Br, Cl, I, alkoxy, cyano, C₁₋₆alkylcarbonyl, C₁₋₆alkyl, trifluoromethyl, trifluoromethoxy, and C₆₋₁₂arylC₁₋₆alkyl; preferably is hydrogen, deuterium, F, Br, Cl, I, alkoxy, cyano, C₁₋₄alkylcarbonyl, C₁₋₄alkyl, trifluoromethyl, trifluoromethoxy, and C₆₋₁₀arylC₁₋₄alkyl;
   preferably said C₁₋₆alkyl, C₃₋₈cycloalkyl, C₆₋₁₂aryl, heterocyclyl, heteroaryl, or C₆₋₁₂arylC₁₋₆alkyl, can be unsubstituted or substituted with one, two or three Z^{1b};
R¹⁴ is selected from the group consisting of hydrogen, deuterium, halogen, —OR²³, cyano, —C(O)R²³, C₁₋₆alkyl, trifluoromethyl, trifluoromethoxy, C₃₋₈cycloalkyl, C₆₋₁₂aryl, heterocyclyl, heteroaryl, C₆₋₁₂ arylC₁₋₆alkyl, and nitro, wherein said C₁₋₆alkyl, C₃₋₈cycloalkyl, C₆₋₁₂aryl, heterocyclyl, heteroaryl, or C₆₋₁₂arylC₁₋₆alkyl is substituted by one or more Z^{1c}; preferably R¹⁴ is hydrogen, deuterium, halogen, C₁₋₆alkoxy, C₃₋₈cycloalkyloxy, C₆₋₁₂aryloxy, heterocycyloxy, heteroaryloxy, cyano, C₁₋₆ alkylcarbonyl, C₃₋₈cycloalkylcarbonyl, C₆₋₁₂arylcarbonyl, C₁₋₆alkyl, trifluoromethyl, trifluoromethoxy, C₃₋₈cycloalkyl, C₆₋₁₂aryl, heterocyclyl, heteroaryl, C₆₋₁₂arylC₁₋₆alkyl and nitro; preferably is hydrogen, deuterium, F, Br, Cl, I, C₁₋₆alkoxy, C₆₋₁₂aryloxy, cyano, C₁₋₆alkylcarbonyl, C₁₋₆alkyl, trifluoromethyl, trifluoromethoxy, C₃₋₈cycloalkyl, C₆₋₁₂aryl, heterocyclyl, heteroaryl and C₆₋₁₂arylC₁₋₆alkyl; preferably is hydrogen, deuterium, F, Br, Cl, I, C₁₋₄alkoxy, C₆₋₁₀aryloxy, cyano, C₁₋₄alkylcarbonyl, C₁₋₄alkyl, trifluoromethyl, trifluoromethoxy, C₃₋₆cycloalkyl, C₆₋₁₀aryl, heterocyclyl, heteroaryl and C₆₋₁₀arylC₁₋₄alkyl; preferably is hydrogen, deuterium, F, Br, Cl, I, alkoxy, cyano, C₁₋₆alkylcarbonyl, C₁₋₆alkyl, trifluoromethyl, trifluoromethoxy, and C₁₋₁₂arylC₁₋₆alkyl; preferably is hydrogen, deuterium, F, Br, Cl, I, alkoxy, cyano, C₁₋₄alkylcarbonyl, C₁₋₄alkyl, trifluoromethyl, trifluoromethoxy, and C₆₋₁₀arylC₁₋₄alkyl;
   preferably said C₁₋₆alkyl, C₃₋₈cycloalkyl, C₆₋₁₂aryl, heterocyclyl, heteroaryl, or C₆₋₁₂arylC₁₋₆alkyl, can be unsubstituted or substituted with one, two or three Z^{1c};
R¹⁵ is selected from the group consisting of hydrogen, deuterium, halogen, —OR²³, cyano, —C(O)R²³, C₁₋₆alkyl, trifluoromethyl, trifluoromethoxy, C₃₋₈cycloalkyl, C₆₋₁₂aryl, heterocyclyl, heteroaryl, C₆₋₁₂ arylC₁₋₆alkyl, and nitro, wherein said C₁₋₆alkyl, C₃₋₈cycloalkyl, C₆₋₁₂aryl, heterocyclyl, heteroaryl, or C₆₋₁₂arylC₁₋₆alkyl is substituted by one or more Z^{1d}; preferably R¹⁵ is hydrogen, deuterium, halogen, C₁₋₆alkoxy, C₃₋₈cycloalkyloxy, C₆₋₁₂aryloxy, heterocycyloxy, heteroaryloxy, cyano, C₁₋₆ alkylcarbonyl, C₃₋₈cycloalkylcarbonyl, C₆₋₁₂arylcarbonyl, C₁₋₆alkyl, trifluoromethyl, trifluoromethoxy, C₃₋₈cycloalkyl, C₆₋₁₂aryl, heterocyclyl, heteroaryl, C₆₋₁₂arylC₁₋₆alkyl and nitro; preferably is hydrogen, deuterium, F, Br, Cl, I, C₁₋₆alkoxy, C₆₋₁₂aryloxy, cyano, C₁₋₆alkylcarbonyl, C₁₋₆alkyl, trifluoromethyl, trifluoromethoxy, C₃₋₈cycloalkyl, C₆₋₁₂aryl, heterocyclyl, heteroaryl and C₆₋₂arylC₁₋₆alkyl; preferably is hydrogen, deuterium, F, Br, Cl, I, C₁₋₄alkoxy, C₆₋₁₀aryloxy, cyano, C₁₋₄alkylcarbonyl, C₁₋₄alkyl, trifluoromethyl, trifluoromethoxy, C₃₋₆cycloalkyl, C₆₋₁₀aryl, heterocyclyl, heteroaryl and C₆₋₁₀arylC₁₋₄alkyl; preferably is hydrogen, deuterium, F, Br, Cl, I, alkoxy, cyano, C₁₋₆alkylcarbonyl, C₁₋₆alkyl, trifluoromethyl, trifluoromethoxy, and C₆₋₁₂arylC₁₋₆alkyl; preferably is hydrogen, deuterium, F, Br, Cl, I, alkoxy, cyano, C₁₋₄alkylcarbonyl, C₁₋₄alkyl, trifluoromethyl, trifluoromethoxy, and C₆₋₁₀ arylC₁₋₄alkyl;
   preferably said C₁₋₆alkyl, C₃₋₈cycloalkyl, C₆₋₁₂aryl, heterocyclyl, heteroaryl, or C₆₋₁₂arylC₁₋₆alkyl, can be unsubstituted or substituted with one, two or three Z^{1d};
R¹⁶ is selected from the group consisting of hydrogen, deuterium, C₁₋₆alkyl, halogen, and —OR²³: preferably is hydrogen, deuterium, C₁₋₆alkyl, halogen, C₁₋₆alkoxy, C₃₋₈cycloalkyloxy, C₆₋₁₂aryloxy, heterocycyloxy, and heteroaryloxy; preferably is hydrogen, deuterium, C₁₋₆alkyl, F, Br, Cl, I, C₁₋₆ alkoxy, C₃₋₆cycloalkyloxy, C₆₋₁₂aryloxy, heterocycyloxy, and heteroaryloxy; preferably is hydrogen, deuterium, C₁₋₄alkyl, F, Br, Cl, I, C₁₋₄alkoxy, C₃₋₆cycloalkyloxy, C₆₋₁₀aryloxy, heterocycyloxy, and heteroaryloxy; preferably is hydrogen, deuterium, alkyl, F, Br, Cl, I, and C₁₋₆alkoxy; preferably is hydrogen, deuterium, alkyl, F, Br, Cl, I, and C₁₋₄alkoxy;
R¹⁷ is selected from the group consisting of hydrogen, deuterium, C₁₋₆alkyl, halogen, and —OR²³: preferably is hydrogen, deuterium, C₁₋₆alkyl, halogen, C₁₋₆alkoxy, C₃₋₈cycloalkyloxy, C₆₋₁₂aryloxy, heterocycyloxy, and heteroaryloxy; preferably is hydrogen, deuterium, C₁₋₆alkyl, F, Br, Cl, I, C₁₋₆alkoxy, C₃₋₈cycloalkyloxy, C₆₋₁₂aryloxy, heterocycyloxy, and heteroaryloxy; preferably is hydrogen, deuterium, C₁₋₄alkyl, F, Br, Cl, I, C₁₋₄alkoxy, C₃₋₆cycloalkyloxy, C₆₋₁₀aryloxy, heterocycyloxy, and heteroaryloxy; preferably is hydrogen, deuterium, alkyl, F, Br, Cl, I, and C₁₋₆alkoxy; preferably is hydrogen, deuterium, alkyl, F, Br, Cl, I, and C₁₋₄alkoxy;
or R¹⁶ and R¹⁷ together with the carbon atom to which they are attached from a group selected from —C═CH—C₁₋₆alkyl, —C═N—OH, —C═N—O—Si(CH₃)₂C(CH₃)₃, or a saturated or unsaturated 3-, 4-, 5-, 6- or 7-membered ring; preferably —C═CH—C₁₋₆alkyl, —C═N—OH, —C═N—O—Si(CH₃)₂C(CH₃)₃, or a saturated or unsaturated 3-, 4-, 5-, or 6-membered ring; preferably —C═CH—C₁₋₄alkyl, —C═N—OH, —C═N—O—Si(CH₃)₂C(CH₃)₃, or a saturated or unsaturated 3-, 4-, 5-, or 6-membered ring; preferably —C═CH—CH₃, —C═CH—CH₂CH₃, —C═N—OH, —C═N—O—Si(CH₃)₂C(CH₃)₃, or a saturated 3-, 4-, 5-, or 6-membered ring;
R¹⁸ is selected from the group consisting of halogen, —NH₂, —NHR²², C₁₋₆alkyl, deuterium, C₆₋₁₂arylC₁₋₆alkyl, —S(O)₂R²², —C(O)OR²³, —S(O)R²², —(CH₂)₂—O—(CH₂)₂—NH₂, heteroaryl, C₃₋₈cycloalkyl, C₆₋₁₂aryl, heterocyclyl; and wherein said C₁₋₆alkyl, C₆₋₁₂arylarylC₁₋₆alkyl, heteroaryl, C₃₋₈cycloalkyl, C₆₋₁₂aryl, heterocyclyl, or C₆₋₁₂arylC₁₋₆alkyl can be unsubstituted or substituted with one or more Z²; preferably halogen, —NH₂, mono-C₁₋₆alkylamino, mono-C₆₋₁₂arylamino, mono-C₃₋₈cycloalkylamino, mono-heteroarylamino, mono-heterocyclcylamino, C₁₋₆alkyl, deuterium, C₆₋₁₂arylC₁₋₆alkyl, C₁₋₆alkyloxycarbonyl, C₁₋₆alkylsulfonyl, C₃₋₈cycloalkylsulfonyl, C₆₋₁₂arylsulfonyl, —S(O)R²², —(CH₂)₂—O—(CH₂)₂—NH₂, heteroaryl, C₃₋₈cycloalkyl, C₆₋₁₂aryl, and heterocyclyl; preferably halogen, —NH₂, mono-C₁₋₆alkylamino, mono-C₆₋₁₂arylamino, mono-C₃₋₈cycloalkylamino, C₁₋₆alkyl, deuterium, C₆₋₁₂arylC₁₋₆alkyl, C₁₋₆alkyloxycarbonyl, C₁₋₆alkylsulfonyl, C₃₋₈cycloalkylsulfonyl, heteroaryl, C₃₋₈cycloalkyl, C₆₋₁₂aryl, and heterocyclyl; preferably F, Br, Cl, I, —NH₂, mono-C₁₋₆alkylamino, C₁₋₆alkyl, deuterium, C₆₋₁₂arylC₁₋₆alkyl, C₁₋₆alkyloxycarbonyl, C₁₋₆alkylsulfonyl, —(CH₂)₂—O—(CH₂)₂—NH₂, heteroaryl, C₃₋₈ cycloalkyl, C₆₋₁₂aryl, and heterocyclyl; preferably F, Br, Cl, I, —NH₂, mono-C₁₋₄alkylamino, C₁₋₄ alkyl, deuterium, C₆₋₁₀arylC₁₋₄alkyl, C₁₋₄alkyloxycarbonyl, C₁₋₄alkylsulfonyl, —(CH₂)₂—O—(CH₂)₂—NH₂, heteroaryl, C₃₋₆cycloalkyl, C₆₋₁₀aryl, and heterocyclyl; preferably F, Br, Cl, I, —NH₂, mono-C₁₋₆alkylamino, C₁₋₆alkyl, deuterium, C₁₋₆alkyloxycarbonyl, C₁₋₆alkylsulfonyl, —(CH₂)₂—O—(CH₂)₂—NH₂, heteroaryl, and C₆₋₁₂aryl; preferably F, Br, Cl, I, —NH₂, mono-C₁₋₄alkylamino, C₁₋₄alkyl, deuterium, C₁₋₄alkyloxycarbonyl, C₁₋₄alkylsulfonyl, —(CH₂)₂—O—(CH₂)₂—NH₂, heteroaryl, and C₆₋₁₀aryl; preferably said C₁₋₆alkyl, arylC₁₋₆alkyl, heteroaryl, cycloalkyl, aryl, heterocyclyl, or arylC₁₋₆alkyl can be unsubstituted or substituted with one, two or three Z²;
   preferably said C₁₋₆alkyl or aryl can be unsubstituted or substituted with one, two or three Z²;
R¹⁹ is selected from the group consisting of hydrogen. C₁₋₆alkyl, halogen, and —OR²; preferably is hydrogen, deuterium, C₁₋₆alkyl, halogen, C₁₋₆alkoxy, C₃₋₈cycloalkyloxy, C₆₋₁₂aryloxy, heterocycyloxy, and heteroaryloxy; preferably is hydrogen, deuterium, C₁₋₆alkyl, F, Br, Cl, I, C₁₋₆ alkoxy, C₃₋₈cycloalkyloxy, C₆₋₁₂aryloxy, heterocycyloxy, and heteroaryloxy; preferably is hydrogen, deuterium, C₁₋₄alkyl, F, Br, Cl, I, C₁₋₄alkoxy, C₃₋₆cycloalkyloxy, C₆₋₁₀ aryloxy, heterocycyloxy, and heteroaryloxy; preferably is hydrogen, deuterium, C₁₋₆alkyl, F, Br, Cl, I, and C₁₋₆alkoxy; preferably is hydrogen, deuterium, C₁₋₄alkyl, F, Br, Cl, I, and C₁₋₄alkoxy;
R²⁰ is selected from the group consisting of hydrogen, C₁₋₆alkyl, halogen, and —OR²³; preferably is hydrogen, deuterium, C₁₋₆alkyl, halogen, C₁₋₆alkoxy, C₃₋₈cycloalkyloxy, C₆₋₁₂aryloxy, heterocycyloxy, and heteroaryloxy; preferably is hydrogen, deuterium, C₁₋₆alkyl, F, Br, Cl, I, C₁₋₆alkoxy, C₃₋₈cycloalkyloxy, C₆₋₁₂aryloxy, heterocycyloxy, and heteroaryloxy; preferably is hydrogen, deuterium, C₁₋₄alkyl, F, Br, Cl, I, C₁₋₄alkoxy, C₃₋₆cycloalkyloxy, C₆₋₁₀ aryloxy, heterocycyloxy, and heteroaryloxy; preferably is hydrogen, deuterium, C₁₋₆alkyl, F, Br, Cl, I, and C₁₋₆alkoxy; preferably is hydrogen, deuterium, C₁₋₄alkyl, F, Br, Cl, I, and C₁₋₄alkoxy;
R²¹ is selected from the group consisting of —OH, COOR²³, —C(O)NH₂, hydrogen, and —OR²³; preferably —OH, C₁₋₆alkyloxycarbonyl, C₃₋₈cycloalkyloxycarbonyl, C₆₋₁₂aryloxycarbonyl, —C(O)NH₂, hydrogen, C₁₋₆alkoxy, C₃₋₈cycloalkyloxy, C₆₋₁₂aryloxy, heterocycyloxy, and heteroaryloxy; preferably —OH, C₁₋₆alkyloxycarbonyl, —C(O)NH₂, hydrogen, C₁₋₆alkoxy, C₃₋₈cycloalkyloxy, and C₆₋₁₂aryloxy; preferably —OH, C₁₋₄alkyloxycarbonyl, —C(O)NH₂, hydrogen, C₁₋₄alkoxy, C₃₋₆cycloalkyloxy, and C₆₋₁₀aryloxy; preferably —OH, C₁₋₆alkyloxycarbonyl, hydrogen, and C₁₋₆alkoxy; preferably —OH, C₁₋₄ alkyloxycarbonyl, hydrogen, and C₁₋₄alkoxy;
each R²² is independently selected from the group consisting of C₁₋₆alkyl, tolyl, C₆₋₁₂aryl, C₃₋₈cycloalkyl, C₆₋₁₂arylC₁₋₆alkyl, heterocyclyl, and heteroaryl; preferably C₁₋₆alkyl, C₆₋₁₂aryl, tolyl, C₃₋₈cycloalkyl, and C₆₋₁₂arylC₁₋₆alkyl; preferably C₁₋₄alkyl, C₆₋₁₀aryl, C₃₋₆cycloalkyl, tolyl and C₆₋₁₀aryl C₁₋₄alkyl; preferably C₁₋₆alkyl, C₆₋₁₂aryl, tolyl and C₃₋₈cycloalkyl; preferably C₁₋₄alkyl, C₆₋₁₀aryl, and C_{3- 6}cycloalkyl;
each R²³ is independently selected from the group consisting of hydrogen, C₁₋₆alkyl, C₆₋₁₂aryl, C₃₋₈cycloalkyl, C₆₋₁₂arylC₁₋₆alkyl, heterocyclyl, and heteroaryl; preferably C₁₋₆alkyl, C₆₋₁₂aryl, C₃₋₈cycloalkyl, and C₆₋₁₂aryl C₁₋₆alkyl; preferably C₁₋₄alkyl, C₆₋₁₀aryl, C₃₋₆cycloalkyl, and C₆₋₁₀aryl C₁₋₄alkyl; preferably C₁₋₆alkyl, C₆₋₁₂aryl, and C₃₋₈cycloalkyl; preferably C₁₋₄alkyl, C₆₋₁₀aryl, and C₃₋₆cycloalkyl;
R²⁴ is selected from the group consisting of hydrogen, deuterium, halogen, —OR²³, cyano, —C(O)R²³, C₁₋₆alkyl, trifluoromethyl, trifluoromethoxy, C₃₋₈cycloalkyl, C₆₋₁₂aryl, heterocyclyl, heteroaryl, C₆₋₁₂arylC₁₋₆alkyl, and nitro; preferably hydrogen, deuterium, halogen, C₁₋₆alkoxy, C₃₋₈cycloalkyloxy, C₆₋₁₂aryloxy, heterocycyloxy, heteroaryloxy, cyano, C₁₋₆alkylcarbonyl, C₃₋₈cycloalkylcarbonyl, C₆₋₁₂arylcarbonyl, C₁₋₆alkyl, trifluoromethyl, trifluoromethoxy, C₃₋₈cycloalkyl, C₆₋₁₂aryl, heterocyclyl, heteroaryl, C₆₋₁₂arylC₁₋₆alkyl, and nitro; preferably hydrogen, deuterium, F. Br, Cl, I, C₁₋₆alkoxy, C₃₋₈cycloalkyloxy, C₆₋₁₂aryloxy, cyano, C₁₋₆alkylcarbonyl, C₁₋₆alkyl, trifluoromethyl, trifluoromethoxy, C₃₋₈cycloalkyl, C₆₋₁₂aryl, heterocyclyl, heteroaryl, and C₆₋₁₂arylC₁₋₆alkyl; preferably hydrogen, deuterium, F, Br, Cl, I, C₁₋₄alkoxy, C₃₋₆cycloalkyloxy, C₆₋₁₀aryloxy, cyano, C₁₋₄alkylcarbonyl, C₁₋₆ alkyl, trifluoromethyl, trifluoromethoxy, C₃₋₆cycloalkyl, C₆₋₁₀aryl, heterocyclyl, heteroaryl, and C₆₋₁₀arylC₁₋₄alkyl; preferably hydrogen, deuterium, F, Br, Cl, I, C₁₋₆alkoxy, cyano, C₁₋₆alkylcarbonyl, C₁₋₆alkyl, trifluoromethyl, trifluoromethoxy, C₃₋₈cycloalkyl, and C₆₋₁₂aryl; preferably hydrogen, 4deuterium, F, Br, Cl, I, C₁₋₆alkoxy, cyano, C₁₋₄alkylcarbonyl, C₁₋₆alkyl, trifluoromethyl, trifluoromethoxy, C₃₋₆cycloalkyl, and C₆₋₁₀aryl; R²⁵ is selected from the group consisting of hydrogen, deuterium, halogen, —OR²³, cyano, —C(O)R²³, C₁₋₆ alkyl, trifluoromethyl, trifluoromethoxy, C₃₋₈cycloalkyl, C₆₋₁₂aryl, heterocyclyl, heteroaryl, C₆₋₁₂ arylC₁₋₆alkyl, and nitro; preferably hydrogen, deuterium, halogen, C₁₋₆alkoxy, C₃₋₈cycloalkyloxy, C₆₋₁₂aryloxy, heterocycyloxy, heteroaryloxy, cyano, C₁₋₆alkylcarbonyl, C₃₋₈cycloalkylcarbonyl, C₆₋₁₂ arylcarbonyl, C₁₋₆alkyl, trifluoromethyl, trifluoromethoxy, C₃₋₈cycloalkyl, C₆₋₁₂aryl, heterocyclyl, heteroaryl, C₆₋₁₂arylC₁₋₆alkyl, and nitro; preferably hydrogen, deuterium, F, Br, Cl, I, C₁₋₆alkoxy, C₃₋₈cycloalkyloxy, C₆₋₁₂aryloxy, cyano, C₁₋₆alkylcarbonyl, C₁₋₆alkyl, trifluoromethyl, trifluoromethoxy, C₃₋₈cycloalkyl, C₆₋₁₂aryl, heterocyclyl, heteroaryl, and C₆₋₁₂arylC₁₋₆alkyl; preferably hydrogen, deuterium, F, Br, Cl, I, C₁₋₄alkoxy, C₃₋₆cycloalkyloxy, C₆₋₁₀aryloxy, cyano, C₁₋₄alkylcarbonyl, C₁₋₆ alkyl, trifluoromethyl, trifluoromethoxy, C₃₋₆cycloalkyl, C₆₋₁₀aryl, heterocyclyl, heteroaryl, and C₆₋₁₀arylC₁₋₄alkyl; preferably hydrogen, deuterium, F, Br, Cl, I, C₁₋₆alkoxy, cyano, C₁₋₆alkylcarbonyl, C₁₋₆alkyl, trifluoromethyl, trifluoromethoxy, C₃₋₈cycloalkyl, and C₆₋₁₂aryl; preferably hydrogen, 4deuterium, F, Br, Cl, I, C₁₋₆alkoxy, cyano, C₁₋₄alkylcarbonyl, C₁₋₆alkyl, trifluoromethyl, trifluoromethoxy, C₃₋₆cycloalkvl, and C₆₋₁₀aryl;
each Z¹ is independently selected from the group consisting of —OR²³, halogen, C₁₋₆alkyl, —NH₂, —NHR²², —COOR²³, C₃₋₈cycloalkyl, trifluoromethyl, trifluoromethoxy, C₆₋₁₂aryl, C₆₋₁₂arylC₁₋₆alkyl, heterocyclyl, heteroaryl, —OH, cyano and nitro; preferably C₁₋₆alkoxy, C₃₋₈cycloalkyloxy, C₆₋₁₂aryloxy, heterocycyloxy, heteroaryloxy, halogen, C₁₋₆alkyl, —NH₂, mono-C₁₋₆alkylamino, mono-C₆₋₁₂arylamino, mono-C₃₋₈cycloalkylamino, mono-heteroarylamino, mono-heterocyclcylamino, C₁₋₆alkyloxycarbonyl, C₃₋₈cycloalkyloxycarbonyl, C₆₋₁₂aryloxycarbonyl, C₃₋₈cycloalkyl,trifluoromethyl, trifluoromethoxy, C₆₋₁₂aryl, C₆₋₁₂arylC₁₋₆alkyl, heterocyclyl, heteroaryl, —OH, cyano and nitro; preferably C₁₋₆alkoxy, C₃₋₈cycloalkyloxy, C₆₋₁₂aryloxy, halogen, C₁₋₆alkyl, —NH₂, mono-C₁₋₆alkylamino, mono-C₃₋₈cycloalkylamino, C₁₋₆alkyloxycarbonyl, C₃₋₈cycloalkyl, trifluoromethyl, trifluoromethoxy, C₆₋₁₂aryl, C₆₋₁₂arylC₁₋₆alkyl, heterocyclyl, heteroaryl, —OH, cyano and nitro; preferably C₁₋₆alkoxy, C₃₋₈cycloalkyloxy, C₆₋₁₂aryloxy, F, Br, Cl, I, C₁₋₆alkyl, —NH₂, mono-C₁₋₆alkylamino, C₁₋₆alkyloxycarbonyl, C₃₋₈cycloalkyl, trifluoromethyl, trifluoromethoxy, C₆₋₁₂aryl, C₆₋₁₂arylC₁₋₆alkyl, heterocyclyl, heteroaryl, and —OH; preferably C₁₋₄alkoxy, C₃₋₆cycloalkyloxy, C₆₋₁₀aryloxy, F, Br, Cl, I, C₁₋₄alkyl, —NH₂, mono-C₁₋₄alkylamino, C₁₋₄alkyloxycarbonyl, C₃₋₆cycloalkyl, trifluoromethyl, trifluoromethoxy, C₆₋₁₀aryl, C₆₋₁₀ arylC₁₋₄alkyl, heterocyclyl, heteroaryl, and —OH; preferably C₁₋₆alkoxy, F, Br, Cl, I, alkyl, —NH₂, mono-C₁₋₆alkylamino, C₁₋₆alkyloxycarbonyl, C₃₋₈cycloalkyl, trifluoromethyl, trifluoromethoxy, C₆₋₁₂aryl, and —OH; preferably C₁₋₄alkoxy, F, Br, Cl, I, alkyl, —NH₂, mono-C₁₋₄alkylamino, C₁₋₄alkyloxycarbonyl, C₃₋₆cycloalkyl, trifluoromethyl, trifluoromethoxy, C₆₋₁₀aryl, and —OH;
each Z^{1a} is independently selected from the group consisting of —OR²³, halogen, C₁₋₆alkyl, —NH₂, —NHR²², —COOR²³, C₃₋₈cycloalkyl, trifluoromethyl, trifluoromethoxy, C₆₋₁₂aryl, C₆₋₁₂arylC₁₋₆alkyl, heterocyclyl, heteroaryl, —OH, cyano and nitro; preferably C₁₋₆alkoxy, C₃₋₈cycloalkyloxy, C₆₋₁₂aryloxy, heterocycyloxy, heteroaryloxy, halogen, C₁₋₆alkyl, —NH₂, mono-C₁₋₆alkylamino, mono-C₆₋₁₂arylamino, mono-C₃₋₈cycloalkylamino, mono-heteroarylamino, mono-heterocyclcylamino, C₁₋₆alkyloxycarbonyl, C₃₋₈cycloalkyloxycarbonyl, C₆₋₁₂aryloxycarbonyl, C₃₋₈cycloalkyl, trifluoromethyl, trifluoromethoxy, C₆₋₁₂aryl, C₆₋₁₂arylC₁₋₆alkyl, heterocyclyl, heteroaryl, —OH, cyano and nitro; preferably C₁₋₆alkoxy, C₃₋₈cycloalkyloxy, C₆₋₁₂aryloxy, halogen, C₁₋₆alkyl, —NH₂, mono-C₁₋₆alkylamino, mono-C₃₋₈cycloalkylamino, C₁₋₆alkyloxycarbonyl, C₃₋₈cycloalkyl, trifluoromethyl, trifluoromethoxy, C₆₋₁₂aryl, C₆₋₁₂arylC₁₋₆alkyl, heterocyclyl, heteroaryl, —OH, cyano and nitro; preferably C₁₋₆alkoxy, C₃₋₈cycloalkyloxy, C₆₋₁₂aryloxy, F, Br, Cl, I, C₁₋₆alkyl, —NH₂, mono-C₁₋₆alkylamino, C₁₋₆alkyloxycarbonyl, C₃₋₈cycloalkyl, trifluoromethyl, trifluoromethoxy, C₆₋₁₂aryl, C₆₋₁₂arylC₁₋₆alkyl, heterocyclyl, heteroaryl, and —OH; preferably C₁₋₄alkoxy, C₃₋₆cycloalkyloxy, C₆₋₁₀aryloxy, F, Br, Cl, I, C₁₋₄alkyl, —NH₂, mono-C₁₋₄alkylamino, C₁₋₄alkyloxycarbonyl, C₃₋₆cycloalkyl, trifluoromethyl, trifluoromethoxy, C₆₋₁₀aryl, C₆₋₁₀ arylC₁₋₄alkyl, heterocyclyl, heteroaryl, and —OH; preferably C₁₋₆alkoxy, F, Br, Cl, I, alkyl, —NH₂, mono-C₁₋₆alkylamino, C₁₋₆alkyloxycarbonyl, C₃₋₈cycloalkyl, trifluoromethyl, trifluoromethoxy, C₆₋₁₂aryl, and —OH; preferably C₁₋₄alkoxy, F, Br, Cl, I, alkyl, —NH₂, mono-C₁₋₄alkylamino, C₁₋₄alkyloxycarbonyl, C₃₋₆cycloalkyl, trifluoromethyl, trifluoromethoxy, C₆₋₁₀aryl, and —OH;
each Z^{1b} is independently selected from the group consisting of —OR²³, halogen, C₁₋₆alkyl, —NH₂, —NHR²², —COOR²³, C₃₋₈cycloalkyl, trifluoromethyl, trifluoromethoxy, C₆₋₁₂aryl, C₆₋₁₂arylC₁₋₆alkyl, heterocyclyl, heteroaryl, —OH, cyano and nitro; preferably C₁₋₆alkoxy, C₃₋₈cycloalkyloxy, C₆₋₁₂aryloxy, heterocycyloxy, heteroaryloxy, halogen, C₁₋₆alkyl, —NH₂, mono-C₁₋₆alkylamino, mono-C₆₋₁₂arylamino, mono-C₃₋₈cycloalkylamino, mono-heteroarylamino, mono-heterocyclcylamino, C₁₋₆alkyloxycarbonyl, C₃₋₆cycloalkyloxycarbonyl, C₆₋₁₂aryloxycarbonyl, C₃₋₈cycloalkyl, trifluoromethyl, trifluoromethoxy, C₆₋₁₂aryl, C₆₋₁₂arylC₁₋₆alkyl, heterocyclyl, heteroaryl, —OH, cyano and nitro; preferably C₁₋₆alkoxy, C₃₋₈cycloalkyloxy, C₆₋₁₂aryloxy, halogen, C₁₋₆alkyl, —NH₂, mono-C₁₋₆alkylamino, mono-C₃₋₈cycloalkylamino, C₁₋₆alkyloxycarbonyl, C₃₋₈cycloalkyl, trifluoromethyl, trifluoromethoxy, C₆₋₁₂aryl, C₆₋₁₂arylC₁₋₆alkyl, heterocyclyl, heteroaryl, —OH, cyano and nitro; preferably C₁₋₆alkoxy, C₃₋₈cycloalkyloxy, C₆₋₁₂aryloxy, F, Br, Cl, I, C₁₋₆alkyl, —NH₂, mono-C₁₋₆alkylamino, C₁₋₆alkyloxycarbonyl, C₃₋₈cycloalkyl, trifluoromethyl, trifluoromethoxy, C₆₋₁₂aryl, C₆₋₁₂arylC₁₋₆alkyl, heterocyclyl, heteroaryl, and —OH; preferably C₁₋₄alkoxy, C₃₋₆cycloalkyloxy, C₆₋₁₀aryloxy, F, Br, Cl, I, C₁₋₄alkyl, —NH₂, mono-C₁₋₄alkylamino, C₁₋₄alkyloxycarbonyl, C₃₋₆cycloalkyl, trifluoromethyl, trifluoromethoxy, C₆₋₁₀aryl, C₆₋₁₀ arylC₁₋₄alkyl, heterocyclyl, heteroaryl, and —OH; preferably C₁₋₆alkoxy, F, Br, Cl, I, alkyl, —NH₂, mono-C₁₋₆alkylamino, C₁₋₆alkyloxycarbonyl, C₃₋₈cycloalkyl, trifluoromethyl, trifluoromethoxy, C₆₋₁₂aryl, and —OH; preferably C₁₋₄alkoxy, F, Br, Cl, I, alkyl, —NH₂, mono-C₁₋₄alkylamino, C₁₋₄alkyloxycarbonyl, C₃₋₆cycloalkyl, trifluoromethyl, trifluoromethoxy, C₆₋₁₀aryl, and —OH;
each Z^{1c} is independently selected from the group consisting of —OR²³, halogen, C₁₋₆alkyl, —NH₂, —NHR²², —COOR²³, C₃₋₈cycloalkyl, trifluoromethyl, trifluoromethoxy, C₆₋₁₂aryl, C₆₋₁₂arylC₁₋₆alkyl, heterocyclyl, heteroaryl, —OH, cyano and nitro; preferably C₁₋₆alkoxy, C₃₋₈cycloalkyloxy, C₆₋₁₂aryloxy, heterocycyloxy, heteroaryloxy, halogen, C₁₋₆alkyl, —NH₂, mono-C₁₋₆alkylamino, mono-C₆₋₁₂arylamino, mono-C₃₋₈cycloalkylamino, mono-heteroarylamino, mono-heterocyclcylamino, C₁₋₆alkyloxycarbonyl, C₃₋₈cycloalkyloxycarbonyl, C₆₋₁₂aryloxycarbonyl, C₃₋₈cycloalkyl, trifluoromethyl, trifluoromethoxy, C₆₋₁₂aryl, C₆₋₁₂arylC₁₋₆alkyl, heterocyclyl, heteroaryl, —OH, cyano and nitro; preferably C₁₋₆alkoxy, C₃₋₈cycloalkyloxy, C₆₋₁₂aryloxy, halogen, C₁₋₆alkyl, —NH₂, mono-C₁₋₆alkylamino, mono-C₃₋₈cycloalkylamino, C₁₋₆alkyloxycarbonyl, C₃₋₈cycloalkyl, trifluoromethyl, trifluoromethoxy, C₆₋₁₂aryl, C₆₋₁₂arylC₁₋₆alkyl, heterocyclyl, heteroaryl, —OH, cyano and nitro; preferably C₁₋₆alkoxy, C₃₋₈cycloalkyloxy, C₆₋₁₂aryloxy, F, Br, Cl, I, C₁₋₆alkyl, —NH₂, mono-C₁₋₆alkylamino, C₁₋₆alkyloxycarbonyl, C₃₋₈cycloalkyl, trifluoromethyl, trifluoromethoxy, C₆₋₁₂aryl, C₆₋₁₂arylC₁₋₆alkyl, heterocyclyl, heteroaryl, and —OH; preferably C₁₋₄alkoxy, C₃₋₆cycloalkyloxy, C₆₋₁₀aryloxy, F, Br, Cl, I, C₁₋₄alkyl, —NH₂, mono-C₁₋₄alkylamino, C₁₋₄alkyloxycarbonyl, C₃₋₆cycloalkyl, trifluoromethyl, trifluoromethoxy, C₆₋₁₀aryl, C₆₋₁₀arylC₁₋₄alkyl, heterocyclyl, heteroaryl, and —OH; preferably C₁₋₆alkoxy, F, Br, Cl, I, alkyl, —NH₂, mono-C₁₋₆alkylamino, C₁₋₆alkyloxycarbonyl, C₃₋₈cycloalkyl, trifluoromethyl, trifluoromethoxy, C₆₋₁₂aryl, and —OH; preferably C₁₋₄alkoxy, F, Br, Cl, I, alkyl, —NH₂, mono-C₁₋₄alkylamino, C₁₋₄alkyloxycarbonyl, C₃₋₆cycloalkyl, trifluoromethyl, trifluoromethoxy, C₆₋₁₀aryl, and —OH;
each Z^{1d} is independently selected from the group consisting of —OR²³, halogen, C₁₋₆alkyl, —NH₂, —NHR²², —COOR²³, C₃₋₈cycloalkyl, trifluoromethyl, trifluoromethoxy, C₆₋₁₂aryl, C₆₋₁₂arylC₁₋₆alkyl, heterocyclyl, heteroaryl, —OH, cyano and nitro; preferably C₁₋₆alkoxy, C₃₋₈cycloalkyloxy, C₆₋₁₂aryloxy, heterocycyloxy, heteroaryloxy, halogen, C₁₋₆alkyl, —NH₂, mono-C₁₋₆alkylamino, mono-C₆₋₁₂arylamino, mono-C₃₋₈cycloalkylamino, mono-heteroarylamino, mono-heterocyclcylamino, C₁₋₆alkyloxycarbonyl, C₃₋₈cycloalkyloxycarbonyl, C₆₋₁₂aryloxycarbonyl, C₃₋₈cycloalkyl, trifluoromethyl, trifluoromethoxy, C₆₋₁₂aryl, C₁₋₁₂arylC₁₋₆alkyl, heterocyclyl, heteroaryl, —OH, cyano and nitro; preferably C₁₋₆alkoxy, C₃₋₈cycloalkyloxy, C₆₋₁₂aryloxy, halogen, C₁₋₆alkyl, —NH₂, mono-C₁₋₆alkylamino, mono-C₃₋₈cycloalkylamino, C₁₋₆alkyloxycarbonyl, C₃₋₈cycloalkyl, trifluoromethyl, trifluoromethoxy, C₆₋₁₂aryl, C₆₋₁₂arylC₁₋₆alkyl, heterocyclyl, heteroaryl, —OH, cyano and nitro; preferably C₁₋₆alkoxy, C₃₋₈cycloalkyloxy, C₆₋₁₂aryloxy, F, Br, Cl, I, C₁₋₆alkyl, —NH₂, mono-C₁₋₆alkylamino, C₁₋₆alkyloxycarbonyl, C₃₋₈cycloalkyl, trifluoromethyl, trifluoromethoxy, C₆₋₁₂aryl, C₆₋₁₂arylC₁₋₆alkyl, heterocyclyl, heteroaryl, and —OH; preferably C₁₋₄alkoxy, C₃₋₆cycloalkyloxy, C₆₋₁₀aryloxy, F, Br, Cl, I, C₁₋₄alkyl, —NH₂, mono-C₁₋₄alkylamino, C₁₋₄alkyloxycarbonyl, C₃₋₆cycloalkyl, trifluoromethyl, trifluoromethoxy, C₆₋₁₀aryl, C₆₋₁₀ arylC₁₋₄alkyl, heterocyclyl, heteroaryl, and —OH; preferably C₁₋₆alkoxy, F, Br, Cl, I, alkyl, —NH₂, mono-C₁₋₆alkylamino, C₁₋₆alkyloxycarbonyl, C₃₋₈cycloalkyl, trifluoromethyl, trifluoromethoxy, C₆₋₁₂aryl, and —OH; preferably C₁₋₄alkoxy, F, Br, Cl, I, alkyl, —NH₂, mono-C₁₋₄alkylamino, C₁₋₄alkyloxycarbonyl. C₃₋₆cycloalkyl, trifluoromethyl, trifluoromethoxy, C₆₋₁₀aryl, and —OH;
each Z² is independently selected from the group consisting of —OR²³, halogen, C₁₋₆alkyl, —NH₂, —NHR²², —COOR²³, C₃₋₈cycloalkyl, trifluoromethyl, trifluoromethoxy, C₆₋₁₂aryl, C₆₋₁₂arylC₁₋₆alkyl, heterocyclyl, heteroaryl, —OH, cyano and nitro; preferably C₁₋₆alkoxy, C₃₋₈cycloalkyloxy, C₆₋₁₂aryloxy, heterocycyloxy, heteroaryloxy, halogen, C₁₋₆alkyl, —NH₂, mono-C₁₋₆alkylamino, mono-C₆₋₁₂arylamino, mono-C₃₋₈cycloalkylamino, mono-heteroarylamino, mono-heterocyclcylamino, C₁₋₆alkyloxycarbonyl, C₃₋₈cycloalkyloxycarbonyl, C₆₋₁₂aryloxycarbonyl, C₃₋₈cycloalkyl, trifluoromethyl, trifluoromethoxy, C₆₋₁₂aryl, C₆₋₁₂arylC₁₋₆alkyl, heterocyclyl, heteroaryl, —OH, cyano and nitro; preferably C₁₋₆alkoxy, C₃₋₈cycloalkyloxy, C₆₋₁₂aryloxy, halogen, C₁₋₆alkyl, —NH₂, mono-C₁₋₆alkylamino, mono-C₃₋₈cycloalkylamino, C₁₋₆alkyloxycarbonyl, C₃₋₈cycloalkyl, trifluoromethyl, trifluoromethoxy, C₆₋₁₂aryl, C₆₋₁₂arylC₁₋₆alkyl, heterocyclyl, heteroaryl, —OH, cyano and nitro; preferably C₁₋₆alkoxy, C₃₋₈cycloalkyloxy, C₆₋₁₂aryloxy, F, Br, Cl, I, C₁₋₆alkyl, —NH₂, mono-C₁₋₆alkylamino, C₁₋₆alkyloxycarbonyl, C₃₋₈cycloalkyl, trifluoromethyl, trifluoromethoxy, C₆₋₁₂aryl, C₆₋₁₂ arylC₁₋₆alkyl, heterocyclyl, heteroaryl, and —OH; preferably C₁₋₄alkoxy, C₃₋₆cycloalkyloxy, C₆₋₁₀ aryloxy, F, Br, Cl, I, C₁₋₄alkyl, —NH₂, mono-C₁₋₄alkylamino, C₁₋₄alkyloxycarbonyl, C₃₋₆cycloalkyl, trifluoromethyl, trifluoromethoxy, C₆₋₁₀aryl, C₆₋₁₀ arylC₁₋₄alkyl, heterocyclyl, heteroaryl, and —OH; preferably C₁₋₆alkoxy, F, Br, Cl, I, alkyl, —NH₂, mono-C₁₋₆alkylamino, C₁₋₆alkyloxycarbonyl, C₃₋₈cycloalkyl, trifluoromethyl, trifluoromethoxy, C₆₋₁₂aryl, and —OH; preferably C₁₋₄alkoxy, F, Br, Cl, I, alkyl, —NH₂, mono-C₁₋₄alkylamino, C₁₋₄alkyloxycarbonyl. C₃₋₆cycloalkyl, trifluoromethyl, trifluoromethoxy, C₆₋₁₀aryl, and —OH. The present invention also provides a compound of formula (1A) (1B) or (1C); or a stereoisomer, enantiomer, racemic, thereof,

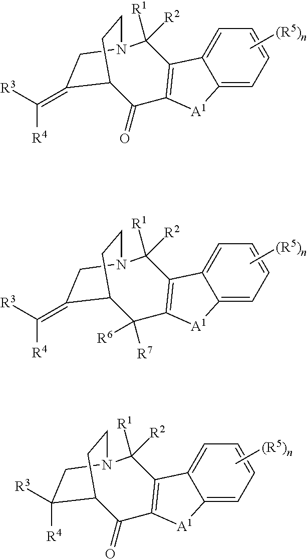

wherein,
n is an integer selected from 0, 1, 2 or 3;
A¹ is selected from the group consisting of a substituted nitrogen or carbon atom, substituents selected from the group consisting of hydrogen, deuterium, alkyl, heteroatom substituted alkyl, alkenyl, aryl, heteroaryl, cycloalkyl, heteroatom substituted cycloalkyl, S, SO, SO₂, OR⁹, NR⁹;
R¹ is selected from the group consisting of hydrogen, deuterium, alkyl, heteroatom substituted alkyl, alkenyl, aryl, heteroaryl, cycloalkyl and heteroatom substituted cycloalkyl;
R² is selected from the group consisting of hydrogen, deuterium, alkyl, heteroatom substituted alkyl, alkenyl, aryl, heteroaryl, cycloalkyl and heteroatom substituted cycloalkyl;
R³ is selected from the group consisting of hydrogen, deuterium, alkyl, heteroatom substituted alkyl, alkenyl, aryl, heteroaryl, halogen, cycloalkyl and heteroatom substituted cycloalkyl;
R⁴ is selected from the group consisting of hydrogen, deuterium, alkyl, heteroatom substituted alkyl, alkenyl, aryl, heteroaryl, halogen, and cycloalkyl and heteroatom substituted cycloalkyl;
or R³ and R⁴ together with the atom to which they are attached can form a saturated or unsaturated 5-, 6-, or 7-membered ring;
R⁵ is selected from the group consisting of deuterium, halogen, alkyl, heteroatom substituted alkyl, alkenyl, aryl, heteroaryl, cycloalkyl and heteroatom substituted cycloalkyl;
R⁶ is selected from the group consisting of hydrogen, deuterium. NH₂, NR⁸R⁹, OR⁹, and R¹;
R⁷ is selected from the group consisting of hydrogen, deuterium, NH₂, NR⁸R⁹, OR⁹, and R¹;
or R⁶ and R⁷ together with the carbon atom to which they are attached from a group selected from the group consisting of —CH═CH₂, —CH═CH-alkyl, and —CH═N—OH;
R⁸ is selected from the group consisting of deuterium, alkyl, heteroatom substituted alkyl, alkenyl, aryl, heteroaryl, cycloalkyl and heteroatom substituted cycloalkyl;
R⁹ is selected from the group consisting of hydrogen, deuterium, alkyl, heteroatom substituted alkyl, alkenyl, aryl, heteroaryl, cycloalkyl and heteroatom substituted cycloalkyl;
or a solvate, hydrate, pharmaceutically acceptable salt or prodrug thereof,
for use in the treatment of distress dysfunction diseases or conditions, cancers, atherosclerotic vascular disease, cardiovascular diseases, fibrosis (e.g. cardiac fibrosis), inflammatory or autoimmune diseases and conditions, conditions of excessive or abnormal vascularization (e.g. wound healing), stem cell differentiation and mobilization disorders, brain and neuronal dysfunctions (e.g. Alzheimer's disease, multiple sclerosis and demyelinating diseases), kidney dysfunction, renal dysfunction, preeclampsia, human immunodeficiency virus (HIV) infection and obesity in a subject.

In some embodiments the compound for use has structural formulae (1AA), (1BB) or (1CC)

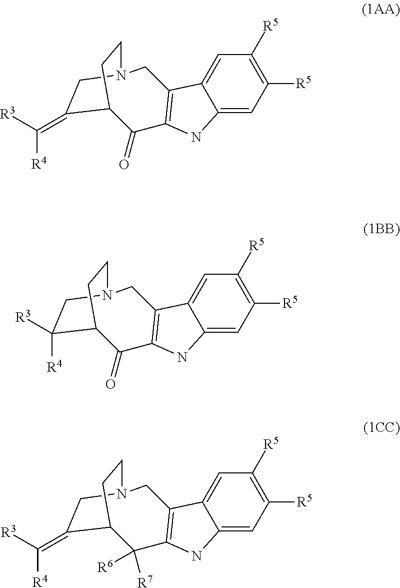

wherein R³, R⁴, R⁵, R⁶, and R⁷ have the same meaning as that defined in hereinabove.

According to particular embodiments, the present invention provides compounds for use of formula (1A) (1B) or (1C), and any subgroup thereof such as (1AA), (1BB) or (1CC) wherein, n is an integer selected from 0, 1, 2 or 3; preferably n is 0, 1 or 2; preferably n is 0 or 1;
A¹ is selected from the group consisting of a substituted nitrogen or carbon atom, substituents selected from the group consisting of hydrogen, deuterium, alkyl, heteroatom substituted alkyl, alkenyl, aryl, heteroaryl, cycloalkyl, heteroatom substituted cycloalkyl. S, SO, SO₂, OR⁹, NR⁹; preferably the substituents are selected from hydrogen deuterium, alkyl, aryl, heteroaryl, cycloalkyl, heteroatom substituted cycloalkyl, and SO₂; preferably the substituents are selected from hydrogen deuterium, alkyl, cycloalkyl, and SO₂;
R¹ is selected from the group consisting of hydrogen, deuterium, alkyl, heteroatom substituted alkyl, alkenyl, aryl, heteroaryl, cycloalkyl and heteroatom substituted cycloalkyl; preferably hydrogen, deuterium, alkyl, heteroatom substituted alkyl, cycloalkyl and heteroatom substituted cycloalkyl; preferably hydrogen, deuterium, alkyl, and heteroatom substituted alkyl;
R² is selected from the group consisting of hydrogen, deuterium, alkyl, heteroatom substituted alkyl, alkenyl, aryl, heteroaryl, cycloalkyl and heteroatom substituted cycloalkyl; preferably hydrogen, deuterium, alkyl, heteroatom substituted alkyl, cycloalkyl and heteroatom substituted cycloalkyl; preferably hydrogen, deuterium, alkyl, and heteroatom substituted alkyl;
R³ is selected from the group consisting of hydrogen, deuterium, alkyl, heteroatom substituted alkyl, alkenyl, aryl, heteroaryl, halogen, cycloalkyl and heteroatom substituted cycloalkyl; preferably hydrogen, deuterium, alkyl, heteroatom substituted alkyl, halogen, cycloalkyl and heteroatom substituted cycloalkyl; preferably hydrogen, deuterium, alkyl, heteroatom substituted alkyl and halogen; preferably hydrogen, deuterium, alkyl, heteroatom substituted alkyl, F, Cl and I;
R⁴ is selected from the group consisting of hydrogen, deuterium, alkyl, heteroatom substituted alkyl, alkenyl, aryl, heteroaryl, halogen, cycloalkyl and heteroatom substituted cycloalkyl; preferably hydrogen, deuterium, alkyl, heteroatom substituted alkyl, halogen, cycloalkyl and heteroatom substituted cycloalkyl; preferably hydrogen, deuterium, alkyl, heteroatom substituted alkyl and halogen; preferably hydrogen, deuterium, alkyl, heteroatom substituted alkyl, F, Cl and I;
or R³ and R⁴ together with the atom to which they are attached can form a saturated or unsaturated 5-, 6-, or 7-membered ring; preferably a saturated 5-, or 6-membered ring;
R⁵ is selected from the group consisting of deuterium, halogen, alkyl, heteroatom substituted alkyl, alkenyl, aryl, heteroaryl, cycloalkyl and heteroatom substituted cycloalkyl; preferably deuterium, halogen, alkyl, heteroatom substituted alkyl, cycloalkyl and heteroatom substituted cycloalkyl; preferably deuterium, halogen, alkyl, and heteroatom substituted alkyl; preferably deuterium, F, Br, Cl, IF, Br, Cl, I, and alkyl;
R⁶ is selected from the group consisting of hydrogen, deuterium, NH₂, NR⁸R⁹, OR⁹, and R¹; preferably hydrogen, deuterium, NH₂, NR⁸R⁹, OR⁹, alkyl, heteroatom substituted alkyl, alkenyl, aryl, heteroaryl, cycloalkyl and heteroatom substituted cycloalkyl; preferably hydrogen, deuterium, NH₂, NR⁸R⁹, OR⁹, alkyl, heteroatom substituted alkyl, cycloalkyl and heteroatom substituted cycloalkyl; preferably hydrogen, deuterium, NH₂, alkyl, and heteroatom substituted alkyl;
R⁷ is selected from the group consisting of hydrogen, deuterium, NH₂, NR⁸R⁹, OR⁹, and R¹; preferably hydrogen, deuterium, NH₂, NR⁸R⁹, OR⁹, alkyl, heteroatom substituted alkyl, alkenyl, aryl, heteroaryl, cycloalkyl and heteroatom substituted cycloalkyl; preferably hydrogen, deuterium, NH₂, mono- or dialkylamino, hydroxyl, alkoxy, aryloxy, alkyl, heteroatom substituted alkyl, cycloalkyl and heteroatom substituted cycloalkyl; preferably hydrogen, deuterium, NH₂, hydroxyl, alkoxy, alkyl, and heteroatom substituted alkyl;
or R⁶ and R⁷ together with the carbon atom to which they are attached from a group selected from the group consisting of —CH═CH₂, —CH═CH-alkyl, and —CH═N—OH; preferably —CH═CH₂, and —CH═CH— alkyl;
R⁸ is selected from the group consisting of deuterium, alkyl, heteroatom substituted alkyl, alkenyl, aryl, heteroaryl, cycloalkyl and heteroatom substituted cycloalkyl; preferably deuterium, alkyl, heteroatom substituted alkyl, aryl, cycloalkyl and heteroatom substituted cycloalkyl; preferably deuterium, alkyl, and heteroatom substituted alkyl;
R⁹ is selected from the group consisting of hydrogen, deuterium, alkyl, heteroatom substituted alkyl, alkenyl, aryl, heteroaryl, cycloalkyl and heteroatom substituted cycloalkyl; preferably hydrogen, deuterium, alkyl, heteroatom substituted alkyl, aryl, cycloalkyl and heteroatom substituted cycloalkyl; preferably hydrogen, deuterium, alkyl, and heteroatom substituted alkyl.

According to particular embodiments, the present invention provides compounds for use of formula (1A) (1B) or (1C), and any subgroup thereof such as (1AA), (1BB) or (1CC) wherein, wherein,
n is an integer selected from 0, 1, 2 or 3;
A¹ is selected from the group consisting of a substituted nitrogen or carbon atom, substituents selected from the group consisting of hydrogen, deuterium, C₁₋₆alkyl, heteroatom substituted C₁₋₆alkyl, C₂₋₆alkenyl, C₆₋₁₂aryl, heteroaryl, C₃₋₈cycloalkyl, heteroatom substituted C₃₋₈cycloalkyl, S, SO, SO₂, OR⁹, NR⁹;
R¹ is selected from the group consisting of hydrogen, deuterium, C₁₋₆alkyl, heteroatom substituted C₁₋₆alkyl, C₂₋₆alkenyl, C₆₋₁₂aryl, heteroaryl, C₃₋₈cycloalkyl, and heteroatom substituted C₃₋₈cycloalkyl;
R² is selected from the group consisting of hydrogen, deuterium, C₁₋₆alkyl, heteroatom substituted C₁₋₆alkyl, C₂₋₆alkenyl, C₆₋₁₂aryl, heteroaryl, C₃₋₈cycloalkyl, and heteroatom substituted C₃₋₈cycloalkyl;
R³ is selected from the group consisting of hydrogen, deuterium, C₁₋₆alkyl, heteroatom substituted C₁₋₆alkyl, C₂₋₆alkenyl, C₆₋₁₂aryl, heteroaryl, C₃₋₈cycloalkyl, and heteroatom substituted C₃₋₈cycloalkyl;
R⁴ is selected from the group consisting of hydrogen, deuterium, C₁₋₆alkyl, heteroatom substituted C₁₋₆alkyl, C₂₋₆alkenyl, C₆₋₁₂aryl, heteroaryl, C₃₋₈cycloalkyl and heteroatom substituted C₃₋₈cycloalkyl;
or R³ and R⁴ together with the atom to which they are attached can form a saturated or unsaturated 5-, 6-, or 7-membered ring;
R⁵ is selected from the group consisting of deuterium, halogen, C₁₋₆alkyl, heteroatom substituted C₁₋₆alkyl, C₂₋₆alkenyl, C₆₋₁₂aryl, heteroaryl, C₃₋₈cycloalkyl and heteroatom substituted C₃₋₈cycloalkyl;
R⁶ is selected from the group consisting of hydrogen, deuterium, NH₂, NR⁸R⁹, OR⁹, and R¹;
R⁷ is selected from the group consisting of hydrogen, deuterium, NH₂, NR⁸R⁹, OR⁹, and R¹;
or R⁶ and R⁷ together with the carbon atom to which they are attached from a group selected from the group consisting of —CH═CH₂, —CH═CH—C₁₋₆alkyl, and —CH═N—OH;
R⁸ is selected from the group consisting of deuterium, C₁₋₆alkyl, heteroatom substituted C₁₋₆alkyl, C₂₋₆alkenyl, C₆₋₁₂aryl, heteroaryl, C₃₋₈cycloalkyl and heteroatom substituted C₃₋₈cycloalkyl;
R⁹ is selected from the group consisting of hydrogen, deuterium, C₁₋₆alkyl, heteroatom substituted C₁₋₆alkyl, C₂₋₆alkenyl, C₆₋₁₂aryl, heteroaryl, C₃₋₈cycloalkyl and heteroatom substituted C₃₋₈cycloalkyl.

According to particular embodiments, the present invention provides compounds for use of formula (1A) (1B) or (1C), and any subgroup thereof such as (1AA), (1BB) or (1CC) wherein, n is an integer selected from 0, 1, 2 or 3; preferably n is 0, 1 or 2; preferably n is 0 or 1;
A¹ is selected from the group consisting of a substituted nitrogen or carbon atom, substituents selected from the group consisting of hydrogen, deuterium, C₁₋₆alkyl, heteroatom substituted C₁₋₆alkyl, C₂₋₆alkenyl, C₆₋₁₂aryl, heteroaryl, C₃₋₈cycloalkyl, heteroatom substituted C₃₋₈cycloalkyl, S, SO, SO₂, OR⁹, NR⁹; preferably the substituents are selected from C₁₋₆alkyl, heteroatom substituted C₁₋₆alkyl, C₂₋₆alkenyl, C₆₋₁₂aryl, heteroaryl, C₃₋₈cycloalkyl, heteroatom substituted C₃₋₈cycloalkyl, S, SO, SO₂, hydroxyl, C₁₋₆alkoxy, C₆₋₁₂aryloxy, mono- or diC₁₋₆alkylamino; preferably the substituents are selected from hydrogen deuterium, C₁₋₆alkyl, C₆₋₁₂aryl, heteroaryl, C₃₋₈cycloalkyl, heteroatom substituted C₃₋₈cycloalkyl, and SO₂; preferably the substituents are selected from hydrogen deuterium, C₁₋₆alkyl, C₃₋₈cycloalkyl, and SO₂;
R¹ is selected from the group consisting of hydrogen, deuterium, C₁₋₆alkyl, heteroatom substituted C₁₋₆alkyl, C₂₋₆alkenyl, C₆₋₁₂aryl, heteroaryl, C₃₋₈cycloalkyl, and heteroatom substituted C₃₋₈cycloalkyl; preferably hydrogen, deuterium, C₁₋₆alkyl, heteroatom substituted C₁₋₆alkyl, C₃₋₈cycloalkyl, and heteroatom substituted C₃₋₈cycloalkyl; preferably hydrogen, deuterium, C₁₋₄alkyl, heteroatom substituted C₁₋₄alkyl, C₃₋₆cycloalkyl, and heteroatom substituted C₃₋₆cycloalkyl; preferably hydrogen, deuterium, C₁₋₆alkyl, and heteroatom substituted C₁₋₆alkyl; preferably hydrogen, deuterium, C₁₋₄alkyl, and heteroatom substituted C₁₋₄alkyl; preferably hydrogen, deuterium, C₁₋₆alkyl, and heteroatom substituted C₁₋₆alkyl;
R² is selected from the group consisting of hydrogen, deuterium, C₁₋₆alkyl, heteroatom substituted C₁₋₆alkyl, C₂₋₆alkenyl, C₆₋₁₂aryl, heteroaryl, C₃₋₈cycloalkyl, and heteroatom substituted C₃₋₈cycloalkyl; preferably hydrogen, deuterium, C₁₋₆alkyl, heteroatom substituted C₁₋₆alkyl, C₃₋₈cycloalkyl, and heteroatom substituted C₃₋₈cycloalkyl; preferably hydrogen, deuterium, C₁₋₄alkyl, heteroatom substituted C₁₋₄alkyl, C₃₋₆cycloalkyl, and heteroatom substituted C₃₋₆cycloalkyl; preferably hydrogen, deuterium, C₁₋₆alkyl, and heteroatom substituted C₁₋₆alkyl; preferably hydrogen, deuterium, C₁₋₄alkyl, and heteroatom substituted C₁₋₄alkyl;
R³ is selected from the group consisting of hydrogen, deuterium, C₁₋₆alkyl, heteroatom substituted C₁₋₆alkyl, C₂₋₆alkenyl, C₆₋₁₂aryl, heteroaryl, halogen, C₃₋₈cycloalkyl, and heteroatom substituted C₃₋₈cycloalkyl: preferably hydrogen, deuterium, C₁₋₆alkyl, heteroatom substituted C₁₋₆alkyl, halogen, C₃₋₈ cycloalkyl, and heteroatom substituted C₃₋₈cycloalkyl; preferably hydrogen, deuterium, C₁₋₄alkyl, heteroatom substituted C₁₋₄alkyl, halogen, C₃₋₆cycloalkyl, and heteroatom substituted C₃₋₆cycloalkyl; preferably hydrogen, deuterium, C₁₋₆alkyl, heteroatom substituted C₁₋₆alkyl, and halogen; preferably hydrogen, deuterium, C₁₋₆alkyl, heteroatom substituted C₁₋₆alkyl, F, Cl and I; preferably hydrogen, deuterium, C₁₋₄alkyl, heteroatom substituted C₁₋₄alkyl, F, Cl and I;
R⁴ is selected from the group consisting of hydrogen, deuterium, C₁₋₆alkyl, heteroatom substituted C₁₋₆alkyl, C₂₋₆alkenyl, C₆₋₁₂aryl, heteroaryl, halogen, C₃₋₈cycloalkyl, and heteroatom substituted C₃₋₈cycloalkyl; preferably hydrogen, deuterium, C₁₋₆alkyl, heteroatom substituted C₁₋₆alkyl, halogen, C₃₋₈cycloalkyl, and heteroatom substituted C₃₋₈cycloalkyl; preferably hydrogen, deuterium, C₁₋₄alkyl, heteroatom substituted C₁₋₄alkyl, halogen, C₃₋₆cycloalkyl, and heteroatom substituted C₃₋₆cycloalkyl; preferably hydrogen, deuterium, C₁₋₆alkyl, heteroatom substituted C₁₋₆alkyl, and halogen; preferably hydrogen, deuterium, C₁₋₆alkyl, heteroatom substituted C₁₋₆alkyl, F, Cl and I; preferably hydrogen, deuterium, C₁₋₄alkyl, heteroatom substituted C₁₋₄alkyl, F, Cl and I;
or R³ and R⁴ together with the atom to which they are attached can form a saturated or unsaturated 5-, 6-, or 7-membered ring; preferably a saturated 5-, or 6-membered ring;
R⁵ is selected from the group consisting of deuterium, halogen, C₁₋₆alkyl, heteroatom substituted C₁₋₆alkyl, C₂₋₆alkenyl, C₆₋₁₂aryl, heteroaryl, C₃₋₈cycloalkyl and heteroatom substituted C₃₋₈cycloalkyl; preferably deuterium, halogen, C₁₋₆alkyl, heteroatom substituted C₁₋₆alkyl, C₃₋₈cycloalkyl and heteroatom substituted C₃₋₈cycloalkyl; preferably deuterium, halogen, C₁₋₄alkyl, heteroatom substituted C₁₋₄alkyl, C₃₋₆cycloalkyl and heteroatom substituted C₃₋₆cycloalkyl; preferably deuterium, halogen, C₁₋₆alkyl, and heteroatom substituted C₁₋₆alkyl; preferably deuterium, F, Br, Cl, IF, Br, Cl, I, and C₁₋₆alkyl; preferably deuterium, halogen, C₁₋₄alkyl, and heteroatom substituted C₁₋₄alkyl; preferably deuterium, F, Br, Cl, IF, Br, Cl, I, and C₁₋₆alkyl;
R⁶ is selected from the group consisting of hydrogen, deuterium, NH₂, NR⁸R⁹, OR⁹, and R¹; preferably hydrogen, deuterium, NH₂, NR⁸R⁹, OR⁹, C₁₋₆alkyl, heteroatom substituted C₁₋₆alkyl, C₂₋₆alkenyl, C₆₋₁₂aryl, heteroaryl, C₃₋₈cycloalkyl and heteroatom substituted C₃₋₈cycloalkyl; preferably hydrogen, deuterium, NH₂, NR⁸R⁹, OR⁹, C₁₋₆alkyl, heteroatom substituted C₁₋₆alkyl, C₃₋₈cycloalkyl and heteroatom substituted C₃₋₈cycloalkyl; preferably hydrogen, deuterium, NH₂, NR⁸R⁹, OR⁹, C₁₋₄alkyl, heteroatom substituted C₁₋₄alkyl, C₃₋₆cycloalkyl and heteroatom substituted C₃₋₆cycloalkyl; preferably hydrogen, deuterium, NH₂, C₁₋₆alkyl, and heteroatom substituted C₁₋₆alkyl; preferably hydrogen, deuterium, NH₂, C₁₋₄alkyl, and heteroatom substituted C₁₋₄alkyl;
R⁷ is selected from the group consisting of hydrogen, deuterium, NH₂, NR⁸R⁹, OR⁹, and R¹; preferably hydrogen, deuterium, NH₂, NR⁸R⁹, OR⁹, C₁₋₆alkyl, heteroatom substituted C₁₋₆alkyl, C₂₋₆alkenyl, C₆₋₁₂aryl, heteroaryl, C₃₋₈cycloalkyl and heteroatom substituted C₃₋₈cycloalkyl; preferably hydrogen, deuterium, NH₂, NR⁸R⁹, OR⁹, C₁₋₆alkyl, heteroatom substituted C₁₋₆alkyl, C₃₋₈cycloalkyl and heteroatom substituted C₃₋₈cycloalkyl; preferably hydrogen, deuterium, NH₂, NR⁸R⁹, OR⁹, C₁₋₄alkyl, heteroatom substituted C₁₋₄alkyl, C₃₋₆cycloalkyl and heteroatom substituted C₃₋₆cycloalkyl; preferably hydrogen, deuterium, NH₂, C₁₋₆alkyl, and heteroatom substituted C₁₋₆alkyl; preferably hydrogen, deuterium, NH₂, C₁₋₄alkyl, and heteroatom substituted C₁₋₄alkyl;
or R⁶ and R⁷ together with the carbon atom to which they are attached from a group selected from the group consisting of —CH═CH₂, —CH═CH— C₁₋₆alkyl, and —CH═N—OH; preferably —CH═CH₂, and —CH═CH— C₁₋₆alkyl; preferably —CH═CH₂, and —CH═CH— C₁₋₄alkyl;
R⁸ is selected from the group consisting of deuterium, C₁₋₆alkyl, heteroatom substituted C₁₋₆alkyl, C₂₋₆alkenyl, C₆₋₁₂aryl, heteroaryl, C₃₋₈cycloalkyl and heteroatom substituted C₃₋₈cycloalkyl; preferably deuterium, C₁₋₆alkyl, heteroatom substituted C₁₋₆alkyl, C₆₋₁₂aryl, C₃₋₈cycloalkyl and heteroatom substituted C₃₋₈cycloalkyl; preferably deuterium, C₁₋₄alkyl, heteroatom substituted C₁₋₄alkyl, C₆₋₁₀aryl, C₃₋₆cycloalkyl and heteroatom substituted C₃₋₆cycloalkyl; preferably deuterium, C₁₋₆alkyl, and heteroatom substituted C₁₋₆alkyl; preferably deuterium, C₁₋₄alkyl, and heteroatom substituted C₁₋₄ alkyl;
R⁹ is selected from the group consisting of hydrogen, deuterium, C₁₋₆alkyl, heteroatom substituted C₁₋₆ alkyl, C₂₋₆alkenyl, C₆₋₁₂aryl, heteroaryl, cycloalkyl and heteroatom substituted cycloalkyl; preferably hydrogen, deuterium, C₁₋₆alkyl, heteroatom substituted C₁₋₆alkyl, C₆₋₁₂aryl, C₃₋₈cycloalkyl and heteroatom substituted C₃₋₈cycloalkyl; preferably hydrogen deuterium, C₁₋₄alkyl, heteroatom substituted C₁₋₄alkyl, C₆₋₁₀ aryl, C₃₋₆cycloalkyl and heteroatom substituted C₃₋₆cycloalkyl; preferably hydrogen, deuterium, C₁₋₆alkyl, and heteroatom substituted C₁₋₆alkyl; preferably hydrogen, deuterium, C₁₋₄alkyl, and heteroatom substituted C₁₋₄alkyl.

According to particular embodiments, the present invention provides compounds of formula (2), and any subgroup thereof such as (2A), (2B), (2C), (2D), (2E), (2F), (2G), (2H), (2I), (2J), (2K), (2L), (2M), (2N), (2P) for use as a medicament.

According to particular embodiments, the present invention provides compounds of formula (2), and any subgroup thereof such as (2A), (2B), (2C), (2D), (2E), (2F), (2G), (2H), (2I), (2J), (2K), (2L), (2M), (2N), (2P) for use in the treatment of pain and treatment of distress dysfunction diseases or conditions, cancers, atherosclerotic vascular disease, cardiovascular diseases, fibrosis (e.g. cardiac fibrosis), inflammatory or autoimmune diseases and conditions, conditions of excessive or abnormal vascularization (e.g. wound healing), stem cell differentiation and mobilization disorders, brain and neuronal dysfunctions (e.g. Alzheimer's disease, multiple sclerosis and demyelinating diseases), kidney dysfunction, renal dysfunction, preeclampsia, human immunodeficiency virus (HIV) infection and obesity in a subject.

According to particular embodiments, the present invention provides a pharmaceutical composition comprising a compound of formula (2), or any subgroup thereof such as (2A), (2B), (2C), (2D), (2E), (2F), (2G), (2H), (2I), (2J), (2K), (2L), (2M), (2N), (2P).

According to particular embodiments, the present invention provides compounds of formula (2), and any subgroup thereof such as (2A), (2B), (2C), (2D), (2E), (2F), (2G), (2H), (2I), (2J), (2K), (2L), (2M), (2N), (2P)
wherein,
o is an integer selected from 0, 1, 2 or 3;
p is an integer selected from 0, 1, 2, 3 or 4;
A² is selected from N or CR¹⁹;
A³ is selected from N or CR²⁰;
A⁴ is selected from NR¹¹, O, S or CR²⁴ R²⁵;
A⁵ is selected from N or CR¹²;
A⁶ is selected from N or CR¹³;
A⁷ is selected from N or CR¹⁴;
A⁸ is selected from N or CR¹⁵;
wherein at least one of A² or A³ is N;
wherein at most one of A⁵ to A⁸ is N;
L is selected from —C═O, —C(O)—NH—, and CHR²¹;
R¹¹ is selected from the group consisting of hydrogen, deuterium, C₁₋₆alkyl, —S(O)₂R²², C₆₋₁₂aryl, —S(O)R²², and —SO₂NR²²R²³; and wherein said C₁₋₆alkyl or C₆₋₁₂aryl can be unsubstituted or substituted with one or more Z¹;
R¹² is selected from the group consisting of hydrogen, deuterium, halogen, —OR²³, cyano, —C(O)R²³, C₁₋₆alkyl, trifluoromethyl, trifluoromethoxy, C₃₋₈cycloalkyl, C₆₋₁₂aryl, heterocyclyl, heteroaryl, C₆₋₁₂arylC₁₋₆alkyl, and nitro, wherein said alkyl, cycloalkyl, aryl, heterocyclyl, heteroaryl, or arylalkyl is substituted by one or more Z^{1a};
R¹³ is selected from the group consisting of hydrogen, deuterium, halogen, —OR²³, cyano, —C(O)R²³, C₁₋₆alkyl, trifluoromethyl, trifluoromethoxy, C₃₋₈cycloalkyl, C₆₋₁₂aryl, heterocyclyl, heteroaryl, C₆₋₁₂arylC₁₋₆alkyl, and nitro, wherein said alkyl, cycloalkyl, aryl, heterocyclyl, heteroaryl, or arylalkyl is substituted by one or more Z^{1b};
R¹⁴ is selected from the group consisting of hydrogen, deuterium, halogen, —OR²³, cyano, —C(O)R²³, C₁₋₆alkyl, trifluoromethyl, trifluoromethoxy, C₃₋₈cycloalkyl, C₆₋₁₂aryl, heterocyclyl, heteroaryl, C₆₋₁₂arylC₁₋₆alkyl, and nitro, wherein said alkyl, cycloalkyl, aryl, heterocyclyl, heteroaryl, or arylalkyl is substituted by one or more Z^{1c};
R¹⁵ is selected from the group consisting hydrogen, deuterium, halogen, —OR²³, cyano, —C(O)R²³, C₁₋₆alkyl, trifluoromethyl, trifluoromethoxy, C₃₋₈cycloalkyl, C₆₋₁₂aryl, heterocyclyl, heteroaryl, C₆₋₁₂arylC₁₋₆alkyl, and nitro, wherein said alkyl, cycloalkyl, aryl, heterocyclyl, heteroaryl, or arylalkyl is substituted by one or more Z^{ca};
R¹⁶ is selected from the group consisting of hydrogen, deuterium, C₁₋₆alkyl, halogen, and —OR²³;
R¹⁷ is selected from the group consisting of hydrogen, deuterium, C₁₋₆alkyl, halogen, and —OR²³;
or R¹⁶ and R¹⁷ together with the carbon atom to which they are attached from a group selected from —C═CH—C₁₋₆alkyl, —C═N—OH, —C═N—O—Si(CH₃)₂C(CH₃)₃, or a saturated or unsaturated 3-, 4-, 5-, 6- or 7-membered ring;
R¹⁸ is selected from the group consisting of hydrogen, deuterium, halogen, —NH₂, —NHR²², C₁₋₆alkyl, deuterium, C₆₋₁₂arylC₁₋₆alkyl, —S(O)₂R²², —C(O)OR²³, —S(O)R²², —(CH₂)₂—O—(CH₂)₂—NH₂, heteroaryl, C₃₋₈cycloalkyl, C₆₋₁₂aryl, heterocyclyl; and wherein said C₁₋₆alkyl, C₆₋₁₂arylarylC₁₋₆alkyl, heteroaryl, C₃₋₈cycloalkyl, C₆₋₁₂aryl, heterocyclyl, or C₆₋₁₂arylC₁₋₆alkyl can be unsubstituted or substituted with one or more Z²
R¹⁹ is selected from the group consisting of hydrogen, C₁₋₆alkyl, halogen, and —OR²³;
R²⁰ is selected from the group consisting of hydrogen, C₁₋₆alkyl, halogen, and —OR²³;
R²¹ is selected from the group consisting of —OH, —COOR²³, —C(O)NH, hydrogen, and —OR²³;
each R²² is independently selected from the group consisting of C₁₋₆alkyl, C₆₋₁₂aryl, tolyl, C₃₋₈cycloalkyl, C₆₋₁₂arylC₁₋₆alkyl, heterocyclyl, and heteroaryl;
each R²³ is independently selected from the group consisting of C₁₋₆alkyl, C₆₋₁₂aryl, C₃₋₈cycloalkyl, C₆₋₁₂arylC₁₋₆alkyl, heterocyclyl, and heteroaryl;
R²⁴ is selected from the group consisting of hydrogen, deuterium, halogen, —OR²³, cyano, —C(O)R²³, C₁₋₆alkyl, trifluoromethyl, trifluoromethoxy, C₃₋₈cycloalkyl, C₆₋₁₂aryl, heterocyclyl, heteroaryl, C₆₋₁₂arylC₁₋₆alkyl, and nitro;
R²⁵ is selected from the group consisting of hydrogen, deuterium, halogen, —OR²³, cyano, —C(O)R²³, C₁₋₆alkyl, trifluoromethyl, trifluoromethoxy, C₃₋₈cycloalkyl, C₆₋₁₂aryl, heterocyclyl, heteroaryl, C₆₋₁₂arylC₁₋₆alkyl, and nitro;
each Z¹ is independently selected from the group consisting of —OR²³, halogen, C₁₋₆alkyl, —NH₂, —NHR²², —COOR²³, C₃₋₈cycloalkyl, trifluoromethyl, trifluoromethoxy, C₆₋₁₂aryl, C₆₋₁₂arylC₁₋₆alkyl, heterocyclyl, heteroaryl, —OH, cyano and nitro;
each Z^{1a} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z^{1b} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z^{1c} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z^{1d} is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z² is independently selected from the group consisting of OR²³, halogen, C₁₋₆alkyl, —NH₂, —NHR²², —COOR²³, C₃₋₈cycloalkyl, trifluoromethyl, trifluoromethoxy, C₆₋₁₂aryl, C₆₋₁₂arylC₁₋₆alkyl, heterocyclyl, heteroaryl, —OH, cyano and nitro;
or ethyl {(3Z)-3-({[tert-butyl(dimethyl)silyl]oxy}imino-1-[(4-methylphenyl)sulfonyl]piperidin-4-yl}(3-formyl-1H-indol-2-yl)acetate;
or ethyl (3-formyl-1H-indol-2-yl){(3Z)-3-(hydroxyamino)-1-[(4-methylphenyl)sulfonyl]piperidin-4-yl}acetate;
or a solvate, hydrate, pharmaceutically acceptable salt or prodrug thereof,
for use in the treatment of pain and treatment of distress dysfunction diseases or conditions, cancers, atherosclerotic vascular disease, cardiovascular diseases, fibrosis (e.g. cardiac fibrosis), inflammatory or autoimmune diseases and conditions, conditions of excessive or abnormal vascularization (e.g. wound healing), stem cell differentiation and mobilization disorders, brain and neuronal dysfunctions (e.g. Alzheimer's disease, multiple sclerosis and demyelinating diseases), kidney dysfunction, renal dysfunction, preeclampsia, human immunodeficiency virus (HIV) infection and obesity in a subject.

Particularly preferred compounds of the invention are those compounds listed in Table 1.

TABLE 1

| Compound | Structure |
|---|---|
| WW-1 | 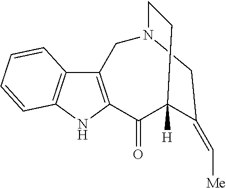 [SMILES: C/C=C1/CN2CC[C@H]1C(=O)c1[nH]c3ccccc3c1C2] |
| WW-2 | 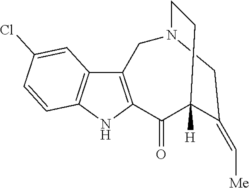 [SMILES: C/C=C1/CN2CC[C@H]1C(=O)c1[nH]c3ccc(Cl)cc3c1C2] |
| WW-3 | 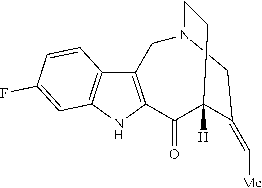 [SMILES: C/C=C1/CN2CC[C@H]1C(=O)c1[nH]c3cc(F)ccc3c1C2] |
| WW-4 | 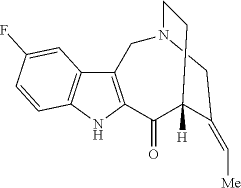 [SMILES: C/C=C1/CN2CC[C@H]1C(=O)c1[nH]c3ccc(F)cc3c1C2] |
| WW-5 | 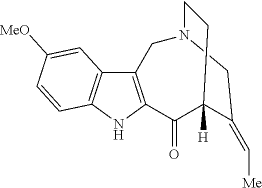 [SMILES: C/C=C1/CN2CC[C@H]1C(=O)c1[nH]c3ccc(OC)cc3c1C2] |
| WW-6 | 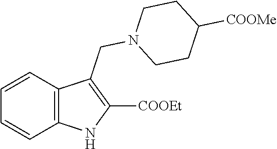 [SMILES: CCOC(=O)c1[nH]c2ccccc2c1CN1CCC(C(=O)OC)CC1] |
| WW-7 | 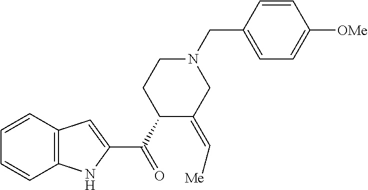 [SMILES: C/C=C1/CN(Cc2ccc(OC)cc2)CC[C@H]1C(=O)c1cc2ccccc2[nH]1] |
| WW-8 | 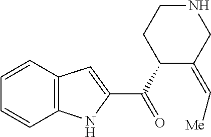 [SMILES: C/C=C1/CNCC[C@H]1C(=O)c1cc2ccccc2[nH]1] |
| WW-9 | 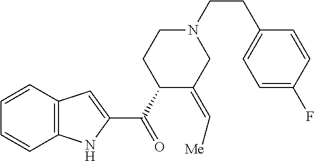 [SMILES: C/C=C1/CN(CCc2ccc(F)cc2)CC[C@H]1C(=O)c1cc2ccccc2[nH]1] |
| WW-10 | 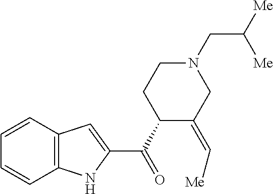 [SMILES: C/C=C1/CN(CC(C)C)CC[C@H]1C(=O)c1cc2ccccc2[nH]1] |
| WW-12 | 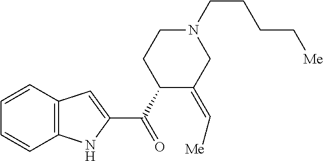 [SMILES: C/C=C1/CN(CCCCC)CC[C@H]1C(=O)c1cc2ccccc2[nH]1] |
| WW-14 | 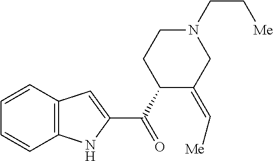 [SMILES: C/C=C1/CN(CCC)CC[C@H]1C(=O)c1cc2ccccc2[nH]1] |
| WW-15 | 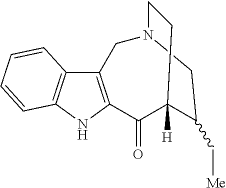 [SMILES: CCC1CN2CC[C@H]1C(=O)c1[nH]c3ccccc3c1C2] |
| WW-16 | 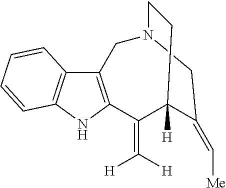 [SMILES: C=C1c2[nH]c3ccccc3c2CN2CC[C@@H]1/C(=C\C)C2] |
| WW-17 | 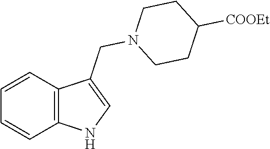 [SMILES: CCOC(=O)C1CCN(Cc2c[nH]c3ccccc23)CC1] |
| WW-18 | 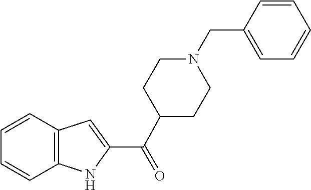 [SMILES: O=C(c1cc2ccccc2[nH]1)C1CCN(Cc2ccccc2)CC1] |
| WW-19 | 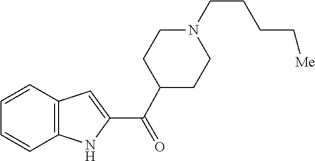 [SMILES: CCCCCN1CCC(C(=O)c2cc3ccccc3[nH]2)CC1] |
| WW-20 | 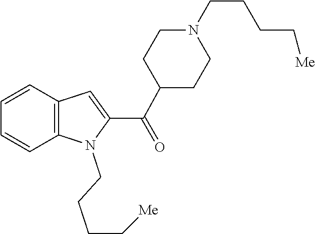 [SMILES: CCCCCN1CCC(C(=O)c2cc3ccccc3n2CCCCC)CC1] |
| WW-21 | 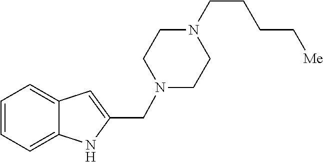 [SMILES: CCCCCN1CCN(Cc2cc3ccccc3[nH]2)CC1] |
| WW-22 | 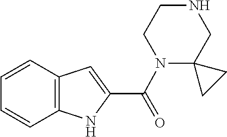 [SMILES: O=C(c1cc2ccccc2[nH]1)N1CCNCC12CC2] |
| WW-23 | 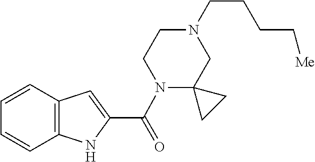 [SMILES: CCCCCN1CCN(C(=O)c2cc3ccccc3[nH]2)C2(CC2)C1] |
| WW-24 | 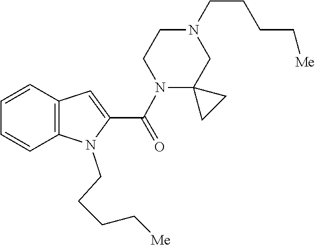 [SMILES: CCCCCN1CCN(C(=O)c2cc3ccccc3n2CCCCC)C2(CC2)C1] |
| WW-25 | 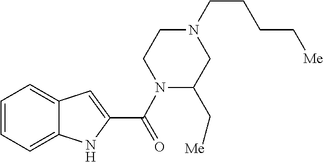 [SMILES: CCCCCN1CCN(C(=O)c2cc3ccccc3[nH]2)C(CC)C1] |
| WW-26 | 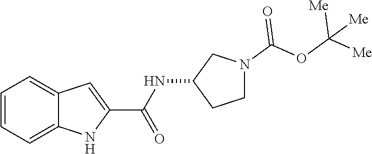 [SMILES: CC(C)(C)OC(=O)N1CC[C@H](NC(=O)c2cc3ccccc3[nH]2)C1] |
| WW-27 | 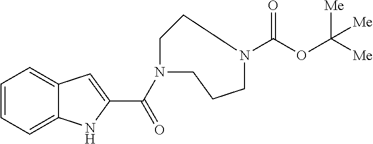 [SMILES: CC(C)(C)OC(=O)N1CCCN(C(=O)c2cc3ccccc3[nH]2)CC1] |
| WW-28 | 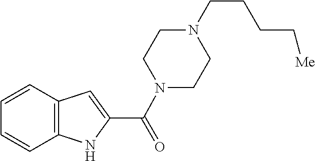 [SMILES: CCCCCN1CCN(C(=O)c2cc3ccccc3[nH]2)CC1] |
| WW-29 | 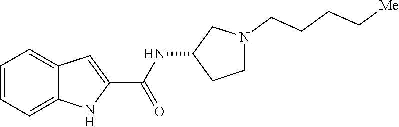 [SMILES: CCCCCN1CC[C@H](NC(=O)c2cc3ccccc3[nH]2)C1] |
| WW-30 | 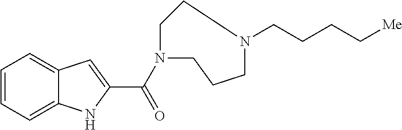 [SMILES: CCCCCN1CCCN(C(=O)c2cc3ccccc3[nH]2)CC1] |
| WW-31 | 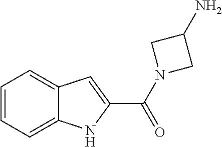 [SMILES: NC1CN(C(=O)c2cc3ccccc3[nH]2)C1] |
| WW-32 | 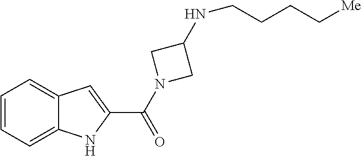 [SMILES: CCCCCNC1CN(C(=O)c2cc3ccccc3[nH]2)C1] |
| WW-33 | 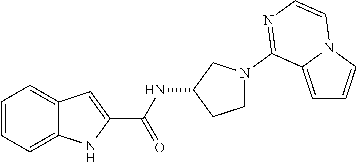 [SMILES: O=C(N[C@H]1CCN(c2nccn3cccc23)C1)c1cc2ccccc2[nH]1] |
| WW-43 | 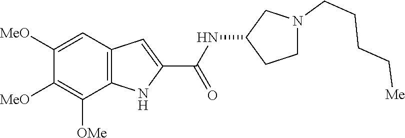 [SMILES: CCCCCN1CC[C@H](NC(=O)c2cc3cc(OC)c(OC)c(OC)c3[nH]2)C1] |
| WW-44 | 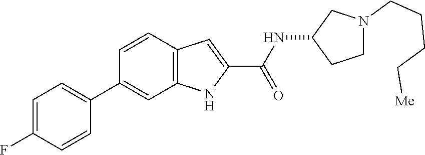 [SMILES: CCCCCN1CC[C@H](NC(=O)c2cc3ccc(-c4ccc(F)cc4)cc3[nH]2)C1] |
| WW-45 | 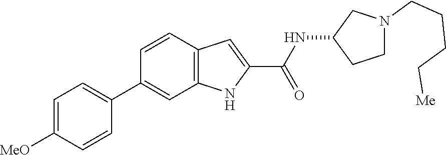 [SMILES: CCCCCN1CC[C@H](NC(=O)c2cc3ccc(-c4ccc(OC)cc4)cc3[nH]2)C1] |
| WW-46 | 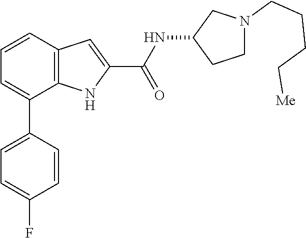 [SMILES: CCCCCN1CC[C@H](NC(=O)c2cc3cccc(-c4ccc(F)cc4)c3[nH]2)C1] |
| WW-47 | 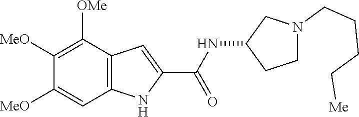 [SMILES: CCCCCN1CC[C@H](NC(=O)c2cc3c(OC)c(OC)c(OC)cc3[nH]2)C1] |
| WW-54 | 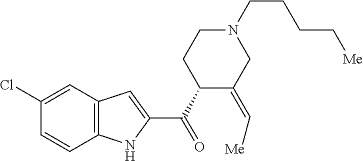 [SMILES: C/C=C1/CN(CCCCC)CC[C@H]1C(=O)c1cc2cc(Cl)ccc2[nH]1] |
| WW-55 | 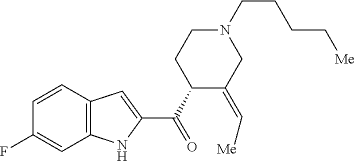 [SMILES: C/C=C1/CN(CCCCC)CC[C@H]1C(=O)c1cc2ccc(F)cc2[nH]1] |
| WW-57 | 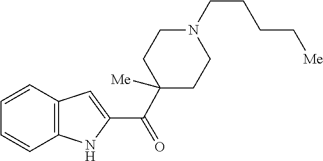 [SMILES: CCCCCN1CCC(C)(C(=O)c2cc3ccccc3[nH]2)CC1] |
| WW-58 | 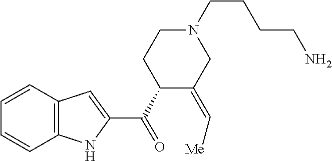 [SMILES: C/C=C1/CN(CCCCN)CC[C@H]1C(=O)c1cc2ccccc2[nH]1] |
| WW-59 | 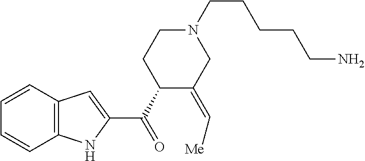 [SMILES: C/C=C1/CN(CCCCCN)CC[C@H]1C(=O)c1cc2ccccc2[nH]1] |
| WW-60 | 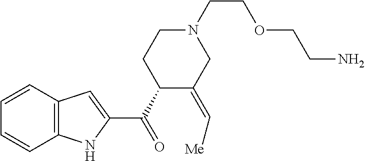 [SMILES: C/C=C1/CN(CCOCCN)CC[C@H]1C(=O)c1cc2ccccc2[nH]1] |
| WW-61 | 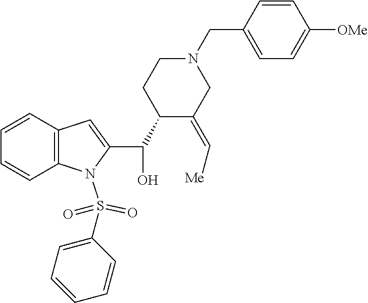 [SMILES: C/C=C1/CN(Cc2ccc(OC)cc2)CC[C@H]1C(O)c1cc2ccccc2n1S(=O)(=O)c1ccccc1] |
| WW-62 | 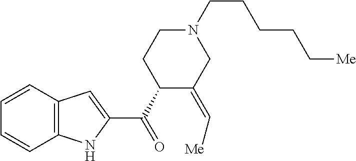 [SMILES: C/C=C1/CN(CCCCCC)CC[C@H]1C(=O)c1cc2ccccc2[nH]1] |
| WW-63 | 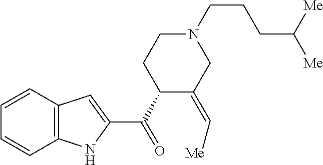 [SMILES: C/C=C1/CN(CCCC(C)C)CC[C@H]1C(=O)c1cc2ccccc2[nH]1] |
| WW-64 | 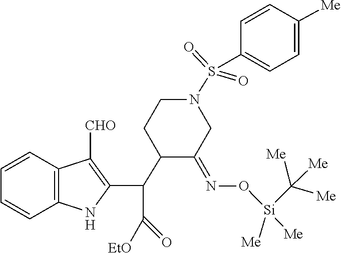 [SMILES: CCOC(=O)C(c1[nH]c2ccccc2c1C=O)C1CCN(S(=O)(=O)c2ccc(C)cc2)C/C1=N\O[Si](C)(C)C(C)(C)C] |
| WW-65 | 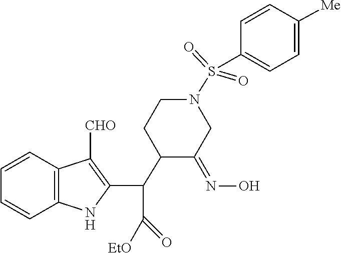 [SMILES: CCOC(=O)C(c1[nH]c2ccccc2c1C=O)C1CCN(S(=O)(=O)c2ccc(C)cc2)C/C1=N\O] |
| WW-66 | 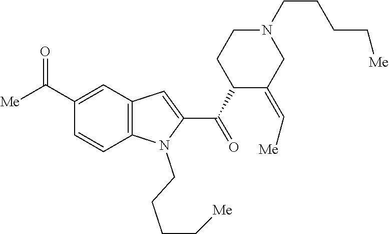 |
| WW-67 | 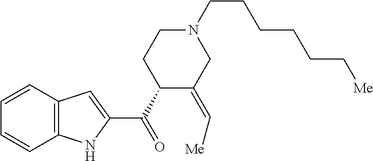 [SMILES: C/C=C1/CN(CCCCCCC)CC[C@H]1C(=O)c1cc2ccccc2[nH]1] |
| WW-68 | 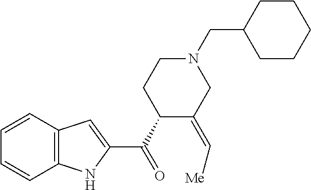 [SMILES: C/C=C1/CN(CC2CCCCC2)CC[C@H]1C(=O)c1cc2ccccc2[nH]1] |
| WW-69 | 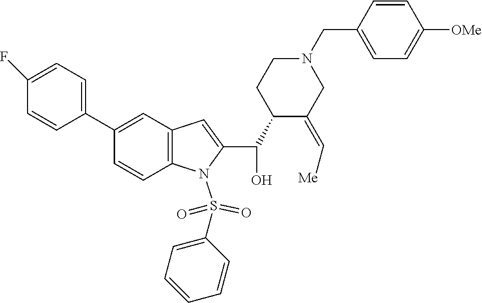 [SMILES: C/C=C1/CN(Cc2ccc(OC)cc2)CC[C@H]1C(O)c1cc2cc(-c3ccc(F)cc3)ccc2n1S(=O)(=O)c1ccccc1] |
| WW-70 | 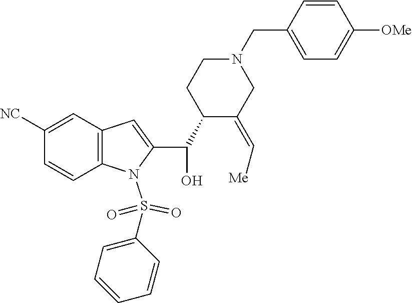 [SMILES: C/C=C1/CN(Cc2ccc(OC)cc2)CC[C@H]1C(O)c1cc2cc(C#N)ccc2n1S(=O)(=O)c1ccccc1] |
| WW-71 | 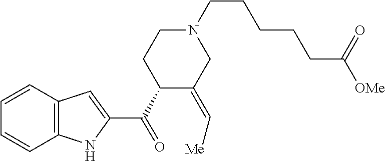 [SMILES: C/C=C1/CN(CCCCCC(=O)OC)CC[C@H]1C(=O)c1cc2ccccc2[nH]1] |
| WW-72 | 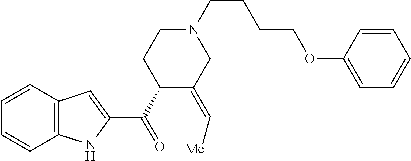 [SMILES: C/C=C1/CN(CCCCOc2ccccc2)CC[C@H]1C(=O)c1cc2ccccc2[nH]1] |
| WW-73 | 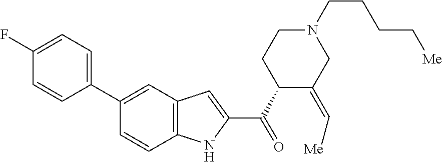 [SMILES: C/C=C1/CN(CCCCC)CC[C@H]1C(=O)c1cc2cc(-c3ccc(F)cc3)ccc2[nH]1] |
| WW-74 | 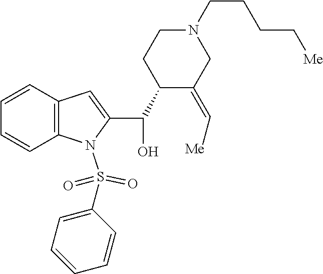 [SMILES: C/C=C1/CN(CCCCC)CC[C@H]1C(O)c1cc2ccccc2n1S(=O)(=O)c1ccccc1] |

The compounds of the invention may be in the form of pharmaceutically acceptable salts, as generally described below. Some preferred, but non-limiting examples of suitable pharmaceutically acceptable organic and/or inorganic acids are as hydrochloric acid, hydrobromic acid, sulfuric acid, nitric acid, acetic acid and citric acid, as well as other pharmaceutically acceptable acids known per se (for which reference is made to the prior art referred to below).

When the compounds of the invention contain an acidic group as well as a basic group the compounds of the invention may also form internal salts, and such compounds are within the scope of the invention. When the compounds of the invention contain a hydrogen-donating heteroatom (e.g. NH), the invention also covers salts and/or isomers formed by transfer of said hydrogen atom to a basic group or atom within the molecule.

Pharmaceutically acceptable salts of the compounds of the present invention include the acid addition and base salts thereof. Suitable acid addition salts are formed from acids which form non-toxic salts.

Examples include the acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

Pharmaceutically acceptable salts of compounds of the present invention may be prepared by one or more of these methods:
(i) by reacting the compounds of the present invention with the desired acid;
(ii) by reacting the compound of the present invention with the desired base;
(iii) by removing an acid- or base-labile protecting group from a suitable precursor of the compound of the present invention or by ring-opening a suitable cyclic precursor, for example, a lactone or lactam, using the desired acid; or
(iv) by converting one salt of the compound of the present invention to another by reaction with an appropriate acid or by means of a suitable ion exchange column.

All these reactions are typically carried out in solution. The salt, may precipitate from solution and be collected by filtration or may be recovered by evaporation of the solvent. The degree of ionization in the salt may vary from completely ionized to almost non-ionized.

The compounds of the invention may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Britain, Marcel Dekker, 1995), incorporated herein by reference. Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N+(CH₃)₃) polar head group. For more information, see Crystals and the Polarizing Microscope by N. H. Hartshorne and A. Stuart, 4^{th} Edition (Edward Arnold, 1970), incorporated herein by reference.

All references to compounds of the present invention include references to salts, solvates, multi-component complexes and liquid crystals thereof and to solvates, multi-component complexes and liquid crystals of salts thereof.

The compounds of the invention include compounds of the present invention as hereinbefore defined, including all polymorphs and crystal habits thereof, prodrugs and isomers thereof (including optical, geometric and tautomeric isomers) as hereinafter defined and isotopically-labeled compounds of the present invention

The terms “bind”, “interact”, “specifically bind” or “specifically interact” as used throughout this specification mean that an agent binds to or influences one or more desired molecules or analytes substantially to the exclusion of other molecules which are random or unrelated, and optionally substantially to the exclusion of other molecules that are structurally related. The terms do not necessarily require that an agent binds exclusively to its intended target(s). For example, an agent may be said to specifically bind to target(s) of interest if its affinity for such intended target(s) under the conditions of binding is at least about 2-fold greater, preferably at least about 5-fold greater, more preferably at least about 10-fold greater, yet more preferably at least about 25-fold greater, still more preferably at least about 50-fold greater, and even more preferably at least about 100-fold or more greater, such as, e.g., at least about 1000-fold or more greater, at least about 1×10⁴-fold or more greater, or at least about 1×10⁵-fold or more greater, than its affinity for a non-target molecule.

The binding or interaction between the agent and its intended target(s) may be covalent (i.e., mediated by one or more chemical bonds that involve the sharing of electron pairs between atoms) or, more typically, non-covalent (i.e., mediated by non-covalent forces, such as for example, hydrogen bridges, dipolar interactions, van der Waals interactions, and the like). Preferably, the agent may bind to or interact with its intended target(s) with affinity constant (K_{A}) of such binding K_{A}≥1×10⁶ M⁻¹, more preferably K_{A}≥1×10⁷ M⁻¹, yet more preferably K_{A}≥1×10⁸ M⁻¹, even more preferably K_{A}≥1×10⁹ M⁻¹, and still more preferably K_{A}≥1×10¹⁰ M⁻¹ or K_{A}≥1×10¹¹ M⁻¹, wherein K_{A}=[A_T]/[A][T], A denotes the agent, T denotes the intended target. Determination of K_{A} can be carried out by methods known in the art, such as for example, using equilibrium dialysis and Scatchard plot analysis.

A compound is said to “specifically bind to” a particular target when that compound has affinity for, specificity for, and/or is specifically directed against that target (i.e., against at least one part or fragment thereof).

The “specificity” of a compound as taught herein can be determined based on affinity. The “affinity” of a compound is represented by the equilibrium constant for the dissociation of the compound and ACKR3, preferably human ACKR3 (e.g. as annotated under NCBI Genbank accession number NP_064707.1). The lower the KD value, the stronger the binding strength between the compound and ACKR3. Alternatively, the affinity can also be expressed in terms of the affinity constant (KA), which corresponds to 1/KD. A KD value greater than about 1 millimolar is generally considered to indicate non-binding or non-specific binding.

The binding of an agent, such as a compound, as described herein to a target and the affinity and specificity of said binding may be determined by any methods known in the art. Non-limiting examples thereof include binding competition assays using fluorescently labelled or radiolabelled ligands (e.g. fluorescently labelled or radiolabelled chemokines, such as CXCL12), co-immunoprecipitation, bimolecular fluorescence complementation, affinity electrophoresis, label transfer, phage display, proximity ligation assay (PLA), Tandem affinity purification (TAP), in-silico docking and calculation of the predicted Gibbs binding energy and competition binding assays.

The compounds as taught herein have the ability to recruit β-arrestin-1 and β-arrestin-2 to the ACKR3 receptor when being used at nanomolar concentrations or even at subnanomolar concentrations.

In particular embodiments, the compound as taught herein has a potency for ACKR3 that is characterized by an EC₅₀ of 10 nM or less, 9 nM or less, 8 nM or less, 7 nM or less, 6 nM or less, 5 nM or less, 4 nM or less, 3 nM or less, 2 nM or less, 1 nM or less, 0.95 nM or less, 0.90 nM or less, 0.85 nM or less, 0.80 nM or less, 0.75 nM or less, 0.70 nM or less or 0.65 nM or less, preferably an EC₅₀ of 5 nM or less, more preferably an EC₅₀ of 1 nM or less. For example, the compound as taught herein has a potency for ACKR3 that is characterized by an EC₅₀ of 0.61 nM. The EC₅₀ in the context of the present invention was determined based on β-arrestin recruitment assay. β-arrestin recruitment can be determined by any methods known in the art such as by nanoluciferase complementation assays (e.g. NanoBiT, Promega), for instance using ACKR3 C-terminally fused to SmBiT and the β-arrestin N-terminally fused to LgBiT.

In particular embodiments, the compound as taught herein inhibits, reduces and/or prevents the interaction between ACKR3 and ACKR3 endogenous or exogenous ligands, such as endogenous opioid peptides (e.g. BAM-22), endogenous chemokines (e.g. CXCL12 or CXCL11), or exogenous opioid peptides.

In particular embodiments, the compound as taught herein inhibits, reduces and/or prevents the interaction between ACKR3 and an endogenous opioid peptide, such as an endogenous opioid peptide derived from proenkephalin, prodynorphin, proopiomelanocortin or prepronociceptin.

Preferably, an endogenous opioid peptide selected from the group consisting of BAM-22, BAM-18, Peptide E, adrenorphin, dynorphin A or fragments thereof (e.g. dynorphin 1-13, dynorphin 2-17), dynorphin B, big dynorphin or a fragment thereof, nociceptin or a fragment thereof.

In particular embodiments, the compound as taught herein inhibits, reduces and/or prevents the interaction between ACKR3 and an endogenous chemokine selected from the group consisting of CXCL12 (e.g. with Uniprot accession number P48061) and CXCL11 (e.g. with Uniprot accession number O14625).

The inhibition, reduction and/or prevention of the interaction between ACKR3 and endogenous ACKR3 ligands by the compound as taught herein can be determined by any means known in the art. It was previously found that ACKR3, in contrast to the known opioid receptors and in contrast to what was proposed by Ikeda et al. (Ikeda et al., 2013, Modulation of circadian glucocorticoid oscillation through adrenal opioid-CXCR7 signaling alters emotional behavior, Cell. 155(6): 1323-1336), is unable to activate downstream signalling pathways, for instance via G proteins or β-arrestins, in response to endogenous opioid peptides, but rather acts as a scavenger, regulating their local and/or systemic concentrations and thus availability for the classical opioid receptors. The absence or presence of downstream signalling pathway activation may be determined using methods known in the art, such as using a whole-cell biosensing approach based on dynamic mass redistribution, determining the recruitment of mini G proteins to the receptor, determining the phosphorylation level of ERK1/2, and determining activation of SRE (ERK1/2) and NFAT-RE (Ca²⁺) signalling cascades.

Accordingly, in particular embodiments, the compound as taught herein does not induce G-protein-mediated signalling mediated by ACKR3. The absence or presence of G-protein-mediated signalling may be determined using methods known in the art, such as determining the recruitment of mini G proteins to the receptor, determining the phosphorylation level of ERK1/2, whole cell biosensing approaches based on dynamic mass redistribution and determining activation of SRE (ERK1/2) and NFAT-RE (Ca²⁺) signalling cascades.

In more particular embodiments, the compound as taught does not induce recruitment of mini G proteins (mGs) (e.g. G_{αs}, G_{αi/o}, G_{αq/11} and/or G_{α12/13}) to ACKR3. The recruitment of mGs to ACKR3 (or the absence thereof) can be determined by any established analytical technique for determining protein-protein binding, such as co-immunoprecipitation, bimolecular fluorescence complementation, label transfer, tandem affinity purification, chemical cross-linking, fluorescence resonance energy transfer and nanoluciferase complementation assays (e.g. NanoBiT, Promega), for instance using ACKR3 C-terminally fused to SmBiT and mGs N-terminally fused to LgBiT. Protein binding assays may be performed in a cell-free system or in a cell lysate or in isolated or cultured cells or in an isolated or cultured tissue.

In particular embodiments, the compound as taught herein does not activate any signalling pathway (e.g. cAMP signalling and/or the MAPK/ERK signalling pathway) as a result of the recruitment of β-arrestin- and/or β-arrestin-2 to the ACKR3 receptor.

In particular embodiments, the compound as taught herein is not capable of interacting with and/or activating mu(μ)-type opioid receptor (MOR), delta (δ)-type opioid receptor (DOR), kappa (κ)-type opioid receptor (KOR) and non-classical nociceptin receptor (NOP). In more particular embodiments, the compound as taught herein does not reduce the recruitment of β-arrestin 1 and β-arrestin 2 to the MOR, DOR, KOR and/or NOP receptor(s) induced by a known ligand of the MOR, DOR, KOR and/or NOP receptor(s), respectively. In more particular embodiments, the compound as taught herein does not induce G-protein-mediated signalling via the MOR, DOR, KOR and/or NOP receptor(s). In more particular embodiments, the compound as taught herein is not capable of inducing the recruitment of 3-arrestin 1 and β-arrestin 2 to the MOR, DOR, KOR and/or NOP receptor(s). The absence of G-protein-mediated signalling can be determined by any methods known in the art such as determining the phosphorylation level of ERK1/2 upon contacting an agent with ACKR3, wherein a lack of phosphorylated ERK1/2 is indicative of the absence of G-protein-mediated signalling.

In particular embodiments, the compound as disclosed herein is not capable of inducing the recruitment of β-arrestin-1 and β-arrestin-2 to the MOR, DOR, KOR or NOP receptor. In more particular embodiments, the compound as disclosed herein does not enhance or even reduces β-arrestin-1 or D-arrestin-2 recruitment to the MOR, DOR, KOR or NOP receptor compared to the baseline β-arrestin-1 or β-arrestin-2 recruitment or background β-arrestin-1 or β-arrestin-2 recruitment induced by a neutral substance or negative control. As described elsewhere herein, the recruitment of β-arrestin-1 and β-arrestin-2 to the MOR, DOR, KOR or NOP receptor can be measured by a nanoluciferase complementation assays.

Any existing, available or conventional separation, detection and quantification methods may be used herein to measure the presence or absence (e.g., readout being present vs. absent; or detectable amount vs. undetectable amount) and/or quantity (e.g., readout being an absolute or relative quantity, such as, for example, absolute or relative concentration) of peptides, polypeptides, proteins in samples. For example, such methods may include biochemical assay methods, immunoassay methods, mass spectrometry analysis methods, or chromatography methods, or combinations thereof.

In particular embodiments, the compound as taught herein is not capable of inducing the recruitment of β-arrestin-1 and β-arrestin-2 to any other chemokine receptor than ACKR3, more particularly a chemokine receptor selected from the group consisting of C-C chemokine receptor type 1 (CCR1) (e.g. with UniProt accession number P32246), C-C chemokine receptor type 2 (CCR2) (e.g. with UniProt accession number P41597) such as CCRtype 2A (CCR2A) or CCRtype 2B (CCR2B), C-C chemokine receptor type 3 (CCR3) (e.g. with UniProt accession number P51677), C-C chemokine receptor type 4 (CCR4) (e.g. with UniProt accession number P51679), C-C chemokine receptor type 5 (CCR5) (e.g. with UniProt accession number P51681), C-C chemokine receptor type 6 (CCR6) (e.g. with UniProt accession number P51684), C-C chemokine receptor type 7 (CCR7) (e.g. with UniProt accession number P32248), C-C chemokine receptor type 8 (CCR8) (e.g. with UniProt accession number P51685), C-C chemokine receptor type 9 (CCR9) (e.g. with UniProt accession number P51686), C-C chemokine receptor type 10 (CCR10) (e.g. with UniProt accession number P46092), C-X-C motif chemokine receptor 1 (CXCR1) (e.g. with UniProt accession number P25024), C-X-C motif chemokine receptor 2 (CXCR2) (e.g. with UniProt accession number P25025), C-X-C motif chemokine receptor 3 (CXCR3) (e.g. with UniProt accession number P49682) such as CXCR type 3A (CXCR3A) and CXCR type 3B (CXCR3B), C-X-C motif chemokine receptor 4 (CXCR4) (e.g. with UniProt accession number P61073), C-X-C motif chemokine receptor 5 (CXCR5) (e.g. with UniProt accession number P32302), C-X-C motif chemokine receptor 6 (CXCR6) (e.g. with UniProt accession number O00574), C-X-C motif chemokine receptor 8 (CXCR8) (e.g. with UniProt accession number Q9HC97), X—C motif chemokine receptor 1 (XCR1) (e.g. with UniProt accession number P46094), C-X3-C motif chemokine receptor 1 (CX3CR1) (e.g. with UniProt accession number P49238), atypical chemokine receptor 1 (ACKR1) (e.g. with UniProt accession number Q16570), atypical chemokine receptor 2 (ACKR2) (e.g. with UniProt accession number 000590) and atypical chemokine receptor 4 (ACKR4) (e.g. with UniProt accession number Q9NPB9).

In particular embodiments, the compound as disclosed herein is not capable of inducing the recruitment of β-arrestin-1 and β-arrestin-2 to the CCR1, CCR2A, CCR2B, CCR3, CCR4, CCR5, CCR6, CCR7, CCR8, CCR9, CCR10, CXCR1, CXCR2, CXCR3A, CXCR3B, CXCR4, CXCR5, CXCR6, CXCR8, XCR1, CX3CR1, ACKR1, ACKR2 or ACKR4 receptor. In even more particular embodiments, the compound as disclosed herein does not enhance or reduces β-arrestin-1 or β-arrestin-2 recruitment to the CCR1, CCR2A, CCR2B, CCR3, CCR4, CCR5, CCR6, CCR7, CCR8, CCR9, CCR10, CXCR1, CXCR2, CXCR3A, CXCR3B, CXCR4, CXCR5, CXCR6, CXCR8, XCR1, CX3CR1, ACKR1, ACKR2 or ACKR4 receptor compared to the baseline β-arrestin-1 or β-arrestin-2 recruitment or background β-arrestin-1 or β-arrestin-2 recruitment induced by a neutral substance or negative control. As described elsewhere herein, the recruitment of β-arrestin-1 and β-arrestin-2 to the CCR1, CCR2A, CCR2B, CCR3, CCR4, CCR5, CCR6, CCR7, CCR8, CCR9, CCR10, CXCR1, CXCR2, CXCR3A, CXCR3B, CXCR4, CXCR5, CXCR6, CXCR8, XCR1, CX3CR1, ACKR1, ACKR2 or ACKR4 receptor can be measured by a nanoluciferase complementation assays.

By means of additional guidance, atypical chemokine receptor 3 (ACKR3) is also known in the art as chemokine receptor 7 (CXCR7). By means of an example, human ACKR3 mRNA is annotated under NCBI Genbank accession number NM_020311.2. Human ACKR3 polypeptide is annotated under NCBI Genbank (http://www.ncbi.nlm.nih.gov/) accession number NP_064707.1, and Uniprot accession number P25106.

By means of additional guidance, mu(μ)-type opioid receptor (MOR) is also known in the art as OPRM, LMOR or MOP. By means of an example, human MOR protein is annotated under NCBI Genbank (http://www.ncbi.nlm.nih.gov/) accession number AY521028.1 and Uniprot accession number P35372. By means of additional guidance, delta (δ)-type opioid receptor (DOR) is also known in the art as OPRD or DOP. By means of an example, human DOR protein is annotated under NCBI Genbank (http://www.ncbi.nlm.nih.gov/) accession number NM_000911.4 and Uniprot accession number P41143.

By means of additional guidance, kappa (κ)-type opioid receptor (KOR) is also known in the art as OPRK or KOP. By means of an example, human KOR protein is annotated under NCBI Genbank (http://www.ncbi.nlm.nih.gov/) accession number AF498922.1 and Uniprot accession number P41145. By means of additional guidance, non-classical nociceptin receptor (NOP) is also known in the art as orphanin FQ receptor, OPRL and opioid related nociceptin receptor 1. By means of an example, human NOP protein is annotated under NCBI Genbank (http://www.ncbi.nlm.nih.gov/) accession number AY268428.1 and Uniprot accession number P41146.

A skilled person can appreciate that any sequences represented in sequence databases or in the present specification may be of precursors ofthe respective peptides, polypeptides, proteins or nucleic acids and may include parts which are processed away from mature molecules.

References to any peptides, polypeptides, proteins or nucleic acids denote the respective peptides, polypeptides, proteins or nucleic acids as commonly known under the respective designations in the art.

More particularly, the references to “ACKR3”, “MOR”, “DOR”, “KOR”, “NOP”, “CCR1”, “CCR2A”, “CCR2B”, “CCR3”, “CCR4”, “CCR5”, “CCR6”, “CCR7”, “CCR8”, “CCR9”, “CCR10”, “CXCR1”, “CXCR2”, “CXCR3A”, “CXCR3B”, “CXCR4”, “CXCR5”, “CXCR6”, “CXCR8”, “XCR1”, “CX3CR1”, “ACKR1”, “ACKR2” or “ACKR4” denote the respective peptides, polypeptides, proteins or nucleic acids, as apparent from the context, as commonly known under said designations in the art.

The terms encompass the peptides, polypeptides, proteins or nucleic acids when forming a part of a living organism, organ, tissue or cell, when forming a part of a biological sample, as well as when at least partly isolated from such sources. The terms also encompass the peptides, polypeptides, proteins or nucleic acids when produced by recombinant or synthetic means.

The reference to any peptides, polypeptides, proteins or nucleic acids encompass such peptides, polypeptides, proteins or nucleic acids of any organism where found, and particularly of animals, preferably warm-blooded animals, more preferably vertebrates, yet more preferably mammals, including humans and non-human mammals, still more preferably of humans.

Hence, in certain embodiments, one or more and preferably all of ACKR3, MOR, DOR, KOR, NOP, CCR1, CCR2A, CCR2B, CCR3, CCR4, CCR5, CCR6, CCR7, CCR8, CCR9, CCR10, CXCR1, CXCR2, CXCR3A, CXCR3B, CXCR4, CXCR5, CXCR6, CXCR8, XCR1, CX3CR1, ACKR1, ACKR2 and ACKR4 as employed herein is or are of animal origin, preferably warm-blooded animal origin, more preferably vertebrate origin, yet more preferably mammalian origin, including human origin and non-human mammalian origin, still more preferably human origin.

For biological peptides, polypeptides, proteins or nucleic acids the native sequences may differ between different species due to genetic divergence between such species. Moreover, native sequences may differ between or within different individuals of the same species due to normal genetic diversity (variation) within a given species. Also, native sequences may differ between or even within different individuals of the same species due to somatic mutations, or post-transcriptional or post-translational modifications. Any such variants or isoforms of peptides, polypeptides, proteins or nucleic acids are intended herein. Accordingly, all sequences of peptides, polypeptides, proteins or nucleic acids found in or derived from nature are considered “native”.

Unless otherwise apparent from the context, reference herein to any peptide, polypeptide, protein or nucleic acid also encompasses modified forms of said peptide, polypeptide, protein or nucleic acid, such as forms bearing post-expression modifications including, for example, phosphorylation, glycosylation, lipidation, methylation, cysteinylation, sulphonation, glutathionylation, acetylation, oxidation of methionine to methionine sulphoxide or methionine sulphone, and the like.

The term “protein” as used throughout this specification generally encompasses macromolecules comprising one or more polypeptide chains, i.e., polymeric chains of amino acid residues linked by peptide bonds. The term may encompass naturally, recombinantly, semi-synthetically or synthetically produced proteins. The term also encompasses proteins that carry one or more co- or post-expression-type modifications of the polypeptide chain(s), such as, without limitation, glycosylation, acetylation, phosphorylation, sulphonation, methylation, ubiquitination, signal peptide removal, N-terminal Met removal, conversion of pro-enzymes or pre-hormones into active forms, etc. The term further also includes protein variants or mutants which carry amino acid sequence variations vis-à-vis a corresponding native proteins, such as, e.g., amino acid deletions, additions and/or substitutions. The term contemplates both full-length proteins and protein parts or fragments, e.g., naturally occurring protein parts that ensue from processing of such full-length proteins.

The term “polypeptide” as used throughout this specification generally encompasses polymeric chains of amino acid residues linked by peptide bonds. Hence, especially when a protein is only composed of a single polypeptide chain, the terms “protein” and “polypeptide” may be used interchangeably herein to denote such a protein. The term is not limited to any minimum length of the polypeptide chain. The term may encompass naturally, recombinantly, semi-synthetically or synthetically produced polypeptides. The term also encompasses polypeptides that carry one or more co- or post-expression-type modifications of the polypeptide chain, such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes polypeptide variants or mutants, which carry amino acid sequence variations vis-à-vis a corresponding native polypeptide, such as, e.g., amino acid deletions, additions and/or substitutions. The term contemplates both full-length polypeptides and polypeptide parts or fragments, e.g., naturally occurring polypeptide parts that ensue from processing of such full-length polypeptides.

A polypeptide or protein can be naturally occurring, e.g., present in or isolated from nature, e.g., produced or expressed natively or endogenously by a cell or tissue and optionally isolated therefrom. A polypeptide or protein can be recombinant, i.e., produced by recombinant DNA technology, and/or can be, partly or entirely, chemically or biochemically synthesised. Without limitation, polypeptide or protein can be produced recombinantly by a suitable host or host cell expression system and optionally isolated therefrom (e.g., a suitable bacterial, yeast, fungal, plant or animal host or host cell expression system), or produced recombinantly by cell-free translation or cell-free transcription and translation, or non-biological polypeptide or protein synthesis.

The selectivity and high affinity of the compounds as taught herein for ACKR3 provides the compounds as taught herein with many valuable in vitro, ex vivo and in vivo applications.

ACKR3 acts as a scavenger for chemokines, regulating the local and/or systemic concentrations and thus availability for the other chemokine receptors. Accordingly, the compound as taught herein could be used to regulate the availability of endogenous (such as CXCL11 or CXCL12) or exogenous (such as vCCL2 (vMIP-II)) chemokines for other chemokine receptors, including the CCR1, CCR2A, CCR2B, CCR3, CCR4, CCR5, CCR6, CCR7, CCR8, CCR9, CCR10, CXCR1, CXCR2, CXCR3A, CXCR3B, CXCR4, CXCR5, CXCR6, CXCR8, XCR1, CX3CR1, ACKR1, ACKR2 and ACKR4 polypeptides. As a result thereof, the compounds or the pharmaceutical composition as taught herein could be used in the treatment of diseases or conditions in which these endogenous or exogenous chemokines play a role. Also, has been found that ACKR3 acts as a scavenger for endogenous opioid peptides, regulating the local and/or systemic concentrations and thus availability for the classical opioid receptors. Accordingly, all agents specifically binding to the ACKR3 polypeptide and/or specifically inducing β-arrestin-1 and/or β-arrestin-2 recruitment to the ACKR3 polypeptide can be used to regulate the availability of endogenous opioid peptides for other opioid receptors, including the MOR, KOR, DOR and NOP polypeptides.

Accordingly, a further aspect provides the novel compounds as taught herein or the pharmaceutical composition as taught herein for use as a medicament.

A further aspect provides the compounds as taught herein as a therapeutic or prophylactic agent for use in the treatment of a disease or condition in a subject, wherein said therapeutic or prophylactic agent is capable of modulating (e.g. inducing or antagonizing), preferably capable of inducing, β-arrestin-1 and/or β-arrestin-2 recruitment to the ACKR3 polypeptide and is not capable of inducing β-arrestin-1 and/or β-arrestin-2 recruitment to any other receptor polypeptide, including any opioid receptor polypeptide selected from the group consisting of the MOR, DOR, KOR and NOP receptor, and any chemokine receptor polypeptide selecting from the group consisting of CCR1, CCR2A, CCR2B, CCR3, CCR4, CCR5, CCR6, CCR7, CCR8, CCR9, CCR10, CXCR1, CXCR2, CXCR3A, CXCR3B, CXCR4, CXCR5, CXCR6, CXCR8, XCR1, CX3CR1, ACKR1, ACKR2 and ACKR4.

The terms “treat” or “treatment” encompass both the therapeutic treatment of an already developed disease or condition, as well as prophylactic or preventive measures, wherein the aim is to prevent or lessen the chances of incidence of an undesired affliction. Beneficial or desired clinical results may include, without limitation, alleviation of one or more symptoms or one or more biological markers, diminishment of extent of disease, stabilised (i.e., not worsening) state of disease, delay or slowing of disease progression, amelioration or palliation of the disease state, and the like. “Treatment” can also mean prolonging survival as compared to expected survival if not receiving treatment.

A related aspect provides a method for treating a dysfunction disease or condition in a subject comprising administering a therapeutically and/or prophylactically effective amount of a therapeutic or prophylactic agent to said subject, wherein said therapeutic or prophylactic agent is capable of inducing β-arrestin-1 and/or β-arrestin-2 recruitment to the ACKR3 polypeptide, and is not capable of inducing β-arrestin-1 and/or β-arrestin-2 recruitment to any other receptor polypeptide, including any opioid receptor polypeptide selected from the group consisting of the MOR, DOR, KOR and NOP receptor, and any chemokine receptor polypeptide selecting from the group consisting of CCR1, CCR2A, CCR2B, CCR3, CCR4, CCR5, CCR6, CCR7, CCR8, CCR9, CCR10, CXCR1, CXCR2, CXCR3A, CXCR3B, CXCR4, CXCR5, CXCR6, CXCR8, XCR1, CX3CR1, ACKR1, ACKR2 and ACKR4.

The term “therapeutically effective amount” as used herein, refers to an amount of therapeutic agent that elicits the biological or medicinal response in a subject that is being sought by a surgeon, researcher, veterinarian, medical doctor or other clinician, which may include inter alia alleviation of the symptoms of the disease or condition being treated. The term “prophylactically effective amount” refers to an amount of the prophylactic agent that inhibits or delays in a subject the onset of a disorder as being sought by a researcher, veterinarian, medical doctor or other clinician. Methods are known in the art for determining therapeutically and/or prophylactically effective amounts of the therapeutic or prophylactic agent as described herein.

The compound as taught herein can be used in the treatment of diseases or conditions that are, at least in part, dependent on ACKR3 activity. In particular embodiments, the compounds of the invention are envisaged for the treatment of a disease or condition characterized by an aberrant level of ACKR3 polypeptide.

In particular embodiments, the compounds of the invention are envisaged for the treatment of a distress dysfunction disease or condition. In particular embodiments, the distress dysfunction disease or condition is selected from the group consisting of anxiety disorders, depression, anger, insomnia, mood disorders, substance and behavioural addictions (e.g. opiate, cocaine or alcohol abuse and/or dependence), and eating disorders (e.g. anorexia). In preferred embodiments, the distress dysfunction disease or condition is selected from the group consisting of anxiety disorders and depression ACKR3 plays a key role in controlling the angiogenic process, for example, in cancers. Accordingly, the present invention encompasses decreasing angiogenesis in any subject in need thereof (e.g. subject having a disease or condition involving excessive or abnormal angiogenesis) by administering the compounds as taught herein.

ACKR3 (CXCR7)-mediated viral entry has been demonstrated for several clinical HIV isolates as well as laboratory strains. Thus, the compounds of the present invention can be used as inhibitors of CXCR7 receptor-mediated HIV entry and replication, in the treatment of HIV.

ACKR3 plays a key role in regulating the availability of endogenous opioid peptides for other opioid receptors. Accordingly, a further aspect provides the use of the compound as taught herein for use in the treatment of pain. More particularly, the application provides the use of known and novel ACKR-3 modulators for use in the treatment of pain. In particular embodiments, the compounds are not conolidine derivatives. More particularly, the compounds are not compounds disclosed in WO2012088402.

A further aspect thus provides the compound as taught herein, or the pharmaceutical composition as taught herein for use in the treatment of a disease or condition selected from the group consisting of distress dysfunction diseases or conditions, pain, cancers, atherosclerotic vascular disease (or atherosclerosis), cardiovascular diseases, fibrosis (e.g. cardiac fibrosis), inflammatory or autoimmune diseases and conditions, conditions of excessive or abnormal vascularization (e.g. wound healing), stem cell differentiation and mobilization disorders, brain and neuronal dysfunctions (e.g. Alzheimer's disease, multiple sclerosis and demyelinating diseases), kidney dysfunction, renal dysfunction, preeclampsia, human immunodeficiency virus (HIV) infection and obesity, preferably a disease or condition selected from the group consisting of distress dysfunction diseases or conditions, cancers, atherosclerotic vascular disease, cardiovascular diseases and fibrosis, in a subject.

Non-limiting examples of inflammatory or autoimmune diseases and conditions include inflammatory bowel disease, rheumatoid arthritis, osteoarthritis, psoriatic arthritis, polyarticular arthritis, renal inflammatory disorders, multiple sclerosis, colitis, allergic diseases, psoriasis, atopic dermatitis and asthma.

A related aspect provides a method for treating a disease or conditions described above in a subject comprising administering a therapeutically and/or prophylactically effective amount of the compound as taught herein or the pharmaceutical composition as taught herein to said subject.

A further aspect provides the use of the compound as taught herein, or the pharmaceutical composition as taught herein for reducing tumour cell proliferation, tumour formation, tumour vascularization and metastasis.

A further aspect provides the use of the compound as taught herein or the pharmaceutical composition as taught herein for increasing T cell recruitment in a subject.

A further aspect provides the use of the compound as taught herein or the pharmaceutical composition as taught herein for reducing viral reproduction in a subject.

In particular embodiment, the compound as taught herein or the pharmaceutical composition as taught herein are used in combination with agents known to be used in the treatment of the diseases or conditions listed above.

In particular embodiments, the compound as taught herein may be fused to an agent.

In the context of present invention, the term “coupled” as used herein is synonymous with “connected”, “bound”, “fused”, “joined” and refers to a physical or chemical link between at least two elements or components. In some embodiments the term “coupled” or “bound” refers to a covalent link.

As used herein, the term “agent” broadly refers to any chemical (e.g., inorganic or organic), biochemical or biological substance, molecule or macromolecule (e.g., biological macromolecule), a combination or mixture thereof, a sample of undetermined composition, or an extract made from biological materials such as bacteria, fungi, plants, or animal cells or tissues. Preferred though non-limiting “agents” include nucleic acids, oligonucleotides, ribozymes, peptides, polypeptides, proteins, peptidomimetics, antibodies, antibody fragments, antibody-like protein scaffolds, aptamers, photoaptamers, spiegelmers, chemical substances, preferably organic molecules, more preferably small organic molecules, lipids, carbohydrates, polysaccharides, etc., and any combinations thereof.

In particular embodiments, the compound as taught herein may be coupled to an agent selected from the group consisting of a chemical substance, an antibody, an antibody fragment, an antibody-like protein scaffold, a protein or polypeptide and a peptide, a peptidomimetic, an aptamer, a photoaptamer, a spiegelmer and a nucleic acid.

As used herein, the term “chemical substance” is used in its broadest sense and generally refers to any substantially pure substance that has a constant chemical composition and characteristic properties. The chemical substance may be an organic molecule, preferably a small organic molecule. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da.

The term “antibody” is used herein in its broadest sense and generally refers to any immunologic binding agent, such as a whole antibody, including without limitation a chimeric, humanized, human, recombinant, transgenic, grafted and single chain antibody, and the like, or any fusion proteins, conjugates, fragments, or derivatives thereof that contain one or more domains that selectively bind to an antigen of interest. The term antibody thereby includes a whole immunoglobulin molecule, a monoclonal antibody, a chimeric antibody, a humanized antibody, a human antibody, or an immunologically effective fragment of any of these. The term thus specifically encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest), as well as multivalent and/or multi-specific composites of such fragments. The term “antibody” is not only inclusive of antibodies generated by methods comprising immunisation, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro, in cell culture, or in vivo.

In particular embodiments, the compound as taught herein may be fused (i.e. covalently linked) to a detectable label.

The term “label” refers to any atom, molecule, moiety or biomolecule that may be used to provide a detectable and preferably quantifiable read-out or property, and that may be attached to or made part of an entity of interest, such as the compound as taught herein. Labels may be suitably detectable by for example mass spectrometric, spectroscopic, optical, colourimetric, magnetic, photochemical, biochemical, immunochemical or chemical means. Labels include without limitation dyes; radiolabels such as ³²P, ³³P, ³⁵S, ¹²⁵I, ¹³¹I; electron-dense reagents; enzymes (e.g., horse-radish peroxidase or alkaline phosphatase as commonly used in immunoassays); binding moieties such as biotin-streptavidin; haptens such as digoxigenin; luminogenic, phosphorescent or fluorogenic moieties; mass tags; and fluorescent dyes (e.g., fluorophores such as fluorescein, carboxyfluorescein (FAM), tetrachloro-fluorescein, TAMRA, ROX, Cy3, Cy3.5, Cy5, Cy5.5, Texas Red, etc.) alone or in combination with moieties that may suppress or shift emission spectra by fluorescence resonance energy transfer (FRET).

In some embodiments, the compound as taught herein may be provided with a tag that permits detection with another agent (e.g., with a probe binding partner). Such tags may be, for example, biotin, streptavidin, his-tag, myc tag, maltose, maltose binding protein or any other kind of tag known in the art that has a binding partner. Example of associations which may be utilised in the probe:binding partner arrangement may be any, and includes, for example biotin:streptavidin, his-tag:metal ion (e.g., Ni²⁺), maltose:maltose binding protein, etc.

In particular embodiments, the label may be Large BiT (LgBiT) or Small BiT (SmBiT) or HiBiT of NanoLuc® Binary Technology (NanoBiT).

The compound as taught herein may be associated with or attached to a detection agent to facilitate detection. Examples of detection agents include, but are not limited to, luminescent labels; colorimetric labels, such as dyes; fluorescent labels (e.g. green fluorescent protein (GFP)); or chemical labels, such as electroactive agents (e.g., ferrocyanide); enzymes; radioactive labels; or radiofrequency labels. The detection agent may be a particle. Examples of such particles include, but are not limited to, colloidal gold particles; colloidal sulphur particles; colloidal selenium particles; colloidal barium sulphate particles; colloidal iron sulphate particles; metal iodate particles; silver halide particles; silica particles; colloidal metal (hydrous) oxide particles; colloidal metal sulfide particles; colloidal lead selenide particles; colloidal cadmium selenide particles; colloidal metal phosphate particles; colloidal metal ferrite particles; any of the above-mentioned colloidal particles coated with organic or inorganic layers; protein or peptide molecules; liposomes; or organic polymer latex particles, such as polystyrene latex beads.

In particular embodiments, the compound as taught herein may be coupled to the agent by one or more linkers.

As used herein, the term “linker” refers to a connecting element that serves to link other elements. The linker may be a rigid linker or a flexible linker. In particular embodiments, the linker is a covalent linker, achieving a covalent bond. The terms “covalent” or “covalent bond” refer to a chemical bond that involves the sharing of one or more electron pairs between two atoms. For many molecules, the sharing of electrons allows each atom to attain the equivalent of a full outer electron shell, corresponding to a stable electronic configuration. Covalent bonds include different types of interactions, including σ-bonds, π-bonds, metal-to-metal bonds, agostic interactions, bent bonds and three-center two-electron bonds.

In particular embodiments, the linker is a (poly) peptide linker or a non-peptide linker, such as a non-peptide polymer, such as a non-biological polymer. Preferably, the linkage(s) between the compound as taught herein and the peptide, protein or polypeptide may be hydrolytically stable linkage(s), i.e., substantially stable in water at useful pH values, including in particular under physiological conditions, for an extended period of time, e.g., for days. In particular embodiments, the linker is a peptide linker of one or more amino acids.

A further aspect provides a pharmaceutical composition comprising the compound as taught herein and optionally a pharmaceutically acceptable carrier.

The term “pharmaceutically acceptable” as used herein is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, antioxidants, tonicity controlling agents, absorption delaying agents, and the like. The use of such media and agents for pharmaceutical active substances is well known in the art. Except insofar as any conventional media or agent is incompatible with the active substance, its use in the therapeutic compositions may be contemplated.

Illustrative, non-limiting carriers for use in formulating the pharmaceutical compositions include, for example, oil-in-water or water-in-oil emulsions, aqueous compositions with or without inclusion of organic co-solvents suitable for intravenous (IV) use, liposomes or surfactant-containing vesicles, microspheres, microbeads and microsomes, powders, tablets, capsules, suppositories, aqueous suspensions, aerosols, and other carriers apparent to one of ordinary skill in the art.

Pharmaceutical compositions as intended herein may be formulated for essentially any route of administration, such as without limitation, oral administration (such as, e.g., oral ingestion or inhalation), intranasal administration (such as, e.g., intranasal inhalation or intranasal mucosal application), parenteral administration (such as, e.g., subcutaneous, intravenous (I.V.), intramuscular, intraperitoneal or intrasternal injection or infusion), transdermal or transmucosal (such as, e.g., oral, sublingual, intranasal) administration, topical administration, rectal, vaginal or intra-tracheal instillation, and the like. In this way, the therapeutic effects attainable by the methods and compositions can be, for example, systemic, local, tissue-specific, etc., depending of the specific needs of a given application. In preferred embodiments, the compound or the pharmaceutical composition as taught herein is administered parenterally. More preferably, the compound or the pharmaceutical composition as taught herein is administered intravenously, for example by infusion.

The dosage or amount of the agent as taught herein, optionally in combination with one or more other active compounds to be administered, depends on the individual case and is, as is customary, to be adapted to the individual circumstances to achieve an optimum effect. Thus, the unit dose and regimen depend on the nature and the severity of the disorder to be treated, and also on factors such as the species of the subject, the sex, age, body weight, general health, diet, mode and time of administration, immune status, and individual responsiveness of the human or animal to be treated, efficacy, metabolic stability and duration of action of the compounds used, on whether the therapy is acute or chronic or prophylactic, or on whether other active compounds are administered in addition to the agent of the invention. In order to optimize therapeutic efficacy, the compound or the pharmaceutical composition as taught herein can be first administered at different dosing regimens. Typically, levels of the agent in a tissue can be monitored using appropriate screening assays as part of a clinical testing procedure, e.g., to determine the efficacy of a given treatment regimen. The frequency of dosing is within the skills and clinical judgement of medical practitioners (e.g., doctors, veterinarians or nurses). Typically, the administration regime is established by clinical trials which may establish optimal administration parameters. However, the practitioner may vary such administration regimes according to the one or more of the aforementioned factors, e.g., subject's age, health, weight, sex and medical status. The frequency of dosing can be varied depending on whether the treatment is prophylactic or therapeutic.

Toxicity and therapeutic efficacy of the agent as described herein or pharmaceutical compositions comprising the same can be determined by known pharmaceutical procedures in, for example, cell cultures or experimental animals. These procedures can be used, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Pharmaceutical compositions that exhibit high therapeutic indices are preferred. While pharmaceutical compositions that exhibit toxic side effects can be used, care should be taken to design a delivery system that targets such compounds to the site of affected tissue in order to minimize potential damage to normal cells (e.g., non-target cells) and, thereby, reduce side effects. The data obtained from the cell culture assays and animal studies can be used in formulating a range of dosage for use in appropriate subjects. The dosage of such pharmaceutical compositions lies generally within a range of circulating concentrations that include the ED50 with little or no toxicity. The dosage may vary within this range depending upon the dosage form employed and the route of administration utilised. For a pharmaceutical composition used as described herein, the therapeutically effective dose can be estimated initially from cell culture assays. A dose can be formulated in animal models to achieve a circulating plasma concentration range that includes the IC50 (i.e., the concentration of the pharmaceutical composition which achieves a half-maximal inhibition of symptoms) as determined in cell culture. Such information can be used to more accurately determine useful doses in humans. Levels in plasma can be measured, for example, by high performance liquid chromatography.

In particular embodiment, the compound as taught herein is the main or only active ingredient of the pharmaceutical composition.

A further aspect provides the use of the compound as described herein in stabilizing the ACKR3 polypeptide, for instance, during nuclear magnetic resonance (NMR) analysis. Conformational flexibility of receptors can be an obstacle in protein production and crystallography studies. As the compound as taught herein specifically recognizes ACKR3 polypeptide, the compound could be used to specifically target agents, such as detectable labels, pharmaceuticals, or toxins, to the ACKR3 polypeptide.

Accordingly, a further aspect provides the use of the compound as described herein for targeted delivery of an agent, such a pharmaceutical or toxin, to the ACKR3 polypeptide.

For example, ACKR3 is expressed in various cells such as B and T lymphocytes, neurons and endothelial cells and plays a role in many types of cancer, cardiovascular and neuronal development, cardiac and immune pathophysiology and migration and homing of hematopoietic stem/progenitor cells. ACKR3 is expressed in various cancer cell types (such as colorectal cancer, breast cancer, prostate cancer, lung cancer, liver cancer, lymphoma, leukaemia, glioblastoma and head and neck cancer) as well as on tumour-associated vasculature and is involved in metastasis development. ACKR3 is also upregulated upon infection by several cancer-inducing viruses including HHV-8, EBV, HTLV-1 and plays an important role in cell transformation and proliferation.

Accordingly, in particular embodiments, the compound as taught herein fused (i.e. covalently linked) to a toxin, is used in the treatment of cancer.

A related aspect provides the use of the compound as described herein as a tracer, for instance a tracer for in vivo, ex vivo or in vitro imaging. For instance, the compound as taught herein, when fused to a detectable label, could be used for visualizing cells, tissues and/or organs expressing ACKR3 (e.g. certain types of cancer cells). Accordingly, the compound as taught herein, when fused to a detectable label, could also be used for visualizing diseases or conditions related to ACKR3, such as cancer, diseases or conditions involving excessive or abnormal angiogenesis, and inflammatory or autoimmune diseases and conditions (e.g. arthritis). More particularly, the compound as taught herein, when fused to a detectable label, could be used for visualizing cancer, atherosclerotic vascular disease, cardiac fibrosis, or brain and neuronal dysfunction (e.g. Alzheimer's disease, multiple sclerosis and demyelinating diseases), in vivo, ex vivo or in vitro. Non-limiting examples of cancers which can be visualised using the compound as taught herein include carcinomas, gliomas, mesotheliomas, melanomas, lymiphomas, leukaemias, adenocarcinomas, breast cancer, ovarian cancer, cervical cancer, glioblastoma, prostate cancer, Burkitt's lymphoma, head and neck cancer, colon cancer, colorectal cancer, non-small cell lung cancer, small-cell lung cancer, cancer of the oesophagus, stomach cancer, pancreatic cancer, hepatobiliary cancer, cancer of the gallbladder, cancer of the small intestine, rectal cancer, kidney cancer, bladder cancer, penile cancer, urethral cancer, testicular cancer, vaginal cancer, uterine cancer, thyroid cancer, parathyroid cancer, adrenal cancer, pancreatic endocrine cancer, carcinoid cancer, bone cancer, skin cancer, retinoblastomas, Hodgkin's lymphoma, and non-Hodgkin's lymphoma.

A further aspect provides a method for in vitro or ex vivo detecting and/or determining the level of the ACKR3 polypeptide in a biological sample, comprising the steps of
obtaining a biological sample obtained from a subject,
contacting said biological sample with the compound as taught herein, wherein said compound is fused or covalently linked to a detectable label,
detecting and/or determining the level of the ACKR3 polypeptide in said biological sample by detecting the compound as taught herein.

The terms “level”, “quantity”, “amount” and are synonymous and generally well-understood in the art.

The terms as used herein may particularly refer to an absolute quantification of a molecule or an analyte in a sample, or to a relative quantification of a molecule or analyte in a sample, i.e., relative to another value such as relative to a reference value as taught herein, or to a range of values indicating a base-line expression of the molecule or analyte. These values or ranges can be obtained from a single patient or from a group of patients.

A related aspect provides the compound as taught herein for use in a method of diagnosis, prediction, prognosis and/or monitoring of a disease or condition characterized by an aberrant level of ACKR3 polypeptide in a subject, wherein the compound is fused to a detectable label and corresponding methods of use.

Except when noted, the terms “subject” or “patient” can be used interchangeably and refer to animals, preferably warm-blooded animals, more preferably vertebrates, even more preferably mammals, still more preferably primates, and specifically includes human patients and non-human mammals and primates. Preferred subjects are human subjects. The terms “subject” or “patient” include subjects in need of treatment, more particularly subjects that would benefit from treatment of a given condition. Such subjects may include, without limitation, those that have been diagnosed with said condition, those prone to develop said condition and/or those in who said condition is to be prevented.

An absolute quantity of a molecule or analyte in a sample may be advantageously expressed as weight or as molar amount, or more commonly as a concentration, e.g., weight per volume or mol per volume. A relative quantity of a molecule or analyte in a sample may be advantageously expressed as an increase or decrease or as a fold-increase or fold-decrease relative to said another value, such as relative to a reference value as taught herein. Performing a relative comparison between first and second parameters (e.g., first and second quantities) may but need not require first to determine the absolute values of said first and second parameters. For example, a measurement method can produce quantifiable readouts (such as, e.g., signal intensities) for said first and second parameters, wherein said readouts are a function of the value of said parameters, and wherein said readouts can be directly compared to produce a relative value for the first parameter vs. the second parameter, without the actual need first to convert the readouts to absolute values of the respective parameters.

The terms “predicting” or “prediction”, “diagnosing” or “diagnosis” and “prognosticating” or “prognosis” are commonplace and well-understood in medical and clinical practice. It shall be understood that the phrase “a method for the diagnosis, prediction and/or prognosis” a given disease or condition may also be interchanged with phrases such as “a method for diagnosing, predicting and/or prognosticating” of said disease or condition or “a method for making (or determining or establishing) the diagnosis, prediction and/or prognosis” of said disease or condition, or the like.

By means of further explanation and without limitation, “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

The terms “diagnosing” or “diagnosis” generally refer to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition). As used herein, “diagnosis of” the diseases or conditions as taught herein in a subject may particularly mean that the subject has such, hence, is diagnosed as having such. “Diagnosis of no” diseases or conditions as taught herein in a subject may particularly mean that the subject does not have such, hence, is diagnosed as not having such. A subject may be diagnosed as not having such despite displaying one or more conventional symptoms or signs reminiscent of such.

The terms “prognosticating” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such. Hence, prediction or prognosis of a disease or condition can inter alia allow to predict or make a prognosis of the occurrence of the disease or condition, or to predict or make a prognosis of the progression, aggravation, alleviation or recurrence of the disease or condition or response to treatment or to other external or internal factors, situations or stressors, etc.

Further, monitoring a disease or condition can inter alia allow to predict the occurrence of the disease or condition, or to monitor the progression, aggravation, alleviation or recurrence of the disease or condition, or response to treatment or to other external or internal factors, situations or stressors, etc. Advantageously, monitoring may be applied in the course of a medical treatment of a subject, preferably medical treatment aimed at alleviating the so-monitored disease or condition. Such monitoring may be comprised, e.g., in decision making whether a patient may be discharged, needs a change in treatment or needs further hospitalisation. As intended herein, a reference to monitoring of a disease or condition also specifically includes monitoring of the probability, risk or chance of a subject to develop the disease or condition, i.e., monitoring change(s) in said probability, risk or chance over time.

A related aspect provides a method for in vitro or ex vivo diagnosis, prediction, prognosis and/or monitoring of a disease or condition characterized by an aberrant level of ACKR3 polypeptide, comprising the steps of
obtaining a biological sample obtained from a subject,
contacting said biological sample with the compound as taught herein, wherein said compound is fused to a detectable label,
determining the level of ACKR3 polypeptide in said biological sample by detecting the compound as taught herein, and
diagnosing, predicting, prognosing and/or monitoring the disease or condition based on the level of ACKR3 protein.

The term “in vitro” generally denotes outside, or external to, a body, e.g., an animal or human body. The term also encompasses “ex vivo”. One example of “in vitro” is in tissue cell culture.

The terms “sample” or “biological sample” as used herein include any biological specimen obtained and isolated from a subject. Samples may include, without limitation, organ tissue (i.e., tumour tissue, more particular breast tumour tissue), whole blood, plasma, serum, whole blood cells, red blood cells, white blood cells (e.g., peripheral blood mononuclear cells), saliva, urine, stool (i.e., faeces), tears, sweat, sebum, nipple aspirate, ductal lavage, tumour exudates, synovial fluid, cerebrospinal fluid, lymph, fine needle aspirate, amniotic fluid, any other bodily fluid, cell lysates, cellular secretion products, inflammation fluid, semen and vaginal secretions. Preferably, a sample may be readily obtainable by minimally invasive methods, such as blood collection or tissue biopsy, allowing the removal/isolation/provision of the sample from the subject. The term “tissue” as used herein encompasses all types of cells of the human body including cells of organs but also including blood and other body fluids recited above. The term “contact” or “contacting” as used herein means bringing one or more first components (such as one or more molecules, biological entities, cells, or materials) together with one or more second components (such as one or more molecules, biological entities, cells, or materials) in such a manner that the first component(s) can—if capable thereof—bind or modulate the second component(s) or that the second component(s) can—if capable thereof—bind or modulate the first component(s). Such modulation may occur either directly, i.e., by way of direct interaction between the first and second component(s); or indirectly, e.g., when the first component(s) interact with or modulate one or more further component(s), one or more of which in turn interact with or modulate the second component(s), or vice versa. The term “contacting” may depending on the context be synonymous with “exposing”, “incubating”, “mixing”, “reacting”, “treating”, or the like.

In particular embodiments, the compound as taught herein for use or the method may comprise a step of comparing the level of ACKR3 polypeptide in a biological sample from a subject with a given reference value; finding a deviation or no deviation between the level of ACKR3 polypeptide in the biological sample from the subject and the reference value; and attributing said finding of a deviation or no deviation to a particular diagnosis, prediction or prognosis of the disease or condition characterized by aberrant levels of ACKR3 polypeptide.

Such comparison may generally include any means to determine the presence or absence of at least one difference and optionally of the size of such difference between values or profiles being compared. A comparison may include a visual inspection, an arithmetical or statistical comparison of measurements. Such statistical comparisons include, but are not limited to, applying an algorithm.

Reference values for the level of ACKR3 polypeptide may be established according to known procedures previously employed for other biomarkers. For example, a reference value of the amount of ACKR3 polypeptide for a particular diagnosis, prediction, prognosis and/or monitoring of a proliferative disease as taught herein may be established by determining the quantity or expression level of ACKR3 polypeptide in sample(s) from one individual or from a population of individuals characterised by said particular diagnosis, prediction, prognosis and/or monitoring of said disease or condition. Such population may comprise without limitation ≥2, ≥10, ≥100, or even several hundred individuals or more.

The skilled person will understand that the reference value is dependent on whether diagnosis, prediction, prognosis and/or monitoring of a disease or condition characterized by an aberrant level of ACKR3 polypeptide is envisioned. For instance, distinct reference values may represent the diagnosis of a disease or condition characterized by an aberrant level of ACKR3 polypeptide vs. the absence of a disease or condition characterized by an aberrant level of ACKR3 polypeptide (such as, e.g., healthy or recovered from a disease or condition characterized by an aberrant level of ACKR3 polypeptide).

A “deviation” of a first value from a second value may generally encompass any direction (e.g., increase: first value>second value; or decrease: first value<second value) and any extent of alteration.

Preferably, a deviation may refer to a statistically significant observed alteration. For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

In a further embodiment, a deviation may be concluded if an observed alteration is beyond a given threshold or cut-off Such threshold or cut-off may be selected as generally known in the art to provide for a chosen sensitivity and/or specificity of the prediction methods.

In the methods provided herein the observation of a deviation between the ACKR3 polypeptide level in a biological sample from a subject and a reference value can lead to the conclusion that the diagnosis, prediction and/or prognosis of said proliferative disease in said subject is different from that represented by said reference value. Similarly, when no deviation is found between the quantity or expression level of the ACKR3 polypeptide level in a biological sample from a subject and a reference value, the absence of such deviation can lead to the conclusion that the diagnosis, prediction and/or prognosis of said proliferative disease in said subject is substantially the same as that represented by said reference value. The ACKR3 polypeptide is preferentially expressed in cancer cells over normal (non-cancer) cells. Accordingly, in particular embodiments, the disease characterized by aberrant level of ACKR3 polypeptide is a proliferative disease, preferably a cancer, more preferably a cancer selected from the group consisting of carcinomas, gliomas, mesotheliomas, melanomas, lymphomas, leukemias, adenocarcinomas, breast cancer, ovarian cancer, cervical cancer, glioblastoma, prostate cancer, Burkitt's lymphoma, head and neck cancer, colon cancer, colorectal cancer, non-small cell lung cancer, small cell lung cancer, cancer of the oesophagus, stomach cancer, pancreatic cancer, hepatobiliary cancer, cancer of the gallbladder, cancer of the small intestine, rectal cancer, kidney cancer, bladder cancer, penile cancer, urethral cancer, testicular cancer, vaginal cancer, uterine cancer, thyroid cancer, parathyroid cancer, adrenal cancer, pancreatic endocrine cancer, carcinoid cancer, bone cancer, skin cancer, retinoblastomas, Hodgkin's lymphoma, and non-Hodgkin's lymphoma. In further particular embodiments, the disease characterized by aberrant level of ACKR3 polypeptide is fibrosis. In further particular embodiments, the disease characterized by aberrant level of ACKR3 polypeptide is atherosclerosis or atherosclerotic plaque formation.

A further aspect of the invention relates to a kit for diagnosing, predicting, prognosing and/or monitoring a disease or condition characterized by an aberrant level of ACKR3 polypeptide in a subject, the kit comprising:
(a) a compound as taught herein, preferably wherein said compound is fused to a detectable label; and
(b) a reference value of the level of ACKR3 polypeptide, wherein said reference value represents a known diagnosis, prediction and/or prognosis of the disease or condition characterized by an aberrant level of ACKR3 polypeptide, such as wherein said reference value corresponds to the level of ACKR3 polypeptide in a tissue not affected by the disease or condition characterized by an aberrant level of ACKR3 polypeptide, such as in a healthy tissue, or wherein said reference value corresponds to the level of ACKR3 polypeptide in a tissue affected by the disease or condition characterized by an aberrant level of ACKR3 polypeptide.

The kit for diagnosing, predicting, prognosing and/or monitoring a disease or condition characterized by an aberrant level of ACKR3 polypeptide in a subject may further comprise ready-to use substrate solutions, wash solutions, dilution buffers and instructions. The diagnostic kit may also comprise positive and/or negative control samples.

Preferably, the instructions included in the diagnostic kit are unambiguous, concise and comprehensible to those skilled in the art. The instructions typically provide information on kit contents, how to collect the tissue sample, methodology, experimental read-outs and interpretation thereof and cautions and warnings.

In particular embodiments, the kit further comprises means for detecting said compound as taught herein.

The means for measuring the level of ACKR3 polypeptide in a tissue sample from a subject may comprise binding agents as discussed elsewhere in this specification and/or carriers which allow visualization and/or a qualitative read-out of the measurement, for example, by spectrophotometry. Optionally, these carriers allow for cascade testing. Non-limiting examples of carriers are translucent microtiter plates, translucent stripwells or translucent tubes.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The herein disclosed aspects and embodiments of the invention are further supported by the following non-limiting examples.

### EXAMPLES

The following examples are provided for the purpose of illustrating the present invention and by no means should be interpreted to limit the scope of the present invention.

### Example 1. Synthesis of the Compounds of the Invention

Synthesis of [(3E,4R)-3-ethylidene-1-pentylpiperidin-4-yl](1H-indol-2-yl)methanone (WW-12): Commercially available 3-acetyl pyridine 1 was reduced in an enantioselective manner to afford secondary alcohol 2. Alcohol 2 was converted to the β,γ-unsaturated aldehyde 6, utilizing a 4-step sequence. This was followed by preparation of 9 in one pot sequence in a modified way. Treatment of 6 with 2-lithiated indole-1-carboxylate 8 (generated in situ from indole 7 and carbon dioxide using Katritzky's procedure, afforded alcohol 9 in one pot in 76% yield. Alcohol 9 was converted to ketone WW-7 by oxidation with MnO₂. Removal of the PMB protecting group resulted in amine WW-8. Alkylation of WW-8 with 1-bromopentane resulted in WW-12.

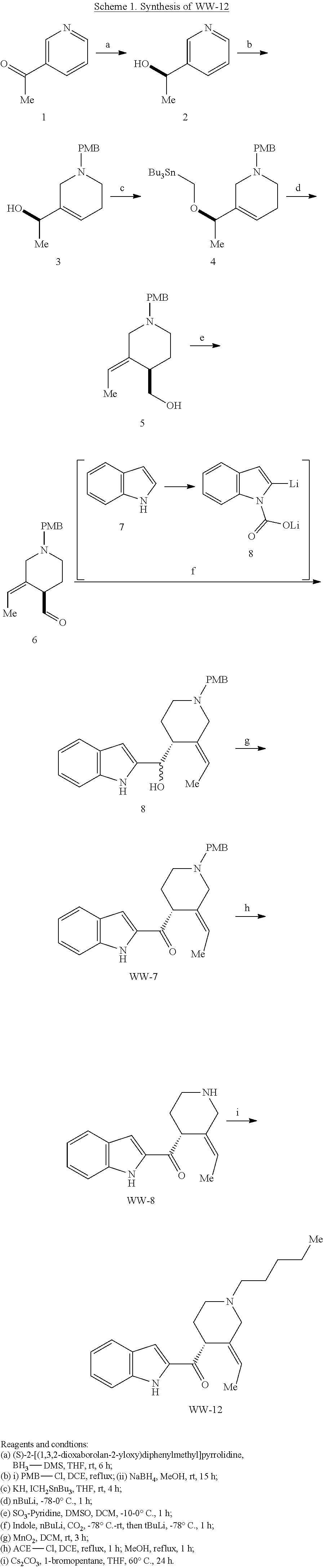

(R)-(+)-1-(3-Pyridyl)-ethanol (2):

A borane-DMS complex (10 M, 1.6 mL, 16.0 mmol) was added to a solution of (S)-2-[(1,3,2-dioxaborolan-2-yloxy)diphenylmethyl]pyrrolidine catalyst (32 mg, 0.10 mmol) in anhydrous THF (5 mL) at room temperature and the mixture was stirred for 1 h. A solution of 3-acetylpyridine (1.21 g, 10.0 mmol) in anhydrous THF (5 mL) was added slowly dropwise via syringe over 5 h. The reaction mixture was stirred at rt 1 h, then cooled at 0° C. and quenched with methanol (10 mL). The mixture was heated at reflux for 12 h. Then the reaction mixture was concentrated under vacuum. The residue was distilled in a Kugelrohr apparatus under vacuum to give 2 as colorless oil (1.18 g; 96% yield).

¹H NMR (300 MHz, CHLOROFORM-d) δ 1.53 (dd, J=6.40, 0.75 Hz, 3H) 1.85 (br. s., 1H) 2.73 (br. s., 1H) 4.95 (q, J=6.53 Hz, 1H) 7.24-7.31 (m, 1H) 7.69-7.79 (m, 1H) 8.48 (d, J=4.71 Hz, 1H) 8.56 (s, 1H)

¹³C NMR (75 MHz, CHLOROFORM-d) δ ppm 25.20, 68.05, 123.47, 133.15, 147.43, 148.71. [α]_{D}=+56.9 (c=1.4, CHCl₃).

(R)-(N-(4-methoxybenzyl)-1,2,5,6-tetrahydropyridin-3-yl)ethanol (3): To a solution of 1-(pyridin-3-yl)ethanol 2 (4.7 g, 38 mmol) in anhydrous dichloroethane (75 mL) was added 4-methoxybenzyl chloride (6.25 g/5.4 mL, 39.9 mmol). The mixture was heated at reflux for 15 h. After allowing to cool to rt, the mixture was concentrated under reduced pressure to afford the N-PMB pyridinium salt, which was dissolved in anhydrous MeOH (100 mL) and cooled to 0° C. To this cold solution was cautiously added NaBH₄ (4.3 g, 114 mmol) in three portions. The mixture was then allowed to warm slowly to rt overnight. Then the reaction mixture was concentrated under reduced pressure to obtain a orange-yellow residue, which was slurried in 1:1 Et₂O—H₂O (120 mL). Potassium carbonate (2 g) was added, and the orange biphasic mixture was stirred at rt for 1 h. The aqueous layer was separated and extracted with Et₂O (60 mL×3). The combined organic extracts were dried (K₂CO₃), filtered and concentrated. The crude product was purified by flash column chromatography on silica gel (ISCO, 80 g Gold, Silica column) eluting with 0%-5% MeOH (containing 1% NH₄OH) in DCM to afford 3 as a pale yellow oil (5.1 g, 54% yield).

¹H NMR (300 MHz, CHLOROFORM-d) δ 1.25 (d, J=6.40 Hz, 3H) 1.74 (br. s., 1H) 2.15 (br. s., 2H) 2.39-2.61 (m, 2H) 2.87-3.09 (m, 2H) 3.49-3.61 (m, 2H) 3.80 (s, 3H) 4.19 (q, J=6.40 Hz, 1H) 5.70 (br. s., 1H) 6.85 (d, J=8.67 Hz, 2H) 7.22-7.30 (m, 2H)

¹³C NMR (75 MHz, CHLOROFORM-d) δ 21.62, 25.60, 49.53, 51.90, 55.26, 62.27, 70.36, 113.64, 119.69, 130.18, 130.41, 140.09, 228.79.

[α]_{D}=+5.4 (c=0.46, MeOH).

(R)-1-(4-methoxybenzyl)-5-(1-((tributylstannyl)methoxy)ethyl)-1,2,3,6-tetrahydropyridine (4): A slurry of KH (˜3.1 g/30% suspension in mineral oil, 23.5 mmol) was washed with hexanes (10 mL×3), and then suspended in anhydrous THF (10 mL). To this suspension was added alcohol 3 (3.4 g, 13.8 mmol) in 20 mL anhydrous THF, and the resulting suspension was stirred at rt for 1 h. To this was slowly added a solution of tributylstannyl methyl iodide (6.26 g, 14.5 mmol) in 20 mL anhydrous THF, and the resulting thick yellow suspension stirred at rt for 4 h. After this, the reaction mixture was quenched with satd aqueous NaHCO₃ (30 mL). The aqueous layer was extracted with Et₂O (50 mL×3), and the combined organic extracts were dried (K₂CO₃), filtered, and concentrated under reduced pressure. The crude product was purified by flash chromatography on silica gel (ISCO, 80 g, Gold, Silica) eluting with 0%-5% MeOH (containing 1% NH₄OH) in DCM to afford 4 as a pale yellow oil (7.17 g, 94% yield).

¹H NMR (300 MHz, CHLOROFORM-d) δ 0.77-0.99 (m, 15H) 1.17 (d, J=6.59 Hz, 3H) 1.29 (dq, J=14.69, 7.22 Hz, 6H) 1.38-1.61 (m, 6H) 2.02-2.30 (m, 2H) 2.50 (dt, J=8.57, 5.79 Hz, 2H) 2.89 (br. s., 2H) 3.45-3.58 (m, 4H) 3.66 (d, J=10.36 Hz, 1H) 3.79 (s, 3H) 5.62 (br. s., 1H) 6.80-6.90 (m, 2H) 7.21-7.32 (m, 2H)

¹³C NMR (75 MHz, CHLOROFORM-d) δ 9.01, 13.73, 19.83, 25.66, 27.31, 29.17, 49.61, 51.28, 55.21, 58.5, 62.26, 81.91, 82.19, 82.47, 113.57, 121.49, 130.35, 130.47, 137.75, 158.7.

(3E,4R)-(3-ethylidene-1-(4-methoxybenzyl)piperidin-4-yl)methanol (5): To a solution of stannane 4 (2.8 g, 5.1 mmol) in anhydrous THF (50 mL) at −78° C. was slowly added nBuLi solution (10.2 mL/2.5 M in hexanes, 25.5 mmol). The reaction mixture was slowly allowed to warm to 0° C. and was stirred at 0° C. for 1 h. After this, the reaction was quenched by the dropwise addition of satd. aqueous NaHCO₃ solution (25 mL). The aqueous layer was separated and was extracted with Et₂O (75 mL×3). Combined organic extracts were dried (K₂CO₃), filtered, and concentrated under reduced pressure. The crude product was purified by flash chromatography on silica gel (ISCO, 24 g, Gold, Silica) eluting with 0%-5% MeOH (containing 1% NH₄OH) in DCM to afford 5 as a pale orange oil (3 g, 85% yield).

¹H NMR (300 MHz, CHLOROFORM-d) δ 1.64 (d, J=6.78 Hz, 3H) 1.68-1.88 (m, 2H) 2.02-2.24 (m, 2H) 2.64-2.78 (m, 2H) 2.94 (q, J=6.84 Hz, 1H) 3.04 (d, J=12.24 Hz, 1H) 3.37-3.51 (m, 2H) 3.56-3.70 (m, 2H) 3.79 (s, 3H) 5.49 (q, J=6.59 Hz, 1H) 6.84 (d, J=8.67 Hz, 2H) 7.22 (d, J=8.48 Hz, 2H)

¹³C NMR (75 MHz, CHLOROFORM-d) δ 12.72, 26.25, 36.14, 49.35, 55.24, 58.48, 62.37, 62.74, 113.61, 122.46, 129.97, 130.46, 134.78, 158.76.

[α]_{D}=−37.5 (c=0.4, MeOH).

(3E,4R)-3-ethylidene-1-(4-methoxybenzyl)piperidine-4-carbaldehyde (6): To a solution of alcohol 5 (1.5 g, 5.7 mmol) in CH₂Cl₂ (100 mL) buffered with diisopropylethyl amine (4 mL) at −10° C. (salt-ice bath) was added anhydrous DMSO (3.7 mL, 57 mmol). To this cold solution was added SO₃-pyridine (4 g, 25 mmol) in 3 portions over 20 minutes. The mixture was allowed to warm to 0° C. over 1 h. After allowing to warm to rt, pH 7.0 phosphate buffer (100 mL) was added. The aqueous layer was separated and extracted with CH₂Cl₂ (75 mL×3). The combined organic extracts were washed with pH 7.0 phosphate buffer (50 ml×4) and concentrated under reduced pressure to obtain 6 as an orange oil (1.5 g, crude, 99%). This material was used immediately in the next step without further purification.

((3E,4R)-3-ethylidene-1-(4-methoxybenzyl)piperidin-4-yl)(1H-indol-2-yl)methanol (9): To a solution of indole 7 (0.67 g, 5.7 mmol) in anhydrous THF (30 mL) at −78° C. was added nBuLi solution (2.4 mL/2.5 M in hexanes, 6 mmol), and the mixture was stirred at −78° C. for 0.5 h. To this solution was added dry ice (2 g, excess) and the mixture was stirred at −78° C. for 10 min. The reaction mixture was then slowly allowed to warm to rt over 0.5 h. The solvent was evaporated, and excess CO₂ was removed under reduced pressure to obtain a residue, which was redissolved in anhydrous THF (15 mL). The solution was cooled to −78° C. To this was slowly added tBuLi solution (3.5 mL/1.7 M in pentane, 6 mmol), and the mixture was stirred at −78° C. for 1 h to obtain a lithiated indole intermediate 8 (in situ). In another rb flask was suspended 6 (1.5 g, 5.7 mmol) in anhydrous THF (15 mL), and the mixture was cooled to −78° C. To this cold mixture was cannulated the above lithiated indole intermediate 8 over 2 min. The reaction was stirred at −78° C. for 1 h and then quenched by the addition of satd. aqueous NaHCO₃ solution (25 mL). After warming to rt, the aqueous layer was separated and extracted with CH₂Cl₂ (50 mL×3). The combined organic layers were washed with brine (50 mL) and concentrated under reduced pressure to obtain a crude product (1.63 g, crude, 76%) (Diagnostic doublet integrating for 1H observed at 5.00 ppm in ¹H NMR), which was used in the next reaction without further purification.

(3E,4R)-(3-ethylidene-1-(4-methoxybenzyl)piperidin-4-yl)(1H-indol-2-yl)methanone (WW-7): To the solution of alcohol 9 (1.6 g, 4.2 mmol, crude) in CH₂Cl₂ (100 mL) was added activated MnO₂ (9.1 g, 106 mmol) in two portions over 1 h. The black suspension was stirred at rt for 3 h. The reaction mixture was filtered through a pad of Celite, washing with 10% MeOH in CH₂Cl₂. The filtrate was concentrated under reduced pressure to obtain an orange-brown foam, which was purified by flash column chromatography on silica gel (ISCO, 40 g Gold, Silica) eluting with 0%-5% MeOH (containing 1% NH₄OH) in DCM to afford WW-7 as a light brown solid (1.3 g, 85% yield).

¹H NMR (300 MHz, CHLOROFORM-d) δ 1.67-1.73 (m, 3H) 2.02 (s, 1H) 2.07-2.24 (m, 2H) 2.45-2.61 (m, 1H) 2.84 (d, J=11.49 Hz, 1H) 3.15-3.30 (m, 2H) 3.54 (d, J=2.07 Hz, 2H) 3.78-3.82 (m, 3H) 4.45 (dd, J=5.75, 1.60 Hz, 1H) 5.57 (d, J=6.78 Hz, 1H) 6.82-6.88 (m, 2H) 7.25 (d, J=3.01 Hz, 1H) 7.26-7.30 (m, 2H) 7.34 (td, J=7.63, 1.13 Hz, 1H) 7.40-7.47 (m, 1H) 7.70 (d, J=8.10 Hz, 1H) 9.31 (br. s., 1H)

(3E,4R)-(3-ethylidenepiperidin-4-yl)(1H-indol-2-yl)methanone (WW-8): To a solution of WW-7 (0.16 g, 0.435 mmol) in dichloroethane (10 mL) was added alpha-chloroethyl chloroformate (0.1 mL). The mixture was heated at reflux for 4 h. After this, the mixture was concentrated under reduced pressure to obtain a crude carbamate intermediate, which was dissolved in anhydrous MeOH (10 mL). The solution was heated at reflux for 1 h. After removal of solvent, the crude product was redissolved in MeOH, followed by the addition of basic alumina (0.5 g). After concentrating the mixture, a solid plug was obtained which was purified by flash column chromatography on silica gel (ISCO, 4 g Gold, Silica) eluting with 1%-10% MeOH (containing 1% NH₄OH) in DCM to afford amine WW-8 as a white solid (0.105 g, 95% yield).

¹H NMR (300 MHz, CHLOROFORM-d) δ 1.72 (dd, J=6.78, 1.13 Hz, 3H) 1.83-2.00 (m, 1H) 2.21 (dd, J=13.66, 2.17 Hz, 1H) 2.85-3.04 (m, 2H) 3.09-3.20 (m, 1H) 3.26-3.45 (m, 2H) 3.68-3.77 (m, 1H) 4.53 (d, J=5.65 Hz, 1H) 5.53 (q, J=6.84 Hz, 1H) 7.16 (t, J=7.54 Hz, 1H) 7.27 (s, 1H) 7.31-7.39 (m, 1H) 7.40-7.46 (m, 1H) 7.71 (d, J=8.10 Hz, 1H) 9.11 (br. s., 1H)

¹³C NMR (75 MHz, CHLOROFORM-d) δ 13.02, 31.63, 43.12, 43.34, 53.20, 108.87, 112.06, 120.68, 121.01, 123.1, 126.35, 127.67, 134.51, 135.35, 137.18, 193.6.

General Procedure A:

Synthesis of [(3E,4R)-3-ethylidene-1-pentylpiperidin-4-yl](1H-indol-2-yl)methanone (WW-12): To the solution of amine WW-8 (38 mg, 0.15 mmol) in anhydrous THF (3 mL) under nitrogen was added Cs₂CO₃ (147 mg, 0.45 mmol) followed by 1-bromopentane (23 μL, 0.225 mmol). The reaction mixture was heated at 60° C. for 24 h. After allowing to cool to rt, the reaction mixture was diluted with EtOAc (10 mL) and was filtered. To the filtrate was added celite (0.5 g) and the solvent was evaporated under reduced pressure to obtain a plug. Purification was done using flash column chromatography on silica gel (ISCO, 4 g Gold, Silica) eluting with 0%-10% MeOH (containing 1% NH₄OH) in DCM to afford amine WW-12 as an off-white crystalline solid (13.2 mg, 27% yield).

ESI MS: 325.4 (M+H)⁺

¹H NMR (300 MHz, CHLOROFORM-d) δ 0.89 (t, J=6.88 Hz, 3H) 1.23-1.38 (m, 4H) 1.52 (dt, J=15.26, 7.44 Hz, 2H) 1.71 (dd, J=6.78, 1.70 Hz, 3H) 2.02 (s, 2H) 2.04-2.15 (m, 1H) 2.23 (dd, J=13.66, 1.98 Hz, 1H) 2.33-2.51 (m, 3H) 2.85 (d, J=11.68 Hz, 1H) 3.06-3.15 (m, 1H) 3.21-3.29 (m, 1H) 4.45 (d, J=5.46 Hz, 1H) 5.63 (q, J=6.59 Hz, 1H) 7.14 (td, J=7.54, 0.75 Hz, 1H) 7.29-7.37 (m, 2H) 7.46 (dd, J=8.38, 0.66 Hz, 1H) 7.71 (d, J=8.10 Hz, 1H) 9.67 (br. s., 1H)

¹³C NMR (75 MHz, CHLOROFORM-d) δ 13.28, 14.03, 22.63, 26.78, 29.23, 29.95, 42.61, 50.41, 58.70, 60.59, 76.64, 77.07, 77.27, 77.49, 109.13, 112.31, 120.93, 122.29, 123.05, 126.27, 127.62, 133.71, 134.5, 137.43, 172.7, 193.89.

The free base was converted to its hydrochloride salt by treating with 2 M HCl in diethyl ether to obtain an off-white solid. Elemental Anal. Calcd. for C₂₁H₂₈N₂O·HCl: C, 69.88; H, 8.10; N, 7.76. found: C, 69.68; H, 8.00; N, 7.64. [α]_{D}=−2.7 (c=0.15, MeOH).

Synthesis of [(3E,4R)-3-ethylidene-1-(4-fluorophenethyl)piperidin-4-yl](1H-indol-2-yl)methanone (WW-9): Using the General Procedure A described above, WW-9 was obtained from amine WW-8 (22.5 mg, 0.088 mmol) with 4-fluorophenethyl bromide (23 mg, 0.115 mmol) as a light yellow oil (16 mg, 40% yield).

¹H NMR (300 MHz, CHLOROFORM-d) δ 9.05-9.26 (m, 1H), 7.31-7.40 (m, 2H), 7.11-7.19 (m, 4H), 6.96 (t, J=8.38 Hz, 2H), 6.75-6.84 (m, 1H), 5.59-5.72 (m, 1H), 4.39-4.51 (m, 1H), 3.73-3.79 (m, 2H), 2.74-2.97 (m, 4H), 2.54-2.67 (m, 4H), 2.18-2.29 (m, 1H), 2.04-2.15 (m, 2H), 2.02 (s, 2H), 1.74 (dd, J=1.60, 6.69 Hz, 3H)

ESI MS: 377.3 (M+H)⁺

The free base was converted to its hydrochloride salt by treating with 2 M HCl in diethyl ether to obtain a white solid.

[(3E,4R)-3-ethylidene-1-(2-methylpropyl)piperidin-4-yl](1H-indol-2-yl)methanone (WW-10): Using the General Procedure A described above, WW-10 was obtained from amine WW-8 (25.4 mg, 0.1 mmol) with isobutyl bromide (16 μL, 0.15 mmol) as a light yellow oil (12.5 mg, 40% yield).

¹H NMR (300 MHz, CHLOROFORM-d) δ 9.13 (br. s., 1H), 7.71 (dd, J=0.75, 8.10 Hz, 1H), 7.41-7.45 (m, 1H), 7.34 (dt, J=1.22, 7.58 Hz, 1H), 7.29 (d, J=1.32 Hz, 1H), 7.15 (ddd, J=1.04, 6.92, 8.05 Hz, 1H), 5.55-5.66 (m, 1H), 4.42 (d, J=7.16 Hz, 1H), 3.03-3.22 (m, 2H), 2.77 (d, J=11.68 Hz, 1H), 2.40 (dt, J=3.11, 12.01 Hz, 1H), 2.12-2.19 (m, 3H), 2.03 (s, 1H), 1.81 (td, J=6.78, 13.56 Hz, 1H), 1.70 (dd, J=1.79, 6.88 Hz, 3H), 0.91 (d, J=6.59 Hz, 6H)

The free base was converted to its hydrochloride salt by treating with 2 M HCl in diethyl ether to obtain an off-white solid.

[(3E,4R)-3-ethylidene-1-propylpiperidin-4-yl](1H-indol-2-yl)methanone (WW-14): Using the General Procedure A described above, WW-14 was obtained from amine WW-8 (25.4 mg, 0.1 mmol) with 1-bromopropane (14 μL, 0.15 mmol) as a light brown solid (7.8 mg, 26% yield).

¹H NMR (300 MHz, CHLOROFORM-d) δ 9.06-9.06 (m, OH), 9.11 (br. s., 1H), 7.71 (d, J=8.10 Hz, 1H), 7.40-7.46 (m, 1H), 7.35 (dt, J=1.04, 7.58 Hz, 1H), 7.29 (d, J=1.32 Hz, 1H), 7.12-7.19 (m, 1H), 5.63 (d, J=6.59 Hz, 1H), 4.42 (d, J=5.65 Hz, 1H), 3.03-3.27 (m, 2H), 2.85 (d, J=11.68 Hz, 1H), 2.39-2.50 (m, 1H), 2.31-2.39 (m, 2H), 2.17-2.27 (m, 1H), 2.04-2.14 (m, 1H), 2.03 (s, 2H), 1.72 (dd, J=1.70, 6.78 Hz, 3H), 1.48-1.61 (m, 2H), 0.90 (t, J=7.35 Hz, 3H)

The free base was converted to its hydrochloride salt by treating with 2 M HCl in diethyl ether to obtain an off-white solid.

[(3E,4R)-3-ethylidene-1-hexylpiperidin-4-yl](1H-indol-2-yl)methanone (WW-62): Using the General Procedure A described above, WW-62 was obtained from amine WW-8 (22 mg, 0.086 mmol) with 1-bromohexane (18 μL, 0.13 mmol) as a light yellow oil (9.6 mg, 33% yield).

¹H NMR (300 MHz, CHLOROFORM-d) δ 9.17 (br. s., 1H), 7.71 (d, J=8.10 Hz, 1H), 7.40-7.46 (m, 1H), 7.31-7.39 (m, 1H), 7.29 (d, J=0.94 Hz, 1H), 7.12-7.19 (m, 1H), 5.63 (q, J=6.72 Hz, 1H), 4.43 (d, J=5.65 Hz, 1H), 3.21-3.29 (m, 1H), 3.04-3.14 (m, 1H), 2.86 (d, J=11.68 Hz, 1H), 2.32-2.52 (m, 3H), 2.16-2.27 (m, 1H), 2.04-2.14 (m, 1H), 1.86-2.00 (m, 2H), 1.72 (dd, J=1.51, 6.78 Hz, 3H), 1.51 (d, J=7.35 Hz, 2H), 1.20-1.38 (m, 8H), 0.85-0.91 (m, 3H)

The free base was converted to its hydrochloride salt by treating with 2 M HCl in diethyl ether to obtain an off-white solid.

[(3E,4R)-3-ethylidene-1-(4-methylpentyl)piperidin-4-yl](1H-indol-2-yl)methanone (WW-63): Using the General Procedure A described above, WW-63 was obtained from amine WW-8 (22 mg, 0.086 mmol) with 1-Bromo-4-methylpentane (19 μL, 0.13 mmol) as a yellow-orange solid (13 mg, 45% yield).

¹H NMR (300 MHz, CHLOROFORM-d) δ 9.21 (br. s., 1H), 7.72 (d, J=7.91 Hz, 1H), 7.41-7.46 (m, 1H), 7.35 (dt, J=0.94, 7.63 Hz, 1H), 7.30 (d, J=1.32 Hz, 1H), 7.13-7.19 (m, 1H), 5.69 (q, J=6.59 Hz, 1H), 4.47 (t, J=3.67 Hz, 1H), 3.24-3.40 (m, 2H), 2.98 (d, J=11.30 Hz, 1H), 2.65 (d, J=7.16 Hz, 1H), 2.43-2.53 (m, 2H), 2.17-2.25 (m, 2H), 1.74 (dd, J=1.41, 6.88 Hz, 3H), 1.50-1.68 (m, 4H), 1.13-1.30 (m, 3H), 0.88 (d, J=6.59 Hz, 6H)

The free base was converted to its hydrochloride salt by treating with 2 M HCl in diethyl ether to obtain an off-white solid.

1-(2-{[(3E,4R)-3-ethylidene-1-pentylpiperidin-4-yl]carbonyl}-1-pentyl-1H-indol-5-yl)ethanone (WW-66): Using the General Procedure A described above, reaction of 1-(2-{[(3E,4R)-3-ethylidenepiperidin-4-yl]carbonyl}-1H-indol-5-yl)ethenone (60 mg, 0.22 mmol) with 1-bromopentane (34 μL, 0.33 mmol) resulted in WW-66 as a light brown oil (35 mg, 36% yield).

¹H NMR (300 MHz, CHLOROFORM-d) δ 8.37 (d, J=1.13 Hz, 1H), 8.01 (dd, J=1.60, 8.95 Hz, 1H), 7.46 (s, 1H), 7.42 (d, J=9.04 Hz, 1H), 5.62 (q, J=6.78 Hz, 1H), 4.44-4.61 (m, 3H), 3.20-3.30 (m, 1H), 3.03-3.13 (m, 1H), 2.85 (d, J=11.68 Hz, 1H), 2.67 (s, 3H), 2.33-2.48 (m, 3H), 2.05-2.22 (m, 2H), 1.68 (dd, J=1.41, 6.88 Hz, 3H), 1.53 (td, J=7.46, 15.02 Hz, 2H), 1.19-1.41 (m, 9H), 0.82-0.94 (m, 6H)

¹³C NMR (75 MHz, CHLOROFORM-d) δ 196.9, 194.2, 141.0, 134.8, 132.7, 130.1, 124.7, 124.6, 121.8, 111.8, 109.9, 59.8, 58.0, 49.7, 44.7, 43.2, 29.6, 29.2, 28.6, 28.3, 26.1, 25.8, 21.9, 21.8, 13.3, 13.3, 12.6 ESI MS: 437.2 (M+H)⁺

The free base was converted to its hydrochloride salt by treating with 2 M HCl in diethyl ether to obtain an off-white solid.

[(3E,4R)-3-ethylidene-1-heptylpiperidin-4-yl](1H-indol-2-yl)methanone (WW-67): Using the General Procedure A described above, WW-67 was obtained from amine WW-8 (25.4 mg, 0.1 mmol) with 1-bromoheptane (24 μL, 0.15 mmol) as a colorless oil (8 mg, 23% yield).

¹H NMR (300 MHz, CHLOROFORM-d) δ 9.16 (br. s., 1H), 9.08-9.11 (m, OH), 7.71 (d, J=8.10 Hz, 1H), 7.40-7.47 (m, 1H), 7.34 (dt, J=0.94, 7.63 Hz, 1H), 7.29 (d, J=1.32 Hz, 1H), 7.12-7.19 (m, 1H), 5.64 (q, J=6.72 Hz, 1H), 4.43 (d, J=5.09 Hz, 1H), 3.22-3.29 (m, 1H), 3.05-3.15 (m, 1H), 2.87 (d, J=11.87 Hz, 1H), 2.34-2.53 (m, 3H), 2.17-2.27 (m, 1H), 1.99-2.16 (m, 2H), 1.72 (dd, J=1.51, 6.97 Hz, 3H), 1.47-1.59 (m, 2H), 1.28 (br. s., 10H), 0.84-0.92 (m, 3H) The free base was converted to its hydrochloride salt by treating with 2 M HCl in diethyl ether to obtain an off-white solid. [(3E,4R)-1-(cyclohexylmethyl)-3-ethylidenepiperidin-4-yl](1H-indol-2-yl)methanone (WW-68): Using the General Procedure A described above, WW-68 was obtained from amine WW-8 (25.4 mg, 0.1 mmol) with (bromomethyl)cyclohexane (21 μL, 0.15 mmol) as a colorless semisolid (11 mg, 31% yield).

¹H NMR (300 MHz, CHLOROFORM-d) δ 9.11 (br. s., 1H), 7.71 (d, J=8.10 Hz, 1H), 7.40-7.45 (m, 1H), 7.34 (dt, J=0.94, 7.63 Hz, 1H), 7.29 (d, J=1.32 Hz, 1H), 7.12-7.19 (m, 1H), 5.55-5.65 (m, 1H), 4.41 (d, J=5.09 Hz, 1H), 3.01-3.22 (m, 2H), 2.77 (d, J=11.68 Hz, 1H), 2.32-2.45 (m, 1H), 2.15-2.22 (m, 3H), 2.04-2.13 (m, 1H), 1.70 (dd, J=1.60, 6.88 Hz, 3H), 1.11-1.28 (m, 4H), 0.81-0.96 (m, 2H)

The free base was converted to its hydrochloride salt by treating with 2 M HCl in diethyl ether to obtain a white solid.

Methyl 6-[(3E,4R)-3-ethylidene-4-(1H-indol-2-ylcarbonyl)piperidin-1-yl]hexanoate (WW-71): Using the General Procedure A described above, WW-71 was obtained from amine WW-8 (25.4 mg, 0.1 mmol) with methyl 6-bromohexanoate (24 μL, 0.15 mmol) as a colorless oil (17 mg, 44% yield).

¹H NMR (300 MHz, CHLOROFORM-d) δ 9.16 (br. s., 1H), 7.71 (d, J=7.91 Hz, 1H), 7.40-7.46 (m, 1H), 7.32-7.38 (m, 1H), 7.29 (d, J=1.13 Hz, 1H), 7.15 (t, J=7.44 Hz, 1H), 5.58-5.69 (m, 1H), 4.43 (d, J=5.27 Hz, 1H), 3.66 (s, 3H), 3.18-3.29 (m, 1H), 3.03-3.15 (m, 1H), 2.85 (d, J=11.68 Hz, 1H), 2.27-2.51 (m, 6H), 2.17-2.26 (m, 1H), 2.04-2.14 (m, 1H), 1.72 (dd, J=1.32, 6.78 Hz, 3H), 1.59-1.68 (m, 4H), 1.27-1.39 (m, 3H)

The free base was converted to its hydrochloride salt by treating with 2 M HCl in diethyl ether to obtain a buff colored solid.

(3E,4R)-(3-ethylidene-1-(4-phenoxybutyl)piperidin-4-yl)(1H-indol-2-yl)methanone (WW-72): Using the General Procedure A described above, WW-72 was obtained from amine WW-8 (25.4 mg, 0.1 mmol) with 4-bromobutyl phenyl ether (34.3 mg, 0.15 mmol) as a yellow-orange oil (15 mg, 37% yield).

¹H NMR (300 MHz, CHLOROFORM-d) δ 9.07 (br. s., 1H), 7.71 (d, J=7.91 Hz, 1H), 7.40-7.46 (m, 1H), 7.32-7.38 (m, 1H), 7.30 (s, 1H), 7.27 (d, J=1.13 Hz, 1H), 7.23-7.26 (m, 1H), 7.12-7.19 (m, 1H), 6.87-6.95 (m, 3H), 5.62 (d, J=6.78 Hz, 1H), 4.43 (d, J=5.27 Hz, 1H), 3.98 (t, J=6.12 Hz, 2H), 3.19-3.29 (m, 1H), 3.06-3.15 (m, 1H), 2.86 (d, J=11.30 Hz, 1H), 2.41-2.51 (m, 3H), 2.17-2.27 (m, 1H), 2.05 (d, J=4.14 Hz, 1H), 1.77-1.84 (m, 2H), 1.72 (dd, J=1.51, 6.78 Hz, 3H)

The free base was converted to its hydrochloride salt by treating with 2 M HCl in diethyl ether to obtain an off-white solid.

(3E,4R)-(3-ethylidene-1-(4-aminobutyl)piperidin-4-yl)(1H-indol-2-yl)methanone (WW-58): Using the General Procedure A described above, reaction of amine WW-8 (50.8 mg, 0.2 mmol) with 4-(tert-butoxycarbonylamino)butyl bromide (75.6 mg, 0.3 mmol) afforded tert-butyl {4-[(3E)-3-ethylidene-4-(1H-indol-2-ylcarbonyl)piperidin-1-yl]butyl}carbamate as a light yellow solid (37 mg, 57% yield).

¹H NMR (300 MHz, CHLOROFORM-d) δ 9.24 (br. s., 1H), 7.71 (d, J=8.10 Hz, 1H), 7.41-7.47 (m, 1H), 7.34 (dt, J=0.94, 7.63 Hz, 1H), 7.29 (d, J=1.32 Hz, 1H), 7.12-7.19 (m, 1H), 5.62 (d, J=6.78 Hz, 1H), 4.98-5.15 (m, 1H), 4.43 (d, J=5.46 Hz, 1H), 3.06-3.27 (m, 4H), 2.84 (d, J=11.49 Hz, 1H), 2.35-2.52 (m, 3H), 2.00-2.26 (m, 3H), 1.72 (dd, J=1.51, 6.78 Hz, 3H), 1.44 (s, 9H)

¹³C NMR (75 MHz, CHLOROFORM-d) δ 193.6, 156.1, 137.3, 134.4, 133.4, 129.0, 127.7, 126.4, 123.1, 122.5, 121.0, 112.1, 109.0, 60.3, 57.9, 50.3, 42.5, 29.0, 28.5, 28.1, 24.4, 13.3

Treatment of the solution of tert-butyl {4-[(3E)-3-ethylidene-4-(1H-indol-2-ylcarbonyl)piperidin-1-yl]butyl}carbamate (35 mg, 0.082 mmol) in DCM (1 mL) with TFA (0.5 mL) resulted in WW-58 as an orange-brown oil (28 mg, 99%).

¹H NMR (300 MHz, CHLOROFORM-d) δ 9.36 (br. s., 1H), 7.71 (d, J=8.10 Hz, 1H), 7.41-7.46 (m, 1H), 7.34 (dt, J=0.94, 7.63 Hz, 1H), 7.29 (s, 1H), 7.12-7.18 (m, 1H), 5.63 (q, J=6.47 Hz, 1H), 4.44 (d, J=5.65 Hz, 1H), 3.73-3.79 (m, 1H), 3.61-3.68 (m, 1H), 3.20-3.28 (m, 1H), 3.06-3.15 (m, 1H), 2.85 (d, J=11.68 Hz, 1H), 2.73 (br. s., 2H), 2.36-2.52 (m, 3H), 2.17-2.26 (m, 2H), 2.01-2.14 (m, 2H), 1.72 (dd, J=1.32, 6.78 Hz, 3H)

¹³C NMR (75 MHz, CHLOROFORM-d) δ 192.0, 135.7, 132.9, 131.8, 126.1, 124.8, 121.5, 120.9, 119.4, 110.6, 107.5, 70.8, 69.6, 60.1, 58.8, 56.7, 48.7, 40.9, 40.4, 28.1, 27.5, 22.9, 11.7 ESI MS: 326 (M+H)⁺

The free base was converted to its hydrochloride salt by treating with 2 M HCl in diethyl ether to obtain an orange-brown solid.

(3E,4R)-(3-ethylidene-1-(5-aminopentyl)piperidin-4-yl)(1H-indol-2-yl)methanone (WW-59): Using the General Procedure A described above, reaction of amine WW-8 (50.8 mg, 0.2 mmol) with 5-(tert-butoxycarbonylamino)pentyl bromide (79.8 mg, 0.3 mmol) afforded tert-butyl {5-[(3E)-3-ethylidene-4-(1H-indol-2-ylcarbonyl)piperidin-1-yl]pentyl}carbamate as a light yellow semisolid (38 mg, 56% yield).

¹H NMR (300 MHz, CHLOROFORM-d) δ 9.46 (br. s., 1H), 7.71 (d, J=7.91 Hz, 1H), 7.41-7.48 (m, 1H), 7.28-7.38 (m, 2H), 7.11-7.18 (m, 1H), 5.63 (d, J=6.78 Hz, 1H), 4.59 (br. s., 1H), 4.44 (d, J=5.84 Hz, 1H), 3.20-3.27 (m, 1H), 3.06-3.15 (m, 3H), 2.84 (d, J=11.68 Hz, 1H), 2.34-2.51 (m, 3H), 2.22 (dd, J=1.70, 13.56 Hz, 1H), 2.00-2.14 (m, 1H), 1.71 (dd, J=1.22, 6.88 Hz, 3H), 1.44 (s, 9H), 1.22-1.38 (m, 3H)

¹³C NMR (75 MHz, CHLOROFORM-d) δ 191.3, 134.9, 132.1, 131.2, 125.2, 123.9, 120.7, 120.0, 118.6, 109.8, 106.7, 58.1, 56.0, 47.9, 40.1, 27.6, 26.7, 26.1, 24.3, 22.5, 10.9 ESI MS: 440.2 (M+H)⁺

Treatment of the solution of tert-butyl {5-[(3E)-3-ethylidene-4-(1H-indol-2-ylcarbonyl)piperidin-1-yl]pentyl}carbamate (37 mg, 0.084 mmol) in DCM (1 mL) with TFA (0.5 mL) resulted in WW-59 as an orange-brown oil (31 mg, 99%).

¹H NMR (300 MHz, CHLOROFORM-d) δ 9.53 (br. s., 1H), 7.71 (d, J=8.10 Hz, 1H), 7.41-7.48 (m, 1H), 7.28-7.38 (m, 2H), 7.15 (t, J=7.44 Hz, 1H), 5.63 (q, J=6.47 Hz, 1H), 4.44 (d, J=5.84 Hz, 1H), 3.20-3.27 (m, 1H), 3.05-3.16 (m, 1H), 2.84 (d, J=11.49 Hz, 1H), 2.70 (t, J=6.78 Hz, 2H), 2.34-2.51 (m, 3H), 2.18-2.26 (m, 1H), 1.98-2.15 (m, 2H), 1.71 (d, J=5.84 Hz, 3H), 1.42-1.57 (m, 4H), 1.27-1.40 (m, 3H)

¹³C NMR (75 MHz, CHLOROFORM-d) δ 193.1, 136.7, 133.9, 133.0, 129.2, 128.4, 127.0, 125.7, 122.5, 121.8, 120.3, 113.4, 113.3, 111.6, 108.4, 59.9, 57.9, 49.8, 42.0, 41.5, 33.1, 29.1, 28.5, 26.3, 24.4, 12.7

ESI MS: 340 (M+H)⁺

The free base was converted to its hydrochloride salt by treating with 2 M HCl in diethyl ether to obtain an orange solid.

(3E,4R)-(3-ethylidene-1-(2-ethoxy-(2-aminoethyl))piperidin-4-yl)(1H-indol-2-yl)methanone (WW-60): Using the General Procedure A described above, reaction of amine WW-8 (50.8 mg, 0.2 mmol) with tert-butyl [2-(2-bromoethoxy)ethyl]carbamate (80.5 mg, 0.3 mmol) afforded tert-butyl (2-{2-[(3E)-3-ethylidene-4-(1H-indol-2-ylcarbonyl)piperidin-1-yl]ethoxy}ethyl)carbamate as a yellow-brown oil (33 mg, 37.5% yield).

¹H NMR (300 MHz, CHLOROFORM-d) δ 9.35 (br. s., 1H), 7.71 (d, J=7.91 Hz, 1H), 7.41-7.48 (m, 1H), 7.27-7.38 (m, 2H), 7.11-7.18 (m, 1H), 5.62 (q, J=6.59 Hz, 1H), 5.19 (br. s., 1H), 4.44 (d, J=4.90 Hz, 1H), 3.61 (t, J=5.75 Hz, 2H), 3.48-3.54 (m, 2H), 3.21-3.35 (m, 4H), 2.86 (d, J=11.11 Hz, 1H), 2.55-2.75 (m, 3H), 2.06-2.25 (m, 2H), 1.72 (d, J=6.78 Hz, 3H), 1.44 (s, 9H)

Treatment of the solution of tert-butyl (2-{2-[(3E)-3-ethylidene-4-(1H-indol-2-ylcarbonyl)piperidin-1-yl]ethoxy}ethyl)carbamate (33 mg, 0.075 mmol) in DCM (1 mL) with TFA (0.5 mL) resulted in WW-60 as an orange-brown oil (25 mg, 99%).

¹H NMR (300 MHz, CHLOROFORM-d) δ 9.52 (br. s., 1H), 7.66-7.75 (m, 1H), 7.39-7.48 (m, 1H), 7.27-7.38 (m, 2H), 7.10-7.19 (m, 1H), 5.62 (q, J=6.34 Hz, 1H), 4.44 (d, J=4.14 Hz, 1H), 3.66-3.79 (m, 3H), 3.53-3.63 (m, 4H), 3.22-3.35 (m, 2H), 2.90 (d, J=11.87 Hz, 2H), 2.59-2.74 (m, 3H), 1.99-2.19 (m, 2H), 1.70 (d, J=6.78 Hz, 3H) ¹³C NMR (75 MHz, CHLOROFORM-d) δ 191.2, 135.2, 132.1, 130.3, 127.5, 126.8, 125.3, 124.2, 124.1, 120.9, 120.8, 118.8, 118.7, 111.7, 111.6, 109.9, 109.9, 107.0, 68.8, 66.2, 65.8, 65.4, 64.8, 57.9, 57.7, 54.7, 54.5, 53.0, 48.0, 47.8, 39.7, 38.9, 37.4, 27.7, 27.4, 26.2, 26.1, 10.9

The free base was converted to its hydrochloride salt by treating with 2 M HCl in diethyl ether to obtain an orange-brown solid.

Synthesis of WW-18 to WW-20;

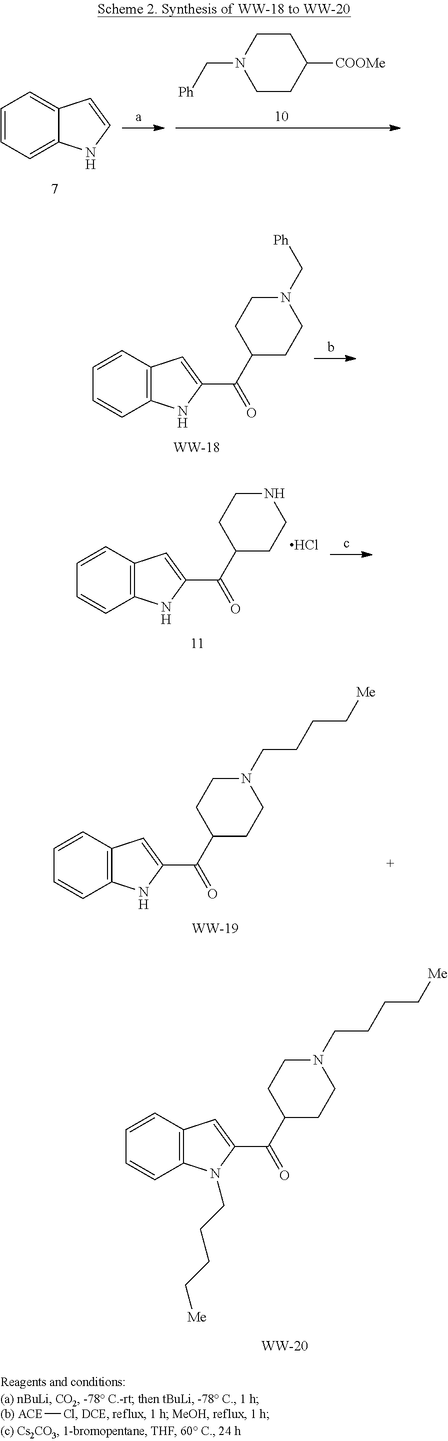

(1-Benzylpiperidin-4-yl)(1H-indol-2-yl)methanone (WW-18): To a solution of indole 7 (0.47 g, 3.8 mmol) in anhydrous THF (15 mL) at −78° C. was added nBuLi solution (1.6 mL/2.5 M in hexanes, 4 mmol), and the mixture was stirred at −78° C. for 0.5 h. To this solution was added dry ice (1.5 g, excess) and the mixture was stirred at −78° C. for 10 min. The reaction mixture was then slowly allowed to warm to rt over 0.5 h. The solvent was evaporated, and excess CO₂ was removed under reduced pressure to obtain a residue, which was redissolved in anhydrous THF (10 mL). The solution was cooled to −78° C. To this was slowly added tBuLi solution (2.3 mL/1.7 M in pentane, 3.94 mmol), and the mixture was stirred at −78° C. for 1 h to obtain a lithiated indole intermediate 8 (in situ). To the solution of 8 at −78° C. was added a cold solution of methyl 1-benzyl-4-piperidinecarboxylate 10 (0.89 g, 3.81 mmol) in anhydrous THF (10 mL). The reaction was stirred at −78° C. for 1 h and then allowed to warm to rt over 1 h. It was then quenched by the addition of satd. aqueous NaHCO₃ solution (20 mL). The aqueous layer was separated and extracted with CH₂Cl₂ (25 mL×3). The combined organic layers were washed with brine (25 mL) and concentrated under reduced pressure to obtain a crude product. Purification was done using flash column chromatography on silica gel (ISCO, 12 g Gold, Silica) eluting with 0%-5% MeOH in DCM to afford amine WW-18 as an off-white solid (386 mg, 32% yield).

¹H NMR (300 MHz, CHLOROFORM-d) δ 9.11 (br. s., 1H), 7.70 (dd, J=0.75, 8.10 Hz, 1H), 7.39-7.46 (m, 1H), 7.29-7.38 (m, 6H), 7.15 (ddd, J=1.13, 6.92, 7.96 Hz, 1H), 3.56 (s, 2H), 3.11-3.25 (m, 1H), 2.95-3.06 (m, 2H), 2.14 (dt, J=3.20, 11.30 Hz, 2H), 1.82-2.03 (m, 5H)

1H-indol-2-yl(piperidin-4-yl)methanone hydrochloride (11): To a solution of WW-18 (0.1 g, 0.314 mmol) in dichloroethane (8 mL) was added alpha-chloroethyl chloroformate (0.07 mL). The mixture was heated at reflux for 4 h. After this, the mixture was concentrated under reduced pressure to obtain a crude carbamate intermediate, which was dissolved in anhydrous MeOH (8 mL). The solution was heated at reflux for 1 h. After removal of the solvent, the crude product was slurried in diethyl ether (15 mL) and filtered to obtain 1H-indol-2-yl(piperidin-4-yl)methanone as hydrochloride salt (11), which was used in the next step without further purification.

1H-indol-2-yl(1-pentylpiperidin-4-yl)methanone (WW-19) and (1-pentyl-1H-indol-2-yl(1-pentylpiperidin-4-yl)methanone (WW-20): Using the General Procedure A described above, reaction of 1H-indol-2-yl(piperidin-4-yl)methanone hydrochloride 11 (72 mg, 0.2 mmol) with 1-bromopentane (45 μL, 0.4 mmol) afforded WW-19 as an off-white solid (37 mg, 62% yield) and WW-20 as a yellow-orange oil (28 mg, 37% yield) after chromatographic purification (ISCO, 12 g Gold, Silica).

WW-19: ¹H NMR (300 MHz, CHLOROFORM-d) δ 9.11 (br. s., 1H), 7.70 (d, J=8.10 Hz, 1H), 7.39-7.46 (m, 1H), 7.34 (dt, J=0.94, 7.63 Hz, 1H), 7.22 (d, J=1.13 Hz, 1H), 7.15 (dt, J=1.04, 7.49 Hz, 1H), 3.10-3.26 (m, 1H), 3.05 (d, J=11.30 Hz, 2H), 2.31-2.43 (m, 2H), 1.86-2.14 (m, 6H), 1.52 (td, J=7.54, 15.07 Hz, 2H), 1.23-1.40 (m, 4H), 0.91 (t, J=6.97 Hz, 3H) ¹³C NMR (75 MHz, CHLOROFORM-d) δ 195.6, 137.3, 134.3, 127.6, 126.2, 123.0, 121.0, 112.1, 108.7, 59.1, 53.4, 44.8, 29.9, 29.2, 26.7, 22.6, 14.0.

The free base was converted to its hydrochloride salt by treating with 2 M HCl in diethyl ether to obtain an off white solid.

WW-20: ¹H NMR (300 MHz, CHLOROFORM-d) δ 7.69 (d, J=8.10 Hz, 1H), 7.31-7.42 (m, 2H), 7.29 (s, 1H), 7.14 (ddd, J=1.41, 6.40, 8.01 Hz, 1H), 4.46-4.61 (m, 2H), 3.14-3.30 (m, 1H), 3.04 (d, J=11.49 Hz, 2H), 2.29-2.41 (m, 2H), 2.02-2.14 (m, 2H), 1.84-1.96 (m, 4H), 1.73 (quin, J=7.39 Hz, 2H), 1.52 (td, J=7.39, 14.98 Hz, 2H), 1.21-1.42 (m, 8H), 0.78-0.97 (m, 6H)

¹³C NMR (75 MHz, CHLOROFORM-d) δ 196.5, 139.5, 133.6, 125.8, 125.5, 122.7, 120.5, 110.9, 110.6, 58.9, 53.4, 46.0, 44.9, 30.2, 29.8, 29.3, 29.0, 26.6, 22.5, 22.4, 13.9, 13.9

The free base was converted to its hydrochloride salt by treating with 2 M HCl in diethyl ether to obtain an off white solid.

Synthesis of WW-22 and WW-23;

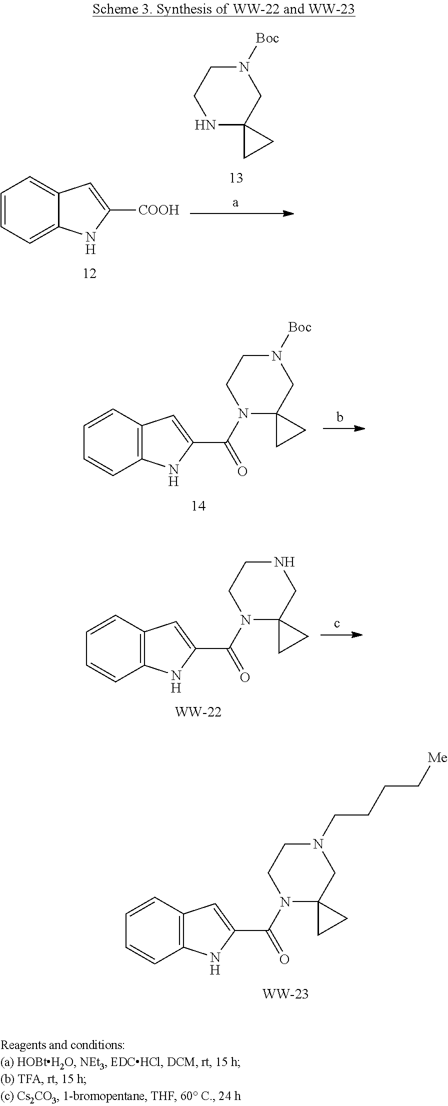

General Procedure B: Synthesis of tert-butyl 4-(1H-indol-2-ylcarbonyl)-4,7-diazaspiro[2.5]octane-7-carboxylate (14): To a solution of indole-2-acetic acid 12 (0.19 g, 1.17 mmol) in DCM (20 mL) was added tert-butyl 4,7-diazaspiro[2.5]octane-7-carboxylate 13 (0.27 g, 1.29 mmol), followed by HOBt (0.22 g, 1.61 mmol), EDC·HCl (0.31 g, 1.61 mmol) and NEt₃ (0.45 mL, 3.22 mmol). The reaction mixture was stirred at rt under nitrogen atmosphere for 15 h. The reaction was quenched by the addition of aqueous satd. NaHCO₃ solution (10 mL) and the layers separated. The aqueous layer was extracted with DCM (20 mL×3). The combined organic extracts were washed with brine (20 mL), dried (Na₂SO₄), and concentrated under reduced pressure. Purification was done bt column chromatography on silica gel using ISCO (12 g, Gold, Silica) eluting with 3%-40% EtOAc in hexanes to obtain tert-butyl 4-(1H-indol-2-ylcarbonyl)-4,7-diazaspiro[2.5]octane-7-carboxylate 14 as a white foam (374 mg, 90% yield).

2-(4,7-diazaspiro[2.5]oct-4-ylcarbonyl)-1H-indole (WW-22): To a solution of 14 (0.15 g, 0.42 mmol) in DCM (8 mL) was added TFA (0.5 mL) and the reaction was stirred at rt for 15 h. The reaction was quenched by the addition of aqueous satd. NaHCO₃ solution (5 mL) and the layers separated. The aqueous layer was extracted with DCM (10 mL×3). The combined organic extracts were washed with brine (10 mL), dried (Na₂SO₄), and concentrated under reduced pressure to obtain 2-(4,7-diazaspiro[2.5]oct-4-ylcarbonyl)-1H-indole (WW-22) as an yellow-orange solid (100 mg, 93% yield).

¹H NMR (300 MHz, CHLOROFORM-d) δ 9.57 (br. s., 1H), 7.65 (d, J=8.10 Hz, 1H), 7.43 (dd, J=0.66, 8.19 Hz, 1H), 7.26-7.31 (m, 1H), 7.09-7.16 (m, 1H), 6.81-6.90 (m, 1H), 3.98 (d, J=18.65 Hz, 1H), 2.88-3.11 (m, 3H), 1.88 (br. s., 2H), 0.85-1.10 (m, 4H).

The free base was converted to its hydrochloride salt by treating with 2 M HCl in diethyl ether to obtain a yellow solid.

2-[(7-pentyl-4,7-diazaspiro[2.5]oct-4-yl)carbonyl]-1H-indole (WW-23) and 1-pentyl-2-[(7-pentyl-4,7-diazaspiro[2.5]oct-4-yl)carbonyl]-1H-indole (WW-24): Using the General Procedure A described above, reaction of 2-(4,7-diazaspiro[2.5]oct-4-ylcarbonyl)-1H-indole 14 (106 mg, 0.41 mmol) with 1-bromopentane (65 μL, 0.62 mmol) afforded WW-23 as a light yellow oil (28 mg, 21% yield) and WW-24 as a colorless oil (25 mg, 15% yield) after chromatographic purification (ISCO, 4 g Gold, Silica).

### 2-[(7-pentyl-4,7-diazaspiro[2.5]oct-4-yl)carbonyl]-1H-indole (WW-23)

¹H NMR (300 MHz, CHLOROFORM-d) δ 9.70 (br. s., 1H), 7.64 (d, J=7.91 Hz, 1H), 7.43 (dd, J=0.57, 8.29 Hz, 1H), 7.27 (ddd, J=1.04, 6.50, 8.85 Hz, 1H), 7.08-7.15 (m, 1H), 6.89 (s, 1H), 4.08 (br. s., 1H), 2.55 (br. s., 3H), 2.31-2.41 (m, 2H), 1.49 (quin, J=7.39 Hz, 2H), 1.24-1.38 (m, 4H), 0.94-1.16 (m, 4H), 0.84-0.94 (m, 3H)

¹³C NMR (75 MHz, CHLOROFORM-d) δ 135.8, 130.3, 127.5, 124.3, 121.9, 120.4, 111.8, 105.9, 59.8, 58.4, 54.4, 38.5, 29.7, 26.4, 22.6, 15.8, 14.0

The free base was converted to its hydrochloride salt by treating with 2 M HCl in diethyl ether to obtain an off white solid.

### 1-pentyl-2-[(7-pentyl-4,7-diazaspiro[2.5]oct-4-yl)carbonyl]-1H-indole (WW-24)

¹H NMR (300 MHz, CHLOROFORM-d) δ 7.61 (d, J=7.91 Hz, 1H), 7.34-7.39 (m, 1H), 7.23-7.30 (m, 1H), 7.08-7.15 (m, 1H), 6.59-6.68 (m, 1H), 4.23-4.40 (m, 2H), 3.89 (br. s., 1H), 2.48 (br. s., 3H), 2.29-2.39 (m, 2H), 1.72-1.82 (m, 2H), 1.48 (quin, J=7.39 Hz, 2H), 1.20-1.39 (m, 9H), 0.89 (q, J=6.72 Hz, 10H)

¹³C NMR (75 MHz, CHLOROFORM-d) δ 137.5, 132.0, 126.4, 123.2, 121.7, 120.0, 110.1, 104.6, 60.2, 58.4, 54.6, 44.2, 30.4, 29.7, 29.2, 26.4, 22.6, 22.4, 14.6, 14.0, 14.0

The free base was converted to its hydrochloride salt by treating with 2 M HCl in diethyl ether to obtain an off white solid.

2-[(2-ethyl-4-pentylpiperazin-1-yl)carbonyl]-1H-indole (WW-25): Using the procedure described for the synthesis of WW-23 above, 2-[(2-ethyl-4-pentylpiperazin-1-yl)carbonyl]-1H-indole (WW-25) was obtained from indole-2-acetic acid 12 (0.16 g, 1 mmol) and tert-butyl 3-ethylpiperazine-1-carboxylate (0.24 g, 1.1 mmol) followed by Boc-deprotection and General Procedure A, as a white solid (0.29 g, 80% yield).

¹H NMR (300 MHz, CHLOROFORM-d) δ 9.69 (br. s., 1H), 7.64 (d, J=7.91 Hz, 1H), 7.39-7.46 (m, 1H), 7.22-7.31 (m, 1H), 7.07-7.16 (m, 1H), 6.75 (d, J=1.32 Hz, 1H), 4.68 (br. s., 1H), 4.51 (d, J=13.00 Hz, 1H), 3.13-3.76 (m, 1H), 2.79-2.99 (m, 2H), 2.20-2.41 (m, 2H), 1.85-2.16 (m, 4H), 1.48 (quin, J=7.16 Hz, 2H), 1.24-1.40 (m, 4H), 0.85-1.01 (m, 6H)

¹³C NMR (75 MHz, CHLOROFORM-d) δ 162.5, 135.7, 129.7, 127.5, 124.2, 121.7, 120.4, 111.8, 104.7, 58.4, 55.1, 53.8, 29.6, 26.5, 23.2, 22.6, 14.1, 10.7

2-[(4-pentylpiperazin-1-yl)carbonyl]-1H-indole (WW-28): Reaction ofindole-2-acetic acid 12 (1.49 g, 9.2 mmol) and 1-benzylpiperazine (1.8 g, 10.2 mmol) resulted in 2-[(4-benzylpiperazin-1-yl)carbonyl]-1H-indole (white solid; 2 g, 69% yield) after chromatographic purification using ISCO (24 g, Gold, Silica) eluting with 5% MeOH in DCM. Debenzylation using the procedure described above for the synthesis of WW-8, resulted in 2-(piperazin-1-ylcarbonyl)-1H-indole hydrochloride. Alkylation of 2-(piperazin-1-ylcarbonyl)-1H-indole hydrochloride (79 mg, 0.3 mmol) with 1-bromopentane using the General Procedure A afforded 2-[(4-pentylpiperazin-1-yl)carbonyl]-1H-indole (WW-28) as a white solid (38 mg, 42% yield).

¹H NMR (300 MHz, CHLOROFORM-d) δ 9.87 (br. s., 1H), 7.63 (d, J=7.91 Hz, 1H), 7.43 (d, J=8.29 Hz, 1H), 7.21-7.30 (m, 1H), 7.06-7.15 (m, 1H), 6.77 (d, J=1.51 Hz, 1H), 3.97 (br. s., 4H), 2.45-2.61 (m, 4H), 2.27-2.43 (m, 2H), 1.52 (quin, J=7.35 Hz, 2H), 1.22-1.41 (m, 4H), 0.81-0.98 (m, 3H)

¹³C NMR (75 MHz, CHLOROFORM-d) δ 160.3, 133.7, 127.2, 125.3, 122.1, 119.6, 118.3, 109.7, 103.0, 56.5, 51.1, 27.6, 24.3, 20.5, 11.9

ESI MS: 300.2 (M+H)⁺

The free base was converted to its hydrochloride salt by treating with 2 M HCl in diethyl ether to obtain an off white solid.

2-[(4-pentylpiperazin-1-yl)methyl]-1H-indole (WW-21): To a solution of 2-(piperazin-1-ylcarbonyl)-1H-indole (0.11 g, 0.5 mmol) in anhydrous THF (10 mL) was slowly added LiAlH₄ (1M solution in THF; 1 mL, 1 mmol) at rt, and the solution was heated at reflux for 12 h. After cooling down to rt, the reaction was quenched by careful addition of water (0.5 mL), 2 N NaOH (1 mL), followed by water (1.5 mL) sequentially. The aqueous layer was extracted with EtOAc (10 mL×3), dried (Na₂SO₄), and concentrated. Recrystallization with DCM:hexanes (1:10) resulted in pure 2-(piperazin-1-ylmethyl)-1H-indole as an off-white crystalline solid (36 mg, 34% yield). Alkylation of 2-(piperazin-1-ylmethyl)-1H-indole with 1-bromopentane using the General Procedure A resulted in 2-[(4-pentylpiperazin-1-yl)methyl]-1H-indole (WW-21).

¹H NMR (300 MHz, CHLOROFORM-d) δ 8.68 (br. s., 1H), 7.54 (d, J=7.72 Hz, 1H), 7.28-7.36 (m, 1H), 7.02-7.19 (m, 2H), 6.35 (d, J=0.94 Hz, 1H), 2.29-2.37 (m, 2H), 1.48 (td, J=7.49, 15.16 Hz, 2H), 1.20-1.39 (m, 4H), 0.89 (t, J=6.88 Hz, 3H) The free base was converted to its hydrochloride salt by treating with 2 M HCl in diethyl ether to obtain an off white solid.

Tert-butyl (3S)-3-[(1H-indol-2-ylcarbonyl)amino]pyrrolidine-1-carboxylate (WW-26): Reaction of indole-2-acetic acid 12 (0.16 g, 1 mmol) and tert-butyl (3S)-3-aminopyrrolidine-1-carboxylate (0.2 g, 1.1 mmol) using the procedure described for the synthesis of 14, resulted in tert-butyl (3S)-3-[(1H-indol-2-ylcarbonyl)amino]pyrrolidine-1-carboxylate (WW-26) as a white solid (0.32 g, 97% yield).

¹H NMR (300 MHz, CHLOROFORM-d) δ 10.13 (br. s., 1H), 7.62 (d, J=7.91 Hz, 1H), 7.43 (d, J=8.29 Hz, 1H), 7.22-7.32 (m, 1H), 7.08-7.16 (m, 1H), 4.71 (br. s., 1H), 3.27-3.81 (m, 4H), 2.15-2.30 (m, 1H), 2.12 (s, OH), 1.48 (br. s., 9H)

¹³C NMR (75 MHz, CHLOROFORM-d) δ 229.0, 161.8, 154.7, 136.6, 130.5, 127.6, 124.5, 122.0, 120.6, 112.1, 103.1, 79.9, 53.4, 44.1, 28.5

ESI MS: 328.2 (M−1)

N-[(3S)-1-pentylpyrrolidin-3-yl]-1H-indole-2-carboxamide (WW-29): Treatment of WW-26 with TFA to remove the Boc-group followed by alkylation of the resulting intermediate secondary amine (31 mg, 0.135 mmol) with 1-bromopentane using Procedure A described above resulted in N-[(3S)-1-pentylpyrrolidin-3-yl]-1H-indole-2-carboxamide (WW-29) as a white solid (14 mg, 35% yield).

¹H NMR (300 MHz, CHLOROFORM-d) δ 9.84 (br. s., 1H), 7.63 (d, J=7.91 Hz, 1H), 7.43 (d, J=8.29 Hz, 1H), 7.23-7.31 (m, 1H), 7.08-7.15 (m, 1H), 6.91 (d, J=1.32 Hz, 1H), 5.35-6.04 (m, 2H), 4.71 (dt, J=1.79, 4.19 Hz, 1H), 2.95-3.09 (m, 1H), 2.86 (d, J=9.98 Hz, 1H), 2.30-2.56 (m, 6H), 2.21 (q, J=8.54 Hz, 1H), 1.71-1.86 (m, 1H), 1.43-1.58 (m, 2H), 1.26-1.35 (m, 4H), 0.90 (t, J=6.78 Hz, 3H) ¹³C NMR (75 MHz, CHLOROFORM-d) δ 161.1, 136.4, 131.0, 127.7, 124.3, 121.9, 120.5, 111.9, 102.4, 61.0, 56.2, 53.0, 48.7, 32.5, 29.8, 28.3, 22.6, 22.6, 14.0 ESI MS: 300 (M+H)⁺

tert-butyl 4-(1H-indol-2-ylcarbonyl)-1,4-diazepane-1-carboxylate (WW-27): Reaction of indole-2-acetic acid 12 (0.16 g, 1 mmol) and tert-butyl homopiperazine-1-carboxylate (0.26 g, 1.1 mmol) using General Procedure B, resulted in WW-27 as a white solid (0.33 g, 96% yield).

¹H NMR (300 MHz, CHLOROFORM-d) δ 9.93 (d, J=13.19 Hz, 1H), 7.65 (d, J=7.91 Hz, 1H), 7.44 (d, J=8.10 Hz, 1H), 7.22-7.33 (m, 1H), 7.07-7.18 (m, 1H), 6.83 (br. s., 1H), 3.75-4.14 (m, 4H), 3.66 (br. s., 2H), 3.38-3.58 (m, 2H), 2.05 (br. s., 2H), 1.46 (br. s., 9H) ESI MS: 342.4 (M−1)⁻

2-[(4-pentyl-1,4-diazepan-1-yl)carbonyl]-1H-indole (WW-30): Removal of the Boc-group of WW-27 resulted in the corresponding secondary amine. Alkylation of the secondary amine (62 mg, 0.26 mmol) with 1-bromopentane using Procedure A described above resulted in 2-[(4-pentyl-1,4-diazepan-1-yl)carbonyl]-1H-indole (WW-30) as an off-white solid (56 mg, 70% yield).

¹H NMR (300 MHz, CHLOROFORM-d) δ 9.79 (br. s., 1H), 7.65 (d, J=8.10 Hz, 1H), 7.44 (d, J=8.29 Hz, 1H), 7.23-7.30 (m, 1H), 7.05-7.17 (m, 1H), 6.79 (d, J=19.40 Hz, 1H), 3.70-4.14 (m, 4H), 2.86 (d, J=17.52 Hz, 2H), 2.68 (br. s., 2H), 2.41-2.54 (m, 2H), 1.49 (quin, J=7.39 Hz, 2H), 1.21-1.38 (m, 4H), 0.82-0.95 (m, 3H)

¹³C NMR (75 MHz, CHLOROFORM-d) δ 163.1, 135.5, 129.9, 127.8, 124.3, 121.9, 120.4, 111.7, 105.4, 58.0, 29.7, 27.1, 22.6, 14.0

ESI MS: 314 (M+H)⁺

1-(1H-indole-2-ylcarbonyl)azetidin-3-amine (WW-31): Reaction of indole-2-acetic acid 12 (0.16 g, 1 mmol) and tert-butyl azetidin-3-ylcarbamate (0.23 g, 1.1 mmol) using the procedure described for the synthesis of 14, followed by removal of the Boc-group afforded 1-(1H-indole-2-ylcarbonyl)azetidin-3-amine (WW-31) as a TFA salt (pink solid; 0.27 g, 82% yield over 2 steps).

¹H NMR (300 MHz, METHANOL-d₄) δ 7.64 (d, J=8.10 Hz, 1H), 7.46 (dd, J=0.75, 8.29 Hz, 1H), 7.25 (dt, J=0.94, 7.63 Hz, 1H), 7.04-7.13 (m, 1H), 6.86 (s, 1H), 4.91-5.03 (m, 1H), 4.55 (br. s., 2H), 4.08-4.34 (m, 2H)

¹³C NMR (75 MHz, METHANOL-d₄) δ 164.5, 138.0, 129.3, 129.2, 125.8, 123.0, 121.4, 113.1, 106.9, 42.5

ESI MS: 216 (M+H)⁺

1-(1H-indole-2-ylcarbonyl)-N-pentylazetidin-3-amine (WW-32): Alkylation of WW-31 (125 mg, 0.28 mmol) with 1-bromopentane using General Procedure A resulted in 1-(1H-indole-2-ylcarbonyl)-N-pentylazetidin-3-amine (WW-32) as an off-white solid (60 mg, 55% yield).

¹H NMR (300 MHz, METHANOL-d₄) δ 7.64 (d, J=7.91 Hz, 1H), 7.46 (d, J=8.29 Hz, 1H), 7.25 (s, 1H), 7.08 (s, 1H), 6.89 (s, 1H), 4.91-5.01 (m, 1H), 4.43-4.69 (m, 2H), 4.14-4.34 (m, 2H), 2.97-3.09 (m, 2H), 1.63-1.80 (m, 2H), 1.35-1.49 (m, 4H), 0.89-1.03 (m, 3H).

N-[(3S)-1-pyrrolo[1,2-a]pyrazin-1-ylpyrrolidin-3-yl]-1H-indole-2-carboxamide (WW-33): Reaction of indole-2-acetic acid 12 (37 mg, 0.23 mmol) and (3S)-1-pyrrolo[1,2-a]pyrazin-1-ylpyrrolidin-3-amine trifluoroacetate (79 mg, 0.25 mmol) using General Procedure B, resulted in WW-33 as a buff colored solid (60 mg, 75% yield).

¹H NMR (300 MHz, CHLOROFORM-d) δ 7.63 (d, J=7.91 Hz, 1H), 7.36-7.47 (m, 1H), 7.21-7.32 (m, 3H), 7.06-7.16 (m, 2H), 6.96 (d, J=4.90 Hz, 1H), 6.84 (d, J=4.14 Hz, 1H), 6.63 (dd, J=2.64, 4.14 Hz, 1H), 4.71-4.85 (m, 1H), 4.14 (dd, J=6.12, 11.02 Hz, 1H), 3.84-4.08 (m, 3H), 2.31-2.47 (m, 1H), 2.09-2.23 (m, 1H)

¹³C NMR (75 MHz, CHLOROFORM-d) δ 162.1, 151.1, 136.5, 130.4, 127.5, 125.3, 124.4, 122.0, 120.4, 120.2, 116.0, 112.0, 111.9, 110.5, 105.1, 103.9, 53.8, 49.5, 46.9, 31.4

ESI MS: 346.2 (M+H)⁺

Synthesis of WW-43 to WW-47;

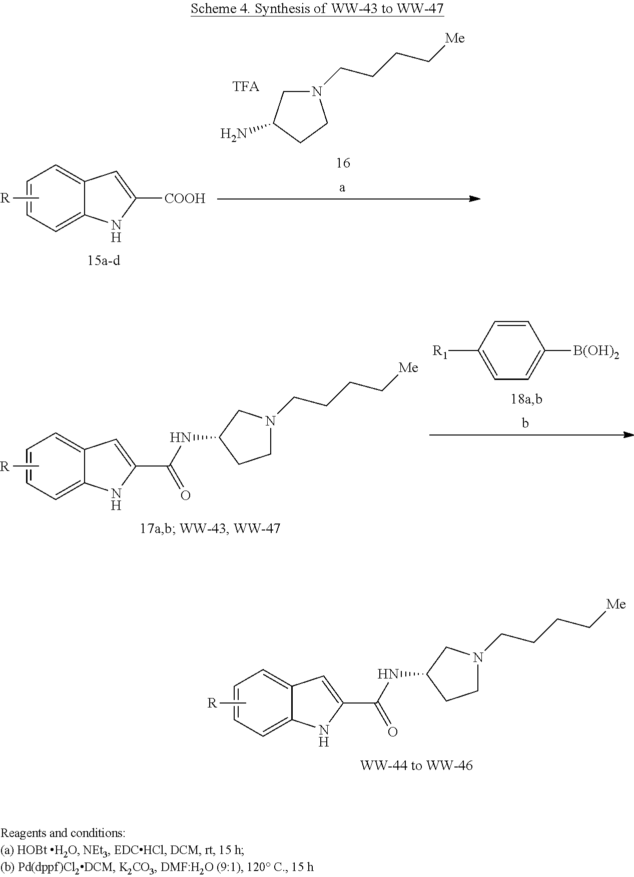

### 5,6,7-Trimethoxy-N-[(3S)-1-pentylpyrrolidin-3-yl]-1H-indole-2-carboxamide (WW-43)

Reaction of 5,6,7-trimethoxyindole-2-acetic acid 15a (63 mg, 0.25 mmol) with (3S)-1-pentylpyrrolidin-3-amine trifluoroacetate 16 (68 mg, 0.25 mmol), using General Procedure B resulted in 5,6,7-Trimethoxy-N-[(3S)-1-pentylpyrrolidin-3-yl]-1H-indole-2-carboxamide (WW-43) as a colorless oil (60 mg, 62% yield).

¹H NMR (300 MHz, CHLOROFORM-d) δ 9.51 (br. s., 1H), 7.15 (d, J=8.29 Hz, 1H), 6.84 (d, J=1.88 Hz, 1H), 6.76 (s, 1H), 4.66-4.80 (m, 1H), 4.28-4.55 (m, 1H), 4.02 (s, 3H), 3.91 (s, 3H), 3.87 (s, 3H), 3.09 (dt, J=3.30, 8.71 Hz, 1H), 2.92 (d, J=10.17 Hz, 1H), 2.57 (dd, J=6.50, 10.27 Hz, 1H), 2.25-2.53 (m, 5H), 1.76-1.89 (m, 1H), 1.45-1.59 (m, 2H), 1.26-1.35 (m, 4H), 0.87-0.92 (m, 3H)

¹³C NMR (75 MHz, CHLOROFORM-d) δ 160.9, 149.9, 139.9, 139.0, 130.7, 125.7, 123.4, 103.0, 97.6, 61.4, 61.0, 60.9, 56.3, 56.1, 52.9, 48.6, 32.3, 29.7, 28.0, 22.5, 13.9

ESI MS: 390 (M+H)⁺

Elemental Anal. Calcd. For C₂₁H₃₁N₃O₄·HCl·1.3H₂O: C, 56.08; H, 7.76; N, 9.34. Found: C, 56.26; H, 7.54; N, 9.12.

### 6-(4-Fluorophenyl)-N-[(3S)-1-pentylpyrrolidin-3-yl]-1H-indole-2-carboxamide (WW-44)

Reaction of 6-bromoindole-2-acetic acid 15c (0.48 g, 2 mmol) with (3S)-1-pentylpyrrolidin-3-amine trifluoroacetate 16 (0.54 g, 2 mmol), using General Procedure B resulted in 6-bromo-N-[(3S)-1-pentylpyrrolidin-3-yl]-1H-indole-2-carboxamide (17a) as a light-yellow oil (0.41 g, 54% yield).

Compound 17a (38 mg, 0.1 mmol) was taken in a flask with 4-flurophenyl boronic acid (98 mg, 0.7 mmol) and K₂CO₃ (194 mg, 1.4 mmol) in degassed DMF-water. To this was added Pd(dppf)Cl₂·DCM (25 mg, 0.03 mmol) under nitrogen. The resulting mixture was heated at 120° C. for 15 h. The crude material was then diluted with EtOAc (20 mL) and filtered through a pad of celite, washing the pad with EtOAc (30 mL). The filtrate was was washed with water (20 mL×3), brine (20 mL), dried (Na₂SO₄), and concentrated. Purification was done with column chromatography using ISCO (4 g, Gold, Silica) eluting with 0%-12% MeOH (containing 1% NH₄OH) in DCM to afford 6-(4-Fluorophenyl)-N-[(3S)-1-pentylpyrrolidin-3-yl]-1H-indole-2-carboxamide (WW-44) as a yellow-orange oil (36 mg, 92% yield).

¹H NMR (300 MHz, CHLOROFORM-d) δ 10.32 (br. s., 1H), 7.65 (d, J=8.29 Hz, 1H), 7.53-7.58 (m, 2H), 7.31 (dd, J=1.32, 8.29 Hz, 1H), 7.06-7.15 (m, 2H), 7.04 (d, J=8.10 Hz, 1H), 6.89 (s, 1H), 4.66-4.85 (m, 1H), 3.06 (dt, J=3.30, 8.71 Hz, 1H), 2.92 (d, J=9.98 Hz, 1H), 2.31-2.61 (m, 4H), 2.24 (q, J=8.48 Hz, 1H), 1.83 (dtd, J=3.86, 8.18, 12.55 Hz, 1H), 1.52 (quin, J=7.35 Hz, 2H), 1.20-1.39 (m, 4H), 0.81-0.95 (m, 3H)

¹³C NMR (75 MHz, CHLOROFORM-d) δ 163.5, 160.7, 160.2, 137.6, 137.6, 136.6, 136.3, 131.1, 128.4, 128.3, 126.5, 121.7, 120.0, 115.2, 114.9, 109.8, 101.8, 60.5, 55.7, 52.5, 48.5, 32.1, 29.3, 27.8, 22.1, 13.6 ESI MS: 394 (M+H)⁺

Elemental Anal. Calcd. For C₂₄H₂₅FN₃O·HC·0.2H₂O: C, 66.38; H, 6.84; N, 9.68. Found: C, 66.31; H, 6.75; N, 9.66.

### 6-(4-methoxyphenyl)-N-[(3S)-1-pentylpyrrolidin-3-yl]-1H-indole-2-carboxamide (WW-45)

Reaction of 17a (38 mg, 0.1 mmol) with 4-methoxyphenyl boronic acid (106 mg, 0.7 mmol) using the procedure described for WW-44 to afford 6-(4-methoxyphenyl)-N-[(3S)-1-pentylpyrrolidin-3-yl]-1H-indole-2-carboxamide (WW-45) as a orange oil (37 mg, 90% yield).

¹H NMR (300 MHz, CHLOROFORM-d) δ 10.03 (br. s., 1H), 7.96 (d, J=6.59 Hz, 1H), 7.60 (d, J=8.48 Hz, 1H), 7.52 (d, J=8.85 Hz, 2H), 7.33 (dd, J=1.41, 8.38 Hz, 1H), 6.94-6.96 (m, 1H), 6.87 (d, J=7.91 Hz, 1H), 4.70 (br. s., 1H), 3.80-3.87 (m, 3H), 3.75-3.77 (m, 2H), 2.92-3.22 (m, 1H), 2.43-2.61 (m, 2H), 2.19-2.41 (m, 1H), 1.44-1.63 (m, 2H), 1.15-1.40 (m, 3H), 0.88 (t, J=6.78 Hz, 2H)

¹³C NMR (75 MHz, CHLOROFORM-d) δ 161.3, 159.0, 137.5, 137.2, 134.4, 131.0, 128.3, 126.6, 122.2, 120.4, 114.2, 113.3, 109.8, 60.6, 56.1, 55.3, 55.0, 53.0, 48.6, 32.0, 29.6, 27.6, 22.5, 14.0

Elemental Anal. Calcd. for C₂₅H₃₁N₃O₂·HCl·0.5H₂O: C, 67.52; H, 7.32; N, 9.45. found: C, 67.40; H, 7.12; N, 9.25.

### 7-(4-Fluorophenyl)-N-[(3S)-1-pentylpyrrolidin-3-yl]-1H-indole-2-carboxamide (WW-46)

Reaction of 7-bromoindole-2-acetic acid 15d (0.48 g, 2 mmol) with (3S)-1-pentylpyrrolidin-3-amine trifluoroacetate 16 (0.54 g, 2 mmol), using General Procedure B resulted in 7-bromo-N-[(3S)-1-pentylpyrrolidin-3-yl]-1H-indole-2-carboxamide (17b) as a white foam (0.43 g, 57% yield).

Reaction of 17b (53 mg, 0.14 mmol) with 4-flurophenyl boronic acid (137 mg, 1 mmol) using the procedure described for WW-44 afforded 7-(4-Fluorophenyl)-N-[(3S)-1-pentylpyrrolidin-3-yl]-1H-indole-2-carboxamide (WW-46).

¹H NMR (300 MHz, CHLOROFORM-d) δ 9.30 (br. s., 1H), 7.47-7.65 (m, 3H), 7.10-7.31 (m, 4H), 6.95 (d, J=2.07 Hz, 1H), 4.54-4.71 (m, 1H), 3.00 (dt, J=3.39, 8.76 Hz, 1H), 2.81 (d, J=9.98 Hz, 1H), 2.52 (dd, J=6.40, 9.98 Hz, 1H), 2.44 (dd, J=6.50, 8.57 Hz, 2H), 2.29-2.39 (m, 1H), 2.17-2.28 (m, 1H), 1.69-1.83 (m, 1H), 1.51 (quin, J=7.39 Hz, 2H), 1.22-1.38 (m, 4H), 0.90 (t, J=6.78 Hz, 3H)

¹³C NMR (75 MHz, CHLOROFORM-d) δ 164.0, 160.8, 160.7, 134.5, 134.4, 134.2, 131.2, 129.9, 129.8, 128.3, 125.5, 124.2, 121.3, 121.2, 116.2, 116.0, 102.8, 60.9, 56.1, 52.8, 48.8, 32.4, 29.8, 28.3, 22.6, 14.0.

ESI MS: 394 (M+H)⁺

Elemental Anal. Calcd. for C₂₄H₂₈FN₃O·HCl·0.15H₂O: C, 66.63; H, 6.83; N, 9.71. found: C, 66.78; H, 6.77; N, 9.56.

### 4,5,6-Trimethoxy-N-[(3S)-1-pentylpyrrolidin-3-yl]-1H-indole-2-carboxamide (WW-47)

Reaction of 4,5,6-trimethoxyindole-2-acetic acid 15b (63 mg, 0.25 mmol) with (3S)-1-pentylpyrrolidin-3-amine trifluoroacetate 16 (68 mg, 0.25 mmol), using General Procedure B resulted in 4,5,6-trimethoxy-N-[(3S)-1-pentylpyrrolidin-3-yl]-1H-indole-2-carboxamide (WW-47) as a colorless oil (66 mg, 68% yield).

¹H NMR (300 MHz, CHLOROFORM-d) δ 10.22 (br. s., 1H), 6.94 (d, J=1.32 Hz, 1H), 6.62 (s, 1H), 4.58-4.84 (m, 1H), 4.08 (s, 3H), 3.86 (d, J=3.20 Hz, 6H), 3.00 (dt, J=3.30, 8.62 Hz, 1H), 2.86 (dd, J=1.88, 9.80 Hz, 1H), 2.55 (dd, J=6.59, 9.98 Hz, 1H), 2.31-2.49 (m, 3H), 2.18-2.29 (m, 1H), 1.72-1.88 (m, 1H), 1.43-1.58 (m, 2H), 1.22-1.39 (m, 4H), 0.84-0.94 (m, 3H)

¹³C NMR (75 MHz, CHLOROFORM-d) δ 161.2, 153.1, 146.2, 136.1, 133.8, 129.2, 115.5, 100.5, 89.5, 61.4, 60.9, 60.8, 56.2, 56.0, 52.9, 48.9, 32.6, 29.8, 28.3, 22.6, 14.0

Elemental Anal. Calcd. for C₂₁H₃₁N₃O₄·HCl·0.6H₂O: C, 57.80; H, 7.66; N, 9.63. found: C, 57.90; H, 7.77; N, 9.63.

1H-Indole-2-yl(4-methyl-1-pentylpiperidin-4-yl)methanone (WW-57): Alkylation of 1H-indole-2-yl(4-methyl-4-piperidinyl)methanone (40 mg, 0.165 mmol) (From Ref) with 1-bromopentane using General Method A afforded 1H-Indole-2-yl(4-methyl-1-pentylpiperidin-4-yl)methanone (WW-57) as a yellow-orange solid (37 mg, 72% yield).

¹H NMR (300 MHz, CHLOROFORM-d) δ 9.13 (br. s., 1H), 7.70 (d, J=8.10 Hz, 1H), 7.38-7.45 (m, 1H), 7.33 (dt, J=1.04, 7.58 Hz, 1H), 7.23 (d, J=1.51 Hz, 1H), 7.14 (dt, J=1.04, 7.49 Hz, 1H), 2.62 (dd, J=4.05, 10.83 Hz, 2H), 2.41-2.54 (m, 2H), 2.20-2.35 (m, 4H), 1.79 (ddd, J=3.49, 9.70, 13.37 Hz, 2H), 1.62 (d, J=4.71 Hz, 2H), 1.48 (s, 3H), 1.21-1.35 (m, 4H), 0.88 (t, J=6.88 Hz, 3H)

¹³C NMR (75 MHz, CHLOROFORM-d) δ 198.2, 135.9, 132.4, 127.8, 126.0, 123.0, 120.9, 111.9, 108.6, 59.1, 50.8, 45.7, 36.0, 29.9, 26.7, 22.6, 14.0

ESI MS: 313 (M+H)⁺

Synthesis of ethyl (3-formyl-1H-indol-2-yl){(3Z)-3-(hydroxyamino)-1-[(4-methylphenyl)sulfonyl]piperidin-4-yl}acetate (WW-65): To a solution of ethyl (3-formyl-1H-indol-2-yl)acetate (0.23 g, 0.99 mmol) in anhydrous THF (13 mL) at −78° C. was added LiHMDS (1.66 mL/1.5 M solution in THF; 2.49 mmol) under nitrogen. The reaction was stirred at −78° C. for 30 min. To this was added a solution of (3E)-4-chloro-1-[(4-methylphenyl)sulfonyl]piperidin-3-one oxime (0.36 g, 1.19 mmol) in anhydrous THF (12 mL). The mixture was stirred at −78° C. for 2 h. It was then quenched with aqueous satd. NaHCO₃ (10 mL). The aqueous layer separated and was extracted with EtOAc (20 mL×3). The combined organic extracts were washed with brine (20 mL), dried (Na₂SO₄) and concentrated. Column chromatography using ISCO (12 g, Gold, Silica) eluting with 1%-50% EtOAc in hexanes afforded ethyl (3-formyl-1H-indol-2-yl){(3Z)-3-(hydroxyamino)-1-[(4-methylphenyl)sulfonyl]piperidin-4-yl}acetate (WW-65) as a yellow solid (0.45 g, 91% yield).

ESI MS: 498 (M+H)⁺

Synthesis of ethyl {(3Z)-3-({[tert-butyl(dimethyl)silyl]oxy}imino-1-[(4-methylphenyl)sulfonyl]piperidin-4-yl}(3-formyl-1H-indol-2-yl)acetate (WW-64): To a solution of WW-65 (0.16 g, 0.32 mmol) in anhydrous DCM (5 mL) was added imidazole (0.15 g, 2.25 mmol), followed by TBDMSCl (0.17 g, 1.12 mmol) at rt. The reaction was stirred at rt for 15 h. It was quenched by addition of water (20 mL). The aqueous layer was extracted with DCM (10 mL×3). The combined organic extracts were washed with brine (10 mL), dried (Na₂SO₄) and concentrated under reduced pressure. Column chromatography using ISCO (12 g, Gold, Silica) eluting with 0%-50% EtOAc in hexanes afforded ethyl {(3Z)-3-({[tert-butyl(dimethyl)silyl]oxy}imino-1-[(4-methylphenyl)sulfonyl]piperidin-4-yl}(3-formyl-1H-indol-2-yl)acetate (WW-64) as a yellow-brown solid (0.124 g, 64% yield).

¹H NMR (300 MHz, CHLOROFORM-d) δ 10.12 (s, 1H), 9.20 (s, 1H), 8.22 (d, J=5.84 Hz, 1H), 7.63 (d, J=8.29 Hz, 2H), 7.27-7.37 (m, 5H), 4.91 (d, J=14.88 Hz, 1H), 4.63 (d, J=9.98 Hz, 1H), 4.08-4.18 (m, 3H), 3.46-3.62 (m, 1H), 3.23 (d, J=14.88 Hz, 1H), 2.99-3.14 (m, 1H), 2.71-2.87 (m, 1H), 2.44 (s, 3H), 2.05 (s, 2H), 1.45 (d, J=5.27 Hz, 2H), 1.12-1.34 (m, 6H), 0.90-1.02 (m, 9H), 0.24 (s, 3H), 0.18 (s, 3H)

ESI MS: 635 (M+Na)⁺

Synthesis of 5-(4-fluorophenyl)-1-(phenylsulfonyl)-1H-indole: To a solution of 5-(4-fluorophenyl)-1H-indole (165 mg, 0.78 mmol) in THF (10 mL) at rt was added NaOH (110 mg, 2.7 mmol), triethylbenzylammonium chloride (89 mg, 0.39 mmol) and the reaction mixture was stirred for 15 min. It was then cooled to 0° C. and to this was added sulfonyl chloride (125 μL, 0.975 mmol) and the reaction stirred at 0° C. for 30 min. It was diluted with EtOAc (20 mL). The aqueous layer was extracted with EtOAc (10 mL×3). The combined organic extract were washed with brine (20 mL), dried (Na₂SO₄) and concentrated to obtain 5-(4-fluorophenyl)-1-(phenylsulfonyl)-1H-indole as buff colored foam (275 mg, quantitative yield).

¹H NMR (300 MHz, CHLOROFORM-d) δ 8.03 (s, 1H), 7.86-7.95 (m, 2H), 7.67 (d, J=1.32 Hz, 1H), 7.60 (d, J=3.77 Hz, 1H), 7.42-7.57 (m, 6H), 7.07-7.16 (m, 2H), 6.71 (s, 1H)

Synthesis of [(3E, 4R)-(3-ethylidene-1-(4-methoxybenzyl)piperidin-4-yl)][5-4-fluorophenyl)-1-(phenylsulfonyl)-1H-indol-2-yl]methanol (WW-69): To a solution of 5-(4-fluorophenyl)-1-(phenylsulfonyl)-1H-indole (0.27 g, 0.77 mmol) in anhydrous THF (10 mL) at −78° C. was added nBuLi solution (0.31 mL/2.5 M in hexanes, 0.77 mmol), and the mixture was stirred at −78° C. for 0.5 h. This cold solution was cannulated to a flask under nitrogen containing a solution of 6 (0.2 g, 0.77 mmol) in anhydrous THF (4 mL) at −78° C. The reaction was stirred at −78° C. for 30 min and warmed to rt over 30 min. It was then quenched by the addition of satd. aqueous NaHCO₃ solution (10 mL). The aqueous layer was separated and extracted with CH₂Cl₂ (15 mL×3). The combined organic layers were washed with brine (15 mL) and concentrated under reduced pressure to obtain a crude product. Chromatographic purification using ISCO (12 g, Gold, Silica) eluting with 0%-5% MeOH (containing 1% NH₄OH) in DCM afforded [(3E, 4R)-(3-ethylidene-1-(4-methoxybenzyl)piperidin-4-yl)][5-4-fluorophenyl)-1-(phenylsulfonyl)-1H-indol-2-yl]methanol (WW-69) as a yellow-orange semisolid (0.2 g, 43% yield).

¹H NMR (300 MHz, CHLOROFORM-d) δ 8.15 (d, J=8.67 Hz, 1H), 7.70-7.79 (m, 2H), 7.32-7.57 (m, 7H), 7.17-7.26 (m, 2H), 7.05-7.15 (m, 2H), 6.90 (d, J=8.67 Hz, 2H), 6.81-6.85 (m, 1H), 5.43 (d, J=5.27 Hz, 1H), 5.38 (d, J=7.16 Hz, 1H), 3.81 (s, 3H), 3.80 (s, 1H), 3.63 (t, J=5.27 Hz, 1H), 3.53 (s, 2H), 3.27 (d, J=13.19 Hz, 1H), 2.87 (d, J=13.37 Hz, 1H), 2.61-2.72 (m, 1H), 2.21-2.33 (m, 1H), 1.79 (d, J=6.78 Hz, 3H)

ESI MS: 611 (M+H)⁺

2-{[(3E, 4R)-(3-ethylidene-1-(4-methoxybenzyl)piperidin-4-yl)](hydroxy)methyl}-1-(phenylsulfonyl)-1H-indole-5-carbonitrile (WW-70): Reaction of 1-phenylsulfonyl-1H-indole-5-carbonitrile (0.105 g, 0.37 mmol) with 6 (96.5 mg, 0.37 mmol) using the procedure described for WW-69 afforded 2-{[(3E, 4R)-(3-ethylidene-1-(4-methoxybenzyl)piperidin-4-yl)](hydroxy)methyl}-1-(phenylsulfonyl)-1H-indole-5-carbonitrile (WW-70) as a light yellow solid (100 mg, 50% yield).

¹H NMR (300 MHz, CHLOROFORM-d) δ 8.22 (dd, J=4.71, 8.67 Hz, 1H), 7.67-7.80 (m, 3H), 7.48-7.56 (m, 2H), 7.37-7.46 (m, 2H), 7.21-7.25 (m, 2H), 6.85-6.93 (m, 3H), 5.46 (dd, J=4.62, 14.60 Hz, 1H), 5.21-5.36 (m, 1H), 3.77-3.84 (m, 5H), 3.52-3.62 (m, 3H), 2.70-2.89 (m, 2H), 2.16-2.37 (m, 2H), 2.02 (s, 1H), 1.74-1.84 (m, 3H), 1.62 (dd, J=1.88, 6.78 Hz, 1H)

ESI MS: 542 (M+H)⁺

[(3E,4R)-3-ethylidene-1-pentylpiperidin-4-yl][5-(4-fluorophenyl)-1H-indol-2-yl]methanone (WW-73): Reaction of [(3E,4R)-3-ethylidenepiperidin-4-yl][5-(4-fluorophenyl)-1H-indol-2-yl]methanone (44 mg, 0.13 mmol) with 1-bromopropane (20 μL, 0.19 mmol) using General Procedure A resulted in [(3E,4R)-3-ethylidene-1-pentylpiperidin-4-yl][5-(4-fluorophenyl)-1H-indol-2-yl]methanone (WW-73) as a pale yellow semisolid (21 mg, 39% yield).

¹H NMR (300 MHz, CHLOROFORM-d) δ 9.19 (br. s., 1H), 7.85 (s, 1H), 7.53-7.63 (m, 3H), 7.44-7.52 (m, 1H), 7.33 (d, J=1.32 Hz, 1H), 7.09-7.19 (m, 2H), 5.66 (q, J=6.78 Hz, 1H), 4.44 (d, J=5.27 Hz, 1H), 3.23-3.33 (m, 1H), 3.07-3.19 (m, 1H), 2.90 (d, J=11.68 Hz, 1H), 2.36-2.58 (m, 3H), 2.05-2.30 (m, 2H), 1.74 (dd, J=1.60, 6.88 Hz, 3H), 1.55 (td, J=7.46, 15.02 Hz, 2H), 1.24-1.35 (m, 4H), 0.85-0.94 (m, 3H)

ESI MS: 419 (M+H)⁺

Synthesis of [(3E,4R)-3-ethylidene-1-(4-methoxybenzyl)piperidin-4-yl][1-(phenylsulfonyl)-1H-indol-2-yl]methanol (WW-61): Reaction of 1-phenylsulfonyl-1H-indole (0.26 g, 1 mmol) with 6 (0.26 g, 0.37 mmol) using the procedure described for WW-69 afforded [(3E,4R)-3-ethylidene-1-(4-methoxybenzyl)piperidin-4-yl][1-(phenylsulfonyl)-1H-indol-2-yl]methanol WW-61 as a yellow-orange solid (0.3 g, 58% yield).

¹H NMR (300 MHz, CHLOROFORM-d) δ 8.09 (s, 1H), 7.66-7.76 (m, 2H), 7.29-7.49 (m, 5H), 7.19-7.24 (m, 2H), 6.89 (d, J=8.67 Hz, 2H), 6.79 (s, 1H), 5.40-5.45 (m, 1H), 5.31-5.40 (m, 1H), 3.82 (s, 3H), 3.58-3.65 (m, 1H), 3.52 (s, 2H), 3.17-3.31 (m, 1H), 2.81-2.91 (m, 1H), 2.59-2.73 (m, 1H), 2.21-2.36 (m, 1H), 1.76 (d, J=6.97 Hz, 3H)

ESI MS: 517 (M+H)⁺

[(3E, 4R)-(3-ethylidene-1-pentylpiperidin-4-yl)][1-(phenylsulfonyl)-1H-indol-2-yl methanol (WW-74): Debenzylation of WW-61 using the procedure described above for the synthesis of WW-8 afforded [(3E,4R)-3-ethylidene-1-piperidin-4-yl][1-(phenylsulfonyl)-1H-indol-2-yl]methanol (ESI MS 397). Alkylation of [(3E,4R)-3-ethylidene-1-piperidin-4-yl][1-(phenylsulfonyl)-1H-indol-2-yl]methanol (75 mg, 0.19 mmol) with 1-bromopentane using General Method A afforded [(3E, 4R)-(3-ethylidene-1-pentylpiperidin-4-yl)][1-(phenylsulfonyl)-1H-indol-2-yl)methanol (WW-74) as an off-white solid (56 mg, 64% yield).

¹H NMR (300 MHz, CHLOROFORM-d) δ 8.08-8.15 (m, 1H), 7.66-7.77 (m, 2H), 7.39-7.47 (m, 2H), 7.35 (d, J=8.10 Hz, 2H), 7.29 (d, J=10.55 Hz, 1H), 7.19-7.24 (m, 1H), 6.85 (s, 1H), 5.35-5.56 (m, 2H), 3.60 (t, J=5.37 Hz, 1H), 3.26 (d, J=13.56 Hz, 1H), 2.87 (d, J=13.00 Hz, 1H), 2.63-2.78 (m, 1H), 2.37-2.48 (m, 2H), 2.30 (br. s., 1H), 1.78 (d, J=6.97 Hz, 3H), 1.48-1.68 (m, 4H), 1.29-1.37 (m, 4H), 0.88-0.95 (m, 3H)

ESI MS: 467 (M+H)⁺

Synthesis of ethyl 3-{[4-(methoxycarbonyl)piperidin-1-yl]methyl}-1H-indole-2-carboxylate (WW-6): To a solution of ethyl 1H-indole-2-carboxylate (0.5 g, 2.6 mmol) in anhydrous MeCN (5.6 mL) was added methyl piperidine-4-carboxylate (0.41 g, 2.8 mmol) and paraformaldehyde (0.14 g, 4.2 mmol), followed by the addition of TFA (0.32 mL, 4.2 mmol) at rt. The mixture was then heated at reflux for 3 h. After cooling to rt, the reaction mixture was concentrated under reduced pressure. To the residue thus obtained was added aqueous satd. NaHCO₃ (20 mL). The aqueous layer was extracted with DCM (50 mL×2). The combined organic extracts were washed with brine (30 mL), dried (Na₂SO₄), and concentrated. Chromatographic purification using ISCO (12 g, Gold, Silica) eluting with 0%-5% MeOH in DCM afforded ethyl 3-{[4-(methoxycarbonyl)piperidin-1-yl]methyl}-1H-indole-2-carboxylate (WW-6) as a white foam (0.86 g, 96% yield).

¹H NMR (300 MHz, CHLOROFORM-d) δ 8.66-9.36 (m, 1H), 7.89 (d, J=7.72 Hz, 1H), 7.29-7.50 (m, 2H), 7.13-7.25 (m, 1H), 4.51-4.80 (m, 1H), 4.39-4.49 (m, 2H), 3.67 (s, 3H), 2.82-3.37 (m, 2H), 1.61-2.76 (m, 8H), 1.36-1.50 (m, 3H)

Synthesis of ethyl 1-(1H-indol-3-ylmethyl)piperidine-4-carboxylate (WW-17): To a solution of indole 7 (117 m, 1 mmol) in EtOH (1.5 mL) was added ethyl piperidine-4-carboxylate (157 mg, 1 mmol), formaldehyde solution (74 μL/37% aqueous, 1 mmol), and ZnCl₂ (204 mg, 1.5 mmol) at rt. The mixture was stirred at rt for 2 h. It was then concentrated under reduced pressure. The residue thus obtained was mixed with DCM (20 mL) and aqueous satd. NaHCO₃ (10 mL) and the layers separated. The aqueous layer was extracted with DCM (10 mL×3). The combined organic extracts were washed with brine (20 mL), dried (Na₂SO₄), and concentrated. Chromatographic purification using ISCO (4 g, Gold, Silica) eluting with 0%-10% MeOH (containing 1% NH₄OH) in DCM afforded ethyl 1-(1H-indol-3-ylmethyl)piperidine-4-carboxylate (WW-17) as an off-white solid (148 mg, 52% yield).

¹H NMR (300 MHz, CHLOROFORM-d) δ 8.47 (br. s., 1H), 7.72 (d, J=7.72 Hz, 1H), 7.28-7.35 (m, 1H), 7.18 (dt, J=1.41, 7.49 Hz, 1H), 7.08-7.14 (m, 1H), 7.06 (d, J=2.45 Hz, 1H), 4.05-4.19 (m, 2H), 3.61-3.80 (m, 2H), 2.85-3.05 (m, 2H), 2.25 (tt, J=4.17, 11.00 Hz, 1H), 2.07 (dt, J=2.64, 11.21 Hz, 2H), 1.68-1.92 (m, 4H), 1.18-1.27 (m, 3H) ESI MS: 287 (M+H)⁺

Synthesis of [(3E,4R)-3-ethylidene-1-(4-methoxybenzyl)piperidin-4-yl](5-chloro-1H-indol-2-yl)methanone: Using the procedure described for the synthesis of 9, reaction of 5-chloroindole (0.15 g, 1 mmol) and 6 (0.26 g, 1 mmol) afforded a secondary alcohol intermediate (0.43 g, crude), which was oxidized using the procedure described for WW-7 to obtain [(3E,4R)-3-ethylidene-1-(4-methoxybenzyl)piperidin-4-yl](5-chloro-1H-indol-2-yl)methanone as a light brown foam (0.23 g, 56% over 2 steps).

¹H NMR (300 MHz, CHLOROFORM-d) δ 9.10 (br. s., 1H), 7.67 (s, 1H), 7.32-7.38 (m, 1H), 7.28-7.32 (m, 1H), 7.19-7.25 (m, 3H), 6.84 (d, J=8.48 Hz, 2H), 5.56 (d, J=6.78 Hz, 1H), 4.39 (d, J=5.46 Hz, 1H), 3.80 (s, 3H), 3.42-3.55 (m, 2H), 3.15-3.25 (m, 1H), 3.02-3.13 (m, 1H), 2.79 (d, J=11.11 Hz, 1H), 2.43 (d, J=2.83 Hz, 1H), 1.98-2.21 (m, 2H), 1.69 (dd, J=1.51, 6.78 Hz, 3H)

¹³C NMR (75 MHz, CHLOROFORM-d) δ 193.5, 158.8, 135.4, 135.3, 133.4, 130.5, 130.2, 128.6, 126.8, 126.6, 122.5, 122.2, 113.6, 113.2, 108.1, 62.3, 60.2, 55.3, 50.0, 42.7, 28.9, 13.3

Synthesis of [(3E,4R)-3-ethylidene-1-(4-methoxybenzyl)piperidin-4-yl](5-methoxy-1H-indol-2-yl)methanone: Using the procedure described for the synthesis of 9, reaction of 5-methoxyindole (0.15 g, 1 mmol) and 6 (0.26 g, 1 mmol) afforded a secondary alcohol intermediate (0.36 g, crude), which was oxidized using the procedure described for WW-7 to obtain [(3E,4R)-3-ethylidene-1-(4-methoxybenzyl)piperidin-4-yl](5-methoxy-1H-indol-2-yl)methanone as a brown foam (0.18 g, 46% yield over 2 steps).

¹H NMR (300 MHz, CHLOROFORM-d) δ 9.47 (br. s., 1H), 7.34 (d, J=8.85 Hz, 1H), 7.25-7.27 (m, 1H), 7.19-7.24 (m, 2H), 6.98-7.10 (m, 2H), 6.80-6.87 (m, 2H), 5.56 (d, J=6.97 Hz, 1H), 4.42 (d, J=5.09 Hz, 1H), 3.84 (s, 3H), 3.79 (s, 3H), 3.43-3.57 (m, 2H), 3.11-3.28 (m, 2H), 2.80 (d, J=11.49 Hz, 1H), 2.47 (dd, J=9.04, 11.87 Hz, 1H), 2.00-2.23 (m, 2H), 1.64-1.72 (m, 3H)

¹³C NMR (75 MHz, CHLOROFORM-d) δ 193.6, 158.7, 154.8, 134.9, 133.6, 132.9, 130.6, 130.1, 127.9, 122.3, 118.2, 113.6, 113.2, 108.7, 102.7, 62.3, 60.3, 55.7, 55.2, 50.0, 42.4, 29.0, 13.3 Synthesis of [(3E,4R)-3-ethylidene-1-(4-methoxybenzyl)piperidin-4-yl](5-fluoro-1H-indol-2-yl)methanone: Using the procedure described for the synthesis of 9, reaction of 5-fluoroindole (0.135 g, 1 mmol) and 6 (0.26 g, 1 mmol) afforded a secondary alcohol intermediate (0.33 g, crude), which was oxidized using the procedure described for WW-7 to obtain [(3E,4R)-3-ethylidene-1-(4-methoxybenzyl)piperidin-4-yl](5-fluoro-1H-indol-2-yl)methanone as an off-white solid (0.22 g, 57% yield over 2 steps).

¹H NMR (300 MHz, CHLOROFORM-d) δ 9.40 (br. s., 1H), 7.29-7.44 (m, 2H), 7.18-7.28 (m, 3H), 7.11 (dt, J=2.35, 9.00 Hz, 1H), 6.78-6.92 (m, 2H), 5.57 (q, J=6.78 Hz, 1H), 4.42 (d, J=6.22 Hz, 1H), 3.74-3.83 (m, 3H), 3.43-3.56 (m, 2H), 3.05-3.27 (m, 2H), 2.80 (d, J=11.49 Hz, 1H), 2.44 (dt, J=2.92, 12.01 Hz, 1H), 2.01-2.23 (m, 2H), 1.63-1.73 (m, 3H)

¹³C NMR (75 MHz, CHLOROFORM-d) δ 193.7, 158.7, 135.8, 133.9, 133.4, 130.5, 130.2, 122.5, 115.7, 115.3, 113.6, 113.3, 113.1, 108.8, 108.7, 107.3, 107.0, 62.4, 60.2, 55.2, 50.0, 42.6, 29.0, 13.3

Synthesis of [(3E,4R)-3-ethylidene-1-(4-methoxybenzyl)piperidin-4-yl](6-fluoro-1H-indol-2-yl)methanone: Using the procedure described for the synthesis of 9, reaction of 6-fluoroindole (0.135 g, 1 mmol) and 6 (0.26 g, 1 mmol) afforded a secondary alcohol intermediate (0.35 g, crude), which was oxidized using the procedure described for WW-7 to obtain [(3E,4R)-3-ethylidene-1-(4-methoxybenzyl)piperidin-4-yl](5-fluoro-1H-indol-2-yl)methanone as an off-white solid (0.23 g, 59% yield over 2 steps).

¹H NMR (300 MHz, CHLOROFORM-d) δ 9.73 (br. s., 1H), 7.64 (dd, J=5.37, 8.76 Hz, 1H), 7.22-7.29 (m, 3H), 7.08-7.17 (m, 1H), 6.93 (dt, J=2.17, 9.09 Hz, 1H), 6.84 (d, J=8.67 Hz, 2H), 5.58 (d, J=6.78 Hz, 1H), 4.43 (d, J=5.27 Hz, 1H), 3.79 (s, 3H), 3.46-3.54 (m, 2H), 3.13-3.30 (m, 2H), 2.81 (d, J=11.49 Hz, 1H), 2.47 (dt, J=2.92, 12.01 Hz, 1H), 2.01-2.23 (m, 3H), 1.66-1.72 (m, 3H)

¹³C NMR (75 MHz, CHLOROFORM-d) δ 193.5, 163.9, 160.7, 158.7, 137.8, 137.6, 135.1, 133.4, 130.6, 130.5, 130.1, 124.5, 124.3, 124.2, 122.5, 113.6, 110.9, 110.6, 109.4, 98.3, 97.9, 62.4, 60.3, 55.2, 50.0, 42.3, 29.1, 13.3

### Synthesis of [(3E,4R)-3-ethylidenepiperidin-4-yl](5-fluoro-1H-indol-2-yl)methanone

Debenzylation of [(3E,4R)-3-ethylidene-1-(4-methoxybenzyl)piperidin-4-yl](5-fluoro-1H-indol-2-yl)methanone (0.21 g, 0.54 mmol), using the procedure described for WW-8, resulted in [(3E,4R)-3-ethylidenepiperidin-4-yl](5-fluoro-1H-indol-2-yl)methanone as an off-white solid (0.125 g, 85% yield).

¹H NMR (300 MHz, CHLOROFORM-d) δ 7.30-7.43 (m, 2H), 7.22 (s, 1H), 7.11 (dt, J=2.54, 9.09 Hz, 1H), 5.55 (q, J=7.16 Hz, 1H), 4.52 (d, J=5.84 Hz, 1H), 3.69 (d, J=13.75 Hz, 1H), 3.39-3.50 (m, 1H), 3.31 (d, J=13.56 Hz, 1H), 3.06-3.21 (m, 1H), 2.88-3.03 (m, 1H), 2.19 (dd, J=2.35, 13.66 Hz, 1H), 1.71 (dd, J=1.70, 6.78 Hz, 3H)

¹³C NMR (75 MHz, CHLOROFORM-d) δ 193.6, 134.6, 121.3, 115.7, 115.3, 113.2, 113.1, 108.7, 108.7, 107.1, 106.8, 52.6, 49.6, 42.9, 42.8, 31.2, 13.0

### Synthesis of [(3E,4R)-3-ethylidenepiperidin-4-yl](5-chloro-1H-indol-2-yl)methanone

Debenzylation of [(3E,4R)-3-ethylidene-1-(4-methoxybenzyl)piperidin-4-yl](5-chloro-1H-indol-2-yl)methanone (0.23 g, 0.55 mmol), using the procedure described for WW-8, resulted in [(3E,4R)-3-ethylidenepiperidin-4-yl](5-chloro-1H-indol-2-yl)methanone as a light brown solid (0.07 g, 44% yield).

¹H NMR (300 MHz, CHLOROFORM-d) δ 8.77-9.35 (m, 1H), 7.68 (s, 1H), 7.28-7.39 (m, 2H), 7.18 (s, 1H), 5.49-5.59 (m, 1H), 4.45-4.52 (m, 1H), 3.61-3.73 (m, 1H), 3.28-3.37 (m, 1H), 3.07-3.19 (m, 1H), 2.92-3.03 (m, 1H), 2.14-2.25 (m, 1H), 1.83-1.97 (m, 1H), 1.71 (dd, J=1.70, 6.78 Hz, 3H) ¹³C NMR (75 MHz, CHLOROFORM-d) δ 196.8, 139.1, 138.6, 137.1, 131.2, 129.5, 129.2, 124.8, 124.7, 116.6, 111.2, 55.2, 45.7, 45.5, 33.8, 15.8

### Synthesis of [(3E,4R)-3-ethylidenepiperidin-4-yl](5-methoxy-1H-indol-2-yl)methanone

Debenzylation of [(3E,4R)-3-ethylidene-1-(4-methoxybenzyl)piperidin-4-yl](5-methoxy-1H-indol-2-yl)methanone (0.2 g, 0.49 mmol), using the procedure described for WW-8, resulted in [(3E,4R)-3-ethylidenepiperidin-4-yl](5-methoxy-1H-indol-2-yl)methanone as a brown solid (0.07 g, 50% yield).

### Synthesis of [(3E,4R)-3-ethylidenepiperidin-4-yl](6-fluoro-1H-indol-2-yl)methanone

Debenzylation of [(3E,4R)-3-ethylidene-1-(4-methoxybenzyl)piperidin-4-yl](6-fluoro-1H-indol-2-yl)methanone (0.11 g, 0.29 mmol), using the procedure described for WW-8, resulted in [(3E,4R)-3-ethylidenepiperidin-4-yl](6-fluoro-1H-indol-2-yl)methanone as an off-white semisolid (0.05 g, 53% yield).

¹H NMR (300 MHz, CHLOROFORM-d) δ 9.20 (br. s., 1H), 7.64 (dd, J=5.27, 8.85 Hz, 1H), 7.25 (br. s., 1H), 7.08 (d, J=9.42 Hz, 1H), 6.93 (dt, J=2.26, 9.14 Hz, 1H), 5.54 (d, J=6.97 Hz, 1H), 4.48 (d, J=6.03 Hz, 1H), 3.70 (d, J=13.56 Hz, 1H), 3.32 (d, J=13.56 Hz, 1H), 3.12 (dd, J=3.01, 12.62 Hz, 1H), 2.92-3.03 (m, 1H), 2.14-2.25 (m, 1H), 1.82-1.98 (m, 1H), 1.71 (dd, J=1.70, 6.78 Hz, 3H)

¹³C NMR (75 MHz, CHLOROFORM-d) δ 193.3, 134.6, 124.4, 124.3, 124.1, 121.3, 110.8, 110.5, 109.2, 98.1, 97.7, 52.6, 42.8, 42.7, 31.2, 13.0

### Synthesis of (4E,5R)-4-ethylidene-1,4,5,7-tetrahydro-2,5-ethanoazocino[4,3-b]indol-6(3H)-one (conolidine; WW-1)

Cyclization of WW-8 (0.29 g, 1.16 mmol) with paraformaldehyde, using the literature procedure for the synthesis of conolidine (Nat. Chem. 2011, 3, 449-453), conolidine (WW-1) was obtained as an off-white solid (0.305 g, 98% yield). A solution of this in DCM (20 mL) was treated with 2 M HCl in diethyl ether to obtain conolidine hydrochloride salt.

¹H NMR (300 MHz, METHANOL-d₄) δ 7.63 (d, J=8.29 Hz, 1H), 7.44-7.54 (m, 1H), 7.39 (d, J=6.97 Hz, 1H), 7.19 (d, J=7.16 Hz, 1H), 5.93-6.11 (m, 1H), 5.22 (d, J=16.95 Hz, 1H), 4.65 (d, J=16.95 Hz, 1H), 4.28-4.43 (m, 1H), 4.11 (br. s., 1H), 3.93 (d, J=13.94 Hz, 1H), 3.66-3.79 (m, 1H), 3.43-3.60 (m, 1H), 2.47-2.67 (m, 1H), 2.11-2.26 (m, 1H), 1.63 (dd, J=1.41, 6.88 Hz, 3H)

Elemental Anal. Calcd. for C₁₇H₁₈N₂O·HCl·0.4H₂O: C, 65.86; H, 6.44; N, 9.04. found: C, 65.69; H, 6.49; N, 8.87.

[α]_{D}=−68 (c=0.15, MeOH).

Synthesis of (4E,5R)-10-chloro-4-ethylidene-1,4,5,7-tetrahydro-2,5-ethanoazocino[4,3-b]indol-6(3H)-one (WW-2): Cyclization of [(3E,4R)-3-ethylidenepiperidin-4-yl](5-chloro-1H-indol-2-yl)methanone (33 mg, 0.11 mmol) with paraformaldehyde, using the literature procedure for the synthesis of conolidine (Nat. Chem. 2011, 3, 449-453), WW-2 was obtained as a white solid (28 mg, 85% yield).

¹H NMR (300 MHz, CHLOROFORM-d) δ 9.08 (br. s., 1H), 7.53 (d, J=0.57 Hz, 1H), 7.26-7.32 (m, 2H), 5.48 (q, J=6.59 Hz, 1H), 4.68 (d, J=18.46 Hz, 1H), 4.23 (d, J=18.65 Hz, 1H), 3.98 (d, J=6.22 Hz, 1H), 3.85 (d, J=16.01 Hz, 1H), 3.41 (ddd, J=3.01, 8.29, 13.75 Hz, 1H), 3.30 (d, J=15.82 Hz, 1H), 2.98-3.14 (m, 1H), 1.98-2.21 (m, 2H), 1.45-1.54 (m, 3H)

¹³C NMR (75 MHz, CHLOROFORM-d) δ 193.3, 134.4, 133.4, 131.2, 128.9, 126.9, 125.8, 123.1, 120.2, 119.9, 112.9, 55.0, 53.2, 48.1, 44.3, 22.9, 12.7

Elemental Anal. Calcd. for C₁₇H₁₇ClN₂O·HCl: C, 57.56; H, 5.67; N, 7.89. found: C, 57.57; H, 5.48; N, 7.75.

The free base was converted to its hydrochloride salt by treating with 2 M HCl in diethyl ether to obtain an off-white solid.

### Synthesis of (4E,5R)-4-ethylidene-9-fluoro-1,4,5,7-tetrahydro-2,5-ethanoazocino[4,3-b]indol-6(3H)-one (WW-3)

Cyclization of [(3E,4R)-3-ethylidenepiperidin-4-yl](6-fluoro-1H-indol-2-yl)methanone (47 mg, 0.17 mmol) with paraformaldehyde, using the literature procedure for the synthesis of conolidine (Nat. Chem. 2011, 3, 449-453), WW-3 was obtained as a white solid (42 mg, 87% yield).

¹H NMR (300 MHz, CHLOROFORM-d) δ 9.21 (br. s., 1H), 7.50 (dd, J=5.27, 8.85 Hz, 1H), 7.03 (dd, J=2.07, 9.42 Hz, 1H), 6.87 (dt, J=2.26, 9.23 Hz, 1H), 5.48 (q, J=6.66 Hz, 1H), 4.66-4.82 (m, 1H), 4.19-4.32 (m, 1H), 3.98 (d, J=5.65 Hz, 1H), 3.86 (d, J=15.82 Hz, 1H), 3.41 (ddd, J=3.01, 8.19, 13.66 Hz, 1H), 3.31 (d, J=16.01 Hz, 1H), 2.99-3.16 (m, 1H), 1.98-2.23 (m, 2H), 1.45-1.57 (m, 3H)

¹³C NMR (75 MHz, CHLOROFORM-d) δ 193.0, 133.4, 124.6, 123.0, 122.3, 122.2, 120.9, 109.9, 109.6, 97.8, 97.4, 55.0, 53.3, 48.0, 44.2, 22.9, 12.7

Elemental Anal. Calcd. for C₁₇H₁₇FN₂O·HCl: C, 63.65; H, 5.66; N, 8.73. found: C, 63.42; H, 5.76; N, 8.52.

The free base was converted to its hydrochloride salt by treating with 2 M HCl in diethyl ether to obtain a white solid.

Synthesis of (4E,5R)-10-fluoro-4-ethylidene-1,4,5,7-tetrahydro-2,5-ethanoazocino[4,3-b]indol-6(3H)-one (WW-4): Cyclization of [(3E,4R)-3-ethylidenepiperidin-4-yl](5-fluoro-1H-indol-2-yl)methanone (92 mg, 0.33 mmol) with paraformaldehyde, using the literature procedure for the synthesis of conolidine (Nat. Chem. 2011, 3, 449-453), WW-4 was obtained as an off-white solid (45 mg, 48% yield).

¹H NMR (300 MHz, CHLOROFORM-d) δ 9.17 (br. s., 1H), 7.31 (dd, J=4.14, 8.85 Hz, 1H), 7.19 (dd, J=2.45, 9.23 Hz, 1H), 7.09 (dt, J=2.45, 8.95 Hz, 1H), 5.48 (q, J=6.91 Hz, 1H), 4.67 (d, J=18.65 Hz, 1H), 4.17-4.29 (m, 1H), 3.99 (d, J=6.22 Hz, 1H), 3.81-3.92 (m, 1H), 3.41 (ddd, J=3.01, 8.29, 13.75 Hz, 1H), 3.25-3.35 (m, 1H), 3.07 (dddd, J=1.70, 8.19, 10.10, 13.73 Hz, 1H), 1.98-2.24 (m, 2H), 1.52 (td, J=1.11, 6.83 Hz, 3H)

¹³C NMR (75 MHz, CHLOROFORM-d) δ 193.3, 159.2, 156.0, 133.4, 132.8, 131.5, 128.1, 127.9, 123.0, 120.3, 120.2, 115.7, 115.4, 112.9, 112.7, 105.3, 105.0, 55.0, 53.3, 48.1, 44.2, 22.9, 12.7

Elemental Anal. Calcd. for C₁₇H₁₇FN₂O·HCl·H₂O: C, 62.60; H, 5.75; N, 8.59. found: C, 62.65; H, 5.98; N, 8.38.

The free base was converted to its hydrochloride salt by treating with 2 M HCl in diethyl ether to obtain an off-white solid.

Synthesis of (4E,5R)-10-methoxy-4-ethylidene-1,4,5,7-tetrahydro-2,5-ethanoazocino[4,3-b]indol-6(3H)-one (WW-5): Cyclization of [(3E,4R)-3-ethylidenepiperidin-4-yl](5-mehoxy-1H-indol-2-yl)methanone (50 mg, 0.19 mmol) with paraformaldehyde, using the literature procedure for the synthesis of conolidine (Nat. Chem. 2011, 3, 449-453), WW-5 was obtained as a white solid (25 mg, 45% yield).

¹H NMR (300 MHz, CHLOROFORM-d) δ 9.00 (br. s., 1H), 7.25-7.29 (m, 1H), 7.01 (dd, J=2.35, 8.95 Hz, 1H), 6.89 (d, J=2.45 Hz, 1H), 5.47 (d, J=6.97 Hz, 1H), 4.70 (d, J=18.46 Hz, 1H), 4.24 (d, J=18.46 Hz, 1H), 3.96 (d, J=6.22 Hz, 1H), 3.86-3.91 (m, 1H), 3.84 (s, 3H), 3.40 (ddd, J=3.11, 8.19, 13.75 Hz, 1H), 3.31 (d, J=15.82 Hz, 1H), 3.09 (d, J=13.75 Hz, 1H), 1.99-2.15 (m, 2H), 1.51 (td, J=1.11, 6.83 Hz, 3H)

¹³C NMR (75 MHz, CHLOROFORM-d) δ 193.2, 154.3, 133.5, 131.6, 130.8, 128.0, 122.9, 119.7, 118.1, 112.8, 100.9, 55.8, 55.1, 53.4, 48.1, 44.3, 23.0, 12.7

The free base was converted to its hydrochloride salt by treating with 2 M HCl in diethyl ether to obtain a white solid.

Synthesis of (5-chloro-1H-indol-2-yl)[(3E,4R)-3-ethylidene-1-pentylpiperidin-4-yl]methanone (WW-54): Reaction of [(3E,4R)-3-ethylidenepiperidin-4-yl](5-chloro-1H-indol-2-yl)methanone (29 mg, 0.1 mmol) with 1-bromopentane using General Method A afforded WW-54 as a light yellow oil (17 mg, 47% yield).

¹H NMR (300 MHz, CHLOROFORM-d) δ 9.24 (br. s., 1H), 7.68 (d, J=1.70 Hz, 1H), 7.33-7.41 (m, 1H), 7.27-7.32 (m, 1H), 7.21 (d, J=1.13 Hz, 1H), 5.65 (q, J=6.97 Hz, 1H), 4.40 (d, J=5.27 Hz, 1H), 3.26 (d, J=12.06 Hz, 1H), 3.07 (d, J=12.06 Hz, 1H), 2.88 (d, J=11.68 Hz, 1H), 2.43-2.52 (m, 1H), 2.34-2.43 (m, 2H), 2.17-2.26 (m, 1H), 2.02-2.16 (m, 1H), 1.72 (dd, J=1.60, 6.88 Hz, 3H), 1.53 (td, J=7.49, 15.16 Hz, 2H), 1.23-1.36 (m, 5H), 0.89 (t, J=6.97 Hz, 3H)

¹³C NMR (75 MHz, CHLOROFORM-d) δ 193.5, 135.4, 135.4, 133.2, 128.6, 126.8, 126.6, 122.7, 122.2, 113.2, 108.1, 60.3, 58.5, 50.2, 42.7, 29.9, 28.8, 26.6, 22.6, 14.0, 13.3

The free base was converted to its hydrochloride salt by treating with 2 M HCl in diethyl ether to obtain a off-white solid.

Synthesis of [(3E,4R)-3-ethylidene-1-pentylpiperidin-4-yl](6-fluoro-1H-indol-2-yl)methanone (WW-55): Reaction of [(3E,4R)-3-ethylidenepiperidin-4-yl](6-fluoro-1H-indol-2-yl)methanone (14 mg, 0.05 mmol) with 1-bromopentane using General Method A afforded WW-55 as a light brown oil (12 mg, 70% yield).

¹H NMR (300 MHz, CHLOROFORM-d) δ 9.30 (br. s., 1H), 7.58-7.73 (m, 1H), 7.02-7.17 (m, 1H), 6.87-7.00 (m, 1H), 5.64 (q, J=6.72 Hz, 1H), 4.39 (d, J=5.84 Hz, 1H), 3.21-3.31 (m, 1H), 3.08 (d, J=12.06 Hz, 1H), 2.87 (d, J=11.49 Hz, 1H), 2.29-2.52 (m, 3H), 2.03-2.27 (m, 2H), 1.64-1.75 (m, 3H), 1.46-1.59 (m, 2H), 1.24-1.36 (m, 4H), 0.85-0.94 (m, 3H)

¹³C NMR (75 MHz, CHLOROFORM-d) δ 193.2, 163.9, 160.7, 137.3, 135.2, 133.5, 124.5, 124.3, 124.3, 122.5, 110.9, 110.6, 109.1, 98.1, 97.7, 60.4, 58.6, 50.3, 42.5, 29.9, 29.0, 26.7, 22.6, 14.0, 13.3

The free base was converted to its hydrochloride salt by treating with 2 M HCl in diethyl ether to obtain a light brown solid.

### Synthesis of (5R)-4-ethyl-1,4,5,7-tetrahydro-2,5-ethanoazocino[4,3-b]indol-6(3H)-one (WW-15)

To a solution of conolidine hydrochloride (25 mg, 0.085 mmol) in EtOH (5 mL) was added 10% Pd/C (20 mg) in a Parr hydrogenation bottle under nitrogen. The reaction material was subjected to hydrogenation at 40 psi H₂ pressure using Parr hydrogenation apparatus for 15 h. After filtering through celite, the filtrate was concentrated under reduced pressure to obtain (5R)-4-ethyl-1,4,5,7-tetrahydro-2,5-ethanoazocino[4,3-b]indol-6(3H)-one hydrochloride (WW-15) as an off-white solid (25 mg, quantitative yield).

¹H NMR (300 MHz, METHANOL-d₄) δ 7.57 (d, J=8.10 Hz, 1H), 7.36-7.43 (m, 1H), 7.26-7.33 (m, 1H), 7.03-7.10 (m, 1H), 4.75 (d, J=18.65 Hz, 1H), 4.28 (d, J=18.46 Hz, 1H), 3.60 (q, J=7.10 Hz, 1H), 3.32-3.37 (m, 2H), 2.58-2.69 (m, 1H), 1.98-2.35 (m, 4H), 1.85-1.94 (m, 1H), 1.13-1.20 (m, 2H), 0.97 (t, J=7.35 Hz, 3H).

The free base was converted to its hydrochloride salt by treating with 2 M HCl in diethyl ether to obtain a light brown solid.

### Synthesis of (4E,5R)-4-ethylidene-6-methylidene-1,3,4,5,6,7-hexahydro-2,5-ethanoazocino[4,3-b]indole (WW-16)

This was synthesized from conolidine using a literature method (Tet. Lett. 2016, 57, 375-378). The spectral data matched with the previously reported data.

¹H NMR (300 MHz, CHLOROFORM-d) δ 7.86 (br. s., 1H), 7.42 (d, J=7.91 Hz, 1H), 7.12-7.23 (m, 2H), 7.01-7.10 (m, 2H), 5.38 (s, 1H), 5.18-5.29 (m, 3H), 4.43-4.55 (m, 1H), 4.19-4.31 (m, 1H), 3.90 (br. s., 1H), 3.75-3.84 (m, 1H), 3.40 (ddd, J=2.26, 7.86, 13.23 Hz, 1H), 3.19 (d, J=16.01 Hz, 1H), 3.06 (dd, J=5.09, 12.24 Hz, 1H), 2.09-2.23 (m, 1H), 1.97-2.04 (m, 1H), 1.84-1.94 (m, 1H), 1.45 (dd, J=2.35, 6.88 Hz, 3H).

The free base was converted to its hydrochloride salt by treating with 2 M HCl in diethyl ether to obtain an off-white solid.

### Example 2. Functional Assays of the Compounds of the Invention

Compounds were evaluated for agonist activity using a β-arrestin recruitment assay based on β-galactosidase complementation (PathHunter, DiscoverX). Stable human ACKR3-β-arrestin-2-CHO cells (DiscoverX) were plated into 96-well white-walled assay plates at 15,000 cells/well in Cell Plating Reagent 2 (DiscoveRx) and incubated at 37° C., 5% CO₂ overnight. The next day, test compounds were prepared at 10× concentration in DPBS/1% DMSO and 10 μL was added to the cells. CXCL-12 was included as a positive agonist control. Following a 1.5 hr incubation at 37° C., 55 μL of detection reagent (prepared according to the manufacturer's specifications) was added to each well. The plate was incubated at room temperature for 1 hr, after which luminescence was measured at 1 sec/well using an EnSpire multimode plate reader (PerkinElmer). Relative luminescence units (RLU) were plotted against the log of compound concentration and data were fit to a three-parameter logistic curve to generate EC₅₀ values (GraphPad Prism). EC₅₀ and % Eₘₐₓ values are reported as means±SEM and are the result of at least two independent experiments performed in duplicate. The CXCR4 assay was run similarly except that human CXCR4-C2Cl2 cells (DiscoverX) were plated at 20,000 cells/well in Cell Plating Reagent 9. The next day, the media was removed and cells were incubated with Cell Plating Reagent 4 for 3 hrs, after which the assay was conducted as described above.

The results are depicted in Table 2.

TABLE 2

| | ACKR3 β-arrestin | | CXCR4 β-arrestin |
|---|---|---|---|
| | | % CXCL12 | % CXCL-12Emax |
| Compound | EC50 ± SEM, nM | Emax ± SEM | (n = 2) |
| Conolidine | 27300 ± 440 | 181 ± 4 | 3 |
| WW-2 | >10,000 | n/a | 4 |
| WW-3 | >10,000 | n/a | 1 |
| WW-4 | >10,000 | n/a | 2 |
| WW-5 | 8150 ± 590 | 171 ± 6 | 7 |
| WW-6 | >10,000 | n/a | 7 |
| WW-7 | 1870 ± 96 | 139 ± 2 | 0 |
| WW-8 | >10,000 | n/a | 4 |
| WW-9 | 2070 ± 180 | 132 ± 6 | 10 |
| WW-10 | >10,000 | n/a | 2 |
| WW-12 | 353 ± 19 | 140 ± 11 | 4 |
| WW-14 | 3280 ± 390 | 84 ± 6 | 8 |
| WW-15 | >10,000 | n/a | 7 |
| WW-16 | 4620 ± 410 | 81 ± 5 | 6 |
| WW-17 | 4810 ± 450 | 146 ± 6 | 7 |
| WW-18 | >10,000 | n/a | 8 |
| WW-19 | >10,000 | n/a | 9 |
| WW-20 | 1770 ± 78 | 154 ± 7 | 11 |
| WW-21 | >10,000 | n/a | 10 |
| WW-22 | >10,000 | n/a | 4 |
| WW-23 | 3040 ± 130 | 156 ± 11 | 11 |
| WW-24 | 6050 ± 530 | 154 ± 17 | 5 |
| WW-25 | >10,000 | n/a | 4 |
| WW-26 | >10,000 | n/a | 9 |
| WW-27 | >10,000 | n/a | 4 |
| WW-28 | >10,000 | n/a | 11 |
| WW-29 | 1920 ± 56 | 145 ± 4 | 15 |
| WW-30 | >10,000 | n/a | 7 |
| WW-31 | >10,000 | n/a | 7 |
| WW-32 | >10,000 | n/a | 9 |
| WW-33 | 377 ± 9 | 150 ± 3 | 6 |
| WW-43 | >10,000 | n/a | 10 |
| WW-44 | 4140 ± 110 | 110 ± 7 | 26 |
| WW-45 | >10,000 | n/a | 6 |
| WW-46 | 504 ± 62 | 119 ± 8 | 7 |
| WW-47 | >10,000 | n/a | 18 |
| WW-54 | 1960 ± 27 | 129 ± 4 | 15 |
| WW-55 | 5000 ± 600 | 90 ± 1 | 10 |
| WW-57 | 6450 ± 720 | 96 ± 4 | 5 |
| WW-58 | >10,000 | n/a | 3 |
| WW-59 | >10,000 | n/a | 9 |
| WW-60 | >10,000 | n/a | 11 |
| WW-61 | 527 ± 130 | 156 ± 7 | 10 |
| WW-62 | 3040 ± 320 | 137 ± 3 | 5 |
| WW-63 | 1780 ± 160 | 152 ± 1 | 15 |
| WW-64 | >10,000 | n/a | 16 |
| WW-65 | >10,000 | n/a | 11 |
| WW-66 | >10,000 | n/a | 8 |
| WW-67 | 2460 ± 270 | 135 ± 4 | 12 |
| WW-68 | 3610 ± 390 | 136 ± 3 | 8 |
| WW-69 | 1680 ± 300 | 79 ± 6 | 11 |
| WW-70 | >10,000 | n/a | −2 |
| WW-71 | >10,000 | n/a | 4 |
| WW-72 | 3780 ± 980 | 145 ± 16 | 4 |
| WW-73 | >10,000 | n/a | 9 |
| WW-74 | 177 ± 10 | 172 ± 6 | 3 |

Compounds were evaluated for kappa (KOR), mu (MOR), and delta (DOR) opioid receptor agonist and antagonist activity in three individual cell lines overexpressing Gα_{q16} (CHO-RD-HGA16, Molecular Devices) and the human kappa, mu, and delta opioid receptors, respectively. The day before the assay, cells were plated into 96-well black-walled assay plates at 30,000 cells/well in Ham's F12 supplemented with 10% fetal bovine serum, and 100 units of penicillin and streptomycin. The cells were incubated overnight at 37° C., 5% CO₂. Prior to the assay, Calcium 5 dye (Molecular Devices) was reconstituted according to the manufacturer's instructions. The reconstituted dye was diluted 1:40 in pre-warmed (37° C.) assay buffer (1×HBSS, 20 mM HEPES, 2.5 mM probenecid, pH 7.4 at 37° C.). Growth medium was removed and the cells were gently washed with 100 μL of pre-warmed (37° C.) assay buffer. The cells were incubated for 45 minutes at 37° C., 5% CO₂ in 200 μL of the diluted Calcium 5 dye. For the agonist screen, a single concentration of each test compound and receptor-specific controls (U69,593, DAMGO, and DPDPE for KOR, MOR, and DOR, respectively) were prepared at 10× the desired final concentration in 1% DMSO/assay buffer, aliquoted into a 96-well polystyrene plate, and warmed to 37° C. After the dye-loading incubation period, pretreatment solution was added to each well (25 μL of 9% DMSO/assay buffer) and, after 15 min, the plate was read with a FlexStation II (Molecular Devices). Calcium-mediated changes in fluorescence were monitored every 1.52 seconds over a 60 second time period, with the FlexStation II adding 50 μL of test compounds at the 19 second time point (excitation at 485 nm, detection at 525 nm). Peak kinetic reduction (SoftMax, Molecular Devices) relative fluorescence units (RFU) were used to calculate % control agonist Eₘₐₓ with the equation % Eₘₐₓ=(test compound RFU/control agonist RFU)×100. For the antagonist screens, the same procedure was followed except that the pretreatment solution consisted of the test compounds (prepared at 9× the desired final concentration in 9% DMSO/assay buffer) and the FlexStation added the EC₆₀ concentration (prepared at 10× the desired final concentration in 1% DMSO/assay buffer) of each respective control agonist. Peak RFU were used to calculate % control EC₆₀ inhibition with the equation % inhibition=((EC₆₀ RFU−test compound RFU)/EC₆₀ RFU)×100. When IC₅₀ determinations were made, the same antagonist screen procedure was followed except that cells were pretreated with 8-pt concentration-response curves of the test compounds. Peak RFU were plotted against the log of compound concentration using non-linear regression analysis to generate IC₅₀ values (GraphPad Prism). Each test sample was analyzed in duplicate in two independent experiments.

Compounds were evaluated for activity at CaV 3.2 using stable human CaV3.2-HEK293 cells and the control agonist calcium chloride. The day before the assay, cells were plated into 96-well black-walled assay plates coated with poly-D-lysine in DMEM-F12 supplemented with 10% fetal bovine serum, 100 units penicillin/streptomycin, and 500 μM sodium pyruvate, and incubated at 37° C., 5% CO₂ overnight. Prior to the assay, Calcium 5 dye (Molecular Devices) was reconstituted according to the manufacturer instructions. The reconstituted dye was diluted 1:10 in pre-warmed (37° C.) assay buffer (1×HBSS, 1 M HEPES, 500 mM calcium chloride, pH 7.4 at 37° C.). The cells were incubated for 45 minutes at 37° C., 5% CO₂ in 200 μL of the diluted Calcium 5 dye. Compounds were prepared as described above and the plate was read with a FLIPR Tetra (Molecular Devices). Calcium-mediated changes in fluorescence were monitored every 1 second over a 60 second time period, with the Tetra adding 25 μL of appropriate compound at the 10 second time point (excitation at 470-495 nm, detection at 515-575 nm). Area under the curve kinetic reduction (ScreenWorks, Molecular Devices) RFU were used to generate the same parameters described above.

The results are depicted in Table 3.

TABLE 3

| | KOR Calcium | | MOR Calcium | | | | |
|---|---|---|---|---|---|---|---|
| | % | % | % | % | DOR Calcium | | CaV 3.2 |
| | U69, 593 | U69, 593 | DAMGO | DAMGO | % DPDPE | % DPDPE | % CaCl2 |
| Compound | Emax | Inhibition | Emax | Inhibition | Emax | Inhibition | Inhibition |
| Conolidine | 9 | 11 | 0 | 3 | 0 | 22 | −7 |
| WW-2 | 1 | 22 | 0 | 5 | 0 | 18 | 5 |
| WW-3 | 1 | 14 | 1 | 1 | 0 | 9 | 1 |
| WW-4 | 1 | 14 | 0 | 0 | 1 | 9 | 6 |
| WW-5 | 20 | 17 | 1 | 13 | 2 | 22 | 9 |
| WW-6 | 0 | 22 | 0 | 11 | 0 | 1 | 19 |
| WW-7 | 14 | 38 | 2 | 14 | 6 | 16 | 50 |
| WW-8 | 3 | 4 | 1 | 3 | 5 | 3 | −14 |
| WW-9 | 1 | 23 | 0 | 34 | 0 | 31 | 45 |
| WW-10 | 7 | 8 | 1 | 0 | 4 | 5 | −3 |
| WW-12 | 7 | 69** | 11 | 61, 11** | 6 | 77, 30** | 42 |
| WW-14 | 3 | 6 | 1 | 1 | 5 | 0 | 5 |
| WW-15 | 20 | 18 | 0 | 7 | 0 | 3 | 2 |
| WW-16 | 2 | 23 | 0 | 15 | 0 | 18 | 21 |
| WW-17 | 1 | 5 | 1 | 4 | 0 | 11 | −11 |
| WW-18 | 0 | 8 | 0 | 2 | 0 | 6 | −2 |

**Activity did not confirm in follow up IC50 experiments.

### Example 3. Evaluation of the Activity of Compounds of the Invention Towards ACKR3 and Opioid Receptors

In an effort to identify the target of conolidine among the GPCR family, we undertook a large scale screening program using a β-arrestin recruitment assay based on β-galactosidase complementation (PathHunter, DiscoverX). Over 240 receptors were tested for their ability to be activated or inhibited by conolidine (10 μM) (data not shown). These included 168 GPCRs from the gpcrMAX panel, covering over 60 distinct receptor families such as adrenergic, dopamine, P2Y or serotonin and 74 GPCRs from the orphanMAX panel. The screening pinpointed ACKR3 as the most responsive receptor modulated by conolidine. No activity was detected towards ADRA2B, ADRA2BC and HRH2, whereas the Mast cells G protein-coupled receptor-X2 (MRGPRX2), the cannabinoid receptor 2 (CNR2) and the melatonin receptor 1B (MTNR1B) were only partially activated by conolidine.

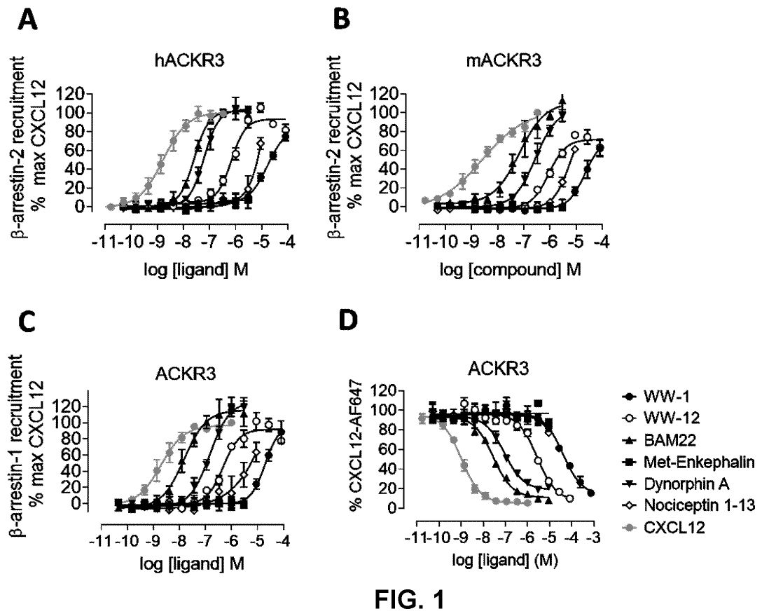

Further confirmation and characterization efforts using the NanoBiT technologies demonstrated that conolidine acts as a full agonist of the newly identified opioid peptide scavenger ACKR3 (FIG. 1). Concentration-response studies were performed with molecules WW-1 (conolidine) and WW-12 on the human and mouse ACKR3 receptors in U87 cells using a NanoBiT-based β-arrestin-2 recruitment assay (PathHunter) to evaluate their efficacy and potency towards the two receptors.

The results are depicted in FIG. 1. For hACKR3, it was observed that all molecules acted as full agonists as compared to the full-agonist chemokine CXCL-12 used as a reference ligand. From concentration-response curves in β-arrestin-2 recruitment assay we obtained EC₅₀ values of 16.6 and 0.7 μM for compounds WW-1 (conolidine) and WW-12 respectively (FIG. 1, section A). Equivalent results were obtained with the NanoBiT-based β-arrestin-1 recruitment assay with the human receptor (FIG. 1, section B). Similar results were obtained on the mouse receptor (mACKR3) (FIG. 1, section C). All molecules acted as full agonists as compared to CXCL-12 and activated the receptor with EC₅₀ values of 22.0 and 0.9 μM for compounds WW-1 (conolidine) and WW-12, respectively. These results show that the compounds of the invention are able to activate both the human and mouse ACKR3 receptors with similar potencies and efficacies. The results depicted in FIG. 1, section D also show that all four compounds compete with CXCL-12, reducing the binding of Alexa647-labelled CXCL-12 to ACKR3. In this binding competition assay, compounds WW-1 and WW-12 competed with CXCL12-AF647 with IC₅₀ values of 39.9 and 2.7 μM, respectively. The unlabelled CXCL-12 had an IC₅₀=1.0 nM. These results show that the compounds according to the invention bind to the orthosteric pocket of ACKR3 and compete with the endogenous ligand of the receptor.

The ability of the different molecules to activate the four opioid receptors MOR, DOR, KOR and NOP that are known to mediate in vivo analgesic effects was tested using the NanoBiT-based β-arrestin-2 recruitment assay in a concentration range of 0.15 nM to 80 μM. For each of these receptors a reference ligand corresponding to an endogenous opioid peptide i.e. Met-enkephalin, BAM22, Dynorphin A and Nociceptin, respectively was included.

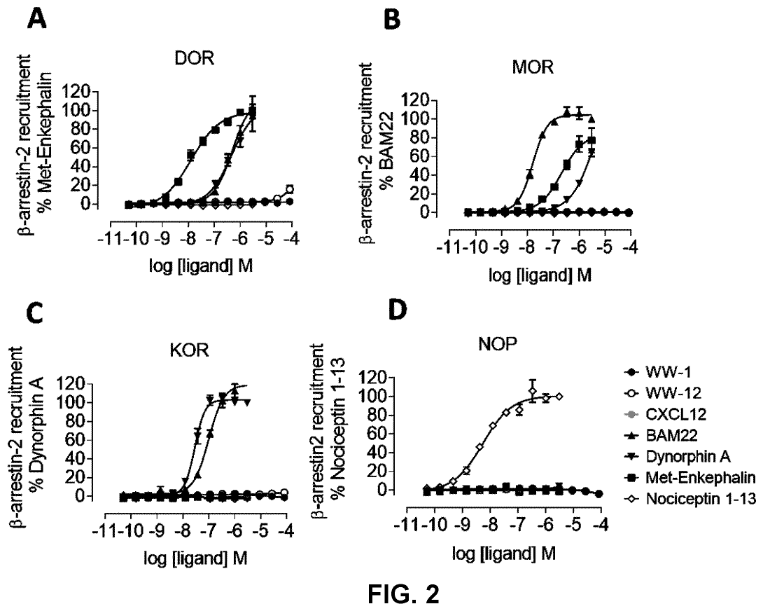

The results are depicted in FIG. 2 section A shows results with receptor DOR, section B shows results with receptor MOR, section C shows results with receptor KOR and section D shows results with receptor NOP. None of the tested compounds (i.e. WW-1 or WW-12) was active in the concentration range tested. These results show that the compounds according to the invention are not active on the classical opioid receptor indicating that analgesic effects can be attributed to the modulation of ACKR3.

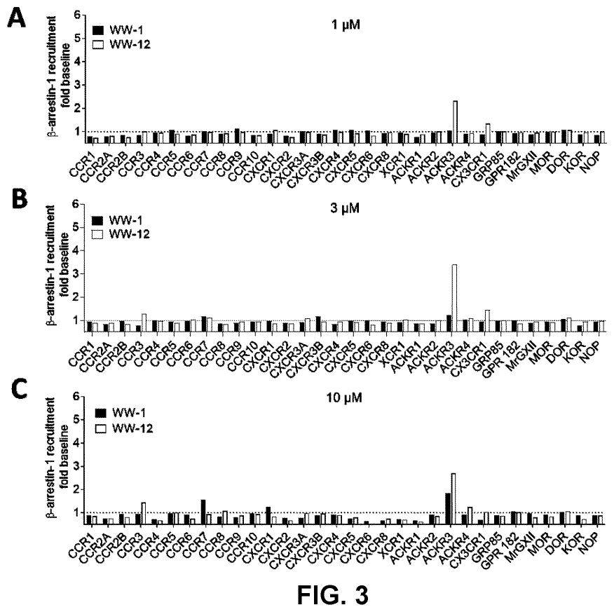

To further assess the selectivity profile of the compounds of the invention towards ACKR3, the ability to activate the 21 classical and 4 atypical chemokines receptors and related receptors was tested using the NanoBiT assay based β-arrestin-1 recruitment described previously. The 4 classical opioid receptors described above were also included in the experimental setup. The compounds WW-1 (conolidine) and WW-12 were tested at three different concentrations, 1 μM, 3 μM and 10 μM. For each receptor, a positive control ligand (cognate chemokine or opioid peptide) at a saturating concentration was added in parallel to verify the receptor functionality At a concentration of 1 μM all four tested compounds activated ACKR3 (FIG. 3, section A), at higher concentrations a slight off-target effect and a weak activation of ACKR4, CX3CR1 and CCR3 were observed (FIG. 3 sections B and C).

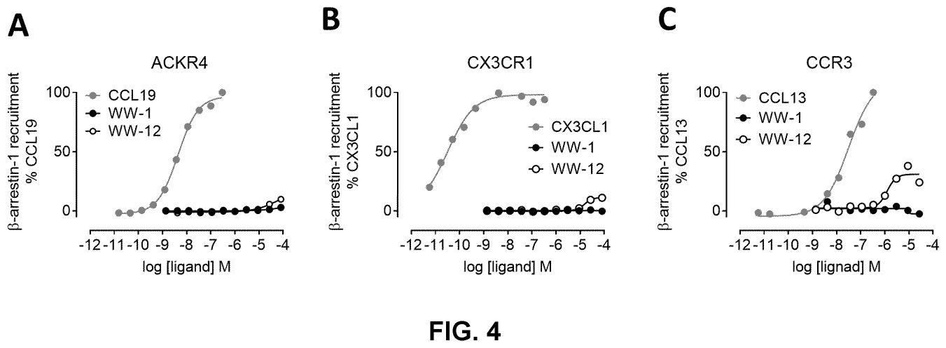

This possible off-target effect was further characterized by comparing the ability of the two molecules to activate receptors ACKR4, CX3CR1 and CCR3 with reference ligands for the receptors, namely CCL19, CX3CL1 and CCL13. The results are depicted in FIG. 4: sections A with receptor ACKR4, section B shows results with receptor CX3CR1 and section C shows results with receptor CCR3. The results demonstrated that the compounds of the invention are selective for the ACKR3 receptor.

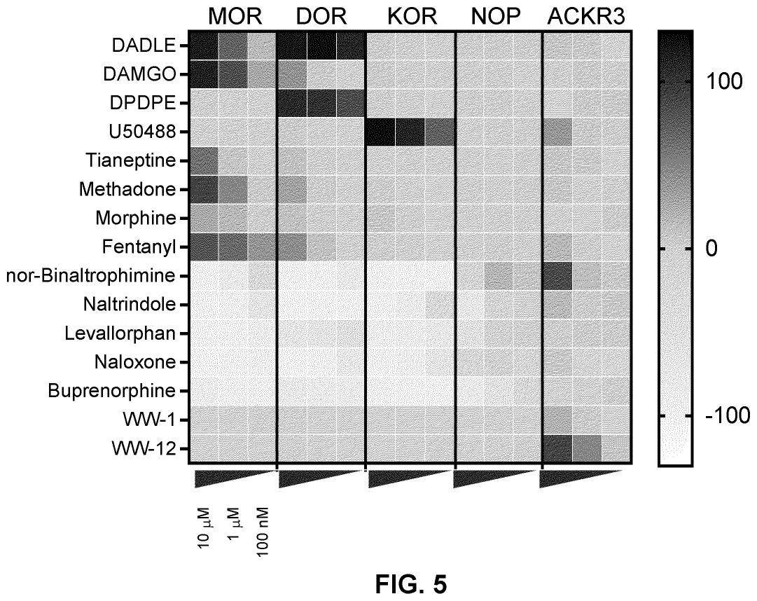

The ability of the compounds of the invention to activate ACKR3 and classical opioid receptors with reference synthetic molecules and approved pain medications such as morphine, fentanyl or buprenorphine that are known to modulate the classical opioid receptors MOR, DOR, KOR and NOP was also tested in NanoBiT based β-arerstin1 recruitment assay. All molecules showed their expected agonist or antagonist activities on their respective opioid receptors. None of them significantly modulated ACKR3 whereas both WW-1 and WW-12 acted as specific ACKR3 activators. The results are depicted in FIG. 5. The results confirm that the compounds of the invention are selective towards the ACKR3 receptor and exhibit a different selectivity profile as compared to reference synthetic molecules and approved pain medications.

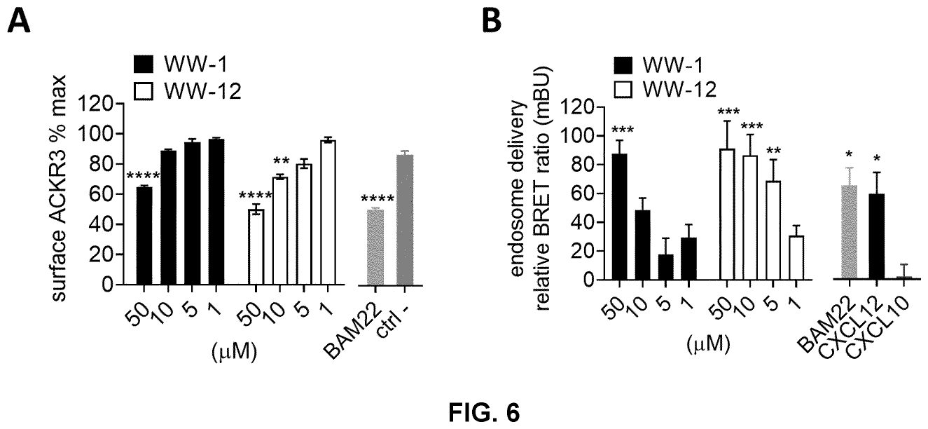

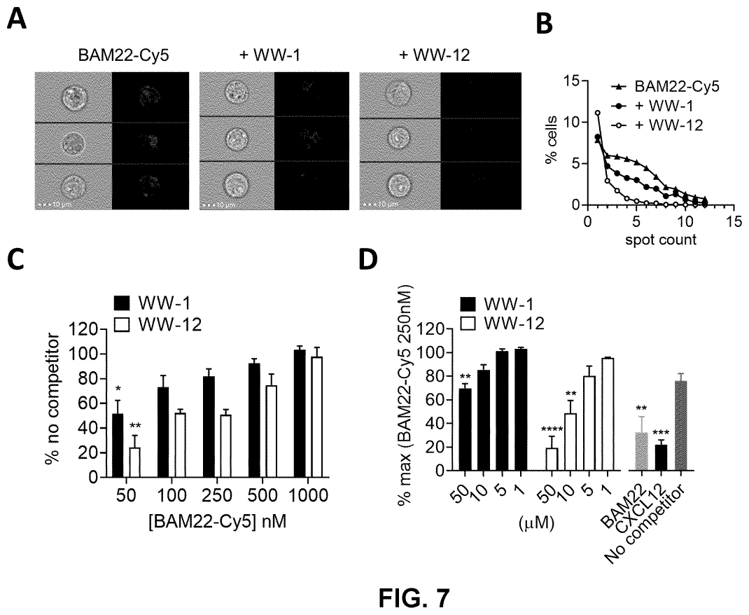

To further characterize the agonist activity of WW-1 (conolidine) and WW-12 and their mode of action towards ACKR3, their ability to induce ACKR3 intracellular internalization (FIG. 6, section A) was evaluated and compared to that of the endogenous agonist opioid ligand BAM22 or an irrelevant peptides used as positive and negative controls, respectively (FIG. 6, section A). WW-1 and WW-12 were able to induce receptor internalization from the plasma membrane. This disappearance was further related to a delivery to the endosomes (FIG. 6 section B) indication that both WW-1 and WW-12 act as ACKR3 agonists. Both WW1 (conolidine) and WW-12 were also shown to be able to block the uptake of labelled endogenous opioid peptide BAM22 (Cy5-BAM22) as visualized by low imaging cytometry (FIG. 7, section). This inhibition was further confirmed by analyzing the ability of WW1 (conolidine) (50 μM) or WW-12 (10 μM) block the uptake of increasing concentrations of Cy5-labelled BAM22 (FIG. 7, section B) or by the ability of increasing concentration of WW-1 (conolidine) or WW-12 to block the uptake of a fixed concentration of Cy5-BAM (250 nM) (FIG. 1 section C) demonstrating that WW-1 and WW-12 are able to restrain the uptake of the endogenous opioid peptide BAM22 by ACKR3 in a concentration-dependent manner.

Material and Methods Used

Nanoluciferase Complementation-Based Assay

Ligand-induced β-arrestin recruitment to chemokine and opioid receptors was monitored by NanoLuc complementation assay (NanoBiT, Promega). In brief, 1.2×10⁶ U87 cells were plated in 10-cm culture dishes and 48 hours later co-transfected with pNBe vectors encoding GPCRs (ACKR3, MOR, DOR, KOR, or NOP) C-terminally tagged with SmBiT and human β-arrestin-1 (arrestin-2) or β-arrestin-2 (arrestin-3) N-terminally fused to LgBiT. 48 hours post-transfection cells were harvested, incubated 25 minutes at 37° C. with Nano-Glo Live Cell substrate diluted 200-fold and distributed into white 96-well plates (5×10⁴ cells per well). Ligand-induced, β-arrestin to GPCRs was evaluated with a Mithras LB940 luminometer (Berthold Technologies, running on MicroWin 2010 5.19 software (Mikrotek Laborsysteme)) for 20 minutes.

For concentration-response curves (FIG. 2), the signal recorded with a saturating concentration of full agonist for each receptor was set as 100%. To evaluate the antagonist properties of ligands, full agonists of each receptor (50 nM BAM22 for MOR, 50 nM dynorphin A for KOR, 70 nM met-enkephalin for DOR, 70 nM nociceptin for NOP and 4 nM CXCL12 for ACKR3) were added after the 20-minute incubation with the ligands. Signal from wells treated with full agonist only was defined as 0% inhibition and signals from wells treated with no agonist were used to set 100% inhibition.

For single dose screening experiments on all chemokine receptors(FIG. 3), the results are represented as percentage of signal monitored with 100 nM of one known agonist chemokine listed in the IUPHAR repository of chemokine receptor ligands which was added as positive control.

Binding Competition Assay

U87-ACKR3 cells were distributed into 96-well plates (1.5×105 cells per well) and incubated with a mixture of 5 nM CXCL12-AF647 and WW-compounds or unlabeled chemokines or opioid peptides at indicated concentrations for 90 minutes on ice, then washed twice with FACS buffer (PBS, 1% BSA, 0.1% NaN3) at 4° C. Dead cells were excluded using Zombie Green viability dye (BioLegend). ACKR3-negative U87 cells were used to evaluate non-specific binding of CXCL12-AF647. 0% receptor binding of CXCL12-AF647 was defined as the signal obtained after addition of 1 μM of unlabeled CXCL12. The signal obtained for CXCL12-AF647 in the absence of unlabeled chemokines was used to define 100% binding. Ligand binding was quantified by mean fluorescence intensity on a BD FACS Fortessa cytometer (BD Biosciences) using FACS Diva 8.01 (BD Biosciences).

Internalization Assay

For determination of receptor surface expression levels by flow cytometry, U87-ACKR3 cells were stimulated with WW-1 or WW-12 (50, 10, 5 1 μM) or BAM22 or ctrl peptides (1 μM) for 45 minutes at 37° C. The remaining surface-bound ligands were then removed by a brief wash with 150 mM NaCl, 50 mM glycine, pH 3 and twice with FACS buffer. Cell surface levels of ACKR3 were then measured by flow cytometry using a saturating concentration of receptor-specific mAb (clones 11G8) and a secondary phycoerythrin-conjugated F(ab′)₂ fragment anti-mouse IgG (Jackson ImmunoResearch). Dead cells were excluded using the Zombie NIR fixable viability dye (BioLegend, catalog #423106, dilution 1:2000). Mean fluorescence intensity was quantified on a Novocyte Quanteon flow cytometer (ACEA Biosciences) using NovoExpress 1.4.1 (ACEA Biosciences).

Ligand-Induced Delivery to Endosomes

Ligand-induced receptor-arrestin delivery to early endosomes was monitored by NanoBRET. In brief, 1.2×106 U87 cells were seeded in 10-cm dishes and co-transfected with plasmids encoding ACKR3, C-terminally tagged with Nanoluciferase and FYVE domain of endofin interacting with phosphatidylinositol 3-phosphate (PI3P) in early endosomes46,47, N-terminally tagged with mNeonGreen. 48h post-transfection, cells were distributed into black 96-well plates (1×105 cells per well) and treated with full-length or processed chemokines. After 2-hour incubation at 37° C., coelenterazine H (10 μM) was added and donor emission (460 nm) and acceptor emission (535 nm) were immediately measured on a GloMax plate reader (Promega).

Visualization of Fluorescently Labeled Opioid-Peptide Uptake

U87-ACKR3 cells were distributed into 96-well plates (2×105 cells per well in Opti-MEM). After 15-minute incubation at 37° C. with WW-1 or WW-12 or controls, Cy5-labeled BAM22 was added, incubated for 40 minutes at 37° C. and washed twice with FACS buffer. Dead cells were excluded using Zombie Green viability dye. Images of 1×104 in-focus living single cells were acquired with an ImageStream MKII imaging flow cytometer (Amnis, running on the INSPIRE Mark II software (EMD Millipore)) using 40× magnification (60× magnification for smNPCs). Samples were analyzed using Ideas6.2 software. The number of spots per cell was determined using a mask-based software wizard.

1.-15. (canceled)

16. A compound of formula (2), optionally wherein the compound further contains a label attached thereto; or a stereoisomer, enantiomer, racemic, thereof: 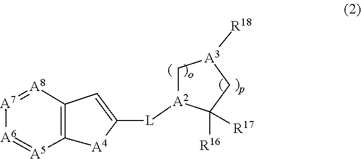
wherein,
o is an integer selected from 0, 1, 2 or 3;
p is an integer selected from 0, 1, 2, 3 or 4;
A² is selected from N or CR¹⁹;
A³ is selected from N or CR²⁰;
A⁴ is selected from NR¹¹, O, S or CR²⁴ R²⁵;
A⁵ is selected from N or CR¹²;
A⁶ is selected from N or CR¹³;
A⁷ is selected from N or CR¹⁴;
A⁸ is selected from N or CR¹⁵;
wherein at least one of A² or A³ is N;
wherein at most one of A⁵ to A⁸ is N;
L is selected from —C═O, —C(O)—NH—, and CHR²¹;
R¹¹ is selected from the group consisting of hydrogen, deuterium, alkyl, —S(O)₂R²², aryl, —S(O)R²², —SO₂NR²²R²³; and wherein said alkyl or aryl can be unsubstituted or substituted with one or more Z¹;
R¹² is selected from the group consisting of hydrogen, deuterium, halogen, —OR²³, cyano, —C(O)R²³ alkyl, trifluoromethyl, trifluoromethoxy, cycloalkyl, aryl, heterocyclyl, heteroaryl, arylalkyl, and nitro;
R¹³ is selected from the group consisting of hydrogen, deuterium, halogen, —OR²³, cyano, —C(O)R²³ alkyl, trifluoromethyl, trifluoromethoxy, cycloalkyl, aryl, heterocyclyl, heteroaryl, arylalkyl, and nitro;
R¹⁴ is selected from the group consisting of hydrogen, deuterium, halogen, —OR²³, cyano, —C(O)R²³ alkyl, trifluoromethyl, trifluoromethoxy, cycloalkyl, aryl, heterocyclyl, heteroaryl, arylalkyl, and nitro;
R¹⁵ is selected from the group consisting of hydrogen, deuterium, halogen, —OR²³, cyano, —C(O)R²³ alkyl, trifluoromethyl, trifluoromethoxy, cycloalkyl, aryl, heterocyclyl, heteroaryl, arylalkyl, and nitro;
R¹⁶ is selected from the group consisting of hydrogen, deuterium, alkyl, halogen, and —OR²³;
R¹⁷ is selected from the group consisting of hydrogen, deuterium, alkyl, halogen, and —OR²³;
or R¹⁶ and R¹⁷ together with the carbon atom to which they are attached from a group selected from —C═CH-alkyl, —C═N—OH, —C═N—O—Si(CH₃)₂C(CH₃)₃, or a saturated or unsaturated 3-, 4-, 5-, 6- or 7-membered ring;
R¹⁸ is selected from the group consisting of halogen, —NH₂, —NHR²², alkyl, deuterium, arylalkyl, —S(O)₂R²², —C(O)OR²³, —S(O)R²², heteroaryl, cycloalkyl, aryl, and heterocyclyl; and wherein said alkyl, arylalkyl, heteroaryl, cycloalkyl, aryl, heterocyclyl, or arylalkyl can be unsubstituted or substituted with one or more Z²;
R¹⁹ is selected from the group consisting of hydrogen, alkyl, halogen, and —OR²³;
R²⁰ is selected from the group consisting of hydrogen, alkyl, halogen, and —OR²³;
R²¹ is selected from the group consisting of —OH, —COOR²³, —C(O)NH₂, hydrogen, and —OR²³;
each R²² is independently selected from the group consisting of alkyl, aryl, cycloalkyl, arylalkyl, heterocyclyl, and heteroaryl;
each R²³ is independently selected from the group consisting of hydrogen, alkyl, aryl, cycloalkyl, arylalkyl, heterocyclyl, and heteroaryl;
R²⁴ is selected from the group consisting of hydrogen, deuterium, halogen, —OR²³, cyano, —C(O)R²³ alkyl, trifluoromethyl, trifluoromethoxy, cycloalkyl, aryl, heterocyclyl, heteroaryl, arylalkyl, and nitro;
R²⁵ is selected from the group consisting of hydrogen, deuterium, halogen, —OR²³, cyano, —C(O)R²³ alkyl, trifluoromethyl, trifluoromethoxy, cycloalkyl, aryl, heterocyclyl, heteroaryl, arylalkyl, and nitro;
each Z¹ is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z² is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
or a solvate, hydrate, pharmaceutically acceptable salt or prodrug thereof,
with the proviso that when A³ is C, then R¹⁸ is not alkyl or benzyl;
with the proviso that when A² is N, then R¹⁴ is not chloro, methyl or trifluoromethyl;
with the proviso that when L is —C(O)—NH—, then R¹⁵ is not bromo, —OR²³, phenyl or pyridyl;
and with the proviso that when A³ is N, then R¹⁸ is not methyl, p-methoxy-benzyl, phenyl sulfone or diphenylmethyl;
and with the proviso that the said compound is not
(5-chloro-1H-indol-2-yl)-(4-methylpiperazin-1-yl)methanone;
(7-amino-5-chloro-1H-indol-2-yl)-(4-methylpiperazin-1-yl)methanone;
(5-chloro-7-methyl-1H-indol-2-yl)-(3-pyrrolidin-1-ylazetidin-1-yl)methanone;
tert-butyl 4-(1H-indole-2-carbonyl)piperazine-1-carboxylate;
tert-butyl 4-(1-methylindole-2-carbonyl)piperazine-1-carboxylate;
1H-indol-2-yl-[4-(1-phenylethyl)piperazin-1-yl]methanone;
(1-methylindol-2-yl)-[4-(1-phenylethyl)piperazin-1-yl]methanone;
[4-(1,3-benzodioxol-5-ylmethyl)piperazin-1-yl]-(1H-indol-2-yl)methanone;
[4-(2-hydroxy-2-methyl-propyl)piperazin-1-yl]-(5-methoxy-1H-indol-2-yl)methanone;
4-benzo[1,2,5]oxadiazol-5-yl-1H-indole-2-carboxylic acid [1-(2-azepan-1-yl-ethyl)-piperidin-4-yl]-amide;
4-benzo[1,3]dioxol-5-yl-1H-indole-2-carboxylic acid [1-(2-azepan-1-yl-ethyl)-piperidin-4-yl]-amide;
4-hydroxy-1H-indole-2-carboxylic acid [1-(2-azepan-1-yl-ethyl)-piperidin-4-yl]-amide;
Etyl 1-(1H-indole-2-carbonyl)piperidine-4-carboxylate; or
1-(1H-indole-2-carbonyl)piperidine-4-carboxylic acid.

17. The compound according to claim 16, wherein the compound is of structural formula (2A):

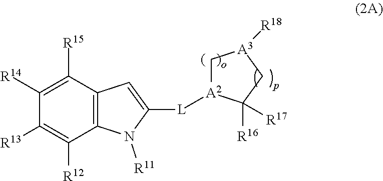

18. The compound according to claim 16, wherein the compound is of structural formulae (2B), (2C), (2D), (2E), or (2F):

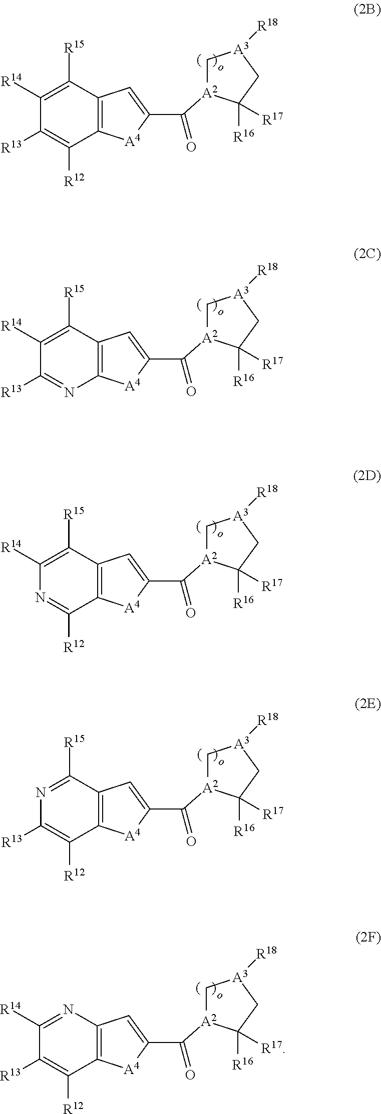

19. The compound according to claim 16, wherein the compound is of structural formulae (2G), or (2H):

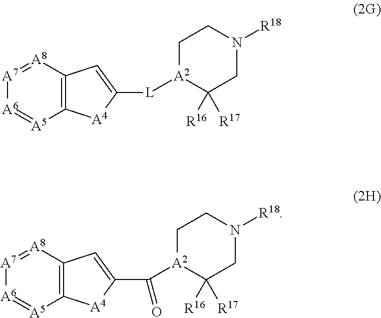

20. The compound according to claim 16, wherein the compound is of structural formulae (2I), (2J), (2K), or (2L):

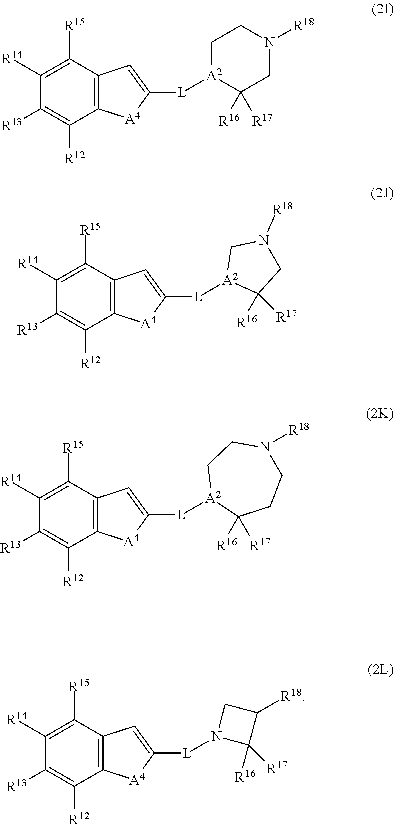

21. A compound selected from:
ethyl 1-(1H-indol-3-ylmethyl)piperidine-4-carboxylate; and
ethyl 3-[(4-methoxycarbonyl-1-piperidyl)methyl]-1H-indole-2-carboxylate, optionally comprising a label.

22. A pharmaceutical composition comprising the compound according to claim 16 and a pharmaceutically acceptable carrier.

23. A method of treating a patient suffering from an ACKR3 mediated disorder, the method comprising administering an effective amount of the compound according to claim 16 to a subject in need thereof.

24. The method according to claim 23, wherein the ACKR3 mediated disorder is selected from pain, distress dysfunction diseases or conditions, cancers, atherosclerotic vascular disease, cardiovascular diseases, fibrosis (e.g. cardiac fibrosis), inflammatory or autoimmune diseases and conditions, conditions of excessive or abnormal vascularization (e.g. wound healing), stem cell differentiation and mobilization disorders, brain and neuronal dysfunctions (e.g. Alzheimer's disease, multiple sclerosis and demyelinating diseases), kidney dysfunction, renal dysfunction, preeclampsia, human immunodeficiency virus (HIV) infection and obesity in a subject

25. A method of treating a subject suffering from pain or a distress dysfunction disease or condition, cancer, atherosclerotic vascular disease, cardiovascular diseases, fibrosis (e.g. cardiac fibrosis), inflammatory or autoimmune disease or conditions, condition of excessive or abnormal vascularization, such as. wound healing, stem cell differentiation and mobilization disorder, brain or neuronal dysfunction such as Alzheimer's disease, multiple sclerosis or demyelinating disease, kidney dysfunction, renal dysfunction, preeclampsia, human immunodeficiency virus (HIV) infection, the method comprising, administering to said subject an effective amount of a compound of formula (2) or a stereoisomer, enantiomer, racemic, thereof: 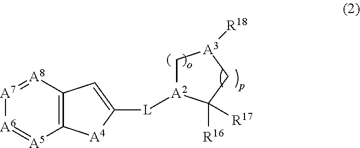
wherein,
o is an integer selected from 0, 1, 2 or 3;
p is an integer selected from 0, 1, 2, 3 or 4;
A² is selected from N or CR⁹;
A³ is selected from NR¹¹ or CR²⁰;
A⁴ is selected from N, O, S or CR²⁴ R²⁵;
A⁵ is selected from N or CR¹²;
A⁶ is selected from N or CR³;
A⁷ is selected from N or CR¹⁴;
A⁸ is selected from N or CR¹⁵;
wherein at least one of A² or A³ is N;
wherein at most one of A⁵ to A⁸ is N;
L is selected from —C═O, —C(O)—NH—, and CHR²¹;
R¹¹ is selected from the group consisting of hydrogen, deuterium, alkyl, —S(O)₂R²², aryl, —S(O)R²², —SO₂NR²²R²³; and wherein said alkyl or aryl can be unsubstituted or substituted with one or more Z¹;
R¹² is selected from the group consisting of hydrogen, deuterium, halogen, —OR²³, cyano, —C(O)R²³ alkyl, trifluoromethyl, trifluoromethoxy, cycloalkyl, aryl, heterocyclyl, heteroaryl, arylalkyl, and nitro;
R¹³ is selected from the group consisting of hydrogen, deuterium, halogen, —OR²³, cyano, —C(O)R²³ alkyl, trifluoromethyl, trifluoromethoxy, cycloalkyl, aryl, heterocyclyl, heteroaryl, arylalkyl, and nitro;
R¹⁴ is selected from the group consisting of hydrogen, deuterium, halogen, —OR²³, cyano, —C(O)R²³ alkyl, trifluoromethyl, trifluoromethoxy, cycloalkyl, aryl, heterocyclyl, heteroaryl, arylalkyl, and nitro;
R¹⁵ is selected from the group consisting of hydrogen, deuterium, halogen, —OR²³, cyano, —C(O)R²³ alkyl, trifluoromethyl, trifluoromethoxy, cycloalkyl, aryl, heterocyclyl, heteroaryl, arylalkyl, and nitro;
R¹⁶ is selected from the group consisting of hydrogen, deuterium, alkyl, halogen, and —OR²³;
R¹⁷ is selected from the group consisting of hydrogen, deuterium, alkyl, halogen, and —OR²³;
or R¹⁶ and R¹⁷ together with the carbon atom to which they are attached from a group selected from —C═CH-alkyl, —C═N—OH, —C═N—O—Si(CH₃)₂C(CH₃)₃, or a saturated or unsaturated 3-, 4-, 5-, 6- or 7-membered ring;
R¹⁸ is selected from the group consisting of hydrogen, deuterium, halogen, —NH₂, —NHR²², alkyl, arylalkyl, —S(O)₂R²², —C(O)OR²³, —S(O)R²², heteroaryl, cycloalkyl, aryl, and heterocyclyl; and
wherein said alkyl, arylalkyl, heteroaryl, cycloalkyl, aryl, heterocyclyl, or arylalkyl can be unsubstituted or substituted with oner or more Z;
R¹⁹ is selected from the group consisting of hydrogen, alkyl, halogen, and —OR²³;
R²⁰ is selected from the group consisting of hydrogen, alkyl, halogen, and —OR²³;
R²¹ is selected from the group consisting of —OH, —COOR²³, —C(O)NH, hydrogen, and —OR²³;
each R²² is independently selected from the group consisting of alkyl, aryl, cycloalkyl, arylalkyl, heterocyclyl, and heteroaryl;
each R²³ is independently selected from the group consisting of hydrogen, alkyl, aryl, cycloalkyl, arylalkyl, heterocyclyl, and heteroaryl;
R²⁴ is selected from the group consisting of hydrogen, deuterium, halogen, —OR²³, cyano, —C(O)R²³ alkyl, trifluoromethyl, trifluoromethoxy, cycloalkyl, aryl, heterocyclyl, heteroaryl, arylalkyl, and nitro;
R²⁵ is selected from the group consisting of hydrogen, deuterium, halogen, —OR²³, cyano, —C(O)R²³ alkyl, trifluoromethyl, trifluoromethoxy, cycloalkyl, aryl, heterocyclyl, heteroaryl, arylalkyl, and nitro;
each Z¹ is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
each Z² is independently selected from the group consisting of —OR²³, halogen, alkyl, —NH₂, —NHR²², —COOR²³, cycloalkyl, trifluoromethyl, trifluoromethoxy, aryl, arylalkyl, heterocyclyl, heteroaryl, —OH, cyano and nitro;
or a solvate, hydrate, pharmaceutically acceptable salt or prodrug thereof, with the proviso that when A³ is N, then R¹⁸ is not diphenylmethyl;
with the proviso that when A³ is C, then R¹⁸ is not alkyl or benzyl;
with the proviso that when A² is N, then R¹⁴ is not chloro, methyl or trifluoromethyl;
with the proviso that when L is —C(O)—NH—, then R¹⁵ is not bromo, —OR²³, phenyl, pyridyl, and with the proviso that the said compound is not
tert-butyl 4-(1H-indole-2-carbonyl)piperazine-1-carboxylate;
tert-butyl 4-(1-methylindole-2-carbonyl)piperazine-1-carboxylate;
1H-indol-2-yl-[4-(1-phenylethyl)piperazin-1-yl]methanone;
(1-methylindol-2-yl)-[4-(1-phenylethyl)piperazin-1-yl]methanone;
[4-(1,3-benzodioxol-5-ylmethyl)piperazin-1-yl]-(1H-indol-2-yl)methanone;
[4-(2-hydroxy-2-methyl-propyl)piperazin-1-yl]-(5-methoxy-1H-indol-2-yl)methanone;
4-benzo[1,2,5]oxadiazol-5-yl-1H-indole-2-carboxylic acid [1-(2-azepan-1-yl-ethyl)-piperidin-4-yl]-amide;
4-benzo[1,3]dioxol-5-yl-1H-indole-2-carboxylic acid [1-(2-azepan-1-yl-ethyl)-piperidin-4-yl]-amide; or
4-hydroxy-1H-indole-2-carboxylic acid [1-(2-azepan-1-yl-ethyl)-piperidin-4-yl]-amide
thereby treating said pain or distress dysfunction disease or condition, cancer, atherosclerotic vascular disease, cardiovascular diseases, fibrosis (e.g. cardiac fibrosis), inflammatory or autoimmune disease or conditions, condition of excessive or abnormal vascularization, such as. wound healing, stem cell differentiation and mobilization disorder, brain or neuronal dysfunction such as Alzheimer's disease, multiple sclerosis or demyelinating disease, kidney dysfunction, renal dysfunction, preeclampsia, human immunodeficiency virus (HIV) infection or obesity in said subject.

26. A method of treating a subject suffering from a distress dysfunction disease or condition, cancer, atherosclerotic vascular disease, cardiovascular diseases, fibrosis such as cardiac fibrosis, inflammatory or autoimmune disease or condition, condition of excessive or abnormal vascularization such as wound healing, stem cell differentiation and mobilization disorder, brain and/or neuronal dysfunction such as Alzheimer's disease, multiple sclerosis or demyelinating disease, kidney dysfunction, renal dysfunction, preeclampsia, human immunodeficiency virus (HIV) infection or obesity, said method comprising administering to said subject an effective amount of a compound of formula (1A) (1B) or (1C); or a stereoisomer, enantiomer, racemic, thereof: 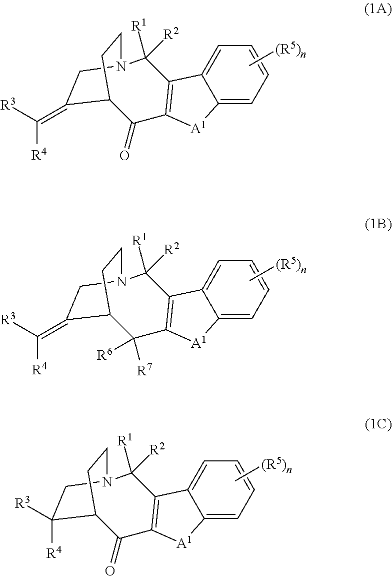
wherein
n is an integer selected from 0, 1, 2 or 3;
A¹ is selected from the group consisting of a substituted nitrogen or carbon atom, substituents selected from the group consisting of hydrogen, deuterium, alkyl, heteroatom substituted alkyl, alkenyl, aryl, heteroaryl, cycloalkyl, heteroatom substituted cycloalkyl, S, SO, SO₂, OR⁹, NR⁹;
R¹ is selected from the group consisting of hydrogen, deuterium, alkyl, heteroatom substituted alkyl, alkenyl, aryl, heteroaryl, cycloalkyl and heteroatom substituted cycloalkyl;
R² is selected from the group consisting of hydrogen, deuterium, alkyl, heteroatom substituted alkyl, alkenyl, aryl, heteroaryl, cycloalkyl and heteroatom substituted cycloalkyl;
R³ is selected from the group consisting of hydrogen, deuterium, alkyl, heteroatom substituted alkyl, alkenyl, aryl, heteroaryl, halogen, cycloalkyl and heteroatom substituted cycloalkyl;
R⁴ is selected from the group consisting of hydrogen, deuterium, alkyl, heteroatom substituted alkyl, alkenyl, aryl, heteroaryl, halogen, and cycloalkyl and heteroatom substituted cycloalkyl;
or R³ and R⁴ together with the atom to which they are attached can form a saturated or unsaturated 5-, 6-, or 7-membered ring;
R⁵ is selected from the group consisting of deuterium, halogen, alkyl, heteroatom substituted alkyl, alkenyl, aryl, heteroaryl, cycloalkyl and heteroatom substituted cycloalkyl;
R⁶ is selected from the group consisting of hydrogen, deuterium, NH₂, NR⁸R⁹, OR⁹, and R¹;
R⁷ is selected from the group consisting of hydrogen, deuterium, NH₂, NR⁸R⁹, OR⁹, and R¹;
or R⁶ and R⁷ together with the carbon atom to which they are attached from a group selected from the group consisting of —CH═CH₂, —CH═CH-alkyl, and —CH═N—OH;
R is selected from the group consisting of deuterium, alkyl, heteroatom substituted alkyl, alkenyl, aryl, heteroaryl, cycloalkyl and heteroatom substituted cycloalkyl;
R⁹ is selected from the group consisting of hydrogen, deuterium, alkyl, heteroatom substituted alkyl, alkenyl, aryl, heteroaryl, cycloalkyl and heteroatom substituted cycloalkyl;
or a solvate, hydrate, pharmaceutically acceptable salt or prodrug thereof,
or a solvate, hydrate, pharmaceutically acceptable salt or prodrug thereof,
thereby treating said distress dysfunction disease or condition, cancer, atherosclerotic vascular disease, cardiovascular diseases, fibrosis such as cardiac fibrosis, inflammatory or autoimmune disease or condition, condition of excessive or abnormal vascularization such as wound healing, stem cell differentiation and mobilization disorder, brain and/or neuronal dysfunction such as Alzheimer's disease, multiple sclerosis or demyelinating disease, kidney dysfunction, renal dysfunction, preeclampsia, human immunodeficiency virus (HIV) infection or obesity in said subject.

27. The method according to claim 26, wherein the compound is of structural formulae (1AA), (1BB) or (1CC):

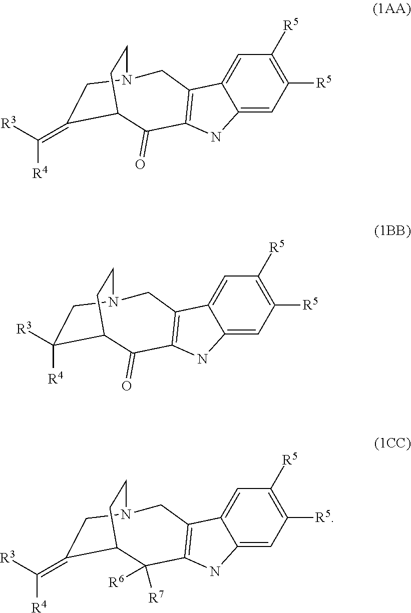

28. A method for in vitro or ex vivo diagnosis, prediction, prognosis and/or monitoring of a disease or condition characterized by an aberrant level of ACKR3 polypeptide, comprising the steps of
obtaining a biological sample obtained from a subject,
contacting said biological sample with a compound according to claim 16, wherein said compound is covalently linked to a detectable label,
determining the level of ACKR3 polypeptide in said biological sample by detecting said compound, and
diagnosing, predicting, prognosing and/or monitoring the disease or condition based on the level of ACKR3 polypeptide.

29. A method for in vitro or ex vivo diagnosis, prediction, prognosis and/or monitoring of a disease or condition characterized by an aberrant level of ACKR3 polypeptide, comprising the steps of
obtaining a biological sample obtained from a subject,
contacting said biological sample with a compound according to claim 17, wherein said compound is covalently linked to a detectable label,
determining the level of ACKR3 polypeptide in said biological sample by detecting said compound, and
diagnosing, predicting, prognosing and/or monitoring the disease or condition based on the level of ACKR3 polypeptide.

30. A method for in vitro or ex vivo diagnosis, prediction, prognosis and/or monitoring of a disease or condition characterized by an aberrant level of ACKR3 polypeptide, comprising the steps of
obtaining a biological sample obtained from a subject,
contacting said biological sample with a compound according to claim 18, wherein said compound is covalently linked to a detectable label,
determining the level of ACKR3 polypeptide in said biological sample by detecting said compound, and
diagnosing, predicting, prognosing and/or monitoring the disease or condition based on the level of ACKR3 polypeptide.

31. A method for in vitro or ex vivo diagnosis, prediction, prognosis and/or monitoring of a disease or condition characterized by an aberrant level of ACKR3 polypeptide, comprising the steps of
obtaining a biological sample obtained from a subject,
contacting said biological sample with a compound according to claim 19, wherein said compound is covalently linked to a detectable label,
determining the level of ACKR3 polypeptide in said biological sample by detecting said compound, and
diagnosing, predicting, prognosing and/or monitoring the disease or condition based on the level of ACKR3 polypeptide.

32. A method for in vitro or ex vivo diagnosis, prediction, prognosis and/or monitoring of a disease or condition characterized by an aberrant level of ACKR3 polypeptide, comprising the steps of
obtaining a biological sample obtained from a subject,
contacting said biological sample with a compound according to claim 20, wherein said compound is covalently linked to a detectable label,
determining the level of ACKR3 polypeptide in said biological sample by detecting said compound, and
diagnosing, predicting, prognosing and/or monitoring the disease or condition based on the level of ACKR3 polypeptide.

33. A method for in vitro or ex vivo diagnosis, prediction, prognosis and/or monitoring of a disease or condition characterized by an aberrant level of ACKR3 polypeptide, comprising the steps of
obtaining a biological sample obtained from a subject,
contacting said biological sample with a compound according to claim 21, wherein said compound is covalently linked to a detectable label,
determining the level of ACKR3 polypeptide in said biological sample by detecting said compound, and
diagnosing, predicting, prognosing and/or monitoring the disease or condition based on the level of ACKR3 polypeptide.

34. The compound according to claim 16, wherein the compound further contains a label attached thereto.

35. A kit for diagnosing, predicting, prognosing and/or monitoring a disease or condition characterized by an aberrant level of ACKR3 polypeptide in a subject, the kit comprising:
(a) the compound according to claim 16; and
(b) a reference value of the level of ACKR3 polypeptide, wherein said reference value represents a known diagnosis, prediction and/or prognosis of the disease or condition characterized by an aberrant level of ACKR3 polypeptide.

36. A kit for diagnosing, predicting, prognosing and/or monitoring a disease or condition characterized by an aberrant level of ACKR3 polypeptide in a subject, the kit comprising:
(a) the compound according to claim 21; and
(b) a reference value of the level of ACKR3 polypeptide, wherein said reference value represents a known diagnosis, prediction and/or prognosis of the disease or condition characterized by an aberrant level of ACKR3 polypeptide.
